(19) United States
(12) Patent Application Publication
(10) Pub. No.: US 2023/0220403 A1
(43) Pub. Date: Jul. 13, 2023

(54) SPECIFIC DECOLONIZATION OF ANTIBIOTIC RESISTANT BACTERIA FOR PROPHYLACTIC PURPOSES

(71) Applicant: Eligo Bioscience, PARIS (FR)

(72) Inventors: Xavier DUPORTET, PARIS (FR); Igor STZEPOURGINSKI, PARIS (FR); Jesus FERNANDEZ RODRIGUEZ, PARIS (FR)

(21) Appl. No.: 18/001,504

(22) PCT Filed: Jun. 14, 2021
(86) PCT No.: PCT/EP2021/065986

§ 371 Date: Dec. 12, 2022

Related U.S. Application Data
(60) Provisional application No. 63/038,386, filed on Jun. 12, 2020.
(60) Provisional application No. 63/048,034, filed on Jul. 3, 2020.
(60) Provisional application No. 63/132,190, filed on Dec. 30, 2020.
(60) Provisional application No. 63/137,989, filed on Jan. 15, 2021.
(60) Provisional application No. 63/148,479, filed on Feb. 11, 2021.

(51) Int. Cl. C12N 15/63; C12N 9/14

(52) U.S. Cl. CPC C12N 15/63; C12N 9/14; C12N 2320/30

(57) ABSTRACT

The invention relates to methods, kits, and compositions for reducing the level of or eliminating antimicrobial resistant (AMR) bacteria in situ. The invention encompasses compositions and methods for selectively eradicating antibiotic resistance in bacteria that carry antibiotic resistance genes in the microbiota using packaged phagemids. The microbiota can be intestinal and the packaged phagemids can be administered to a healthy subject or patient, for example, orally, rectally (e.g., in an enema), vaginally, nasally or to the skin. The phagemid encodes a nuclease or other enzyme that genetically modifies the DNA encoding the antibiotic resistance gene so that the bacteria can then be eliminated with the antibiotic.

### BACKGROUND OF THE INVENTION

Antimicrobial resistant (AMR) infections are poised to represent an increasing threat in the near future, particularly in the hospital setting where they are the most prevalent and put patients at risk as they increase the length and cost of hospitalization, but also more generally in immunocompromised patients, and dramatically impair prognosis.

In most cases the bacteria responsible for AMR infections in hospitalized and/or immunocompromised patients originate from the patients themselves. Indeed, microbial communities that colonize our body (the microbiota) can carry AMR bacteria that do not represent any threat under normal circumstances but can be responsible for difficult-to-treat infections when they disseminate in the body after various triggering events, for example in the hospital setting but also in immunocompromised patients. These triggering events can be invasive procedures (surgery, intubation, catheterization, etc.) or harsh conditioning procedures (immunosuppression, irradiation, etc.) that compromise epithelial barriers and promote dissemination of bacteria and infections in patients.

Indeed, it is now well accepted that asymptomatic carriage of AMR bacteria predisposes to difficult-to-treat infections with the same organism. Patients presenting skin/nasal or intestinal carriage of AMR bacteria (notably Methicillin resistant Staphylococcus aureus-MRSA), and Extended Spectrum beta lactamase (ESBL) and/or Carbapenemase-producing (CP) Enterobacteria, respectively) at the time of hospitalization and/or when suffering from another disease, are more prone to develop AMR infections with the same organism in various situations (ICU, surgery, dialysis, transplants, hematopoietic stem cell transplantation, chemotherapy, irradiation, immune checkpoint inhibitors therapy, etc.) (Sakr et al., 2019; Gargiullo et al., 2019; Stalin et al., 2018; Lambelet et al., 2017).

In this context, strategies aiming at decolonizing AMR bacteria from the microbiota of patients before their invasive or non-invasive medical procedures to reduce the risk of infection have been attempted. The benefit of prophylactic decolonization of MRSA from the skin/nose has already been demonstrated as it was shown to reduce the risk of MRSA infections later during hospitalization (Bode et al., 2010; Perl et al., 2002). The gold standard for such decolonization is topical treatment with mupirocin (Walker et al., 2003) but, more recently, other approaches using lysins have shown similar or better performance in preclinical models (Kokai-kun et al., 2003; Pastagia et al., 2011).

The situation is more complicated for the intestine where the microbiota is much more diverse and complex, and more difficult to access than the skin microbiota. Selective Digestive Decontamination (SDD) consists in treating a patient presenting intestinal carriage with antibiotic resistant bacteria with cocktails of non-absorbable wide spectrum antibiotics (colistin, tobramycin) to eradicate the antibiotic resistant bacteria from their intestines. SDD remains poorly efficient, not durable and presents important side effects (disruption of the microbiota—dysbiosis—and associated infections, selection of additional antibiotic resistance, impaired prognosis in bone marrow transplant patients) that prevent its adoption in the clinic (Rieg et al., 2015; Gargiullo et al., 2019). It was also proposed to administer a selective oral decontamination therapy of carbapenem-resistant Klebsiella pneumoniae with oral gentamicin in hematologic patients as a complementary approach for reducing the risk of severe infection with these antibiotic-resistant bacteria (Lambelet et al., 2017). However, such a therapy also presents important side effects (disruption of the microbiota—dysbiosis—and associated infections, selection of additional antibiotic resistance) that questions its use in the clinic.

Probiotics and live biological products have shown promising results for the elimination of gram positive bacteria (including MRSA and Vancomycin-Resistant Enterococci) in the intestine (Manley et al., 2007; Szachta et al., 2011), but have given no sign of efficacy for antibiotic resistant gram negative bacteria (Tannock et al., 2011; Salomão et al., 2016).

Fecal Material Transplant (FMT) has also been proposed as a potential way to eradicate carriage of antibiotic resistant bacteria as it showed some degree of efficacy in various studies in patients (Saha et al., 2019). However, intrinsic limitations (dependency on stool donors, standardization issues due to donor heterogeneity, limited amount of available material) as well as safety issues (transfer of cryptic pathogens and/or other antibiotic resistant organisms especially in weakened patients at risk of infections) prevents the widespread use of FMT as a drug in the clinic.

Approaches using bacteriophages or bacteriophage parts (lysins, pyocins, etc.) to selectively eradicate bacterial populations at the strain or species level are also considered as potential strategies to eradicate antibiotic resistant bacteria from a patient's intestine. However, these approaches have failed to be proven efficacious in the intestine so far. Moreover, wild-type bacteriophages and bacteriophage parts cannot selectively eradicate antibiotic resistant bacterial strains as their specificity is solely driven by the recognition of molecular patterns exposed at the surface of the target bacteria (outer membrane proteins, serotype, capsule type, etc.) and not on the genes they carry (De Jonge et al, 2019). Antibiotic resistance genes are, for the most part, carried by mobile genetic elements and acquired by horizontal gene transfers: their presence in bacterial strains does not necessarily correlate with their serotype or capsule type (San Millan et al, 2018). In complex ecosystems like the intestine, antibiotic resistant and antibiotic sensitive strains of a given species are thought to occupy the same ecological niche.

Therefore, targeting both antibiotic resistant and antibiotic sensitive strains irrespectively has little chance to lead to eradication of the antibiotic resistant strains as both antibiotic resistant and antibiotic sensitive strains are expected to come back at a similar ratio after bacterial cells that survived the treatment repopulate their niche (FIG. 2A).

Attempts to decolonize antibiotic resistant bacteria from the intestine have failed to be proven efficient. Thus, compositions and methods for decolonizing antibiotic resistant bacteria from the microbiota, and particularly for decolonizing antibiotic resistant bacteria from the intestine, are needed. The present invention fulfills this need.

### BRIEF SUMMARY OF INVENTION

The invention encompasses compositions, kits, and methods for selectively eradicating bacteria that carry antibiotic resistance genes in the microbiota using packaged phagemids or engineered bacteriophages. Compared to wild-type phages used in the prior art, engineered bacteriophages used in the context of the invention can selectively eradicate antibiotic resistant bacterial strains because of their specificity which is not only driven by the recognition of molecular patterns exposed at the surface of the target bacteria (outer membrane proteins, serotype, capsule type, etc.) but also on the genes they carry after engineering, in particular the nucleic acid encoding a DNA modifying enzyme inactivating the antibiotic resistance gene(s) responsible for antibiotic-resistance of bacterial strain(s), as disclosed below.

Preferably, the microbiota is intestinal and the packaged phagemids or engineered bacteriophages are administered orally. Preferably, the phagemid or the genome of engineered bacteriophages encodes a nuclease or other enzyme that genetically modifies the DNA encoding the antibiotic resistance gene so that the bacteria can be eliminated with the antibiotic.

In one embodiment, the invention encompasses a method of selectively removing antibiotic resistance from an antibiotic resistant bacterial strain(s) in a healthy subject carrying antibiotic resistant bacterial strain(s) or in a patient carrying antibiotic resistant bacterial strain(s), wherein the method comprises administering to the subject or patient at least one vector (in particular at least 2, 3, 4, 5, 6, 7, 8, 9, 10 or more vectors), said vector encoding a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said healthy subject or patient and preventing antibiotic resistance related infection in the subject or patient. In the context of the invention, said healthy subject and said patient do not suffer from a bacterial infection, in particular do not suffer from an antibiotic resistance related bacterial infection, i.e. said healthy subject and said patient are asymptomatic to a bacterial infection at the time of administration of said vector.

In one embodiment, the invention encompasses a method of selectively removing antibiotic resistance from an antibiotic resistant bacterial strain(s) in a healthy subject carrying antibiotic resistant bacterial strain(s) or in a patient carrying antibiotic resistant bacterial strain(s), wherein the method comprises administering to the subject or patient at least one recombinant phage, (in particular at least 2, 3, 4, 5, 6, 7, 8, 9, 10 or more recombinant phages), said recombinant phage comprising a nucleic acid encoding a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said healthy subject or patient and preventing antibiotic resistance related infection in the subject or patient. In the context of the invention, said healthy subject and said patient do not suffer from a bacterial infection, in particular do not suffer from an antibiotic resistance related bacterial infection, i.e. said healthy subject and said patient are asymptomatic to a bacterial infection at the time of administration of said vector.

In one embodiment, the invention concerns at least one vector for use in a method of selectively removing antibiotic resistance from an antibiotic resistant bacterial strain(s) in a healthy subject carrying antibiotic resistant bacterial strain(s) or in a patient carrying antibiotic resistant bacterial strain(s), wherein said vector encoding a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns at least one recombinant phage for use in a method of selectively removing antibiotic resistance from an antibiotic resistant bacterial strain(s) in a healthy subject carrying antibiotic resistant bacterial strain(s) or in a patient carrying antibiotic resistant bacterial strain(s), wherein said recombinant phage comprising a nucleic acid encoding a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), wherein the method comprises administering to the subject or patient at least one vector (in particular at least 2, 3, 4, 5, 6, 7, 8, 9, 10 or more vectors), said vector encoding a DNA modifying enzyme inactivating the antibiotic resistance gene (s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), wherein the method comprises administering to the subject or patient at least one recombinant phage (in particular at least 2, 3, 4, 5, 6, 7, 8, 9, 10 or more recombinant phages), said recombinant phage comprising a nucleic acid encoding a DNA modifying enzyme inactivating the antibiotic resistance gene (s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns at least one vector for use in a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), wherein said vector encodes a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns at least one recombinant phage for use in a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), wherein said recombinant phage comprises a nucleic acid encoding a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns the use of at least one vector (in particular at least 2, 3, 4, 5, 6, 7, 8, 9, 10 or more) for the manufacture of a medicament intended for the prevention of antibiotic resistance related infection, in particular infection due to antibiotic-resistant bacteria, in a healthy subject carrying antibiotic resistant bacterial strain(s) or in a patient carrying antibiotic resistant bacterial strain(s), wherein said vector encodes a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns the use of at least one recombinant phage (in particular at least 2, 3, 4, 5, 6, 7, 8, 9, 10 or more recombinant phages) for the manufacture of a medicament intended for the prevention of antibiotic resistance related infection, in particular infection due to antibiotic-resistant bacteria, in a healthy subject carrying antibiotic resistant bacterial strain(s) or in a patient carrying antibiotic resistant bacterial strain(s), wherein said recombinant phage comprises a nucleic acid encoding a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In the context of the invention, all the embodiments disclosed below concerning vectors and their use also apply to recombinant phages and their use.

In one embodiment, said vector further comprises an origin of replication. In another embodiment, said vector further comprises a conditional origin of replication which is inactive in the targeted bacteria but is active in a donor bacterial cell. In a particular embodiment, said conditional origin of replication is an origin of replication, the replication of which depends upon the presence of a given protein, peptid, nucleic acid, RNA, molecule or any combination thereof. In a particular embodiment, said conditional origin of replication is active in said donor bacterial cell because said donor bacterial cell expresses said given protein, peptid, nucleic acid, RNA, molecule or any combination thereof. In a particular embodiment, said conditional origin of replication is an origin of replication derived from phage-inducible chromosomal islands (PICIs). In a particular embodiment, said conditional origin of replication is active in said donor bacterial cell because said donor bacterial cell expresses a rep protein, in particular a primase-helicase. In a particular embodiment, said conditional origin of replication is derived from the origin of replication from the PICI of the Escherichia coli strain CFT073. In a particular embodiment, said conditional origin of replication comprises or consists of the sequence SEQ ID NO: 1 or SEQ ID NO: 2.

In one embodiment, said vector is devoid of any antibiotic resistance marker.

In one embodiment, the method further comprises obtaining a microbiota sample from the subject or patient and confirming the presence of said antibiotic resistant bacterial strain(s) in the sample, either before or after administration.

In one embodiment, the method comprises selectively removing antibiotic resistance from an antibiotic resistant bacterial strain(s) in a healthy subject carrying antibiotic resistant bacterial strain(s) or in a patient carrying antibiotic resistant bacterial strain(s), wherein the method comprises obtaining a microbiota sample from the subject or patient, confirming the presence of said antibiotic resistant bacterial strain(s) in the sample, administering to the subject or patient a payload/vector (comprised or not inside a bacterial delivery vehicle), said payload/vector encoding a DNA modifying enzyme, such as a nuclease or base editor, inactivating the antibiotic resistance gene(s) responsible for antibiotic-resistance of bacterial strain(s), thereby selectively removing antibiotic resistance from an antibiotic resistant bacterial strain(s) in the microbiota of said subject or patient and preventing antibiotic resistance related infection in the subject or patient.

In one embodiment, the invention concerns a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), wherein the method comprises obtaining a microbiota sample from the subject or patient, confirming the presence of said antibiotic resistant bacterial strain(s) in the sample, administering to the subject or patient at least one payload/vector (comprised or not inside a bacterial delivery vehicle) (in particular at least 2, 3, 4, 5, 6, 7, 8, 9, 10 or more), said payload/vector encoding a DNA modifying enzyme, such as a nuclease or base editor, inactivating the antibiotic resistance gene(s) responsible for antibiotic-resistance of bacterial strain(s), thereby selectively removing antibiotic resistance from an antibiotic resistant bacterial strain(s) in the microbiota of the subject or patient and preventing antibiotic resistance related infection in the subject or patient.

In one embodiment, the invention concerns at least one payload/vector for use in a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), wherein said payload/vector encodes a DNA modifying enzyme, such as a nuclease or base editor, inactivating the antibiotic resistance gene(s) responsible for antibiotic-resistance of bacterial strain(s), thereby selectively removing antibiotic resistance from an antibiotic resistant bacterial strain(s) in the microbiota of the subject or patient and preventing antibiotic resistance related infection in the subject or patient, and wherein the method comprises obtaining a microbiota sample from the subject or patient, confirming the presence of said antibiotic resistant bacterial strain(s) in the sample, preferably before administration of said payload/vector.

In one embodiment, antibiotic resistance is removed through targeted elimination of the antibiotic resistant strains when the nuclease is programmed to perform a DNA cleavage, e.g. a double strand DNA break in an antibiotic resistance gene located on the chromosome of the target bacteria or on a plasmid with addictive systems (toxin/antitoxin). In another embodiment, antibiotic resistance is removed by resensitization of the target bacteria to the antibiotic when the nuclease is programmed to perform a DNA cleavage, e.g. a double strand DNA break in the antibiotic resistance gene located on a plasmid without addictive systems. In another embodiment, antibiotic resistance is removed by resensitization of the target bacteria to the antibiotic when the nuclease is programmed to inactivate the antibiotic resistance gene through base editing.

In one embodiment, the payload/vector is a conjugative plasmid not comprised inside a bacterial delivery vehicle but inside a donor cell, and is transferred into the antibiotic resistant bacterial strain(s) by conjugation from the donor cell carrying the conjugative machinery. Alternatively, the payload/vector can be transferred into the antibiotic resistant bacterial strain(s) by bacterial transformation as a naked DNA.

In one embodiment, said payload/vector further comprises a conditional origin of replication which is inactive in the targeted bacteria but is active in a donor bacterial cell. In a particular embodiment, said conditional origin of replication is an origin of replication, the replication of which depends upon the presence of a given protein, peptid, nucleic acid, RNA, molecule or any combination thereof. In a particular embodiment, said conditional origin of replication is active in said donor bacterial cell because said donor bacterial cell expresses said given protein, peptid, nucleic acid, RNA, molecule or any combination thereof. In a particular embodiment, said conditional origin of replication is an origin of replication derived from phage-inducible chromosomal islands (PICIs). In a particular embodiment, said conditional origin of replication is active in said donor bacterial cell because said donor bacterial cell expresses a rep protein, in particular a primase-helicase. In a particular embodiment, said conditional origin of replication is derived from the origin of replication from the PICI of the Escherichia coli strain CFT073. In a particular embodiment, said conditional origin of replication comprises or consists of the sequence SEQ ID NO: 1 or SEQ ID NO: 2.

In one embodiment, said payload/vector comprises an auxotrophic marker.

In one embodiment, antibiotic resistance is removed from the intestine of the healthy subject or of the patient. By “removal of antibiotic resistance” is meant herein that antibiotic resistance gene(s) is(are) removed or inactivated from bacterial strains carrying such gene(s) and/or antibiotic resistant bacterial cells are eliminated. Therefore, in one embodiment, antibiotic resistance gene(s) is(are) removed or inactivated from bacterial strains present in the intestine of the healthy subject or of the patient, and/or antibiotic resistant bacterial strains are eliminated from the intestine of the healthy subject or of the patient.

In one embodiment, antibiotic resistance is removed before the subject or the patient undergoes an invasive medical procedure, such as a surgery. In one embodiment, the surgery is a solid organ transplant surgery. Preferably, the organ transplant is a kidney or liver transplant. In one embodiment, the surgery is a bone marrow transplant or a hematopoietic stem cell transplantation. In one embodiment, antibiotic resistance is removed before the subject or the patient undergoes a non-invasive medical procedure conditioning procedure, such as immunosuppression, irradiation, or chemotherapy or other non-invasive medical procedure.

In one embodiment, the invention concerns a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), said subject or patient being intended to undergo an invasive medical procedure, such as a surgery, or non-invasive medical procedure, such as immunosuppression, irradiation, or chemotherapy, wherein said method comprises administering to the subject or patient, before said subject or patient undergoes an invasive or non-invasive medical procedure, at least one vector (in particular at least 2, 3, 4, 5, 6, 7, 8, 9, 10 or more vectors), said vector encoding a DNA modifying enzyme inactivating the antibiotic resistance gene(s) responsible for antibiotic-resistance of bacterial strain(s), thereby selectively removing antibiotic resistance from an antibiotic resistant bacterial strain(s) in the microbiota and preventing antibiotic resistance related infection in the subject or patient before said subject or patient undergoes said invasive or non-invasive medical procedure.

In one embodiment, the invention concerns at least one vector for use in a method of preventing antibiotic resistance related infection, in particular infection due to antibiotic-resistant bacteria, in a healthy subject carrying antibiotic resistant bacterial strain(s) or in a patient carrying antibiotic resistant bacterial strain(s), said subject or patient being intended to undergo an invasive medical procedure, such as a surgery, or non-invasive medical procedure, such as immunosuppression, irradiation, or chemotherapy, wherein said vector encodes a DNA modifying enzyme inactivating the antibiotic resistance gene(s) responsible for antibiotic-resistance of bacterial strain(s), and wherein said method comprises administering said vector to the subject or patient, before said subject or patient undergoes an invasive or non-invasive medical procedure, thereby selectively removing antibiotic resistance from an antibiotic resistant bacterial strain(s) in the microbiota and preventing antibiotic resistance related infection in the subject or patient before said subject or patient undergoes said invasive or non-invasive medical procedure.

The present invention also concerns the use of at least one vector (in particular at least 2, 3, 4, 5, 6, 7, 8, 9, 10 or more vectors) in the manufacture of a medicament intended for the prevention of antibiotic resistance related infection, in particular infection due to antibiotic-resistant bacteria, in a healthy subject carrying antibiotic resistant bacterial strain(s) or in a patient carrying antibiotic resistant bacterial strain(s), said subject or patient being intended to undergo an invasive medical procedure, such as a surgery, or non-invasive medical procedure, such as immunosuppression, irradiation, or chemotherapy, wherein said vector encodes a DNA modifying enzyme inactivating the antibiotic resistance gene(s) responsible for antibiotic-resistance of bacterial strain(s), and wherein said subject or patient is administered with said vector before undergoing an invasive or non-invasive medical procedure, thereby selectively removing antibiotic resistance from an antibiotic resistant bacterial strain(s) in the microbiota and preventing antibiotic resistance related infection in the subject or patient before said subject or patient undergoes said invasive or non-invasive medical procedure.

In one embodiment, the microbiota is the gut microbiota.

In one embodiment, the antibiotic is from the carbapenem class or beta-lactam class of antibiotics. In one embodiment, the antibiotic is methicillin or vancomycin.

In one embodiment, the nuclease is a CRISPR associated nuclease.

In one embodiment, the payload/vector is a conjugative plasmid not comprised inside a bacterial delivery vehicle but inside a donor cell, and is transferred into the antibiotic resistant bacterial strain(s) by conjugation from a donor cell carrying the conjugative machinery. Alternatively, the payload/vector can be transferred into the antibiotic resistant bacterial strain(s) by bacterial transformation as a naked DNA.

In a particular embodiment, at least two distinct vectors are used, in particular at least 3, 4, 5, 6, 7, 8, 9, 10 or more vectors, preferably four vectors, each vector targeting different bacteria strains or species, in particular different E. coli bacteria strains, and/or encoding a DNA modifying enzyme targeting a different nucleic acid sequence inside the targeted bacteria. In a particular embodiment, at least two recombinant phages are used, in particular at least 3, 4, 5, 6, 7, 8, 9, 10 or more recombinant phages, preferably four recombinant phages, each recombinant phage targeting a different bacteria strains or species, in particular different E. coli bacteria strains, and/or comprising nucleic acids encoding a DNA modifying enzyme targeting a different nucleic acid sequence inside the targeted bacteria.

### BRIEF DESCRIPTION OF DRAWINGS

FIG. 1. Principle of selective intestinal decolonization of AMR bacteria using packaged phagemids for prophylactic purposes. After a patient is diagnosed with intestinal carriage with AMR bacteria (by culture dependent and/or independent methods from a rectal swab and/or a fecal sample) they receive an oral treatment of packaged phagemids that will selectively eradicate AMR bacteria from their intestine without altering the rest of their microbiota. Therefore, if the patient intestinal barrier is later compromised by an invasive or non invasive-associated triggering event (invasive procedure, immunosuppression . . . ) the patient has a lower risk of developing a difficult-to-treat infection, such as multidrug resistant (MDR) infections, with an AMR bacteria they were carrying in their intestine.

FIG. 2A-B. Comparison of sequence-specific and non-specific approaches for the intestinal decolonization of AMR bacteria. (A.) Non-specific approaches do not discriminate between AMR and non-AMR strains from a given species: they lead to a reduction of the whole bacterial population during the treatment. When the treatment stops, in absence of negative selective pressure, both AMR and non-AMR strains repopulate their niche. (B.) On the contrary, sequence-specific approach using packaged phagemids only eliminates AMR strains and leaves the non-AMR strain population intact. In the absence of negative selective pressure against non-AMR strains, they populate the niche vacated by AMR strains, leading to a durable exclusion of AMR strains even after the treatment stops.

FIG. 3A-B. Sequence specific elimination of clinical strains of ESBL E. coli using packaged phagemids. Representation of (A.) a spot assay and (B.) associated cell counts for two clinical E. coli strains carrying different ESBL gene variants (CTX-M1 strain on the left, CTX-M9 strain on the right) transduced with packaged phagemids targeting either CTX-M1 or CTX-M9. The two packaged phagemids consisted in the same delivery vehicle (lambda-derived scaffold, 1A2 gpJ tip protein, stf27 tail fiber protein) containing either the p775 payload (FnCpf1+crRNA against CTX-M1 ESBL gene variant) or the p776 payload (FnCpf1+crRNA against CTX-M9 ESBL gene variant).

FIG. 4. Variation in ESBL E. coli bacterial levels in the feces of mice between TO (before the treatment) and T8h (8 hours after the treatment), expressed as CFU/g of stool. Between 0.5 log₁₀ and 1.0 log₁₀ decrease was observed in all groups.

FIG. 5. Sequence-specific killing of Klebsiella pneumoniae. Black line, K. pneumoniae treated with different MOIs of Lambda-derived particles harboring the chimeric STF118. Grey line, K. pneumoniae treated with different MOIs of Lambda-derived particles harboring the chimeric STF SIEA11.

### DETAILED DESCRIPTION OF THE INVENTION

The invention encompasses methods, kits, and compositions for selectively removing antibiotic resistance from a healthy subject carrying antibiotic resistant bacterial strain(s) or from a patient carrying antibiotic resistant bacterial strain(s). Phagemids, preferably encoding nucleases or other enzymes that genetically modify the DNA encoding an antibiotic resistance gene, can be used to eliminate nucleic acids encoding antibiotic resistance genes or inactivate antibiotic resistance genes. In this way, healthy subjects or patients can be prepared before their invasive or non-invasive medical procedures to reduce the risk of antibiotic resistant infection later on. Typically, after the invasive or non-invasive medical procedure, the bacteria can be eliminated with the antibiotic.

In the context of the invention, the healthy subject or the patient to be treated with the vector of the invention does not suffer from a bacterial infection. In other words, the methods of the invention do not aim at treating a bacterial infection nor at preventing the aggravation of a bacterial infection (for example preventing the onset of sepsis or septicemia). The methods of the present invention typically aim at decolonizing a healthy subject or a patient from antibiotic resistant bacterial strain(s), in particular to avoid the onset of a bacterial infection due to this(these) antibiotic resistant bacterial strain(s) typically when said subject or patient faces a potentially triggering event such as a invasive or non-invasive medical procedures. In the context of the invention, said decolonization can typically be obtained by specifically killing said antibiotic resistant bacterial strain(s), by removing the antibiotic resistance gene(s) from said antibiotic resistant bacterial strain(s) and/or by inactivating the antibiotic resistance gene(s) from said antibiotic resistant bacterial strain(s).

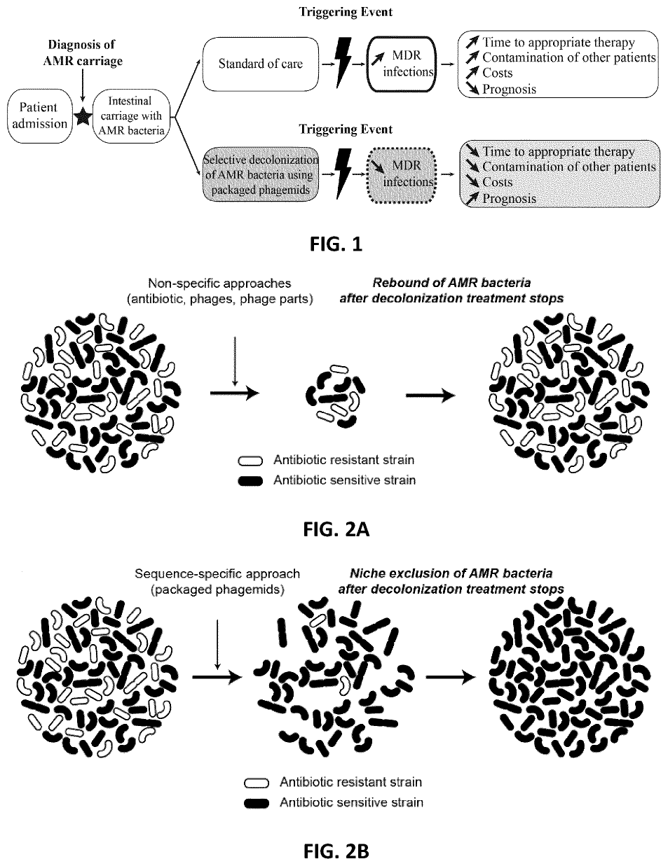

In one embodiment, the invention encompasses a method to selectively eradicate bacteria that carry antibiotic resistance gene(s) from the intestinal microbiota using vectors, in particular packaged phagemids or engineered/recombinant phages, administered to a healthy subject or patient, for example orally, rectally (e.g., in an enema), vaginally, nasally or to the skin. This prophylactic elimination of intestinal carriage of antibiotic resistant bacteria can reduce the risk of antibiotic resistant infections with that same bacteria after various triggering events, and their associated costs (FIG. 1).

In one embodiment, the invention encompasses a method of selectively removing antibiotic resistance from an antibiotic resistant bacterial strain(s) in a healthy subject or patient carrying antibiotic resistant bacterial strain(s), wherein the method comprises administering to the subject or patient at least one vector (in particular at least 2, 3, 4, 5, 6, 7, 8, 9, 10 or more vectors), in particular a packaged phagemid or an engineered/recombinant phage, said vector encoding a DNA modifying enzyme inactivating the antibiotic resistance gene (s) responsible for antibiotic-resistance of bacterial strain(s), thereby selectively removing antibiotic resistance from an antibiotic resistant bacterial strain(s) in the microbiota and preventing antibiotic resistance related infection in the subject.

In one embodiment, the method further comprises obtaining a microbiota sample from the subject or patient and confirming the presence of said antibiotic resistant bacterial strain(s) in the sample, either before or after administration.

In one embodiment, after a healthy subject or patient is diagnosed with intestinal carriage with AMR bacteria (for example by culture dependent and/or independent methods from a rectal swab and/or a fecal sample) they can receive an oral treatment of the vectors of the invention, in particular the packaged phagemids or engineered/recombinant phages, that can selectively remove antibiotic resistance from AMR bacteria from their intestine, and without altering the rest of their microbiota in situation where AMR bacteria are eradicated. Therefore, if the subject or patient intestinal barrier is later compromised by an invasive or non-invasive medical procedure-associated triggering event, the subject or patient has a lower risk of developing a difficult-to-treat infection, such as multidrug resistant (MDR) infections, with an AMR bacteria they were carrying in their intestine.

In one embodiment, healthy subject or patient can optionally be tested for intestinal carriage with AMR bacteria (for example by culture dependent and/or independent methods from a rectal swab and/or a fecal sample) and can receive an administration, for example, an oral, rectal (e.g., in an enema), vaginal, nasal, or skin treatment, of vectors of the invention, in particular of packaged phagemids or engineered/recombinant phages, that can selectively remove antibiotic resistance from AMR bacteria from their intestine, in situation where AMR bacteria are eradicated. Therefore, if the subject or patient intestinal barrier is later compromised by an invasive or non-invasive medical procedure-associated triggering event, the subject or patient has a lower risk of developing a difficult-to-treat infection, such as multidrug resistant (MDR) infections, with an AMR bacteria they were carrying in their intestine.

Packaged phagemids can allow the transduction in the target bacteria of a synthetic DNA payload.

Preferably, the DNA payload encodes a sequence-specific RNA-guided nuclease complex (e.g., type I, II, III or V CRISPR-Cas system) programmed to generate DNA cleavage, e.g. double strand DNA breaks, in antibiotic resistance gene sequences located either on the bacterial chromosome or on a plasmid. When the antibiotic resistance gene is located in the chromosome or on a plasmid with addictive systems (toxin/antitoxin), cleavage of the DNA leads to bacterial death. When the antibiotic resistance gene is located on a plasmid without addiction systems, cleavage can occur either in the antibiotic resistance gene or anywhere else in the plasmid, leading in both situations to resensitization of the target bacteria that can then be eliminated in the presence of the corresponding antibiotic in case of infection.

Alternatively, a DNA modifying enzyme can be programmed to inactivate the antibiotic resistance gene through base editing, leading to the resensitization of the target bacteria to the antibiotic.

All these approaches lead to the same outcome: the elimination of AMR genes from bacterial populations with an unparalleled specificity. They allow the durable decolonization of antibiotic resistant bacteria from complex ecological ecosystems, such as the intestine. In the context where both antibiotic sensitive and resistant strains compete for the same niche/resources, the specific elimination of AMR strains typically leads to the outgrowth of the non-AMR strains that take over the niche and prevent AMR strains from growing back. Alternatively, in the case where all strains from a given species are resistant to the antibiotic, the removal of antibiotic resistance through plasmid curing or gene editing typically results in the majority of bacteria being resensitized to antibiotics. This majority of antibiotic sensitive bacteria typically competes with the remaining antibiotic resistant bacteria, takes over the niche and prevents antibiotic resistant bacteria to grow back to their initial level. In the context where there is a single strain of the target species and that strain is resistant, the specific elimination of the resistance of the target strain can typically lead to the outgrowth of the non-resistant bacteria that can take over the niche and prevent the resistant bacteria from growing back.

Preferably, the target, that is to say the antibiotic resistance gene(s), is on a plasmid and the plasmid is cured. Then, the cured bacteria will compete with the initial resistant bacteria and help replace them. Therefore, this approach providing the sequence-specific elimination of AMR strains allows the leverage of the competitive environment of the GI (Gastro Intestinal) tract to favor their durable exclusion from the microbiota by antibiotic sensitive commensal strains (FIG. 2B).

To further favor the exclusion of AMR strains from their intestinal niche, AMR-colonized subjects or patients can be treated with a combination of vectors of the invention, in particular packaged phagemids, and a probiotic strain. The vector, in particular the packaged phagemid, can specifically eliminate AMR strains from the subject or patient's intestine and the probiotic strain can take over the ecological niche, further preventing AMR strains from bouncing back.

The invention encompasses methods of selectively removing antibiotic resistance bacteria from a healthy subject carrying antibiotic resistant bacterial strain(s) or from a patient carrying antibiotic resistant bacterial strain(s). In one embodiment, the method comprises administering to the subject or patient at least one payload/vector (in particular at least 2, 3, 4, 5, 6, 7, 8, 9, 10 or more), which can be inside one or more bacterial delivery vehicle(s). In one embodiment, the payload/vector encodes a nuclease that can cleave the nucleic acid encoding the antibiotic resistance gene(s) responsible for antibiotic-resistance of bacterial strain(s), thereby selectively removing antibiotic resistant bacteria from the microbiota of the subject or patient, when the antibiotic resistance gene is located on the bacterial chromosome or plasmid with addictive systems or removing antibiotic resistance from antibiotic resistant bacteria of the microbiota of the subject or patient, when the antibiotic resistance gene is located on a plasmid without addictive systems, and preventing antibiotic resistance related infection in the subject or patient.

In one embodiment, the method comprises obtaining a microbiota sample from the subject or patient confirming the presence of said antibiotic resistant bacterial strain(s) in the sample, preferably before the administration. The microbiota sample can also be obtained from the subject or patient after the administration to measure the effectiveness of the administration.

Optionally, the method comprises a step of confirming that the payload/vector works on the patient or subject sample in vitro. Thus a sample of microbiota taken from the patient or subject can be contacted with the payload/vector in vitro and the effect of the payload/vector on antibiotic resistance assessed by routine in vitro procedures.

In one embodiment, the antibiotic resistant bacteria are removed from the gut of the healthy subject or of the patient.

In one embodiment, the selective decolonization (or removal of antibiotic resistance bacteria) occurs before the subject or patient undergoes an invasive or non-invasive medical procedure, such as a surgical procedure or such as a treatment of cancer or such as an immunosuppressive treatment. In another embodiment, the selective decolonization occurs in patients more prone at developing AMR infection such as patients with hematological malignancy, cancer, chronic disease, autoimmune disease or under viral attack.

In one embodiment, the surgical procedure is a bone marrow transplant or a hematopoietic stem cell transplantation.

In one embodiment, the surgery is a solid organ transplant surgery, in particular a kidney transplant surgery and/or a liver transplant surgery.

In one embodiment, the treatment of cancer is selected from the group consisting of cytotoxic agents, chemotherapeutic agents, growth inhibitory agents, agents used in radiation therapy, anti-angiogenesis agents, apoptotic agents, anti-tubulin agents, and other agents to treat cancer, for example, anti-CD20 antibodies, platelet derived growth factor inhibitors (e.g., GLEEVEC™ (imatinib mesylate)), a COX-2 inhibitor (e.g., celecoxib), interferons, cytokines, antagonists (e.g., neutralizing antibodies) that bind to one or more of the following targets PDGFR-6, BlyS, APRIL, BCMA receptor(s), TRAIL/Apo2, other bioactive and organic chemical agents, and combinations thereof.

The term “cytotoxic agent” as used herein refers to a substance that inhibits or prevents the function of cells and/or causes destruction of cells. The term is intended to include radioactive isotopes (e.g., At211, 1131, 1125, Y90, Re186, Re188, Sm153, Bi212, P32, and radioactive isotopes of Lu), chemotherapeutic agents, e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below.

A “chemotherapeutic agent” is a chemical compound useful in the treatment of cancer. Examples of chemotherapeutic agents include alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins; delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I; dynemicin, including dynemicin A; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycin, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, for example taxanes including TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhone-Poulenc Rorer, Antony, France); chloranbucil; gemcitabine (GEMZAR®); 6-thioguanine; mercaptopurine; methotrexate; platinum or platinum-based chemotherapy agents and platinum analogs, such as cisplatin, carboplatin, oxaliplatin (ELOXATIN™), satraplatin, picoplatin, nedaplatin, triplatin, and lipoplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts or acids of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin. Additional chemotherapeutic agents include the cytotoxic agents useful as antibody drug conjugates, such as maytansinoids (DM1, for example) and the auristatins MMAE and MMAF, for example.

Chemotherapeutic agents also include “anti-hormonal agents” or “endocrine therapeutics” that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGFR); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts or acids of any of the above.

Chemotherapeutic agents also include antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories), which is a recombinant exclusively human-sequence, full-length IgG1 A antibody genetically modified to recognize interleukin-12 p40 protein.

Chemotherapeutic agents also include “EGFR inhibitors”, which refers to compounds that bind to or otherwise interact directly with EGFR and prevent or reduce its signaling activity, and is alternatively referred to as an “EGFR antagonist”. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX) and reshaped human 225 (H225); IMC-11F8, a fully human, EGFR-targeted antibody (Imclone); antibodies that bind type II mutant EGFR; humanized and chimeric antibodies that bind EGFR; and human antibodies that bind EGFR, such as ABX-EGF or Panitumumab; EMD 55900; EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6.3, and E7.6.3; MDX-447 (Medarex Inc); and mAb 806 or humanized mAb 806. The anti-EGFR antibody may be conjugated with a cytotoxic agent, thus generating an immunoconjugate. EGFR antagonists include small molecules such as 051-774 (CP-358774, erlotinib, TARCEVA Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitors such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (C1-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines; tryphostins; ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®).

Chemotherapeutic agents also include immune checkpoint inhibitors such as human programmed death receptor-1 (PD-1) blocking antibodies and human cytotoxic T-lymphocyte antigen 4 (CTLA-4)-blocking antibodies. Checkpoint inhibitors include, but are not limited to nivolumab, ipilimumab, pembrolizumab, tremelimumab, and pidilizumab.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (ENBREL®), infliximab (REMICADE®), adalimumab (HUMIRA®), certolizumab pegol (CIMZIA®), golimumab (SIMPONI®), Interleukin 1 (IL-1) blockers such as anakinra (KINERET®), T-cell co-stimulation blockers such as abatacept (ORENCIA®), Interleukin 6 (IL-6) blockers such as tocilizumab (ACTEMERA®); Interleukin 13 (IL-13) blockers such as lebrikizumab; Interferon alpha (IFN) blockers such as rontalizumab; beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); miscellaneous investigational agents such as thioplatin, PS-341, phenylbutyrate, ET-18-OCH3, and farnesyl transferase inhibitors (L-739749, L-744832); polyphenols such as quercetin, resveratrol, piceatannol, epigallocatechine gallate, theaflavins, flavanols, procyanidins, betulinic acid; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-am inocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g., celecoxib or etoricoxib), proteosome inhibitor (e.g., PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts or acids of any of the above; as well as combinations of two or more of the above.

A “growth inhibitory agent” when used herein refers to a compound or composition which inhibits growth and/or proliferation of a cell either in vitro or in vivo. Thus, the growth inhibitory agent may be one that significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as the anthracycline antibiotic doxorubicin ((8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione), epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

In one embodiment, the hematological malignancy is selected from leukemia, lymphoma, and myeloma.

Leukemias include acute lymphoblastic leukemia (ALL), acute myelogenous leukemia (AML), chronic lymphocytic leukemia (CLL), small lymphocytic lymphoma (SLL), chronic myelogenous leukemia (CML), and acute monocytic leukemia (AMOL).

Lymphomas include Hodgkin's lymphomas and Non-Hodgkin's lymphomas. NHL can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, immunoblastic large cell lymphoma, precursor B-lymphoblastic lymphoma, and mantle cell lymphoma. T-cell non-Hodgkin lymphomas include mycosis fungoides, anaplastic large cell lymphoma, and precursor T-lymphoblastic lymphoma.

By “chronic disease” is meant herein conditions that last 1 year or more and require ongoing medical attention or limit activities of daily living or both.

In one embodiment, the chronic disease is selected from arthritis, asthma, cancer, chronic obstructive pulmonary disease, diabetes, Lyme disease, autoimmune diseases, genetic disorders and some viral diseases such as hepatitis C and acquired immunodeficiency syndrome, Alzheimer's Disease and other Dementias, eating disorders, Osteoporosis, Reflex Sympathetic Dystrophy (RSD) Syndrome and tobacco use-related conditions.

In one embodiment, the autoimmune disease is selected from alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune parotitis, autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis, diabetes mellitus, discoid lupus, essential mixed cryoglobulinemia, dystrophic epidermolysis bullosa, epididymitis, fibromyalgia-fibromyositis, glomerulonephritis, Graves' disease, Guillain-Barre syndrome, Hashimoto's thyroiditis, hemolytic anemia, idiopathic pulmonary fibrosis, idiopathic thrombocytopenia purpura (ITP), IgA nephropathy, juvenile chronic arthritis (Still's disease), juvenile rheumatoid arthritis, Meniere's disease, mixed connective tissue disease, multiple sclerosis, myasthenia gravis, myxedema, pemphigus vulgaris, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, spondyloarthropathies, stiff-man syndrome, systemic lupus erythematosus, Takayasu arteritis, temporal arteritis/giant cell arteritis, thyroiditis, ulcerative colitis, uveitis, vasculitis, vitiligo and Wegener's granulomatosis.

In one embodiment, the viral attack is an attack from a virus selected from the group consisting of Poxviridae including Chordopoxvirinae in particular Orthopox viruses (e.g. cowpox virus, monkey pox virus, vaccinia virus, variola virus, smallpox virus), Parapox viruses (e.g. bovine papular stomatitis virus, Orf virus, pseudocowpox virus), Molluscipox viruses (e.g. Molluscum contagiosum virus) and Yatapox viruses (e.g. tanapox virus, yaba monkey tumor virus); Herpesviridae including Alphaherpesvirinae in particular simplexviruses (e.g. human herpesvirus 1 (Herpes simplex virus 1), human herpesvirus 2 (Herpes simplex virus 2)) and varicelloviruses (e.g. human herpesvirus 3 (Varicella-zoster virus)), Betaherpesvirinae in particular cytomegaloviruses (e.g. human herpesvirus 5 (human cytomegalovirus)) and roseoloviruses (e.g. human herpesvirus 6, human herpesvirus 7), and Gammaherpesvirinae, in particular lymphocryptoviruses (e.g. human herpesvirus 4 (Epstein-Barr virus)) and rhadinoviruses (e.g. human herpesvirus 8 (Kaposi's sarcoma-associated herpesvirus)); Adenoviridae including mastadenoviruses (e.g. human adenovirus A, human adenovirus B, human adenovirus C, human adenovirus D, human adenovirus E and human adenovirus F); Polyomaviridae including polyomaviruses (e.g. BK polyomavirus, human polyomavirus, JC polyomavirus, KI polyomavirus, WU polyomavirus, Merkel cell polyomavirus); Papillomaviridae including alphapapillomaviruses (e.g. human papillomavirus 6, human papillomavirus 11, human papillomavirus 16, human papillomavirus 18, human papillomavirus 2, human papillomavirus 10, human papillomavirus 61, human papillomavirus 71, human papillomavirus cand90, human papillomavirus 26, human papillomavirus 30, human papillomavirus 32, human papillomavirus 54, human papillomavirus 7, human papillomavirus 34), betapapillomaviruses (e.g. human papillomavirus 5, human papillomavirus cand92, human papillomavirus cand96, human papillomavirus 9, human papillomavirus 49), gammapapillomaviruses (e.g. human papillomavirus 4, human papillomavirus 50, human papillomavirus 48, human papillomavirus 60, human papillomavirus 88) and mupapillomaviruses (e.g. human papillomavirus 1); Parvoviridae including Parvovirinae in particular dependoviruses (e.g. adeno-associated virus), erythroviruses (e.g. B19 virus) and bocaviruses (e.g. human bocavirus); Hepadnaviridae including orthohepadnaviruses (e.g. hepatitis B virus); Retroviridae including Orthoretrovirinae in particular deltaretroviruses (e.g. human T-lymphotropic virus 1 (HTLV-1), human T-lymphotropic virus 2 (HTLV-2)), and lentiviruses (e.g. human immunodeficiency virus 1, human immunodeficiency virus 2); Reoviridae including coltiviruses (e.g. Colorado tick fever virus), orbiviruses (e.g. African horse sickness virus, Changuinola virus, Corriparta virus, Orungo virus), orthoreoviruses (e.g. mammalian orthoreovirus), rotaviruses (e.g. Rotavirus A, Rotavirus B, Rotavirus C); Mononegavirales including Filoviridae in particular ebolaviruses (e.g. Ivory Coast ebolavirus, Zaire ebolavirus, Sudan ebolavirus, Reston ebolavirus, Bundibugyo ebolavirus) and marburgviruses (e.g. Lake Victoria marburgvirus), Paramyxoviridae in particular Paramyxovirinae comprising henipaviruses (e.g. Hendra virus, Nipah virus), morbilliviruses (e.g. measles virus (Edmonston virus)), respiroviruses (e.g. human parainfluenza virus 1, human parainfluenza virus 3) and rubulaviruses (e.g. mumps virus, human parainfluenza virus 2, human parainfluenza virus 4), Pneumovirinae comprising pneumoviruses (e.g. human respiratory syncytial virus) and metapneumoviruses (e.g. human metapneumovirus), and Rhabdoviridae in particular vesiculoviruses (e.g. vesicular stomatitis Alagoas virus, vesicular stomatitis Indiana virus, vesicular stomatitis New Jersey virus, Chandipura virus, Cocal virus, Isfahan virus, Piry virus) and lyssaviruses (e.g. australian bat lyssavirus, rabies virus); Orthomyxoviridae including influenzaviruses A (e.g. Influenza A virus), influenzaviruses B (e.g. Influenza B virus) and influenzaviruses C (e.g. Influenza C virus); Bunyaviridae including hantaviruses (e.g. Andes virus, Bayou virus, Black Creek Canal virus, Dobrava-Belgrade virus, Hantaan virus, Seoul virus, Sin Nombre virus, New York virus, Puumala virus), nairoviruses (e.g. Crimean-Congo hemorrhagic fever virus, Nairobi sheep disease virus), orthobunyaviruses (e.g. Bunyamwera virus, Bwamba virus, California encephalitis virus, Guama virus, Oriboca virus, Oropouche virus) and phleboviruses (e.g. Rift Valley fever virus, Sandfly fever Naples virus); Arenaviridae including arenaviruses (e.g. Guanarito virus, Junin virus, Lassa virus, lymphocytic choriomeningitis virus, Machupo virus, Sabiá virus); Deltavirus (e.g. Hepatitis delta virus); Nidovirales including Coronaviridae, in particular coronaviruses (e.g. human coronavirus 229E, human coronavirus OC43, human enteric coronavirus, severe acute respiratory syndrom coronavirus) and toroviruses (e.g. human torovirus); Picornaviridae including enteroviruses (e.g. human enterovirus A, human enterovirus B, human enterovirus C, human enterovirus D, poliovirus), rhinoviruses (e.g. human rhinovirus A, human rhinovirus B, human rhinovirus C), hepatoviruses (e.g. Hepatitis A virus), parechoviruses (e.g. human parechovirus), cardioviruses (e.g. Vilyuisk human encephalomyelitis virus), aphtoviruses (e.g. Foot-and-mouth disease virus) and kobuviruses (e.g. Aichi virus); Caliciviridae including noroviruses (e.g. Norwalk virus) and sapoviruses (e.g. Sapporo virus); Hepevirus including Hepatitis E virus; Astroviridae including mamastroviruses (e.g. human astrovirus); Togaviridae including alphaviruses (e.g. Chikungunya virus, O'nyong-nyong virus, Mayaro virus, Ross River virus, Barmah Forest virus, Sindbis virus, venezuelan equine encephalitis virus, western equine encephalitis virus, eastern equine encephalitis virus, Ockelbo virus) and rubiviruses (e.g. rubella virus); Flaviviridae including flaviviruses (e.g. Kyasanur Forest disease virus, Omsk hemorrhagic fever virus, Powassan virus, Louping ill virus, tick-borne encephalitis virus, Dengue virus, japanese encephalitis virus, Murray Valley encephalitis virus, St. Louis encephalitis virus, West Nile virus, Ilheus virus, yellow fever virus, Apoi virus), hepaciviruses (e.g. Hepatitis C virus), GB virus B and GB virus A; and Birnaviridae (e.g. Infectious bursal disease virus).

In one embodiment, the microbiota is the gut microbiota.

In one embodiment, the antibiotic is methicillin, vancomycin, or of the carbapenem class or beta-lactam class of antibiotics.

In one embodiment, the nuclease is a CRISPR associated nuclease.

The invention encompasses compositions, kits and methods for reducing or eliminating an AMR bacteria in situ. The compositions, kits and methods of the invention reduce or eliminate deleterious AMR bacteria within the host microbiome by eliminating or reducing the antibiotic resistance of these AMR bacteria. Preferably, the elimination of AMR bacteria in situ involves the use of phages, recombinant phage, packaged phagemid, introducing a DNA cleavage, e.g. a double strand break in the DNA sequence, with or without the use of antibiotics.

The invention encompasses methods of selectively removing antibiotic resistance from antibiotic resistant bacterial strain(s) in a healthy subject carrying antibiotic resistant bacterial strain(s) or a patient carrying antibiotic resistant bacterial strain(s). In one embodiment, the method comprises administering to the subject or patient at least payload/vector (in particular at least 2, 3, 4, 5, 6, 7, 8, 9, 10 or more), which can be inside a bacterial delivery vehicle. In one embodiment, the payload/vector encodes a DNA modifying enzyme that can modify and inactivate the nucleic acid encoding the antibiotic resistance gene(s) responsible for antibiotic-resistance of bacterial strain(s), thereby selectively removing antibiotic resistance from antibiotic resistant bacteria of the microbiota of the subject or patient, and preventing antibiotic resistance related infection in the subject or patient.

In one embodiment, the method comprises obtaining a microbiota sample from the subject or patient confirming the presence of said antibiotic resistant bacterial strain(s) in the sample, preferably before the administration. The microbiota sample can also be obtained from the subject or patient after the administration to measure the effectiveness of the administration.

Optionally, the method comprises a step of confirming that the payload(s)/vector(s) work(s) on the patient or subject sample in vitro. Thus a sample of microbiota taken from the patient or subject can be contacted with the payload/vector in vitro and the effect of the payload/vector on antibiotic resistance assessed by routine in vitro procedures.

In one embodiment, the antibiotic resistance is removed from antibiotic resistant bacteria of the gut of the healthy subject or of the patient.

In one embodiment, the selective removal of antibiotic resistance occurs before the subject or patient undergoes an invasive or non-invasive medical procedure, such as a surgical procedure.

In one embodiment, the surgical procedure is a bone marrow transplant or a hematopoietic stem cell transplantation.

In one embodiment, the surgery is a solid organ transplant surgery, in particular a kidney transplant surgery and/or a liver transplant surgery.

In one embodiment, the microbiota is the gut microbiota.

In one embodiment, the antibiotic is methicillin, vancomycin, or of the carbapenem class or beta-lactam class of antibiotics.

In one embodiment, the DNA modifying enzyme is a base editor.

The invention encompasses the use of at least one vector (in particular at least 2, 3, 4, 5, 6, 7, 8, 9, 10 or more) that can transfer with high efficiency a nucleic acid, preferably a plasmid, into a bacterial population within the microbiome that allows the expression of an exogenous enzyme that will modify a DNA encoding an antibiotic resistance gene in an AMR bacteria.

### Methods of Prophylactic Treatment

The invention encompasses methods of reducing or eliminating an AMR bacteria in situ. The AMR bacteria can be contacted with a vector in situ. Preferably, the bacteria is a gut bacteria, a skin bacteria, an oral bacteria, or a bacteria on a mucosal surface.

Preferably, the AMR bacteria is reduced or eliminated by eliminating an antibiotic resistance gene. Subsequently, the bacteria will be sensitive to the antibiotic and the bacteria can be killed by contact with the antibiotic.

In one embodiment, the AMR bacteria is genetically modified to modify, reduce or eliminate expression of an antibiotic resistance gene. Subsequent contact of the bacteria with the antibiotic will lead to the death or reduction in growth of the modified bacteria, for example, as described in Bikard et al., Cell Host Microbe, Vol. 12, 177-186 (2012) and Bikard et al., Nature Biotechnology Vol. 32 (11) 1146-51 (2014).

In one embodiment, the method comprises contacting the AMR bacteria with an effective amount of at least one antibiotic, phage, recombinant phage, packaged phagemid, plasmid, or combination thereof.

In one embodiment, bacteria producing the phage, recombinant phage, packaged phagemid or plasmid can be administered. In one embodiment, the phage, recombinant phage, packaged phagemid or plasmid can be directly administered.

In one embodiment, the antibiotic (and corresponding antibiotic resistance gene) is selected from methicillin, streptomycin, vancomycin, clindamycin, metronidazole, sulphadoxine, trimethoprim, or any combination of 1, 2, 3, 4, 5, 6, or 7 of these antibiotics.

In one embodiment, the phage, recombinant phage, packaged phagemid or plasmid, encodes a nuclease selected from CRISPR-Cas and variants, TALENs and variants, zinc finger nuclease (ZFN) and ZFN variants, natural, evolved or engineered meganuclease or recombinase variants.

In a preferred embodiment, the AMR bacteria are contacted in situ with at least one vector (in particular at least 2, 3, 4, 5, 6, 7, 8, 9, 10 or more) that can transfer with high efficiency a nucleic acid into the bacteria to express (i) an exogenous enzyme (such as Cas9 or Cpf1 also known as Cas12a, such as Mad4 or such as Cms1) and/or (ii) crRNA and/or tracrRNA, in the bacteria, that genetically modifies a DNA encoding an antibiotic resistance gene. Alternatively, the vector can transfer with high efficiency a nucleic acid into the bacteria to express crRNA and/or tracrRNA in the bacteria and program an endogenous enzyme (such as a Type I CRISPR nuclease, e.g. a Cas3 nuclease) of the bacteria to genetically modify a DNA encoding an antibiotic resistance gene.

The antibiotic resistance gene can be targeted directly. Alternatively, or in addition, if the antibiotic resistance gene is encoded by a plasmid, plasmid sequences outside of the antibiotic resistance gene can be targeted. For example, a plasmid origin of replication can be targeted.

In a particular embodiment, the exogenous enzyme can result in a genetic modification where a Cas nuclease, such as a Cas9 nuclease, is used to make the desired modification. Thus, the invention contemplates introducing a cleavage, e.g. a double strand break, in the DNA of the bacterial DNA at a specific sequence, for example with a CRISPR/Cas system, together with non-homologous end joining (NHEJ) or homologous recombination (HR) to generate the desired genetic modification. In one embodiment, the DNA cleavage, e.g. a double strand break, is generated in the presence of an editing template comprising homologous region with DNA regions located around the specific sequence located in the bacterial DNA. In a particular embodiment, the genetic modification involves either NHEJ or HR endogenous repair mechanism of the host bacteria.

In a particular embodiment, the genetic modification involves a base editor or a prime editor, in particular a base editor as disclosed in the section “Enzymes/Systems for Inducing Modifications” below or a prime editor as disclosed in the section “Enzymes/Systems for Inducing Modifications” below.

The genetic modification can be a point mutation(s), a deletion(s), insertion(s) or any combination thereof. Preferably, the genetic modification is a point mutation, an insertion or a deletion inside a coding sequence leading to a frameshift mutation or a deletion mutation, preferably in an antibiotic resistance gene. The genetic modification preferably eliminates or reduces the expression of an antibiotic resistance gene. The genetic modification can be in the translated or untranslated regions of a gene. The genetic modification can be in the promoter region of a gene or within any other region involved in gene regulation. In one embodiment, the genetic modification integrates a phage genome or exogenous DNA into the host bacterial chromosome or endogenous plasmid(s). In one embodiment, the genetic modification results in expression of an exogenous protein from an integrated exogenous DNA in the host bacterial chromosome or endogenous plasmid(s).

In some embodiments, the genetic modification results in the change in at least 1, 2, 3, 4, 5, 6, 7, 8, 9, 10, 11, 12, 13, 14, 15, 20, 25, 30, 35, 40, 45, 50, 100, 200, 500, etc. amino acids to a different amino acid. In some embodiments, the genetic modification introduces a stop codon. In some embodiments, the genetic modification is outside protein coding sequences, within RNA, or within regulatory sequences.

### Production of Packaged Phagemids and Bacterial Virus Particles

Generation of packaged phagemids or bacterial virus particles are routine techniques well-known to one skilled in the art. A satellite phage and/or helper phage may be used to promote the packaging of the payload in delivery vehicles of the present invention. Helper phages provide functions in trans and are well known to the man skilled in the art. The helper phage comprises all the genes coding for the structural and functional proteins that are indispensable for the payload to be packaged, according to the invention (i.e. the helper phage provides all the necessary gene products for the assembly of the delivery vehicle). The helper phage may contain a defective origin of replication or packaging signal, or completely lack the latter, and hence it is incapable of self-packaging, thus only bacterial delivery particles carrying the payload or plasmid will be produced. Helper phages may be chosen so that they cannot induce lysis of the host used for the delivery particle production. One skilled in the art would understand that some bacteriophages are defective and need a helper phage for payload packaging. Thus, depending on the bacteriophage chosen in connection with the present invention to prepare the bacterial delivery particles, the person skilled in the art would know if a helper phage is required. Sequences coding for one or more proteins or regulatory processes necessary for the assembly or production of packaged payloads may be supplied in trans. For example, the stf, gpJ and gpH proteins may be provided in a plasmid under the control of an inducible promoter or expressed constitutively. In this case, the phage wild-type sequence may or not contain a deletion of the gene or sequence supplied in trans. Additionally, chimeric or modified phage sequences encoding a new function, like an engineered stf, gpJ or gpH protein, may be directly inserted into the desired position in the genome of the helper phage, hence bypassing the necessity of providing the modified sequence in trans. Methods for both supplying a sequence or protein in trans in the form of a plasmid, as well as methods to generate direct genomic insertions, modifications and mutations are well known to those skilled in the art.

Bacterial Elimination with Antibiotics

Particular bacteria or groups of bacteria such as AMR bacteria that have had the expression of their antibiotic resistance genes modified can be eliminated by treatment with antibiotic(s). Thus, the invention encompasses methods of treating a subject with an antibiotic after treatment to modify an antibiotic resistance gene. Preferably, the level of the AMR bacteria is measured before and after the treatment.

Preferably, the treatment is after the subject has undergone an invasive procedures (surgery, intubation, catheterization, etc.) or harsh conditioning procedures (immunosuppression, irradiation, etc.). In one embodiment, the antibiotic treatment occurs after the subject undergoes surgery. In a preferred embodiment, the surgery is a solid organ transplant surgery of the kidney or liver. In some embodiments, the subject is in the ICU and/or has undergone surgery, dialysis, or a transplant.

In one embodiment, the invention encompasses a method comprising measuring the level of an AMR bacteria, subsequently administering a phage, phagemid, and/or an antibiotic, and measuring the level of the AMR bacteria after the administration(s).

In one embodiment, the method comprises a step of confirming that the payload(s)/vector(s) work(s) on the patient or subject sample in vitro. Thus a sample of microbiota taken from the patient or subject can be contacted with the payload/vector in vitro and the effect of the payload/vector on antibiotic resistance assessed by routine in vitro procedures.

In some embodiments, the antibiotic is methicillin, streptomycin, vancomycin, clindamycin, or metronidazole, alone or in any possible combination. In some embodiments, the antibiotic is sulphadoxine, trimethoprim, or metronidazole, alone or in any possible combination. In some embodiments, the antibiotic is selected from methicillin, streptomycin, vancomycin, clindamycin, metronidazole, sulphadoxine, trimethoprim, or any combination of 1, 2, 3, 4, 5, 6, or 7 of these antibiotics.

In some embodiments, the antibiotic is selected from the group consisting of penicillins such as penicillin G, penicillin K, penicillin N, penicillin 0, penicillin V, methicillin, benzylpenicillin, nafcillin, oxacillin, cloxacillin, dicloxacillin, ampicillin, amoxicillin, pivampicillin, hetacillin, bacampicillin, metampicillin, talampicillin, epicillin, carbenicillin, ticarcillin, temocillin, mezlocillin, and piperacillin; cephalosporins such as cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, loracarbef, cefbuperazone, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; polymyxins such as polysporin, neosporin, polymyxin B, and polymyxin E, rifampicins such as rifampicin, rifapentine, and rifaximin; Fidaxomicin; quinolones such as cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, temafloxacin, tosufloxacin, clinafloxacin, gatifloxacin, gemifloxacin, moxifloxacin, sitafloxacin, trovafloxacin, prulifloxacin, delafloxacin, nemonoxacin, and zabofloxacin; sulfonamides such as sulfafurazole, sulfacetamide, sulfadiazine, sulfadimidine, sulfafurazole, sulfisomidine, sulfadoxine, sulfamethoxazole, sulfamoxole, sulfanitran, sulfadimethoxine, sulfametho-xypyridazine, sulfametoxydiazine, sulfadoxine, sulfametopyrazine, and terephtyl; macrolides such as azithromycin, clarithromycin, erythromycin, fidaxomicin, telithromycin, carbomycin A, josamycin, kitasamycin, midecamycin, oleandomycin, solithromycin, spiramycin, troleandomycin, tylosin, and roxithromycin; ketolides such as telithromycin, and cethromycin; fluoroketolides such as solithromycin; lincosamides such as lincomycin, clindamycin, and pirlimycin; tetracyclines such as demeclocycline, doxycycline, minocycline, oxytetracycline, and tetracycline; aminoglycosides such as amikacin, dibekacin, gentamicin, kanamycin, neomycin, netilmicin, sisomicin, tobramycin, paromomycin, and streptomycin; ansamycins such as geldanamycin, herbimycin, and rifaximin; carbacephems such as loracarbef; carbapenems such as ertapenem, doripenem, imipenem (or cilastatin), and meropenem; glycopeptides such as teicoplanin, vancomycin, telavancin, dalbavancin, and oritavancin; lincosamides such as clindamycin and lincomycin; lipopeptides such as daptomycin; monobactams such as aztreonam; nitrofurans such as furazolidone, and nitrofurantoin; oxazolidinones such as linezolid, posizolid, radezolid, and torezolid; teixobactin, clofazimine, dapsone, capreomycin, cycloserine, ethambutol, ethionamide, isoniazid, pyrazinamide, rifabutin, arsphenamine, chloramphenicol, fosfomycin, fusidic acid, metronidazole, mupirocin, platensimycin, quinupristin (or dalfopristin), thiamphenicol, tigecycline, tinidazole, trimethoprim, alatrofloxacin, fidaxomycin, nalidixic acid, rifampin, derivatives and combination thereof.

In some embodiments, the AMR bacteria is resistant to β-lactams, aminoglycosides, erythromycin or tetracycline. In these cases, the gene encoding the resistance gene for that antibiotic within the AMR bacteria can be modified to make the AMR bacteria susceptible to the antibiotic. In a further embodiment, the modified AMR bacteria is then treated with the specific antibiotic.

### Measurement of Bacterial Elimination

The elimination of AMR bacteria can be assessed by comparison with and without (control sample) the genetic modification treatment either in vitro or in vivo. Untreated samples can serve as control samples. The comparison is preferably performed by assessing the percentage of bacteria before and after the genetic modification treatment at least two timepoints and determining a reduced amount of the targeted AMR bacteria at a later timepoint.

Comparison in vitro can be performed by growing the bacteria in solid or liquid culture and determining the percentages or levels of an AMR bacteria over time. The percentages or levels can be determined by routine diagnostic procedures including antibiotic resistance/sensitivity, ELISA, PCR, High Resolution Melting, and nucleic acid sequencing.

Comparison in vivo can be performed by collecting samples (e.g., stool or swab) over time and determining the percentages or levels of a bacteria over time. The percentages can be determined by routine diagnostic procedures employing immunodetection (e.g. ELISA), nucleic acid amplification (e.g., PCR), High Resolution Melting, and nucleic acid sequencing.

Preferred levels of elimination of AMR bacteria are at least 70%, 80%, 90%, 95%, 97%, 98%, 99%, 99.9%, 99.99%, and 100% of the starting levels of an AMR bacteria. The “elimination” of the AMR bacteria can be either by killing of the bacteria or modification of the antibiotic resistant bacteria to an antibiotic sensitive bacteria. That is, an antibiotic sensitive bacteria is no longer an AMR bacteria.

### Enzymes/Systems for Inducing Modifications

In some embodiments, the genetic modification is made with one or more of the following enzymes/systems. Such base editing approach for modulation of the microbiota is further described in international application PCT/EP2021/059229.

Cytosine base editors (CBE) and Adenosine base editors (ABE), as described in Rees et al. Nat Rev Genet 19, 770-788 (2018) which is hereby incorporated by reference.

So far there are seven types of DNA base editors described:
Cytosine Base Editor (CBE) that convert C:G into T:A (Komor, A et al. Programmable editing of a target base in genomic DNA without double-stranded DNA cleavage. Nature 533:420-4. (2016))
Adenine Base Editor (ABE) that convert A:T into G:C (Gaudelli, N. M. et al. Programmable base editing of AT to GC in genomic DNA without DNA cleavage. Nature 551(7681) 464-471 (2017))
Cytosine Guanine Base Editor (CGBE) that convert C:G into G:C (Chen, L et al. Precise and programmable C:G to G:C base editing in genomic DNA. Biorxiv (2020); Kurt, I et al. CRISPR C-to-G base editors for inducing targeted DNA transversions in human cells. Nature Biotechnology (2020))
Cytosine Adenine Base Editor (CABE) that convert C:G into A:T (Zhao, D et al. New base editors change C to A in bacteria and C to G in mammalian cells. Nature Biotechnology (2020))
Adenine Cytosine Base Editor (ACBE) that convert A:T into C:G (WO2020181180)
Adenine Thymine Base Editor (ATBE) that convert A:T into T:A (WO2020181202)
Thymine Adenine Base Editor (TABE) that convert T:A into A:T (WO2020181193; WO2020181178; WO2020181195).

Base editors differ in the base modification enzymes. CBE rely on ssDNA cytidine deaminase among which: APOBEC1, rAPOBEC1, APOBEC1 mutant or evolved version (evoAPOBEC1), and APOBEC homologs (APOBEC3A (eA3A), Anc689), Cytidine deaminase 1 (CDA1), evoCDA1, FERNY, evoFERNY.

ABE rely on deoxyadenosine deaminase activity of a tandem fusion TadA-TadA* where TadA* is an evolved version of TadA, an E. coli tRNA adenosine deaminase enzyme, able to convert adenosine into Inosine on ssDNA. TadA* include TadA-8a-e and TadA-7.10.

Except from base modification enzyme there has been also modifications implemented to base editor to increase editing efficacy, precision and modularity:
the addition of one or two uracil DNA glycosylase inhibitor domain (UGI) to prevent base excision repair mechanism to revert base edition
the addition of Mu-GAM that decrease insertion-deletion rate by inhibiting Non-homologous end joining mechanism in the cell (NHEJ)
the use of nickase active Cas9 (nCas9 D10A) that, by creating nicks on the non-edited strand favors its repair and consequently the fixation of the edited base.
the use of diverse Cas proteins from for example different organisms, mutants with different PAM motifs or different fidelity or different family (e.g. Cas12a).

Non-limiting examples of DNA-based editor proteins include BE1, BE2, BE3, BE4, BE4-GAM, HF-BE3, Sniper-BE3, Target-AID, Target-AID-NG, ABE, EE-BE3, YE1-BE3, YE2-BE3, YEE-BE3, BE-PLUS, SaBE3, SaBE4, SaBE4-GAM, Sa(KKH)-BE3, VQR-BE3, VRER-BE3, EQR-BE3, xBE3, Cas12a-BE, Ea3A-BE3, A3A-BE3, TAM, CRISPR-X, ABE7.9, ABE7.10, ABE7.10*, xABE, ABESa, VQR-ABE, VRER-ABE, Sa(KKH)-ABE, ABE8e, SpRY-ABE, SpRY-CBE, SpG-CBE4, SpG-ABE, SpRY-CBE4, SpCas9-NG-ABE, SpCas9-NG-CBE4, enAsBE1.1, enAsBE1.2, enAsBE1.3, enAsBE1.4, AsBE1.1, AsBE1.4, CRISPR-Abest, CRISPR-Cbest, eA3A-BE3, AncBE4.

Cytosine Guanine Base Editors (CGBE) consist of a nickase CRISPR fused to:
a. A cytosine deaminase (rAPOBEC) and base excision repair proteins (e.g. rXRCC1) (Chen, L et al. Precise and programmable C:G to G:C base editing in genomic DNA. Biorxiv (2020)).

b. A rat APOBEC1 variant (R33A) protein and an E. coli-derived uracil DNA N-glycosylase (eUNG) (Kurt, I et al. CRISPR C-to-G base editors for inducing targeted DNA transversions in human cells. Nature Biotechnology (2020)).

Cytosine Adenine Base Editors (CABE) consist of a Cas9 nickase, a cytidine deaminase (e.g. AID), and a uracil-DNA glycosylase (Ung) (Zhao, D et al. New base editors change C to A in bacteria and C to G in mammalian cells. Nature Biotechnology (2020)).

ACBE include a nucleic acid programmable DNA-binding protein and an adenine oxidase (WO2020181180).

ATBE consist of a Cas9 nickase and one or more adenosine deaminase or an oxidase domain (WO2020181202).

TABE consist of a Cas9 nickase and an adenosine methyltransferase, a thymine alkyltransferase, or an adenosine deaminase domain (WO2020181193; WO2020181178; WO2020181195).

Base editor molecules can also consist of two or more of the above listed editor enzymes fused to a Cas protein (e.g. combination of an ABE and CBE). These biomolecules are named dual base editors and enable the editing of two different bases (Grunewald, J et al. A dual-deaminase CRISPR base editor enables concurrent adenine and cytosine editing, Nature Biotechnology (2020); Li, C et al. Targeted, random mutagenesis of plant genes with dual cytosine and adenine base editors, Nature Biotechnology (2020)).

Prime editors (PE), as described in Anzalone et al., Nature volume 576, pages 149-157(2019), which is hereby incorporated by reference, consist of a nCas9 fused to a reverse transcriptase used in combination with a prime editing RNA (pegRNA, a guide RNA that includes a template region for the reverse transcription).

Prime Editing allows introduction of insertions, deletions (indels), and 12 base-to-base conversions. Prime editing relies on the ability of a reverse transcriptase (RT), fused to a Cas nickase variant, to convert RNA sequence brought by a prime editing guide RNA (pegRNA) into DNA at the nick site generated by the Cas protein. The DNA flap generated from this process is then included or not in the targeted DNA sequence.

Prime editing systems include:
a Cas nickase variant such as Cas9-H840A fused to a reverse transcriptase domain such as M-MLV RT or its mutant version (M-MLV RT(D200N), M-MLV RT(D200N/L603W), M-MLV RT(D200N/L603W/T330P/T306K/W313F)
a prime editing guide RNA (pegRNA)

To favor editing, the prime editing system can include the expression of an additional sgRNA targeting the Cas nickase activity towards the non-edited DNA strand ideally only after the resolution of the edited strand flap by designing the sgRNA to anneal with the edited strand but not with the original strand.

Non-limiting examples of prime editing systems include PE1, PE1-M1, PE1-M2, PE1-M3, PE1-M6, PE1-M15, PE1-M3inv, PE2, PE3, PE3b.

Cas9 Retron preciSe Parallel Editing via homologY (‘CRISPEY’), a retron RNA fused to the sgRNA and expressed together with Cas9 and the retron proteins including at least the reverse transcriptase (Sharon, E. et al. Functional Genetic Variants Revealed by Massively Parallel Precise Genome Editing. Cell 175, 544-557.e16 (2018)).

The SCRIBE strategy: a retron system expressed in combination with a recombinase promoting the recombination of single stranded DNA, also known as single stranded annealing proteins (SSAPs) (Farzadfard, F. & Lu, T. K. Genomically encoded analog memory with precise in vivo DNA writing in living cell populations. Science 346, 1256272 (2014)). Such recombinases include but are not limited to phage recombinases such as lambda red, recET, Sak, Sak4, and newly described SSAPs described in Wannier et al., (bioRxiv 2020.01.14.906594), which is hereby incorporated by reference.

The targetron system based on group II introns described in Karberg et al., Nature Biotechnology volume 19, pages 1162-1167(2001), which is hereby incorporated by reference, and which has been adapted to many bacterial species.

Other retron based gene targeting approaches, as described in Simon et al., Nucleic Acids Research, Volume 47, Issue 21, 2 Dec. 2019, Pages 11007-11019, which is hereby incorporated by reference.

CRISPR-Cas. The CRISPR system contains two distinct elements, i.e. i) an endonuclease, in this case the CRISPR associated nuclease (Cas or “CRISPR associated protein”) and ii) a guide RNA. Depending on the type of CRISPR system, the guide RNA may be in the form of a chimeric RNA which consists of the combination of a CRISPR (crRNA) bacterial RNA and a tracrRNA (trans-activating RNA CRISPR) (Jinek et al., Science 2012). The guide RNA combines the targeting specificity of the crRNA corresponding to the “spacing sequences” that serve as guides to the Cas proteins, and the conformational properties of the tracrRNA in a single transcript. When the guide RNA and the Cas protein are expressed simultaneously in the cell, the target genomic sequence can be permanently interrupted (and causing disappearance of the targeted and surrounding sequences and/or cell death, depending on the location) or modified. The modification may be guided by a repair matrix.

The CRISPR system includes two main classes depending on the nuclease mechanism of action:
Class 1 is made of multi-subunit effector complexes and includes type I, Ill and IV
Class 2 is made of single-unit effector modules, like Cas9 nuclease, and includes type II (II-A, II-B, II-C, II-C variant), V (V-A, V-B, V-C, V-D, V-E, V-U1, V-U2, V-U3, V-U4, V-U5) and VI (VI-A, VI-B1, VI-B2, VI-C, VI-D)

The sequence of interest according to the present invention comprises a nucleic acid sequence encoding Cas protein. A variety of CRISPR enzymes are available for use as a sequence of interest on the plasmid according to the present invention. In some embodiments, the CRISPR enzyme is a Type II CRISPR enzyme, a Type II-A or Type II-B CRISPR enzyme. In another embodiment, the CRISPR enzyme is a Type I CRISPR enzyme such as a Cas3 nuclease or a Type III CRISPR enzyme. In some embodiments, the CRISPR enzyme catalyzes DNA modification. In some other embodiments, the CRISPR enzyme catalyzes RNA modification. For instance, Cas13-deaminase fusions have been used for RNA base editing thus modifying RNA (David B T Cox et al, Science, 358 (6366) p. 1019-1027, 2017 Nov. 24). In one embodiment, the CRISPR enzymes may be coupled to a guide RNA or single guide RNA (sgRNA). In preferred embodiment, the guide RNA or sgRNA targets an antibiotic resistance gene. In certain embodiments, the guide RNA or sgRNA targets or further targets a gene selected from the group consisting of an antibiotic resistance gene, virulence protein or factor gene, toxin protein or factor gene, a bacterial receptor gene, a membrane protein gene, a structural protein gene, a secreted protein gene, a gene expressing resistance to a drug in general and a gene causing a deleterious effect to the host. Preferably, the CRISPR enzyme makes a double strand break. In some embodiments, the CRISPR enzyme makes a single strand break or nicks. In some embodiments, the CRISPR enzyme does not make any break in the DNA or RNA.

In a particular embodiment, at least two distinct vectors are used, in particular at least 3, 4, 5, 6, 7, 8, 9, 10 or more, each vector comprising guide RNA or sgRNA which targets a different location in the targeted bacteria, in particular targeting different locations within an antibiotic resistance gene, or targeting different antibiotic resistant genes, or targeting different antibiotic resistant genes and other genes of interest, such as virulence protein or factor gene, toxin protein or factor gene, a bacterial receptor gene, a membrane protein gene, a structural protein gene, a secreted protein gene, a gene expressing resistance to a drug in general and a gene causing a deleterious effect to the host.

In another particular embodiment, at least two distinct vectors are used, in particular at least 3, 4, 5, 6, 7, 8, 9, 10 or more, each vector comprising guide RNA or sgRNA which targets different types of targeted bacteria, in particular bacteria with different antibiotic resistant genes.

The sequence of interest may comprise a nucleic acid sequence encoding a guide RNA or sgRNA to guide the Cas protein endogenous to the targeted bacteria, alone or in combination with a Cas protein and/or a guide RNA encoded by the payload.

Non-limiting examples of Cas proteins as part of a multi-subunit effector or as a single-unit effector include Cas1, Cas1B, Cas2, Cas3, Cas4, Cas5, Cas6, Cas7, Cas8, Cas9 (also known as Csn1 and Csx12), Cas10, Cas11 (SS), Cas12a (Cpf1), Cas12b (C2c1), Cas12c (C2c3), Cas12d (CasY), Cas12e (CasX), C2c4, C2c8, C2c5, C2c10, C2c9, Cas13a (C2c2), Cas13b (C2c6), Cas13c (C2c7), Cas13d, Csa5, Csc1, Csc2, Cse1, Cse2, Csyl, Csy2, Csy3, Csf1, Csf2, Csf3, Csf4, Csm1, Csm2, Csm3, Csm4, Csm5, Csm6, Cmr1, Cmr3, Cmr4, Cmr5, Cmr6, Csn2, Csb1, Csb2, Csb3, Csx17, Csx14, Csx10, Csx16, CsaX, Csx13, Csx1, Csx15, SdCpf1, CmtCpf1, TsCpf1, CmaCpf1, PcCpf1, ErCpf1, FbCpf1, UbcCpf1, AsCpf1, LbCpf1, Mad4, Mad7, Cms1, homologues thereof, orthologues thereof, variants thereof, or modified versions thereof. In some embodiments, the CRISPR enzyme cleaves both strands of the target nucleic acid at the Protospacer Adjacent Motif (PAM) site.

In various embodiments, the invention encompasses fusion proteins comprising a Cas9 (e.g., a Cas9 nickase) domain and a deaminase domain. In some embodiments, the fusion protein comprises Cas9 and a cytosine deaminase enzyme, such as APOBEC enzymes, or adenosine deaminase enzymes, such as ADAT enzymes, for example as disclosed in U.S. Patent Publ. 2015/0166980, which is hereby incorporated by reference. In one embodiment, the deaminase is an ACF1/ASE deaminase.

In various embodiments, the APOBEC deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In various embodiments, the fusion protein comprises a Cas9 domain, a cytosine deaminase domain, and a uracil glycosylase inhibitor (UGI) domain.

In one embodiment, the deaminase is an adenosine deaminases that deaminate adenosine in DNA, for example as disclosed in U.S. Pat. No. 10,113,163, which is hereby incorporated by reference. In some embodiments, the fusion proteins further comprise a nuclear localization sequence (NLS), and/or an inhibitor of base repair, such as, a nuclease dead inosine specific nuclease (dISN), for example as disclosed in U.S. Pat. No. 10,113,163. In various embodiments, the invention encompasses fusion proteins comprising a catalytically impaired Cas9 endonuclease fused to an engineered reverse transcriptase, programmed with a prime editing guide RNA (pegRNA) that both specifies the target site and encodes the desired edit, for example as described in Anzalone et al., Nature, Vol. 576, pages 149-157 (2019), which is hereby incorporated by reference.

In a particular embodiment, the CRISPR enzyme is any Cas9 protein, for instance any naturally-occurring bacterial Cas9 as well as any variants, homologs or orthologs thereof.

By “Cas9” is meant a protein Cas9 (also called Csn1 or Csx12) or a functional protein, peptide or polypeptide fragment thereof, i.e. capable of interacting with the guide RNA(s) and of exerting the enzymatic activity (nuclease) which allows it to perform the double-strand cleavage of the DNA of the target genome. “Cas9” can thus denote a modified protein, for example truncated to remove domains of the protein that are not essential for the predefined functions of the protein, in particular the domains that are not necessary for interaction with the gRNA (s). In some embodiments, the CAS9 is a dCas9 (dead-Cas9) or nCas9 (nickase Cas9) lacking double stranded DNA cleavage activity.

The sequence encoding Cas9 (the entire protein or a fragment thereof) as used in the context of the invention can be obtained from any known Cas9 protein (Fonfara et al., 2014; Koonin et al., 2017). Examples of Cas9 proteins useful in the present invention include, but are not limited to, Cas9 proteins of Streptococcus pyogenes (SpCas9), Streptococcus thermophiles (St1Cas9, St3Cas9), Streptococcus mutans, Staphylococcus aureus (SaCas9), Campylobacter jejuni (CjCas9), Francisella novicida (FnCas9) and Neisseria meningitides (NmCas9).

The sequence encoding Cpf1 (Cas12a) (the entire protein or a fragment thereof) as used in the context of the invention can be obtained from any known Cpf1 (Cas12a) protein (Koonin et al., 2017). Examples of Cpf1(Cas12a) proteins useful in the present invention include, but are not limited to, Cpf1(Cas12a) proteins of Acidaminococcus sp, Lachnospiraceae bacteria and Francisella novicida.

The sequence encoding Cas13a (the entire protein or a fragment thereof) as used in the context of the invention can be obtained from any known Cas13a (C2c2) protein (Abudayyeh et al., 2017). Examples of Cas13a (C2c2) proteins useful in the present invention include, but are not limited to, Cas13a (C2c2) proteins of Leptotrichia wadei (LwaCas13a).

The sequence encoding Cas13d (the entire protein or a fragment thereof) as used in the context of the invention can be obtained from any known Cas13d protein (Yan et al., 2018). Examples of Cas13d proteins useful in the present invention include, but are not limited to, Cas13d proteins of Eubacterium siraeum and Ruminococcus sp.

The sequence encoding Mad4 (the entire protein or a fragment thereof) as used in the context of the invention is disclosed in international application WO2018/236548.

The sequence encoding Mad7 (the entire protein or a fragment thereof) as used in the context of the invention is disclosed in international application WO2018/236548.

The sequence encoding Cms1 (the entire protein or a fragment thereof) as used in the context of the invention is disclosed in international patent application WO2017/141173.

In some embodiments, other programmable nucleases can be used. These include an engineered TALEN (Transcription Activator-Like Effector Nuclease) and variants, engineered zinc finger nuclease (ZFN) variants, natural, evolved or engineered meganuclease or recombinase variants, and any combination or hybrids of programmable nucleases. Thus, the programmable nucleases provided herein may be used to selectively modify a DNA encoding a gene of interest, such as an antibiotic resistance gene.

In some embodiments, the genetic modification is made at the RNA level. RNA base editing is based on the same principle as DNA base editing: an enzyme catalyzing the conversion of a RNA base into another must be brought close to the target base to perform its conversion locally. In one embodiment, the enzyme used for RNA editing is an adenosine deaminase from ADAR family that converts Adenosine into Inosine in dsRNA structure. Several seminal studies used this specificity for dsRNA and fused the ADAR deaminase domain (ADAR_{DD}) to an antisense oligo in order to program local RNA base editing. More recently the ability of some CRISPR-Cas systems to bind RNA molecules was repurposed into RNA editing. Using catalytically dead Cas13b enzyme (dPspCas13b) fused to a hyperactive mutant of ADAR2 deaminase domain (ADAR2_{DD}-E4880 for REPAIRv1 and ADAR2DD-E4880-T375G for REPAIRv2) Cox et al improved specificity and efficiency compare to previous RNA editing strategies.

Non-limiting examples of RNA based editor proteins include REPAIRv1, REPAIRv2.

Vectors for Inducing Modifications

In various embodiments, one or more of the following vectors can be used to introduce the exogenous enzyme that results in a genetic modification:
engineered phages (in particular with engineered genome and optionally engineered capsid)
plasmid (e.g., a conjugative plasmid capable of transfer into a host cell), phage genome, phagemid or prophage.
Each vector may be as described above, eg, a phage capable of infecting a host cell or conjugative plasmid capable of introduction into a host cell, which can be introduced either by a phage particle via transduction or by a donor bacteria via conjugation. In an example, the vectors are in combination with an antibiotic agent (eg, a beta-lactam antibiotic) and/or with any other agent.
A bacteriophage whose genome has been genetically engineered for modifying a naturally occurring bacteria in situ and comprising a nucleic acid encoding a gene editing enzyme/system for transduction of a target bacteria in a mixed bacterial population wherein said gene editing enzyme/system modifies the genome of said target bacteria, but does not lead to the death of the target bacteria.

Vectors usable in the context of the invention, inside a bacterial delivery vehicle or not, include without limitation plasmid (e.g. conjugative plasmid), carrier bacteria comprising a plasmid such as conjugative plasmid, phagemid, packaged phagemid, and engineered (or recombinant) phage (with an engineered genome and/or capsid).

In a particular embodiment, the vector(s) used in the context of the invention is(are) inside a bacterial delivery vehicle.

The invention encompasses the use of these vectors wherein the gene modifying/editing enzyme/system targets a DNA within the target bacteria encoding an antibiotic resistance gene.

### Origin of Replication

In particular embodiments, the vector of the invention comprises an origin of replication. Origins of replication known in the art have been identified from species-specific plasmid DNAs (e.g. ColE1, RI, pT181, pSC101, pMB1, R6K, RK2, p15a and the like), from bacterial virus (e.g. TX174, M13, F1 and P4) and from bacterial chromosomal origins of replication (e.g. oriC).

In one embodiment, the vector of the invention does not comprise any functional bacterial origin of replication or contain an origin of replication that is inactive in the targeted bacteria. Thus, the vector of the invention cannot replicate by itself once it has been introduced into a bacterium.

In one embodiment, the origin of replication on the vector to be packaged is inactive in the targeted bacteria, meaning that this origin of replication is not functional in the bacteria transformed/transduced by the vector or in the bacteria being the receiver bacteria of a conjugative vector, thus preventing unwanted vector replication.

In one embodiment, the vector comprises a bacterial origin of replication that is functional in the bacteria used for the production of the vector.

Bacteria-Specific Origins of Replication

Plasmid replication depends on host enzymes and on plasmid-controlled cis and trans determinants. For example, some plasmids have determinants that are recognized in almost all gram-negative bacteria and act correctly in each host during replication initiation and regulation. Other plasmids possess this ability only in some bacteria (Kues, U and Stahl, U 1989 Microbiol Rev 53:491-516).

Plasmids are replicated by three general mechanisms, namely theta type, strand displacement, and rolling circle (reviewed by Del Solar et al. 1998 Microhio and Molec Biol. Rev 62:434-464) that start at the origin of replication. These replication origins contain sites that are required for interactions of plasmid and/or host encoded proteins.

Origins of replication may be moderate copy number, such as ColE1 ori from pBR322 (15-20 copies per cell) or the R6K plasmid (15-20 copies per cell) or may be high copy number, e.g. pUC oris (500-700 copies per cell), pGEM oris (300-400 copies per cell), pTZ oris (>1000 copies per cell) or pBluescript oris (300-500 copies per cell).

Examples of bacterial origins of replication include bacterial origins of replication selected in the group consisting of ColE1, pMB1 and variants (pBR322, pET, pUC, etc), p15a, ColA, ColE2, pOSAK, pSC101, R6K, IncW (pSa etc), IncFII, pT181, P1, F IncP, IncC, IncJ, IncN, IncP1, IncP4, IncQ, IncH11, RSF1010, CloDF13, NTP16, R1, f5, pPS10, pC194, pE194, BBR1, pBC1, pEP2, pWVO1, pLF1311, pAP1, pWKS1, pLS1, pLS11, pUB6060, pJD4, pIJ101, pSN22, pAMbetal, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RA1, TF-FC2, pMV158 and pUB113.

The bacterial origin of replication may be an E. coli origin of replication selected in the group consisting of ColE1, pMB1 and variants (pBR322, pET, pUC, etc), p15a, ColA, ColE2, pOSAK, pSC101, R6K, IncW (pSa etc), IncFII, pT181, P1, F IncP, IncC, IncJ, IncN, IncP1, IncP4, IncQ, IncH11, RSF1010, CloDF13, NTP16, R1, f5, pPS10.

The bacterial origin of replication may be selected in the group consisting of pC194, pE194, BBR1, pBC1, pEP2, pWVO1, pLF1311, pAP1, pWKS1, pLS1, pLS11, pUB6060, pJD4, pIJ101, pSN22, pAMbetal, pIP501, pIP407, ZM6100(Sa), pCU1, RA3, pMOL98, RK2/RP4/RP1/R68, pB10, R300B, pRO1614, pRO1600, pECB2, pCM1, pFA3, RepFIA, RepFIB, RepFIC, pYVE439-80, R387, phasyl, RA1, TF-FC2, pMV158 and pUB113.

More particularly, the bacterial origins of replication may be ColE1 and p15a.

Alternatively, the bacterial origin of replication may be functional in Propionibacterium and Cutibacterium more specifically in Propionibacterium freudenreichii and Cutibacterium acnes and may be selected from the group consisting of pLME108, pLME106, p545, pRGO1, pZGX01, pPG01, pYS1, FRJS12-3, FRJS25-1, pIMPLE-HL096PA1, A_15_1_R1.

Phage Origin of Replication

The vector according to the invention may comprise a phage replication origin which can initiate, with complementation in cis or in trans of a complete or modified phage genome, the replication of the payload for later encapsulation into the different capsids.

The phage origin can also be engineered to act as a bacterial origin of replication without the need to package any phage particles.

A phage origin of replication comprised in the payload/vector of the invention can be any origin of replication found in a phage.

Preferably, the phage origin of replication can be the wild-type or non-wildtype sequence of the M13, f1, φX174, P4, Lambda, P2, 186, Lambda-like, HK022, mEP237, HK97, HK629, HK630, mEP043, mEP213, mEP234, mEP390, mEP460, mEPx1, mEPx2, phi80, mEP234, T2, T4, T5, T7, RB49, phiX174, R17, PRD1 PI-like, P2-like, P22, P22-like, N15 and N15-like bacteriophages.

More preferably, the phage origin of replication is selected in the group consisting of phage origins of replication of M13, f1, φX174, P4, and Lambda.

In a particular embodiment, the phage origin of replication is the P4 origin of replication.

In a particular embodiment, the phage origin of replication is from Propionibacterium phages: BW-like phages such as Doucette, B22, E6, G4, BV-like phages such as Anatole, E1, B3, BX-like phages such as PFR1 and PFR2, filamentous B5 phage, BU-like phages (Cutibacterium acnes phages).

Conditional Origin of Replication

In a particular embodiment, the vector of the invention comprises a conditional origin of replication which is inactive in the targeted bacteria but is active in a donor bacterial cell.

In the context of the invention, a “conditional origin of replication” refers to an origin of replication whose functionality may be controlled by the presence of a specific molecule.

In a particular embodiment, the conditional origin of replication is an origin of replication, the replication of which depends upon the presence of one or more given protein, peptid, RNA, nucleic acid, molecule or any combination thereof.

In a particular embodiment, the replication of the vector comprising said origin of replication may further depend on a process, such as transcription, to activate said replication.

In the context of the invention, said conditional origin of replication is inactive in the targeted bacteria because of the absence of said given protein, peptid, RNA, nucleic acid, molecule or any combination thereof in said targeted bacteria.

In a particular embodiment, said conditional origin of replication is active in said donor bacterial cell because said donor bacterial cell expresses said given protein, peptid, RNA, nucleic acid, molecule or any combination thereof. In a particular embodiment, said protein, peptid, RNA nucleic acid, molecule or any combination thereof is expressed in trans in said donor bacterial cell.

By “in trans” is meant herein that said protein, peptid, RNA, nucleic acid, molecule or any combination thereof is not encoded on the same nucleic acid molecule as the one comprising the origin of replication. In a particular embodiment, said protein, peptid, RNA, nucleic acid, molecule or any combination thereof is encoded on a chromosome or on a plasmid. In a particular embodiment, said plasmid comprises an antibiotic resistance marker or an auxotrophic resistance marker. In an alternative embodiment, said plasmid is devoid of antibiotic resistance marker.

Since said conditional origin of replication is inactive in the targeted bacteria because of the absence of said given protein, peptid, RNA, nucleic acid, molecule or any combination thereof in said targeted bacteria, said conditional origin of replication may be selected depending on the specific bacteria to be targeted.

The conditional origin of replication disclosed herein may originate from plasmids, bacteriophages or PICIs which preferably share the following characteristics: they contain in their origin of replication repeat sequences, or iterons, and they code for at least one protein interacting with said origin of replication (i.e. Rep, protein 0, protein P, pri) which is specific to them.

By way of example, mention may be made of the conditional replication systems of the following plasmids and bacteriophages: RK2, R1, pSC101, F, Rts1, RSF1010, P1, P4, lambda, phi82, phi80.

In a particular embodiment, said conditional origin of replication is selected from the group consisting of the R6KA DNA replication origin and derivatives thereof, the IncPa oriV origin of replication and derivatives thereof, ColE1 origins of replication modified to be under an inducible promoter, and origins of replication from phage-inducible chromosomal islands (PICIs) and derivatives thereof.

In a particular embodiment, said conditional origin of replication is an origin of replication present in less than 50%, or less than 40%, less than 30%, less than 20%, less than 10% or less than 5% of the bacteria of the host microbiome.

In another particular embodiment, said conditional origin of replication comprises or consists of a sequence less than 80% identical, in particular less than 70%, less than 60%, less than 50%, less than 40%, less than 30%, less than 20%, less than 10%, less than 5% or less than 1% identical to the sequences of the origins of replication of the bacteria of the host microbiome, in particular of the bacteria representing more than 50%, more particularly more than 60%, more than 70%, more than 80%, more than 90% or more than 95% of the host microbiome.

As used herein, the term “phage-inducible chromosomal islands” or “PICIs” refers to mobile genetic elements having a conserved gene organization, and encoding a pair of divergent regulatory genes, including a PICI master repressor. Typically, in Gram-positive bacteria, left of rpr, and transcribed in the same direction, PICIs encode a small set of genes including an integrase (int) gene; right of rpr, and transcribed in the opposite direction, the PICIs encode an excision function (xis), and a replication module consisting of a primase homolog (pri) and optionally a replication initiator (rep), which are sometimes fused, followed by a replication origin (ori), next to these genes, and also transcribed in the same direction, PICIs encode genes involved in phage interference, and optionally, a terminase small subunit homolog (terS).

In a particular embodiment, said conditional origin of replication is an origin of replication derived from phage-inducible chromosomal islands (PICIs).

A particular conditional origin of replication has indeed been derived from PICIs.

It was shown that it is possible to derive novel conditionally replicative plasmids, in particular based on the primase-helicase and origin of replication from PICIs. These origins may be relatively rare in target strains, and more advantageously the primase-ori pair may be unique for each PICI, significantly reducing the possibility of undesired recombination or payload spread events. They can further be modified to further limit recombination chances and remove restriction sites to bypass target bacteria defense systems.

In a particular embodiment, said conditional origin of replication is derived from the origin of replication from the PICI of the Escherichia coli strain CFT073, disclosed in Fillol-Salom et al. (2018) The ISME Journal 12:2114-2128.

In a particular embodiment, said conditional origin of replication is the primase ori from the PICI of the Escherichia coli strain CFT073, typically of sequence SEQ ID NO: 3.

In another particular embodiment, said conditional origin of replication is the primase ori from the PICI of the Escherichia coli strain CFT073, devoid of at least 1, at least 2, at least 3, at least 4, at least 5, at least 6, at least 7, at least 8, at least 9, at least 10, at least 11, at least 12, at least 13, at least 14, at least 15 or at least 16 restriction site(s) selected from the group consisting of GAAABCC, GCCGGC, RCCGGY, GCNGC, TWCANNNNNNTGG (SEQ ID NO: 8), TGGCCA, ACCYAC, YGGCCR, AGACC, GCWGC, GGGANGC, GKAGATD, GCCGGYYD, GGCYAC, RGCCGGYYD, and VGCCGGYBD.

In a particular embodiment, said conditional origin of replication is the primase ori from the PICI of the Escherichia coli strain CFT073, devoid of the restriction site GAAABCC (SEQ ID NO: 4). Preferably, said conditional origin of replication is of sequence SEQ ID NO: 1.

In another particular embodiment, said conditional origin of replication is the primase ori from the PICI of the Escherichia coli strain CFT073 devoid of the restriction sites GAAABCC, GCCGGC, RCCGGY, GCNGC, TWCANNNNNNTGG (SEQ ID NO: 8), TGGCCA, ACCYAC, YGGCCR, AGACC, GCWGC, GGGANGC, GKAGATD, GCCGGYYD, GGCYAC, RGCCGGYYD, and VGCCGGYBD. Preferably, said conditional origin of replication is of sequence SEQ ID NO: 2.

In a particular embodiment, wherein said origin of replication is derived from phage-inducible chromosomal islands (PICIs), said conditional origin of replication is active in said donor bacterial cell because said donor bacterial cell expresses a rep protein, in particular a primase-helicase, in particular a primase-helicase of sequence SEQ ID NO: 20, typically encoded by a nucleic acid comprising or consisting of the sequence SEQ ID NO: 21.

It was demonstrated that these specific conditional origins of replication were particularly compatible with lambda-based packaging, leading to sufficiently high titers (>10¹⁰/mL) required for microbiota-related applications.

In a particular embodiment, when said vector is a phagemid, said origin of replication may be derived from a microorganism which is different from the one that is used to encode the structural elements of the capsid packaging said phagemid.

By “donor bacterial cell” is meant herein a bacterium that is capable of hosting a vector as defined above, of producing a vector as defined above and/or which is capable of transferring said vector as defined above to another bacterium. In a particular embodiment, said vector may be a phagemid, and said donor bacterial cell may then be a bacterial cell able to produce said phagemid, more particularly in the form of a packaged phagemid. In an alternative embodiment, said vector may be a plasmid, more particularly a conjugative plasmid, and said donor bacterial cell may then be a bacterium that is capable of transferring said conjugative plasmid to another bacterium, in particular by conjugation.

Preferably, said donor bacterial cell stably comprises said vector and is able to replicate said vector.

In a particular embodiment, when the conditional origin of replication of said vector is an origin of replication, the replication of which depends upon the presence of a given protein, peptid, nucleic acid, RNA, molecule or any combination thereof, said donor bacterial cell expresses said protein, peptid, nucleic acid, RNA, molecule or any combination thereof. Preferably, said protein, peptid, nucleic acid, RNA, molecule or any combination thereof is expressed in trans, as defined above.

In a particular embodiment, said donor bacterial cell stably comprises a nucleic acid encoding said protein, peptid, nucleic acid, RNA, molecule or any combination thereof.

In a particular embodiment, when said origin of replication is derived from phage-inducible chromosomal islands (PICIs), said conditional origin of replication is active in said donor bacterial cell because said donor bacterial cell expresses a rep protein, in particular a primase-helicase, in particular a primase-helicase of sequence SEQ ID NO: 20.

In a particular embodiment, said donor bacterial cell stably comprises a nucleic acid encoding said rep protein, in particular said primase-helicase, said nucleic acid typically comprising or consisting of the sequence SEQ ID NO: 21.

In a particular embodiment, said donor bacterial cell is a production cell line, in particular a cell line producing packaged phagemids including the vector of the invention.

Delivery Vehicle Incapable of Self-Reproduction

In a particular embodiment, the delivery vehicle, in particular the bacteriophage, bacterial virus particle or packaged phagemid, comprising the vector of the invention is incapable of self-reproduction.

In the context of the present invention, “self-reproduction” is different from “self-replication”, “self-replication” referring to the capability of replicating a nucleic acid, whereas “self-reproduction” refers to the capability of having a progeny, in particular of producing new delivery vehicles, said delivery vehicles being either produced empty or with a nucleic acid of interest packaged.

By “delivery vehicle incapable of self-reproduction” is meant herein that at least one, several or all functional gene(s) necessary to produce said delivery vehicle is(are) absent from said delivery vehicle (and from said vector included in said delivery vehicle). In a preferred embodiment, said at least one, several or all functional gene(s) necessary to produce said delivery vehicle is(are) present in the donor cell as defined above, preferably in a plasmid, in the chromosome or in a helper phage present in the donor cell as defined above, enabling the production of said delivery vehicle in said donor cell.

In the context of the invention, said functional gene necessary to produce a delivery vehicle may be absent through (i) the absence of the corresponding gene or (ii) the presence of the corresponding gene but in a non-functional form.

In an embodiment, the sequence of said gene necessary to produce said delivery vehicle is absent from said delivery vehicle. In a preferred embodiment, the sequence of said gene necessary to produce said delivery vehicle has been replaced by a nucleic acid sequence of interest, in particular by a nucleic acid sequence encoding enzymes or systems for inducing genetic modifications, as defined above.

Alternatively, said gene necessary to produce said delivery vehicle is present in said delivery vehicle in a non-functional form, for example in a mutant non-functional form, or in a non-expressible form, for example with deleted or mutated non-functional regulators. In a preferred embodiment, said gene necessary to produce said delivery vehicle is present in said delivery vehicle in a mutated form which renders it non-functional in the target cell, while remaining functional in the donor cell.

In the context of the invention, genes necessary to produce said delivery vehicle encompass any coding or non-coding nucleic acid required for the production of said delivery vehicle.

Examples of genes necessary to produce said delivery vehicle include genes encoding phage structural proteins; phage genes involved in the control of genetic expression; phage genes involved in transcription and/or translation regulation; phage genes involved in phage DNA replication; phage genes involved in production of phage proteins; phage genes involved in phage proteins folding; phage genes involved in phage DNA packaging; and phage genes encoding proteins involved in bacterial cell lysis.

Controlled Self-Inactivation or Self-Destruction of the Vector

In a particular embodiment, the vector of the invention can self-inactivate or self-destroy in a controlled way. In a particular embodiment, the vector of the invention comprises a nucleic acid sequence comprising a gene encoding a DNA modifying enzyme, e.g. a nuclease, which can be expressed in the targeted bacterial cell, wherein the DNA modifying enzyme when expressed from the nucleic acid sequence modifies said nucleic acid at one or multiple locations in the nucleic acid sequence. In some embodiments, modification by the DNA modifying enzyme occurs after the nucleic acid sequence of interest, as defined below, comprised by the vector of the invention, has been transcribed and translated. In some embodiments, the DNA modifying enzyme is a nuclease which cleaves the nucleic acid sequence, and the cleavage occurs after the nucleic acid sequence of interest comprised by the vector has been transcribed and translated.

In a particular embodiment, said controlled self-inactivation or self-destruction is carried out by preventing the transcription or translation of said gene encoding a DNA modifying enzyme for a certain amount of time, allowing transcription or translation of other sequence(s), in particular of the sequence of interest comprised by the vector, during this amount of time, and delaying expression of said DNA modifying enzyme during this amount of time, therefore delaying inactivation or loss of the nucleic acid vector.

Said controlled self-inactivation or self-destruction of the vector of the invention relies on the use of a system, which can be encoded on the nucleic acid vector, that is programmed to lead a DNA modification or cleavage at one or multiple location on the nucleic acid vector, which leads to the inactivation or degradation of the nucleic acid vector. Preferably, the system is independent of any induction system based on external stimuli or molecule and is contained within the same vector as the one carrying the nucleic acid of interest.

In some embodiments, the delayed destruction or inactivation system relies on the expression of the DNA modifying enzyme that is programmed to modify, e.g. cleave, one or multiple sequences that have been added or engineered at specific locations on the nucleic acid vector. In some embodiments, the DNA modifying enzyme is a nuclease. The nuclease can be a restriction enzyme, a meganuclease, a zinc finger or a TALEN. In some embodiments, the nuclease can be an RNA-guided nuclease and can target one or multiple sequences to be cleaved by encoding one or multiple crRNA, and optionally a tracrRNA on the vector.

The targeted sequences can be located at one or multiple locations in the nucleic acid vector. In one embodiment, the targeted sequences are within the nucleic acid of interest carried by the nucleic acid vector. In one embodiment, the targeted sequences are within a nuclease, or other modification enzyme, carried by the nucleic acid vector.

The targeted sequences to be inactivated can be within an origin of replication if an origin of replication is present, within the nucleic acid of interest or at any other essential locations of the nucleic acid vector. If the desired outcome does not rely on nor necessitate the death of the bacteria, the targeted sequences on the nucleic acid vector should be engineered to not be homologous to any sequences from the target bacterial cell chromosome.

To incorporate a delay between the nucleic acid of interest expression and the destruction of the nucleic acid vector, it is herein provided engineered mechanisms to ensure that the production of a correctly folded DNA modifying protein will take longer than the time required for the nucleic acid(s) of interest to be expressed and exercise its/their intended function(s). In some embodiments, this is achieved by engineering a weak promoter for the DNA modifying protein. In some embodiments, this is achieved by engineering a weak RBS for the DNA modifying protein. In some embodiments, this is achieved by recoding the DNA modifying protein sequence leading to a slower translation rate of its corresponding transcribed RNA. In some embodiments, this is achieved by adding a proteolytic degradation tag to the DNA modifying protein. These engineering approaches can be combined to achieve the desired delayed inactivation or cleavage of the nucleic acid vector.

In some embodiments, the sequence targeted by the DNA modifying enzyme on the nucleic acid vector carries mutations reducing the DNA modifying enzyme activity and delaying the loss of the vector. In the case where the DNA modifying enzyme is a nuclease, and more particularly an RNA guided nuclease, mismatches can be introduced between the guide RNA and the target to reduce the nuclease activity and delay the loss of the nucleic acid vector. The nature and position of mismatches that reduce the activity of Cas nucleases has been well characterized in the literature, for example in Jung et al., Cell 170, 35-47, 2017, and Jones et al., biorxiv.org/content/10.1101/696393v1, which are hereby incorporated by reference. Alternatively, the length of the guide RNA can be modulated to reduce the activity of the nuclease, e.g. a shorter or longer guide RNA can be used in comparison to the optimal guide RNA size. Any of these approaches can be combined to achieve the desired delayed cleavage of the nucleic acid vector.

In some embodiment, it is the transcription itself of the DNA modifying enzyme that can be delayed by engineering a genetic cascade where the transcription of the DNA modifying enzyme gene is activated by a protein or a molecule encoded via a gene constitutively expressed on the nucleic acid vector, or whose transcription can itself be controlled by another gene on the nucleic acid vector which can itself be constitutively expressed or controlled via another gene, etc.

In some embodiments, this control can be activated by the displacement, inversion or transposition of a sequence, required for the transcription, in frame with the nuclease gene itself. The displacement, inversion or transposition can be mediated via an exogenous recombinase.

In some embodiments, the nuclease is an endogenous nuclease, naturally found in the target bacteria or not, and not carried by the delivered nucleic acid vector.

Bacterial viruses (also called bacteriophages or phages) are small viruses displaying the ability to infect and kill bacteria while they do not affect cells from other organisms. Initially described almost a century ago by William Twort, and independently discovered shortly thereafter by Félix d'Herelle, more than 6000 different bacterial viruses have been discovered so far and described morphologically. The vast majority of these viruses are tailed while a small proportion are polyhedral, filamentous or pleomorphic. They may be classified according to their morphology, their genetic content (DNA vs. RNA), their specific host, the place where they live (marine virus vs. other habitats), and their life cycle. As intracellular parasites of bacterial cells, phages display different life cycles within the bacterial host: lytic, lysogenic, pseudo-lysogenic, and chronic infection. Lytic phages, once their DNA injected into their host, replicate their own genome and produce new viral particles at the expense of the host. Indeed, they cause lysis of the host bacterial cell as a normal part of the final stage of their life cycles to liberate viral particles. Temperate phages can either replicate by means of the lytic life cycle and cause lysis of the host bacterium, or they can incorporate their DNA into the host bacterial DNA and become non-infectious prophages (lysogenic cycle). In some embodiments, lytic phages are used.

Unlike classical chemically-based antibiotics that are active against a broad spectrum of bacterial species, a bacteriophage can infect and kill only a small number of different closely-related bacteria.

The use of packaged phagemids allows to have a defined and control way of killing the host. Example of packaged phagemids encoding CRISPR-Cas9 or toxins have shown promising results in killing targeted bacterial population (Bikard et al., 2012, Cell Host & Microbe 12, 177-186; Jiang et al., 2013, Nat Biotechnol 31, 233-239; Krom et al., 2015, Nano Letters 15, 4808-4813; Bikard et al, 2014, Nat Biotech 11, Vol. 32, Citorik, R et al, 2014, Nat Biotech 11, Vol. 32).

### Sequence of Interest Under the Control of the Promoter

The vector can comprise a sequence of interest under the control of a promoter.

In one embodiment, the sequence of interest is a programmable nuclease circuit to be delivered to the targeted bacteria, in particular as defined above. This programmable nuclease circuit may be able to mediate in vivo sequence-specific elimination of bacteria that contain a target gene of interest. Some embodiments of the present disclosure relate to engineered variants of the Type II CRISPR-Cas (Clustered Regularly Interspaced Short Palindromic Repeats-CRISPR-associated) system of Streptococcus pyogenes. Other programmable nucleases that can be used include other CRISPR-Cas systems, engineered TALEN (Transcription Activator-Like Effector Nuclease) variants, engineered zinc finger nuclease (ZFN) variants, natural, evolved or engineered meganuclease or recombinase variants, and any combination or hybrids of programmable nucleases.

Other sequences of interest, preferably programmable, can be added to the payload/vector so as to be delivered to targeted bacteria.

Preferably, the sequence of interest circuit added to the payload/vector leads to the reduction or elimination of expression of an antibiotic resistance gene.

In a particular embodiment, the nucleic acid sequence of interest is selected from the group consisting of a sequence encoding a Cas nuclease, a guide RNA, a single guide RNA (sgRNA), a CRISPR locus, a gene encoding an enzyme such as a nuclease, a kinase, a TALEN, a ZFN, a meganuclease, a recombinase, a base editor or a prime editor. These proteins can also be modified or engineered to include extra features, like the addition or removal of a function (e.g. dCas9).

### Targeted Bacteria

The AMR bacteria targeted by a composition of the invention can be present in vivo, in a mammalian organism, or in vitro, for example in liquid or solid culture.

A microbiome may comprise a variety of endogenous AMR bacteria species, any of which may be targeted in accordance with the present disclosure. In some embodiments, the species of targeted AMR bacterial cells may depend on the type of bacteriophages being used for preparing the bacterial virus particles. For example, some bacteriophages exhibit tropism for, or preferentially target, specific host species of bacteria. Other bacteriophages do not exhibit such tropism and may be used to target a number of different genus and/or species of endogenous bacterial cells.

Examples of bacterial cells include, without limitation, cells from bacteria of the genus Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Vibrio spp., Bacillus spp., Erysipelothrix spp., Salmonella spp., Streptomyces spp., Streptococcus spp., Staphylococcus spp., Bacteroides spp., Prevotella spp., Clostridium spp., Bifidobacterium spp., Clostridium spp., Brevibacterium spp., Lactococcus spp., Leuconostoc spp., Actinobacillus spp., Selnomonas spp., Shigella spp., Zymonas spp., Mycoplasma spp., Treponema spp., Leuconostoc spp., Corynebacterium spp., Enterococcus spp., Enterobacter spp., Pyrococcus spp., Serratia spp., Morganella spp., Parvimonas spp., Fusobacterium spp., Actinomyces spp., Porphyromonas spp., Micrococcus spp., Bartonella spp., Borrelia spp., Brucella spp., Campylobacter spp., Chlamydophilia spp., Cutibacterium spp., Propionibacterium spp., Gardnerella spp., Ehrlichia spp., Haemophilus spp., Leptospira spp., Listeria spp., Mycoplasma spp., Nocardia spp., Rickettsia spp., Ureaplasma spp., Lactobacillus spp. and a mixture thereof.

Thus, bacterial virus particles may target (e.g., specifically target) a bacterial cell from any one or more of the foregoing genus of bacteria to specifically deliver the plasmid according to the invention.

Preferably, the targeted bacteria can be selected from the group consisting of Yersinia spp., Escherichia spp., Klebsiella spp., Acinetobacter spp., Pseudomonas spp., Helicobacter spp., Vibrio spp, Salmonella spp., Streptococcus spp., Staphylococcus spp., Bacteroides spp., Clostridium spp., Shigella spp., Enterococcus spp., Enterobacter spp., Listeria spp., Cutibacterium spp., Propionibacterium spp., Fusobacterium spp., Porphyromonas spp. and Gardnerella spp.

In some embodiments, bacterial cells of the present invention are anaerobic bacterial cells (e.g., cells that do not require oxygen for growth). Anaerobic bacterial cells include facultative anaerobic cells such as but not limited to Escherichia coli, Shewanella oneidensi, Gardnerella vaginalis and Listeria. Anaerobic bacterial cells also include obligate anaerobic cells such as, for example, Bacteroides, Clostridium, Cutibacterium, Propionibacterium, Fusobacterium and Porphyromonas species. In humans, anaerobic bacteria are most commonly found in the gastrointestinal tract. In some particular embodiment, the targeted bacteria are thus bacteria most commonly found in the gastrointestinal tract. Bacteriophages used for preparing the bacterial virus particles, and then the bacterial virus particles, may target (e.g., to specifically target) anaerobic bacterial cells according to their specific spectra known by the person skilled in the art to specifically deliver the plasmid.

In some embodiments, the targeted bacterial cells are, without limitation, Bacteroides faecis, Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphilococcus lugdunensis, Leuconostoc oenos, Corynebacterium xerosis, Lactobacillus plantarum, Lactobacillus rhamnosus, Lactobacillus casei, Lactobacillus acidophilus, Enterococcus faecalis, Bacillus coagulans, Bacillus cereus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Streptomyces phaechromogenes, Streptomyces ghanaenis, Klebsiella pneumoniae, Enterobacter cloacae, Enterobacter aerogenes, Serratia marcescens, Morganella morganii, Citrobacter freundii, Propionibacterium freudenreichii, Pseudomonas aerigunosa, Parvimonas micra, Prevotella intermedia, Fusobacterium nucleatum, Prevotella nigrescens, Actinomyces israelii, Porphyromonas endodontalis, Porphyromonas gingivalis Micrococcus luteus, Bacillus megaterium, Aeromonas hydrophila, Aeromonas caviae, Bacillus anthracis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Campylobacter coli, Campylobacter fetus, Chlamydia pneumoniae, Chlamydia trachomatis, Chlamydophila psittaci, Clostridium botulinum, Clostridium difficile, Clostridium perfringens, Clostridium tetani, Corynebacterium diphtheria, Cutibacterium acnes (formerly Propionibacterium acnes), Ehrlichia canis, Ehrlichia chaffeensis, Enterococcus faecium, Francisella tularensis, Haemophilus influenza, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumonia, Neisseria gonorrhoeae, Neisseria meningitides, Nocardia asteroids, Rickettsia rickettsia, Salmonella enteritidis, Salmonella typhi, Salmonella paratyphi, Salmonella typhimurium, Shigella flexnerii, Shigella dysenteriae, Staphylococcus saprophyticus, Streptococcus pneumoniae, Streptococcus pyogenes, Gardnerella vaginalis, Streptococcus viridans, Treponema pallidum, Ureaplasma urealyticum, Vibrio cholera, Vibrio parahaemolyticus, Yersinia pestis, Yersinia enterocolitica, Yersinia pseudotuberculosis, Actinobacter baumanii, Pseudomonas aerigunosa, and a mixture thereof, preferably the bacteria of interest are selected from the group consisting of Escherichia coli, Enterococcus faecium, Staphylococcus aureus, Klebsiella pneumoniae, Acinetobacter baumanii, Pseudomonas aeruginosa, Enterobacter cloacae, and Enterobacter aerogenes, and a mixture thereof.

In some embodiments, the targeted bacterial cells are, without limitation, Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Moryella, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter, Weisella, Clostridium, Bacteroides, Ruminococcus, Faecalibacterium, Treponema, Phascolarctobacterium, Megasphaera, Faecalibacterium, Bifidobacterium, Lactobacillus, Sutterella, and/or Prevotella.

In other embodiments, the targeted bacteria cells are, without limitation, Achromobacter xylosoxidans, Acidaminococcus fermentans, Acidaminococcus intestini, Acidaminococcus sp., Acinetobacter baumannii, Acinetobacter junii, Acinetobacter lwoffii, Actinobacillus capsulatus, Actinomyces naeslundii, Actinomyces neuii, Actinomyces odontolyticus, Actinomyces radingae, Adlercreutzia equolifaciens, Aeromicrobium massiliense, Aggregatibacter actinomycetemcomitans, Akkermansia muciniphila, Aliagarivorans marinus, Alistipes finegoldii, Alistipes indistinctus, Alistipes Mops, Alistipes onderdonkii, Alistipes putredinis, Alistipes senegalensis, Alistipes shahii, Alistipes timonensis, Alloscardovia omnicolens, Anaerobacter polyendosporus, Anaerobaculum hydrogeniformans, Anaerococcus hydrogenalis, Anaerococcus prevotii, Anaerococcus senegalensis, Anaerofustis stercorihominis, Anaerostipes caccae, Anaerostipes hadrus, Anaerotruncus colihominis, Aneurinibacillus aneurinilyticus, Bacillus licheniformis, Bacillus massilioanorexius, Bacillus massiliosenegalensis, Bacillus simplex, Bacillus smithii, Bacillus subtilis, Bacillus thuringiensis, Bacillus timonensis, Bacteroides xylanisolvens, Bacteroides acidifaciens, Bacteroides caccae, Bacteroides capillosus, Bacteroides cellulosilyticus, Bacteroides clarus, Bacteroides coprocola, Bacteroides coprophilus, Bacteroides dorei, Bacteroides eggerthii, Bacteroides faecis, Bacteroides finegoldii, Bacteroides fluxus, Bacteroides fragilis, Bacteroides gallinarum, Bacteroides intestinalis, Bacteroides nordii, Bacteroides oleiciplenus, Bacteroides ovatus, Bacteroides pectinophilus, Bacteroides plebeius, Bacteroides salanitronis, Bacteroides salyersiae, Bacteroides sp., Bacteroides stercoris, Bacteroides thetaiotaomicron, Bacteroides uniformis, Bacteroides vulgatus, Bacteroides xylanisolvens, Bacteroidespectinophilus ATCC, Barnesiella intestinihominis, Bavariicoccus seileri, Bifidobacterium adolescentis, Bifidobacterium angulatum, Bifidobacterium animalis, Bifidobacterium bifidum, Bifidobacterium breve, Bifidobacterium catenulatum, Bifidobacterium dentium, Bifidobacterium gallicum, Bifidobacterium longum, Bifidobacterium pseudocatenulatum, Bifidobacterium stercoris, Bilophila wadsworthia, Blautia faecis, Blautia hansenii, Blautia hydrogenotrophica, Blautia luti, Blautia obeum, Blautia producta, Blautia wexlerae, Brachymonas chironomi, Brevibacterium senegalense, Bryantella formatexigens, butyrate-producing bacterium, Butyricicoccus pullicaecorum, Butyricimonas virosa, Butyrivibrio crossotus, Butyrivibrio fibrisolvens, Caldicoprobacter faecalis, Campylobacter concisus, Campylobacter jejuni, Campylobacter upsaliensis, Catenibacterium mitsuokai, Cedecea davisae, Cellulomonas massiliensis, Cetobacterium somerae, Citrobacter braakii, Citrobacter freundii, Citrobacter pasteurii, Citrobacter sp., Citrobacter youngae, Cloacibacillus evryensis, Clostridiales bacterium, Clostridioides difficile, Clostridium asparagiforme, Clostridium bartlettii, Clostridium boliviensis, Clostridium bolteae, Clostridium hathewayi, Clostridium hiranonis, Clostridium hylemonae, Clostridium leptum, Clostridium methylpentosum, Clostridium nexile, Clostridium orbiscindens, Clostridium ramosum, Clostridium scindens, Clostridium sp, Clostridium sp., Clostridium spiroforme, Clostridium sporogenes, Clostridium symbiosum, Collinsella aerofaciens, Collinsella intestinalis, Collinsella stercoris, Collinsella tanakaei, Coprobacillus cateniformis, Coprobacter fastidiosus, Coprococcus catus, Coprococcus comes, Coprococcus eutactus, Corynebacterium ammoniagenes, Corynebacterium amycolatum, Corynebacterium pseudodiphtheriticum, Cutibacterium acnes, Dermabacter hominis, Desulfitobacterium hafniense, Desulfovibrio fairfieldensis, Desulfovibrio piger, Dialister succinatiphilus, Dielma fastidiosa, Dorea formicigenerans, Dorea longicatena, Dysgonomonas capnocytophagoides, Dysgonomonas gadei, Dysgonomonas mossii, Edwardsiella tarda, Eggerthella lenta, Eisenbergiella tayi, Enorma massiliensis, Enterobacter aerogenes, Enterobacter asburiae, Enterobacter cancerogenus, Enterobacter cloacae, Enterobacter massiliensis, Enterococcus casseliflavus, Enterococcus durans, Enterococcus faecalis, Enterococcus faecium, Enterococcus flavescens, Enterococcus gallinarum, Enterococcus sp., Enterovibrio nigricans, Erysipelatoclostridium ramosum, Escherichia coli, Escherichia sp., Eubacterium biforme, Eubacterium dolichum, Eubacterium hallii, Eubacterium limosum, Eubacterium ramulus, Eubacterium rectale, Eubacterium siraeum, Eubacterium ventriosum, Exiguobacterium marinum, Exiguobacterium undae, Faecalibacterium cf, Faecalibacterium prausnitzii, Faecalitalea cylindroides, Ferrimonas balearica, Finegoldia magna, Flavobacterium daejeonense, Flavonifractor plautii, Fusicatenibacter saccharivorans, Fusobacterium gonidiaformans, Fusobacterium mortiferum, Fusobacterium necrophorum, Fusobacterium nucleatum, Fusobacterium periodonticum, Fusobacterium sp., Fusobacterium ulcerans, Fusobacterium varium, Gallibacterium anatis, Gemmiger formicilis, Gordonibacter pamelaeae, Hafnia alvei, Helicobacter bilis, Helicobacter bills, Helicobacter canadensis, Helicobacter canis, Helicobacter cinaedi, Helicobacter macacae, Helicobacter pametensis, Helicobacter pullorum, Helicobacter pylori, Helicobacter rodentium, Helicobacter winghamensis, Herbaspirillum massiliense, Holdemanella biformis, Holdemania fdiformis, Holdemania filiformis, Holdemania massiliensis, Holdemaniafiliformis, Hungatella hathewayi, Intestinibacter bartlettii, Intestinimonas butyriciproducens, Klebsiella oxytoca, Klebsiella pneumoniae, Kurthia massiliensis, Lachnospira pectinoschiza, Lactobacillus acidophilus, Lactobacillus amylolyticus, Lactobacillus animalis, Lactobacillus antri, Lactobacillus brevis, Lactobacillus buchneri, Lactobacillus casei, Lactobacillus curvatus, Lactobacillus delbrueckii, Lactobacillus fermentum, Lactobacillus gasseri, Lactobacillus helveticus, Lactobacillus hilgardii, Lactobacillus iners, Lactobacillus intestinalis, Lactobacillus johnsonii, Lactobacillus murinus, Lactobacillus paracasei, Lactobacillus plantarum, Lactobacillus reuteri, Lactobacillus rhamnosus, Lactobacillus ruminis, Lactobacillus sakei, Lactobacillus salivarius, Lactobacillus ultunensis, Lactobacillus vaginalis, Lactobacillusplantarum subsp., Leuconostoc mesenteroides, Leuconostoc pseudomesenteroides, Listeria grayi, Listeria innocua, Mannheimia granulomatis, Marvinbryantia formatexigens, Megamonas funiformis, Megamonas hypermegale, Methanobrevibacter smithii, Methanobrevibacter smithiiFI, Micrococcus luteus, Microvirgula aerodenitrificans, Mitsuokella jalaludinii, Mitsuokella multacida, Mollicutes bacterium, Murimonas intestini, Neisseria macacae, Nitriliruptor alkaliphilus, Oceanobacillus massiliensis, Odoribacter laneus, Odoribacter splanchnicus, Ornithobacterium rhinotracheale, Oxalobacter formigenes, Paenibacillus barengoltzii, Paenibacillus chitinolyticus, Paenibacillus lautus, Paenibacillus motobuensis, Paenibacillus senegalensis, Paenisporosarcina quisquiliarum, Parabacteroides distasonis, Parabacteroides goldsteinii, Parabacteroides gordonii, Parabacteroides johnsonii, Parabacteroides merdae, Paraprevotella xylaniphila, Parasutterella excrementihominis, Parvimonas micra, Pediococcus acidilactici, Peptoclostridium difficile, Peptoniphilus harei, Peptoniphilus obesi, Peptoniphilus senegalensis, Peptoniphilus timonensis, Phascolarctobacterium succinatutens, Porphyromonas asaccharolytica, Porphyromonas uenonis, Prevotella baroniae, Prevotella bivia, Prevotella copri, Prevotella dentalis, Prevotella micans, Prevotella multisaccharivorax, Prevotella oralis, Prevotella salivae, Prevotella stercorea, Prevotella veroralis, Propionibacterium acnes, Propionibacterium avidum, Propionibacterium freudenreichii, Propionimicrobium lymphophilum, Proteus mirabilis, Proteuspenneri ATCC, Providencia alcalifaciens, Providencia rettgeri, Providencia rustigianii, Providencia stuartii, Pseudoflavonifractor capillosus, Pseudomonas aeruginosa, Pseudomonas luteola, Ralstonia pickettii, Rheinheimera perlucida, Rheinheimera texasensis, Riemerella columbina, Romboutsia lituseburensis, Roseburia faecis, Roseburia intestinalis, Roseburia inulinivorans, Ruminococcus bicirculans, Ruminococcus bromii, Ruminococcus callidus, Ruminococcus champanellensis, Ruminococcus faecis, Ruminococcus gnavus, Ruminococcus lactaris, Ruminococcus obeum, Ruminococcus sp, Ruminococcus sp., Ruminococcus torques, Sarcina ventriculi, Sellimonas intestinalis, Senegalimassilia anaerobia, Shigella sonnei, Slackia piriformis, Staphylococcus epidermidis, Staphylococcus lentus, Staphylococcus nepalensis, Staphylococcus pseudintermedius, Staphylococcus xylosus, Stenotrophomonas maltophilia, Streptococcus agalactiae, Streptococcus anginosus, Streptococcus australis, Streptococcus caballi, Streptococcus castoreus, Streptococcus didelphis, Streptococcus equinus, Streptococcus gordonii, Streptococcus henryi, Streptococcus hyovaginalis, Streptococcus infantarius, Streptococcus infantis, Streptococcus lutetiensis, Streptococcus merionis, Streptococcus mitis, Streptococcus mutans, Streptococcus oralis, Streptococcus ovis, Streptococcus parasanguinis, Streptococcus plurextorum, Streptococcus porci, Streptococcus pyogenes, Streptococcus salivarius, Streptococcus sobrinus, Streptococcus thermophilus, Streptococcus thoraltensis, Streptomyces albus, Subdoligranulum variabile, Succinatimonas hippei, Sutterella parvirubra, Sutterella wadsworthensis, Terrisporobacter glycolicus, Terrisporobacter mayombei, Thalassobacillus devorans, Timonella senegalensis, Turicibacter sanguinis, unknown sp, unknown sp., Varibaculum cambriense, Veillonella atypica, Veillonella dispar, Veillonella parvula, Vibrio cincinnatiensis, Virgibacillus salexigens, Weissella paramesenteroides, Weissellaparamesenteroides ATCC,

In other embodiments, the targeted bacteria cells are those commonly found on the skin microbiota and are without limitation Acetobacter farinalis, Acetobacter malorum, Acetobacter orleanensis, Acetobacter sicerae, Achromobacter anxifer, Achromobacter denitrificans, Achromobacter marplatensis, Achromobacter spanius, Achromobacter xylosoxidans subsp. xylosoxidans, Acidovorax konjaci, Acidovorax radicis, Acinetobacter johnsonii, Actinomadura citrea, Actinomadura coerulea, Actinomadura fibrosa, Actinomadura fulvescens, Actinomadura jiaoheensis, Actinomadura luteofluorescens, Actinomadura mexicana, Actinomadura nitritigenes, Actinomadura verrucosospora, Actinomadura yumaensis, Actinomyces odontolyticus, Actinomycetospora atypica, Actinomycetospora corticicola, Actinomycetospora rhizophila, Actinomycetospora rishiriensis, Aeromonas australiensis, Aeromonas bestiarum, Aeromonas bivalvium, Aeromonas encheleia, Aeromonas eucrenophila, Aeromonas hydrophila subsp. hydrophila, Aeromonas piscicola, Aeromonas popoffii, Aeromonas rivuli, Aeromonas salmonicida subsp. pectinolytica, Aeromonas salmonicida subsp. smithia, Amaricoccus kaplicensis, Amaricoccus veronensis, Aminobacter aganoensis, Aminobacter cicerones, Aminobacter lissarensis, Aminobacter niigataensis, Ancylobacter polymorphus, Anoxybacillus flavithermus subsp. yunnanensis, Aquamicrobium aerolatum, Archangium gephyra, Archangium gephyra, Archangium minus, Archangium violaceum, Arthrobacter viscosus, Bacillus anthracis, Bacillus australimaris, Bacillus drentensis, Bacillus mycoides, Bacillus pseudomycoides, Bacillus pumilus, Bacillus safensis, Bacillus vallismortis, Bosea thiooxidans, Bradyrhizobium huanghuaihaiense, Bradyrhizobium japonicum, Brevundimonas aurantiaca, Brevundimonas intermedia, Burkholderia aspalathi, Burkholderia choica, Burkholderia cordobensis, Burkholderia diffusa, Burkholderia insulsa, Burkholderia rhynchosiae, Burkholderia terrestris, Burkholderia udeis, Buttiauxella gaviniae, Caenimonas terrae, Capnocytophaga gingivalis, Chitinophaga dinghuensis, Chryseobacterium gleum, Chryseobacterium greenlandense, Chryseobacterium jejuense, Chryseobacterium piscium, Chryseobacterium sediminis, Chryseobacterium tructae, Chryseobacterium ureilyticum, Chryseobacterium vietnamense, Corynebacterium accolens, Corynebacterium afermentans subsp. lipophilum, Corynebacterium minutissimum, Corynebacterium sundsvallense, Cupriavidus metallidurans, Cupriavidus nantongensis, Cupriavidus necator, Cupriavidus pampae, Cupriavidus yeoncheonensis, Curtobacterium flaccumfaciens, Devosia epidermidihirudinis, Devosia riboflavina, Devosia riboflavina, Diaphorobacter oryzae, Dietzia psychralcaliphila, Ensifer adhaerens, Ensifer americanus, Enterococcus malodoratus, Enterococcus pseudoavium, Enterococcus viikkiensis, Enterococcus xiangfangensis, Erwinia rhapontici, Falsirhodobacter halotolerans, Flavobacterium araucananum, Flavobacterium frigidimaris, Gluconobacter frateurii, Gluconobacter thailandicus, Gordonia alkanivorans, Halomonas aquamarina, Halomonas axialensis, Halomonas meridiana, Halomonas olivaria, Halomonas songnenensis, Halomonas variabilis, Herbaspirillum chlorophenolicum, Herbaspirillum frisingense, Herbaspirillum hiltneri, Herbaspirillum huttiense subsp. putei, Herbaspirillum lusitanum, Herminiimonas fonticola, Hydrogenophaga intermedia, Hydrogenophaga pseudoflava, Klebsiella oxytoca, Kosakonia sacchari, Lactobacillus delbrueckii subsp. bulgaricus, Lactobacillus modestisalitolerans, Lactobacillus plantarum subsp. argentoratensis, Lactobacillus xiangfangensis, Lechevalieria roselyniae, Lentzea albida, Lentzea califomiensis, Leuconostoc carnosum, Leuconostoc citreum, Leuconostoc gelidum subsp. gasicomitatum, Leuconostoc mesenteroides subsp. suionicum, Luteimonas aestuarii, Lysobacter antibioticus, Lysobacter koreensis, Lysobacter oryzae, Magnetospirillum moscoviense, Marinomonas alcarazii, Marinomonas primoryensis, Massilia aurea, Massilia jejuensis, Massilia kyonggiensis, Massilia timonae, Mesorhizobium acaciae, Mesorhizobium qingshengii, Mesorhizobium shonense, Methylobacterium haplocladii, Methylobacterium platani, Methylobacterium pseudosasicola, Methylobacterium zatmanii, Microbacterium oxydan, Micromonospora chaiyaphumensis, Micromonospora chalcea, Micromonospora citrea, Micromonospora coxensis, Micromonospora echinofusca, Micromonospora halophytica, Micromonospora kangleipakensis, Micromonospora maritima, Micromonospora nigra, Micromonospora purpureochromogene, Micromonospora rhizosphaerae, Micromonospora saelicesensis, Micro virga subterranea, Micro virga zambiensis, Mycobacterium alvei, Mycobacterium avium subsp. silvaticum, Mycobacterium colombiense, Mycobacterium conceptionense, Mycobacterium conceptionense, Mycobacterium farcinogenes, Mycobacterium fortuitum subsp. fortuitum, Mycobacterium goodii, Mycobacterium insubricum, Mycobacterium Ilatzerense, Mycobacterium neoaurum, Mycobacterium neworleansense, Mycobacterium obuense, Mycobacterium peregrinum, Mycobacterium saopaulense, Mycobacterium septicum, Mycobacterium setense, Mycobacterium smegmatis, Neisseria subflava, Nocardia lijiangensis, Nocardia thailandica, Novosphingobium barchaimii, Novosphingobium lindaniclasticum, Novosphingobium lindaniclasticum, Novosphingobium mathurense, Ochrobactrum pseudogrignonense, Oxalicibacterium solurbis, Paraburkholderia glathei, Paraburkholderia humi, Paraburkholderia phenazinium, Paraburkholderia phytofirmans, Paraburkholderia sordidicola, Paraburkholderia terricola, Paraburkholderia xenovorans, Paracoccus laeviglucosivorans, Patulibacter ginsengiterrae, Polymorphospora rubra, Porphyrobacter colymbi, Prevotella jejuni, Prevotella melaninogenica, Propionibacterium acnes subsp. elongatum, Proteus vulgaris, Providencia rustigianii, Pseudoalteromonas agarivorans, Pseudoalteromonas atlantica, Pseudoalteromonas paragorgicola, Pseudomonas asplenii, Pseudomonas asuensis, Pseudomonas benzenivorans, Pseudomonas cannabina, Pseudomonas cissicola, Pseudomonas congelans, Pseudomonas costantinii, Pseudomonas ficuserectae, Pseudomonas frederiksbergensis, Pseudomonas graminis, Pseudomonas jessenii, Pseudomonas koreensis, Pseudomonas koreensis, Pseudomonas kunmingensis, Pseudomonas marginalis, Pseudomonas mucidolens, Pseudomonas panacis, Pseudomonas plecoglossicida, Pseudomonas poae, Pseudomonas pseudoalcaligenes, Pseudomonas putida, Pseudomonas reinekei, Pseudomonas rhizosphaerae, Pseudomonas seleniipraecipitans, Pseudomonas umsongensis, Pseudomonas zhaodongensis, Pseudonocardia alaniniphila, Pseudonocardia ammonioxydans, Pseudonocardia autotrophica, Pseudonocardia kongjuensis, Pseudonocardia yunnanensis, Pseudorhodoferax soli, Pseudoxanthomonas daejeonensis, Pseudoxanthomonas indica, Pseudoxanthomonas kaohsiungensis, Psychrobacter aquaticus, Psychrobacter arcticus, Psychrobacter celer, Psychrobacter marincola, Psychrobacter nivimaris, Psychrobacter okhotskensis, Psychrobacter okhotskensis, Psychrobacter piscatorii, Psychrobacter pulmonis, Ramlibacter ginsenosidimutans, Rheinheimera japonica, Rheinheimera muenzenbergensis, Rheinheimera soli, Rheinheimera tangshanensis, Rheinheimera texasensis, Rheinheimera tilapiae, Rhizobium alamii, Rhizobium azibense, Rhizobium binae, Rhizobium daejeonense, Rhizobium endophyticum, Rhizobium etli, Rhizobium fabae, Rhizobium freirei, Rhizobium gallicum, Rhizobium loessense, Rhizobium sophoriradicis, Rhizobium taibaishanense, Rhizobium mills, Rhizobium vignae, Rhizobium vignae, Rhizobium yanglingense, Rhodococcus baikonurensis, Rhodococcus enclensis, Rhodoferax saidenbachensis, Rickettsia canadensis, Rickettsia heilongjiangensis, Rickettsia honei, Rickettsia raoultii, Roseateles aquatilis, Roseateles aquatilis, Salmonella enterica subsp. salamae, Serratia ficaria, Serratia myotis, Serratia vespertilionis, Shewanella aestuarii, Shewanella decolorationis, Sphingobium amiense, Sphingobium baderi, Sphingobium barthaii, Sphingobium chlorophenolicum, Sphingobium cupriresistens, Sphingobium czechense, Sphingobium fuliginis, Sphingobium indicum, Sphingobium indicum, Sphingobium japonicum, Sphingobium lactosutens, Sphingomonas dokdonensis, Sphingomonas pseudosanguinis, Sphingopyxis chilensis, Sphingopyxis fribergensis, Sphingopyxis granuli, Sphingopyxis indica, Sphingopyxis witflariensis, Staphylococcus agnetis, Staphylococcus aureus subsp. aureus, Staphylococcus epidermidis, Staphylococcus hominis subsp. novobiosepticus, Staphylococcus nepalensis, Staphylococcus saprophyticus subsp. bovis, Staphylococcus sciuri subsp. carnaticus, Streptomyces caeruleatus, Streptomyces canarius, Streptomyces capoamus, Streptomyces ciscaucasicus, Streptomyces griseorubiginosus, Streptomyces olivaceoviridis, Streptomyces panaciradicis, Streptomyces phaeopurpureus, Streptomyces pseudovenezuelae, Streptomyces resistomycificus, Tianweitania sediminis, Tsukamurella paurometabola, Variovorax guangxiensis, Vogesella alkaliphila, Xanthomonas arboricola, Xanthomonas axonopodis, Xanthomonas cassavae, Xanthomonas cucurbitae, Xanthomonas cynarae, Xanthomonas euvesicatoria, Xanthomonas fragariae, Xanthomonas gardneri, Xanthomonas perforans, Xanthomonas pisi, Xanthomonas populi, Xanthomonas vasicola, Xenophilus aerolatus, Yersinia nurmii, Abiotrophia defectiva, Acidocella aminolytica, Acinetobacter guangdongensis, Acinetobacter parvus, Acinetobacter radioresistens, Acinetobacter soli, Acinetobacter variabilis, Actinomyces cardiffensis, Actinomyces dentalis, Actinomyces europaeus, Actinomyces gerencseriae, Actinomyces graevenitzii, Actinomyces haliotis, Actinomyces johnsonii, Actinomyces massiliensis, Actinomyces meyeri, Actinomyces meyeri, Actinomyces naeslundii, Actinomyces neuii subsp. anitratus, Actinomyces odontolyticus, Actinomyces oris, Actinomyces turicensis, Actinomycetospora corticicola, Actinotignum schaalii, Aerococcus christensenii, Aerococcus urinae, Aeromicrobium flavum, Aeromicrobium massiliense, Aeromicrobium tamlense, Aeromonas sharmana, Aggregatibacter aphrophilus, Aggregatibacter segnis, Agrococcus baldri, Albibacter methylovorans, Alcaligenes faecalis subsp. faecalis, Algoriphagus ratkowskyi, Alkalibacterium olivapovliticus, Alkalibacterium pelagium, Alkalibacterium pelagium, Alloprevotella rava, Alsobacter metallidurans, Amaricoccus kaplicensis, Amaricoccus veronensis, Anaerococcus hydrogenalis, Anaerococcus lactolyticus, Anaerococcus murdochii, Anaerococcus octavius, Anaerococcus prevotii, Anaerococcus vaginalis, Aquabacterium citratiphilum, Aquabacterium olei, Aquabacterium olei, Aquabacterium parvum, Aquincola tertiaricarbonis, Arcobacter venerupis, Arsenicicoccus bolidensis, Arthrobacter russicus, Asticcacaulis excentricus, Atopobium deltae, Atopobium parvulum, Atopobium rimae, Atopobium vaginae, Aureimonas altamirensis, Aureimonas rubiginis, Azospira oryzae, Azospirillum oryzae, Bacillus circulans, Bacillus drentensis, Bacillus fastidiosus, Bacillus lehensis, Bacillus oceanisediminis, Bacillus rhizosphaerae, Bacteriovorax stolpii, Bacteroides coagulans, Bacteroides dorei, Bacteroides fragilis, Bacteroides ovatus, Bacteroides stercoris, Bacteroides uniformis, Bacteroides vulgatus, Bdellovibrio bacteriovorus, Bdellovibrio exovorus, Belnapia moabensis, Belnapia soli, Blautia hansenii, Blautia obeum, Blautia wexlerae, Bosea lathyri, Brachybacterium fresconis, Brachybacterium muris, Brevibacterium ammoniilyticum, Brevibacterium casei, Brevibacterium epidermidis, Brevibacterium iodinum, Brevibacterium luteolum, Brevibacterium paucivorans, Brevibacterium pityocampae, Brevibacterium sanguinis, Brevundimonas albigilva, Brevundimonas diminuta, Brevundimonas vancanneytii, Caenimonas terrae, Calidifontibacter indicus, Campylobacter concisus, Campylobacter gracilis, Campylobacter hominis, Campylobacter rectus, Campylobacter showae, Campylobacter ureolyticus, Capnocytophaga gingivalis, Capnocytophaga leadbetteri, Capnocytophaga ochracea, Capnocytophaga sputigena, Cardiobacterium hominis, Cardiobacterium valvarum, Carnobacterium divergens, Catonella morbi, Caulobacter henricii, Cavicella subterranea, Cellulomonas xylanilytica, Cellvibrio vulgaris, Chitinimonas taiwanensis, Chryseobacterium arachidis, Chryseobacterium daecheongense, Chryseobacterium formosense, Chryseobacterium formosense, Chryseobacterium greenlandense, Chryseobacterium indologenes, Chryseobacterium piscium, Chryseobacterium rigui, Chryseobacterium solani, Chryseobacterium taklimakanense, Chryseobacterium ureilyticum, Chryseobacterium ureilyticum, Chryseobacterium zeae, Chryseomicrobium aureum, Cloacibacterium haliotis, Cloacibacterium normanense, Cloacibacterium normanense, Collinsella aerofaciens, Comamonas denitrificans, Comamonas terrigena, Corynebacterium accolens, Corynebacterium afermentans subsp. lipophilum, Corynebacterium ammoniagenes, Corynebacterium amycolatum, Corynebacterium aurimucosum, Corynebacterium aurimucosum, Corynebacterium coyleae, Corynebacterium durum, Corynebacterium freiburgense, Corynebacterium glaucum, Corynebacterium glyciniphilum, Corynebacterium imitans, Corynebacterium jeikeium, Corynebacterium jeikeium, Corynebacterium kroppenstedtii, Corynebacterium lipophiloflavum, Corynebacterium massiliense, Corynebacterium mastitidis, Corynebacterium matruchotii, Corynebacterium minutissimum, Corynebacterium mucifaciens, Corynebacterium mustelae, Corynebacterium mycetoides, Corynebacterium pyruviciproducens, Corynebacterium simulans, Corynebacterium singulare, Corynebacterium sputi, Corynebacterium suicordis, Corynebacterium tuberculostearicum, Corynebacterium tuberculostearicum, Corynebacterium ureicelerivorans, Corynebacterium variabile, Couchioplanes caeruleus subsp. caeruleus, Cupriavidus metallidurans, Curtobacterium herbarum, Dechloromonas agitata, Deinococcus actinosclerus, Deinococcus antarcticus, Deinococcus caeni, Deinococcus ficus, Deinococcus geothermalis, Deinococcus radiodurans, Deinococcus wulumuqiensis, Deinococcus xinjiangensis, Dermabacter hominis, Dermabacter vaginalis, Dermacoccus nishinomiyaensis, Desemzia incerta, Desertibacter roseus, Dialister invisus, Dialister micraerophilus, Dialister propionicifaciens, Dietzia aurantiaca, Dietzia cercidiphylli, Dietzia timorensis, Dietzia timorensis, Dokdonella koreensis, Dokdonella koreensis, Dolosigranulum pigrum, Eikenella corrodens, Elizabethkingia miricola, Elstera litoralis, Empedobacter brevis, Enhydrobacter aerosaccus, Enterobacter xiangfangensis, Enterococcus aquimarinus, Enterococcus faecalis, Enterococcus olivae, Erwinia rhapontici, Eubacterium eligens, Eubacterium infirmum, Eubacterium rectale, Eubacterium saphenum, Eubacterium sulci, Exiguobacterium mexicanum, Facklamia tabacinasalis, Falsirhodobacter halotolerans, Finegoldia magna, Flavobacterium cutihirudinis, Flavobacterium lindanitolerans, Flavobacterium resistens, Friedmanniella capsulata, Fusobacterium nucleatum subsp. polymorphum, Gemella haemolysans, Gemella morbillorum, Gemella palaticanis, Gemella sanguinis, Gemmobacter aquaticus, Gemmobacter caeni, Gordonia jinhuaensis, Gordonia kroppenstedtii, Gordonia polyisoprenivorans, Gordonia polyisoprenivorans, Granulicatella adiacens, Granulicatella elegans, Haemophilus parainfluenzae, Haemophilus sputorum, Halomonas sulfidaeris, Herpetosiphon aurantiacus, Hydrocarboniphaga effusa, Idiomarina maris, Janibacter anophelis, Janibacter hoylei, Janibacter indicus, Janibacter limosus, Janibacter melonis, Jeotgalicoccus halophilus, Jonquetella anthropi, Kaistia geumhonensis, Kingella denitrificans, Kingella oralis, Klebsiella oxytoca, Knoellia aerolata, Knoellia locipacati, Kocuria atrinae, Kocuria carniphila, Kocuria kristinae, Kocuria palustris, Kocuria turfanensis, Lachnoanaerobaculum saburreum, Lachnoanaerobaculum saburreum, Lactobacillus crispatus, Lactobacillus iners, Lactococcus lactis subsp. lactis, Lactococcus lactis subsp. lactis, Lactococcus piscium, Lapillicoccus jejuensis, Lautropia mirabilis, Legionella beliardensis, Leptotrichia buccalis, Leptotrichia goodfellowii, Leptotrichia hofstadii, Leptotrichia hongkongensis, Leptotrichia shahii, Leptotrichia trevisanii, Leptotrichia wadei, Luteimonas terricola, Lysinibacillus fusiformis, Lysobacter spongiicola, Lysobacter xinjiangensis, Macrococcus caseolyticus, Marmoricola pocheonensis, Marmoricola scoriae, Massilia alkalitolerans, Massilia alkalitolerans, Massilia aurea, Massilia plicata, Massilia timonae, Megamonas rupellensis, Meiothermus silvanus, Methylobacterium dankookense, Methylobacterium goesingense, Methylobacterium goesingense, Methylobacterium isbiliense, Methylobacterium jeotgali, Methylobacterium oxalidis, Methylobacterium platani, Methylobacterium pseudosasicola, Methyloversatilis universalis, Microbacterium foliorum, Microbacterium hydrothermale, Microbacterium hydrothermale, Microbacterium lacticum, Microbacterium lacticum, Microbacterium laevaniformans, Microbacterium paludicola, Microbacterium petrolearium, Microbacterium phyllosphaerae, Microbacterium resistens, Micrococcus antarcticus, Micrococcus cohnii, Micrococcus flavus, Micrococcus lylae, Micrococcus terreus, Microlunatus aurantiacus, Micropruina glycogenica, Micro virga aerilata, Micro virga aerilata, Micro virga subterranea, Micro virga vignae, Micro virga zambiensis, Microvirgula aerodenitrificans, Mogibacterium timidum, Moraxella atlantae, Moraxella catarrhalis, Morganella morganii subsp. morganii, Morganella psychrotolerans, Murdochiella asaccharolytica, Mycobacterium asiaticum, Mycobacterium chubuense, Mycobacterium crocinum, Mycobacterium gadium, Mycobacterium holsaticum, Mycobacterium iranicum, Mycobacterium longobardum, Mycobacterium neoaurum, Mycobacterium neoaurum, Mycobacterium obuense, Negativicoccus succinicivorans, Neisseria bacilliformis, Neisseria oralis, Neisseria sicca, Neisseria subflava, Nesterenkonia lacusekhoensis, Nesterenkonia rhizosphaerae, Nevskia persephonica, Nevskia ramosa, Niabella yanshanensis, Niveibacterium umoris, Nocardia niwae, Nocardia thailandica, Nocardioides agariphilus, Nocardioides dilutus, Nocardioides ganghwensis, Nocardioides hwasunensis, Nocardioides nanhaiensis, Nocardioides sediminis, Nosocomiicoccus ampullae, Noviherbaspirillum malthae, Novosphingobium lindaniclasticum, Novosphingobium rosa, Ochrobactrum rhizosphaerae, Olsenella uli, Ornithinimicrobium murale, Ornithinimicrobium tianjinense, Oryzobacter terrae, Ottowia beijingensis, Paenalcaligenes suwonensis, Paenibacillus agaridevorans, Paenibacillus phoenicis, Paenibacillus xylanexedens, Paludibacterium yongneupense, Pantoea cypripedii, Parabacteroides distasonis, Paraburkholderia andropogonis, Paracoccus alcaliphilus, Paracoccus angustae, Paracoccus kocurii, Paracoccus laeviglucosivorans, Paracoccus sediminis, Paracoccus sphaerophysae, Paracoccus yeei, Parvimonas micra, Parviterribacter multiflagellatus, Patulibacter ginsengiterrae, Pedobacter aquatilis, Pedobacter ginsengisoli, Pedobacter xixiisoli, Peptococcus niger, Peptoniphilus coxii, Peptoniphilus gorbachii, Peptoniphilus harei, Peptoniphilus koenoeneniae, Peptoniphilus lacrimalis, Peptostreptococcus anaerobius, Peptostreptococcus stomatis, Phascolarctobacterium faecium, Phenylobacterium haematophilum, Phenylobacterium kunshanense, Pluralibacter gergoviae, Polymorphobacter multimanifer, Porphyromonas bennonis, Porphyromonas endodontalis, Porphyromonas gingivalis, Porphyromonas gingivicanis, Porphyromonas pasteri, Porphyromonas pogonae, Porphyromonas somerae, Povalibacter uvarum, Prevotella aurantiaca, Prevotella baroniae, Prevotella bivia, Prevotella buccae, Prevotella buccalis, Prevotella copri, Prevotella corporis, Prevotella denticola, Prevotella enoeca, Prevotella histicola, Prevotella intermedia, Prevotella jejuni, Prevotella jejuni, Prevotella maculosa, Prevotella melaninogenica, Prevotella melaninogenica, Prevotella micans, Prevotella multiformis, Prevotella nanceiensis, Prevotella nigrescens, Prevotella oris, Prevotella oulorum, Prevotella pallens, Prevotella pleuritidis, Prevotella saccharolytica, Prevotella salivae, Prevotella shahii, Prevotella timonensis, Prevotella veroralis, Propionibacterium acidifaciens, Propionibacterium acnes subsp. acnes, Propionibacterium acnes subsp. acnes, Propionibacterium acnes subsp. elongatum, Propionibacterium granulosum, Propionimicrobium lymphophilum, Propionispira arcuata, Pseudokineococcus lusitanus, Pseudomonas aeruginosa, Pseudomonas chengduensis, Pseudonocardia benzenivorans, Pseudorhodoplanes sinuspersici, Psychrobacter sanguinis, Ramlibacter ginsenosidimutans, Rheinheimera aquimaris, Rhizobium alvei, Rhizobium daejeonense, Rhizobium larrymoorei, Rhizobium rhizoryzae, Rhizobium soli, Rhizobium taibaishanense, Rhizobium vignae, Rhodanobacter glycinis, Rhodobacter veldkampii, Rhodococcus enclensis, Rhodococcus fascians, Rhodococcus fascians, Rhodovarius lipocyclicus, Rivicola pingtungensis, Roseburia inulinivorans, Rosenbergiella nectarea, Roseomonas aerilata, Roseomonas aquatica, Roseomonas mucosa, Roseomonas rosea, Roseomonas vinacea, Rothia aeria, Rothia amarae, Rothia dentocariosa, Rothia endophytica, Rothia mucilaginosa, Rothia nasimurium, Rubellimicrobium mesophilum, Rubellimicrobium roseum, Rubrobacter bracarensis, Rudaea cellulosilytica, Ruminococcus gnavus, Runella zeae, Saccharopolyspora rectivirgula, Salinicoccus qingdaonensis, Scardovia wiggsiae, Sediminibacterium ginsengisoli, Selenomonas artemidis, Selenomonas infelix, Selenomonas noxia, Selenomonas sputigena, Shewanella aestuarii, Shuttleworthia satelles, Simonsiella muelleri, Skermanella aerolata, Skermanella stibiiresistens, Slackia exigua, Smaragdicoccus niigatensis, Sneathia sanguinegens, Solirubrobacter soli, Sphingobacterium caeni, Sphingobacterium daejeonense, Sphingobacterium hotanense, Sphingobacterium kyonggiense, Sphingobacterium multivorum, Sphingobacterium nematocida, Sphingobacterium spiritivorum, Sphingobium amiense, Sphingobium indicum, Sphingobium lactosutens, Sphingobium subterraneum, Sphingomonas abaci, Sphingomonas aestuarii, Sphingomonas canadensis, Sphingomonas daechungensis, Sphingomonas dokdonensis, Sphingomonas echinoides, Sphingomonas fonticola, Sphingomonas fonticola, Sphingomonas formosensis, Sphingomonas gei, Sphingomonas hankookensis, Sphingomonas hankookensis, Sphingomonas koreensis, Sphingomonas kyeonggiensis, Sphingomonas laterariae, Sphingomonas mucosissima, Sphingomonas oligophenolica, Sphingomonas pseudosanguinis, Sphingomonas sediminicola, Sphingomonas yantingensis, Sphingomonas yunnanensis, Sphingopyxis indica, Spirosoma rigui, Sporacetigenium mesophilum, Sporocytophaga myxococcoides, Staphylococcus auricularis, Staphylococcus epidermidis, Staphylococcus epidermidis, Staphylococcus hominis subsp. novobiosepticus, Staphylococcus lugdunensis, Staphylococcus pettenkoferi, Stenotrophomonas koreensis, Stenotrophomonas rhizophila, Stenotrophomonas rhizophila, Streptococcus agalactiae, Streptococcus canis, Streptococcus cristatus, Streptococcus gordonii, Streptococcus infantis, Streptococcus intermedius, Streptococcus mutans, Streptococcus oligofermentans, Streptococcus oralis, Streptococcus sanguinis, Streptomyces iconiensis, Streptomyces yanglinensis, Tabrizicola aquatica, Tahibacter caeni, Tannerella forsythia, Tepidicella xavieri, Tepidimonas fonticaldi, Terracoccus luteus, Tessaracoccus flavescens, Thermus thermophilus, Tianweitania sediminis, Tianweitania sediminis, Treponema amylovorum, Treponema denticola, Treponema lecithinolyticum, Treponema medium, Turicella otitidis, Turicibacter sanguinis, Undibacterium oligocarboniphilum, Undibacterium squillarum, Vagococcus salmoninarum, Varibaculum cambriense, Vibrio metschnikovii, Xanthobacter tagetidis, Xenophilus aerolatus, Xenophilus arseniciresistens, Yimella lutea, Zimmermannella alba, Zimmermannella bifida, Zoogloea caeni,

In other embodiments, the targeted bacteria cells are those commonly found in the vaginal microbiota and are, without limitation, Acinetobacter antiviralis, Acinetobacter baumannii, Acinetobacter calcoaceticus, Acinetobacter johnsonii, Actinobaculum massiliense, Actinobaculum schaalii, Actinomyces europaeus, Actinomyces graevenitzii, Actinomyces israelii, Actinomyces meyeri, Actinomyces naeslundii, Actinomyces neuii, Actinomyces odontolyticus, Actinomyces turicensis, Actinomyces urogenitalis, Actinomyces viscosus, Aerococcus christensenii, Aerococcus urinae, Aerococcus viridans, Aeromonas encheleia, Aeromonas salmonicida, Afipia massiliensis, Agrobacterium tumefaciens, Algoriphagus aquatilis, Aliivibrio wodanis, Alistipes finegoldii, Alloiococcus otitis, Alloprevotella tannerae, Alloscardovia omnicolens, Altererythrobacter epoxidivorans, Ammoniphilus oxalaticus, Amnibacterium kyonggiense, Anaerococcus hydrogenalis, Anaerococcus lactolyticus, Anaerococcus murdochii, Anaerococcus obesiensis, Anaerococcus prevotii, Anaerococcus tetradius, Anaerococcus vaginalis, Anaeroglobus geminatus, Anoxybacillus pushchinoensis, Aquabacterium parvum, Arcanobacterium phocae, Arthrobacter aurescens, Asticcacaulis excentricus, Atopobium minutum, Atopobium parvulum, Atopobium rimae, Atopobium vaginae, Avibacterium gallinarum, Bacillus acidicola, Bacillus atrophaeus, Bacillus cereus, Bacillus cibi, Bacillus coahuilensis, Bacillus gaemokensis, Bacillus methanolicus, Bacillus oleronius, Bacillus pumilus, Bacillus shackletonii, Bacillus sporothermodurans, Bacillus subtilis, Bacillus wakoensis, Bacillus weihenstephanensis, Bacteroides barnesiae, Bacteroides coagulans, Bacteroides dorei, Bacteroides faecis, Bacteroides forsythus, Bacteroides fragilis, Bacteroides nordii, Bacteroides ovatus, Bacteroides salyersiae, Bacteroides stercoris, Bacteroides uniformis, Bacteroides vulgatus, Bacteroides xylanisolvens, Bacteroides zoogleoformans, Barnesiella viscericola, Bhargavaea cecembensis, Bifidobacterium adolescentis, Bifidobacterium bifidum, Bifidobacterium breve, Bifidobacterium dentium, Bifidobacterium logum subsp. infantis, Bifidobacterium longum, Bifidobacterium pseudocatenulatum, Bifidobacterium scardovii, Bilophila wadsworthia, Blautia hydrogenotrophica, Blautia obeum, Blautia producta, Brachybacterium faecium, Bradyrhizobium japonicum, Brevibacterium mcbrellneri, Brevibacterium otitidis, Brevibacterium paucivorans, Bulleidia extructa, Burkholderia fungorum, Burkholderia phenoliruptix, Caldicellulosiruptor saccharolyticus, Caldimonas taiwanensis, Campylobacter gracilis, Campylobacter hominis, Campylobacter sputorum, Campylobacter ureolyticus, Capnocytophaga ochracea, Cardiobacterium hominis, Catonella morbi, Chlamydia trachomatis, Chlamydophila abortus, Chondromyces robustus, Chryseobacterium aquaticum, Citrobacter youngae, Cloacibacterium normanense, Clostridium cavendishii, Clostridium colicanis, Clostridium jejuense, Clostridium perfringens, Clostridium ramosum, Clostridium sordellii, Clostridium viride, Comamonas terrigena, Corynebacterium accolens, Corynebacterium appendicis, Corynebacterium coyleae, Corynebacterium glucuronolyticum, Corynebacterium glutamicum, Corynebacterium jeikeium, Corynebacterium kroppenstedtii, Corynebacterium lipophiloflavum, Corynebacterium minutissimum, Corynebacterium mucifaciens, Corynebacterium nuruki, Corynebacterium pseudo genitalium, Corynebacterium pyruviciproducens, Corynebacterium singulare, Corynebacterium striatum, Corynebacterium tuberculostearicum, Corynebacterium xerosis, Cryobacterium psychrophilum, Curtobacterium flaccumfaciens, Cutibacterium acnes, Cutibacterium avidum, Cytophaga xylanolytica, Deinococcus radiophilus, Delftia tsuruhatensis, Desulfovibrio desulfuricans, Dialister invisus, Dialister micraerophilus, Dialister pneumosintes, Dialister propionicifaciens, Dickeya chrysanthemi, Dorea longicatena, Eggerthella lenta, Eggerthia catenaformis, Eikenella corrodens, Enhydrobacter aerosaccus, Enterobacter asburiae, Enterobacter cloacae, Enterococcus avium, Enterococcus durans, Enterococcus faecalis, Enterococcus faecium, Enterococcus hirae, Erwinia persicina, Erwinia rhapontici, Erwinia toletana, Escherichia coli, Escherichia fergusonii, Eubacterium brachy, Eubacterium eligens, Eubacterium nodatum, Eubacterium rectale, Eubacterium saphenum, Eubacterium siraeum, Eubacterium sulci, Eubacterium yurii, Exiguobacterium acetylicum, Facklamia ignava, Faecalibacterium prausnitzii, Filifactor alocis, Finegoldia magna, Fusobacterium gonidiaformans, Fusobacterium nucleatum, Fusobacterium periodonticum, Gardnerella vaginalis, Gemella asaccharolytica, Gemella bergeri, Gemella haemolysans, Gemella sanguinis, Geobacillus stearothermophilus, Geobacillus the rmocatenulatus, Geobacillus thermoglucosidasius, Geobacter grbiciae, Granulicatella elegans, Haemophilus ducreyi, Haemophilus haemolyticus, Haemophilus parahaemolyticus, Haemophilus parainfluenzae, Hafnia alvei, Halomonas meridiana, Halomonas phoceae, Halomonas venusta, Herbaspirillum seropedicae, Janthinobacterium lividum, Jonquetella anthropi, Klebsiella granulomatis, Klebsiella oxytoca, Klebsiella pneumoniae, Lactobacillus acidophilus, Lactobacillus amylovorus, Lactobacillus brevis, Lactobacillus coleohominis, Lactobacillus crispatus, Lactobacillus curvatus, Lactobacillus delbrueckii, Lactobacillus fermentum, Lactobacillus gasseri, Lactobacillus helveticus, Lactobacillus iners, Lactobacillus jensenii, Lactobacillus johnsonii, Lactobacillus kalixensis, Lactobacillus kefiranofaciens, Lactobacillus kimchicus, Lactobacillus kitasatonis, Lactobacillus mucosae, Lactobacillus panis, Lactobacillus paracasei, Lactobacillus plantarum, Lactobacillus pontis, Lactobacillus reuteri, Lactobacillus rhamnosus, Lactobacillus salivarius, Lactobacillus ultunensis, Lactobacillus vaginalis, Lactococcus lactis, Leptotrichia buccalis, Leuconostoc carnosum, Leuconostoc citreum, Leuconostoc garlicum, Leuconostoc lactis, Leuconostoc mesenteroides, Lysinimonas kribbensis, Mageeibacillus indolicus, Maribacter orientalis, Marinomonas protea, Marinospirillum insulare, Massilia timonae, Megasphaera elsdenii, Megasphaera micronuciformis, Mesorhizobium amorphae, Methylobacterium radiotolerans, Methylotenera versatilis, Microbacterium halophilum, Micrococcus luteus, Microterricola viridarii, Mobiluncus curtisii, Mobiluncus mulieris, Mogibacterium timidum, Moorella glycerini, Moraxella osloensis, Morganella morganii, Moryella indoligenes, Murdochiella asaccharolytica, Mycoplasma alvi, Mycoplasma genitalium, Mycoplasma hominis, Mycoplasma muris, Mycoplasma salivarium, Negativicoccus succinicivorans, Neisseria flava, Neisseria gonorrhoeae, Neisseria mucosa, Neisseria subflava, Nevskia ramosa, Nevskia soli, Nitriliruptor alkaliphilus, Odoribacter splanchnicus, Oligella urethralis, Olsenella uli, Paenibacillus amylolyticus, Paenibacillus humicus, Paenibacillus pabuli, Paenibacillus pasadenensis, Paenibacillus pini, Paenibacillus validus, Pantoea agglomerans, Parabacteroides merdae, Paraburkholderia caryophylli, Paracoccus yeei, Parastreptomyces abscessus, Parvimonas micra, Pectobacterium betavasculorum, Pectobacterium carotovorum, Pediococcus acidilactici, Pediococcus ethanolidurans, Pedobacter alluvionis, Pedobacter wanjuense, Pelomonas aquatica, Peptococcus niger, Peptoniphilus asaccharolyticus, Peptoniphilus gorbachii, Peptoniphilus harei, Peptoniphilus indolicus, Peptoniphilus lacrimalis, Peptoniphilus massiliensis, Peptostreptococcus anaerobius, Peptostreptococcus massiliae, Peptostreptococcus stomatis, Photobacterium angustum, Photobacterium frigidiphilum, Photobacterium phosphoreum, Porphyromonas asaccharolytica, Porphyromonas bennonis, Porphyromonas catoniae, Porphyromonas endodontalis, Porphyromonas gingivalis, Porphyromonas somerae, Porphyromonas uenonis, Prevotella amnii, Prevotella baroniae, Prevotella bergensis, Prevotella bivia, Prevotella buccae, Prevotella buccalis, Prevotella colorans, Prevotella copri, Prevotella corporis, Prevotella dentalis, Prevotella denticola, Prevotella disiens, Prevotella intermedia, Prevotella loescheii, Prevotella marshii, Prevotella melaninogenica, Prevotella micans, Prevotella nigrescens, Prevotella oris, Prevotella pleuritidis, Prevotella ruminicola, Prevotella shahii, Prevotella stercorea, Prevotella timonensis, Prevotella veroralis, Propionimicrobium lymphophilum, Proteus mirabilis, Pseudomonas abietaniphila, Pseudomonas aeruginosa, Pseudomonas amygdali, Pseudomonas azotoformans, Pseudomonas chlororaphis, Pseudomonas cuatrocienegasensis, Pseudomonas fluorescens, Pseudomonas fulva, Pseudomonas lutea, Pseudomonas mucidolens, Pseudomonas oleovorans, Pseudomonas orientalis, Pseudomonas pseudoalcaligenes, Pseudomonas psychrophila, Pseudomonas putida, Pseudomonas synxantha, Pseudomonas syringae, Pseudomonas tolaasii, Pseudopropionibacterium propionicum, Rahnella aquatilis, Ralstonia pickettii, Ralstonia solanacearum, Raoultella planticola, Rhizobacter dauci, Rhizobium etli, Rhodococcus fascians, Rhodopseudomonas palustris, Roseburia intestinalis, Roseburia inulinivorans, Rothia mucilaginosa, Ruminococcus bromii, Ruminococcus gnavus, Ruminococcus torques, Sanguibacter keddieii, Sediminibacterium salmoneum, Selenomonas bovis, Serratia fonticola, Serratia liquefaciens, Serratia marcescens, Shewanella algae, Shewanella amazonensis, Shigella boydii, Shigella sonnei, Slackia exigua, Sneathia amnii, Sneathia sanguinegens, Solobacterium moorei, Sorangium cellulosum, Sphingobium amiense, Sphingobium japonicum, Sphingobium yanoikuyae, Sphingomonas wittichii, Sporosarcina aquimarina, Staphylococcus aureus, Staphylococcus auricularis, Staphylococcus capitis, Staphylococcus epidermidis, Staphylococcus haemolyticus, Staphylococcus hominis, Staphylococcus lugdunensis, Staphylococcus saprophyticus, Staphylococcus schleiferi, Staphylococcus simiae, Staphylococcus simulans, Staphylococcus warneri, Stenotrophomonas maltophilia, Stenoxybacter acetivorans, Streptococcus agalactiae, Streptococcus anginosus, Streptococcus australis, Streptococcus equinus, Streptococcus gallolyticus, Streptococcus infantis, Streptococcus intermedius, Streptococcus lutetiensis, Streptococcus marimammalium, Streptococcus mitis, Streptococcus mutans, Streptococcus oralis, Streptococcus parasanguinis, Streptococcus phocae, Streptococcus pseudopneumoniae, Streptococcus salivarius, Streptococcus sanguinis, Streptococcus thermophilus, Sutterella wadsworthensis, Tannerella forsythia, Terrahaemophilus aromaticivorans, Treponema denticola, Treponema maltophilum, Treponema parvum, Treponema vincentii, Trueperella bernardiae, Turicella otitidis, Ureaplasma parvum, Ureaplasma urealyticum, Varibaculum cambriense, Variovorax paradoxus, Veillonella atypica, Veillonella dispar, Veillonella montpellierensis, Veillonella parvula, Virgibacillus proomii, Viridibacillus arenosi, Viridibacillus arvi, Weissella cibaria, Weissella soli, Xanthomonas campestris, Xanthomonas vesicatoria, Zobellia laminariae, Zoogloea ramigera,

In one embodiment, the targeted bacteria are Escherichia coli.

In one embodiment, the targeted bacteria are Klebsiella pneumoniae.

In one embodiment, the targeted bacteria are Escherichia coli and/or Klebsiella pneumoniae.

In one embodiment, the targeted bacteria are Klebsiella pneumoniae of the K-type such as K2, K1, K62, K64, K23, K28, K54, K16, K5, K27 or combinations thereof. In one embodiment, the targeted bacteria are Klebsiella pneumoniae of the K-type K2, K1, K62, K64 or combinations thereof. In one embodiment, the targeted bacteria are Klebsiella pneumoniae of the K-type K23, K28, K54, K16, or combinations thereof. In one embodiment, the targeted bacteria are Klebsiella pneumoniae of the K-type K5, K27, or combinations thereof. In one embodiment, the targeted bacteria are Klebsiella pneumoniae of the K-type K24, K2, K23, K71, K62, K60, K41, K28, K22, K37, K17, K16, K64, K1, K15, K50, K51, K58, or combinations thereof. In one embodiment, the targeted bacteria are Klebsiella pneumoniae of the K-type K24, K2, K23, K71, or combinations thereof. In one embodiment, the targeted bacteria are Klebsiella pneumoniae of the K-type K62, K60, K41, K28, K22, K37, K17, K16, K64, K1, K15, K50, K51, K58, or combinations thereof. In one embodiment, the targeted bacteria are Klebsiella pneumoniae of the O-type such as O1, O2, O3, O5, O4, O12, OL101, OL104 or combinations thereof. In one embodiment, the targeted bacteria are Klebsiella pneumoniae of the O-type O1, O2, O3, O5, or combinations thereof. In one embodiment, the targeted bacteria are Klebsiella pneumoniae of the O-type O4, O12, OL101, OL104 or combinations thereof. In one embodiment, the targeted bacteria are Klebsiella pneumoniae strain MLST258.

In one embodiment, the targeted bacteria are Escherichia coli of the K type K1, K5 or combinations thereof. In one embodiment, the targeted bacteria are Escherichia coli of the O-type O1, O2, O6, O25, O18, or combinations thereof. In one embodiment, the targeted bacteria are Escherichia coli of the O-type O1, O2? O4, O6, O7, O8, O16, O25, O75, or combinations thereof. In one embodiment, the targeted bacteria are selected from the group consisting of Escherichia coli strains MLST131, ST73, ST95, ST12, ST10, ST14, ST2278, ST69, and combinations thereof.

In one embodiment, the targeted bacteria are Cutibacterium acnes more specifically the acne related Cutibacterium acnes from the phylogroup IA1 or RT4, RTS, RT8, RT9, RT10 or Clonal Complex(CC) CC1, CC3, CC4, more specifically the ST1, ST3, ST4.

Bacteriophages used for preparing bacterial virus particles such as packaged phagemids, may target (e.g., specifically target) a bacterial cell from any one or more of the disclosed genus and/or species of bacteria to specifically deliver the plasmid.

In one embodiment, the targeted bacteria are pathogenic bacteria. The targeted bacteria can be virulent bacteria.

The targeted bacteria can be antibiotic resistant bacteria, preferably selected from the group consisting of extended-spectrum beta-lactamase-producing (ESBL) Escherichia coli, ESBL Klebsiella pneumoniae, carbapenem-resistant (CR) Escherichia coli, CR Klebsiella pneumoniae, vancomycin-resistant Enterococcus (VRE), methicillin-resistant Staphylococcus aureus (MRSA), multidrug-resistant (MDR) Acinetobacter baumannii, MDR Enterobacter spp., and any combination thereof. Preferably, the targeted bacteria can be selected from the group consisting of extended-spectrum beta-lactamase-producing (ESBL) Escherichia coli strains. Still preferably, the targeted bacteria can be selected from the group consisting of extended-spectrum beta-lactamase-producing (ESBL) Klebsiella pneumoniae strains. Still preferably, the targeted bacteria can be selected from the group consisting of extended-spectrum beta-lactamase-producing (ESBL) Escherichia coli strains, extended-spectrum beta-lactamase-producing (ESBL) Klebsiella pneumoniae strains, and mixtures thereof. Still preferably, the targeted bacteria can be selected from the group consisting of CR Escherichia coli strains. Still preferably, the targeted bacteria can be selected from the group consisting of CR Klebsiella pneumoniae strains. Still preferably, the targeted bacteria can be selected from the group consisting of CR Escherichia coli strains, CR Klebsiella pneumoniae strains, and mixtures thereof.

Alternatively, the targeted bacterium can be a bacterium of the microbiome of a given species, preferably a bacterium of the human microbiota.

Thus, bacterial virus particles may target (e.g., specifically target) a bacterial cell from any one or more of the foregoing species of bacteria to specifically deliver the plasmid/vector/genetic modification according to the invention.

In another particular embodiment, at least two distinct vectors are used, in particular at least 3, 4, 5, 6, 7, 8, 9, 10 or more, each vector targeting a different bacteria as defined above, in particular targeting different bacterial species, different bacterial K-type, different bacterial O-type, and/or different bacterial strains.

In a particularly preferred embodiment, at least two distinct vectors are used, in particular at least 3, 4, 5, 6, 7, 8, 9, 10 or more, each vector targeting a different E. coli bacterium, in particular different E. coli K-type, different E. coli O-type, and/or different E. coli strains.

### Bacterial Viruses

The bacterial virus particles are prepared from bacterial virus. The bacterial viruses are chosen in order to be able to introduce the plasmid into the targeted bacteria.

Bacterial viruses are preferably bacteriophages. Bacteriophages are obligate intracellular parasites that multiply inside bacteria by co-opting some or all of the host biosynthetic machinery. Phage genomes come in a variety of sizes and shapes (e.g., linear or circular). Most phages range in size from 24-200 nm in diameter. Phages contain nucleic acid (i.e., genome) and proteins, and may be enveloped by a lipid membrane. Depending upon the phage, the nucleic acid genome can be either DNA or RNA, and can exist in either circular or linear forms. The size of the phage genome varies depending upon the phage. The simplest phages have genomes that are only a few thousand nucleotides in size, while the more complex phages may contain more than 100,000 nucleotides in their genome, and in rare instances more than 1,000,000. The number and amount of individual types of protein in phage particles will vary depending upon the phage.

Optionally, the bacteriophage is selected from the Order Caudovirales consisting of, based on the taxonomy of Krupovic et al, Arch Virol, 2015:
family Myoviridae (such as, without limitation, genus Cp220virus, Cp8virus, Ea214virus, Felixo1virus, Mooglevirus, Suspvirus, Hp1virus, P2virus, Kayvirus, P100virus, Silviavirus, Spo1virus, Tsarbombavirus, Twortvirus, Cc31virus, Jd18virus, Js98virus, Kp15virus, Moonvirus, Rb49virus, Rb69virus, S16virus, Schizot4virus, Sp18virus, T4virus, Cr3virus, Se1virus, V5virus, Abouovirus, Agatevirus, Agrican357virus, Ap22virus, Arv1 virus, B4virus, Bastillevirus, Bc431virus, Bcep78virus, Bcepmuvirus, Biquartavirus, Bxz1virus, Cd119virus, Cp51virus, Cvm10virus, Eah2virus, Elvirus, Hapunavirus, Jimmervirus, Kpp10virus, M12virus, Machinavirus, Marthavirus, Msw3virus, Muvirus, Myohalovirus, Nitivirus, P1virus, Pakpunavirus, Pbunavirus, Phikzvirus, Rheph4virus, Rsl2virus, Rslunavirus, Secunda5virus, Sep1virus, Spn3virus, Svunavirus, Tg1virus, Vhmlvirus and Wphvirus)
family Podoviridae (such as, without limitation, genus Frilvirus, Kp32virus, Kp34virus, Phikmvvirus, Pradovirus, Sp6virus, T7virus, Cp1virus, P68virus, Phi29virus, Nona33virus, Pocjvirus, 112011virus, Bcep22virus, Bpp1virus, Cba41virus, Df112virus, Ea92virus, Epsilon15virus, F116virus, G7cvirus, Jwalphavirus, Kf1virus, Kpp25virus, Lit1virus, Luz24virus, Luz7virus, N4virus, Nonanavirus, P22virus, Pagevirus, Phieco32virus, Prtbvirus, Sp58virus, Una961virus and Vp5virus)
family Siphoviridae (such as, without limitation, genus Camvirus, Likavirus, R4virus, Acadianvirus, Coopervirus, Pg1virus, Pipefishvirus, Rosebushvirus, Brujitavirus, Che9cvirus, Hawkeyevirus, Plotvirus, Jerseyvirus, K1gvirus, Sp31virus, Lmd1virus, Una4virus, Bongovirus, Reyvirus, Buttersvirus, Charlievirus, Redivirus, Baxtervirus, Nymphadoravirus, Bignuzvirus, Fishburnevirus, Phayoncevirus, Kp36virus, Rogue1virus, Rtpvirus, T1virus, Tlsvirus, Ab18virus, Amigovirus, Anatolevirus, Andromedavirus, Attisvirus, Barnyardvirus, Bernal13virus, Biseptimavirus, Bronvirus, C2virus, C5virus, Cba181virus, Cbastvirus, Cecivirus, Che8virus, Chivirus, Cjw1virus, Corndogvirus, Cronusvirus, D3112virus, D3virus, Decurrovirus, Demosthenesvirus, Doucettevirus, E125virus, Eiauvirus, Ff47virus, Gaiavirus, Gilesvirus, Gordonvirus, Gordtnkvirus, Harrisonvirus, Hk578virus, Hk97virus, Jenstvirus, Jwxvirus, Kelleziovirus, Korravirus, L5virus, Lambdavirus, Laroyevirus, Liefievirus, Marvinvirus, Mudcatvirus, N15virus, Nonagvirus, Np1virus, Omegavirus, P12002virus, P12024virus, P23virus, P70virus, Pa6virus, Pamx74virus, Patiencevirus, Pbi1virus, Pepy6virus, Pfr1 virus, Phic31virus, Phicbkvirus, Phietavirus, Phifelvirus, PhijI1virus, Pis4avirus, Psavirus, Psimunavirus, Rdjlvirus, Rer2virus, Sap6virus, Send513virus, Septima3virus, Seuratvirus, Sextaecvirus, Sfi11virus, Sfi21dt1virus, Sitaravirus, Sk1virus, Slashvirus, Smoothievirus, Soupsvirus, Spbetavirus, Ssp2virus, T5virus, Tankvirus, Tin2virus, Titanvirus, Tm4virus, Tp21virus, Tp84virus, Triavirus, Trigintaduovirus, Vegasvirus, Vendettavirus, Wbetavirus, Wildcatvirus, Wizardvirus, Woesvirus, Xp10virus, Ydn12virus and Yuavirus)
family Ackermannviridae (such as, without limitation, genus Ag3virus, Limestonevirus, Cba120virus and Vi1virus)

Optionally, the bacteriophage is not part of the Order Caudovirales but from families with Unassigned order such as, without limitation, family Tectiviridae (such as genus Alphatectivirus, Betatectivirus), family Corticoviridae (such as genus Corticovirus), family Inoviridae (such as genus Fibrovirus, Habenivirus, Inovirus, Lineavirus, Plectrovirus, Saetivirus, Vespertiliovirus), family Cystoviridae (such as genus Cysto virus), family Leviviridae (such as genus Allolevivirus, Levivirus), family Microviridae (such as genus Alpha3microvirus, G4microvirus, Phix174microvirus, Bdellomicrovirus, Chlamydiamicrovirus, Spiromicrovirus) and family Plasmaviridae (such as genus Plasmavirus).

Optionally, the bacteriophage is targeting Archea not part of the Order Caudovirales but from families with Unassigned order such as, without limitation, Ampullaviridae, FuselloViridae, Globuloviridae, Guttaviridae, Lipothrixviridae, Pleolipoviridae, Rudiviridae, Salterprovirus and Bicaudaviridae.

A non-exhaustive listing of bacterial genera and their known host-specific bacteria viruses is presented in the following paragraphs. Synonyms and spelling variants are indicated in parentheses. Homonyms are repeated as often as they occur (e.g., D, D, d). Unnamed phages are indicated by “NN” beside their genus and their numbers are given in parentheses.

Bacteria of the genus Actinomyces can be infected by the following phages: Av-1, Av-2, Av-3, BF307, CTI, CT2, CT3, CT4, CT6, CT7, CT8 and 1281.

Bacteria of the genus Aeromonas can be infected by the following phages: AA-1, Aeh2, N, PMI, TP446, 3, 4, 11, 13, 29, 31, 32, 37, 43, 43-10T, 51, 54, 55R.1, 56, 56RR2, 57, 58, 59.1, 60, 63, Aehl, F, PM2, 1, 25, 31, 40RR2.8t, (syn=44R), (syn=44RR2.8t), 65, PM3, PM4, PM5 and PM6.

Bacteria of the genus Bacillus can be infected by the following phages: A, aizl, A1-K-I, B, BCJAI, BCI, BC2, BLLI, BLI, BP142, BSLI, BSL2, BSI, BS3, BS8, BS15, BS18, BS22, BS26, BS28, BS31, BS104, BS105, BS106, BTB, B1715V1, C, CK-I, Coll, Corl, CP-53, CS-I, CSi, D, D, D, D5, entl, FP8, FP9, FSi, FS2, FS3, FS5, FS8, FS9, G, GH8, GT8, GV-I, GV-2, GT-4, g3, g12, g13, g14, g16, g17, g21, g23, g24, g29, H2, kenl, KK-88, Kuml, Kyul, J7W-1, LP52, (syn=LP-52), L7, Mexl, MJ-1, mor2, MP-7, MPIO, MP12, MP14, MP15, Neol, N^{o} 2, N5, N6P, PBCI, PBLA, PBPI, P2, S-a, SF2, SF6, ShaI, SilI, SP02, (syn=ΦSPP1), SPR, STI, STi, SU-II, t, TbI, Tb2, Tb5, TbIO, Tb26, Tb51, Tb53, Tb55, Tb77, Tb97, Tb99, Tb560, Tb595, Td8, Td6, TdI5, TgI, Tg4, Tg6, Tg7, Tg9, TgIO, TgII, Tg13, TgI5, Tg21, Tinl, Tin7, Tin8, TinI3, Tm3, Tocl, Togl, toll, TP-I, TP-10vir, TP-15c, TP-16c, TP-17c, TP-19, TP35, TP51, TP-84, Tt4, Tt6, type A, type B, type C, type D, type E, Tφ3, VA-9, W, wx23, wx26, YunI, α, γ, pII, φmed-2, φT, φμ-4, φ3T, T75, φIO5, (syn=T105), IA, IB, 1-97A, 1-97B, 2, 2, 3, 3, 3, 5, 12, 14, 20, 30, 35, 36, 37, 38, 41C, 51, 63, 64, 138D, I, II, IV, NN-Bacillus (13), aleI, ARI, AR2, AR3, AR7, AR9, Bace-11, (syn=11), Bastille, BLI, BL2, BL3, BL4, BLS, BL6, BL8, BL9, BP124, BS28, BS80, Ch, CP-51, CP-54, D-5, darI, denI, DP-7, entl, FoSi, FoS2, FS4, FS6, FS7, G, gall, gamma, GEI, GF-2, GSi, GT-I, GT-2, GT-3, GT-4, GT-5, GT-6, GT-7, GV-6, g15, 19, 110, ISi, K, MP9, MP13, MP21, MP23, MP24, MP28, MP29, MP30, MP32, MP34, MP36, MP37, MP39, MP40, MP41, MP43, MP44, MP45, MP47, MP50, NLP-I, No. I, N17, N19, PBSI, PKI, PMBI, PMB12, PMJI, S, SPOI, SP3, SP5, SP6, SP7, SP8, SP9, SPIO, SP-15, SP50, (syn=SP-50), SP82, SST, subl, SW, Tg8, Tgl2, Tgl3, Tgl4, thuI, thuΛ, thuS, Tin4, Tin23, TP-13, TP33, TP50, TSP-I, type V, type VI, V, Vx, β22, φe, φNR2, φ25, φ63, 1, 1, 2, 2C, 3NT, 4, 5, 6, 7, 8, 9, 10, 12, 12, 17, 18, 19, 21, 138, III, 4 (B. megateriwn), 4 (B. sphaericus), AR13, BPP-IO, BS32, BS107, B1, B2, GA-1, GP-IO, GV-3, GV-5, g8, MP20, MP27, MP49, Nf, PP5, PP6, SF5, Tgl8, TP-1, Versailles, φ15, φ29, 1-97, 837/IV, mi-Bacillus (1), BatIO, BSLIO, BSLI I, BS6, BSII, BS16, BS23, BSIOI, BS102, g18, morl, PBLI, SN45, thu2, thu3, Tml, Tm2, TP-20, TP21, TP52, type F, type G, type IV, HN-BacMus (3), BLE, (syn=θc), BS2, BS4, BS5, BS7, BIO, B12, BS20, BS21, F, MJ-4, PBA12, AP50, AP50-04, AP50-11, AP50-23, AP50-26, AP50-27 and Bam35. The following Bacillus-specific phages are defective: DLP10716, DLP-11946, DPBS, DPB12, DPB21, DPB22, DPB23, GA-2, M, No. IM, PBLB, PBSH, PBSV, PBSW, PBSX, PBSY, PBSZ, phi, SPa, type 1 and μ.

Bacteria of the genus Bacteroides can be infected by the following phages: crAss-phage, ad 12, Baf-44, Baf-48B, Baf-64, Bf-1, Bf-52, B40-8, FI, βI, φAI, φBrOI, φBrO2, 11, 67.1, 67.3, 68.1, mt-Bacteroides (3), Bf42, Bf71, HN-Bdellovibrio (1) and BF-41.

Bacteria of the genus Bordetella can be infected by the following phages: 134 and NN-Bordetella (3).

Bacteria of the genus Borrellia can be infected by the following phages: NN-Borrelia (1) and NN-Borrelia (2).

Bacteria of the genus Brucella can be infected by the following phages: A422, Bk, (syn=Berkeley), BM29, FOi, (syn=F01), (syn=FQI), D, FP2, (syn=FP2), (syn=FD2), Fz, (syn=Fz75/13), (syn=Firenze 75/13), (syn=Fi), Fi, (syn=F1), Fim, (syn=Flm), (syn=Fim), FiU, (syn=FIU), (syn=FiU), F2, (syn=F2), F3, (syn=F3), F4, (syn=F4), F5, (syn=F5), F6, F7, (syn=F7), F25, (syn=F25), (syn=£25), F25U, (syn=F25u), (syn=F25U), (syn=F25V), F44, (syn-F44), F45, (syn=F45), F48, (syn=F48), 1, Im, M, MC/75, M51, (syn=M85), P, (syn=D), S708, R, Tb, (syn=TB), (syn=Tbilisi), W, (syn=Wb), (syn=Weybridge), X, 3, 6, 7, 10/1, (syn=10), (syn=F8), (syn=F8), 12m, 24/11, (syn=24), (syn=F9), (syn=F9), 45/111, (syn=45), 75, 84, 212/XV, (syn=212), (syn=Fi0), (syn=F10), 371/XXIX, (syn=371), (syn=Fn), (syn=FII) and 513.

Bacteria of the genus Burkholderia can be infected by the following phages: CP75, NN-Burkholderia (1) and 42.

Bacteria of the genus Campylobacter can be infected by the following phages: C type, NTCC12669, NTCC12670, NTCC12671, NTCC12672, NTCC12673, NTCC12674, NTCC12675, NTCC12676, NTCC12677, NTCC12678, NTCC12679, NTCC12680, NTCC12681, NTCC12682, NTCC12683, NTCC12684, 32f, 111c, 191, NN-Campylobacter (2), Vfi-6, (syn=V19), VfV-3, V2, V3, V8, V16, (syn=Vfi-1), V19, V20(V45), V45, (syn=V-45) and NN-Campylobacter (1).

Bacteria of the genus Chlamydia can be infected by the following phage: ChpI.

Bacteria of the genus Clostridium can be infected by the following phages: CAKI, CA5, Cal, CER, (syn=1C), CEγ, CldI, c-n71, c-203 Tox-, DER, (syn=ID), (syn=IDtOX+), HM3, KMI, KT, Ms, NAI, (syn=Naltox+), PA135Oe, Pf6, PL73, PL78, PL81, P1, P50, P5771, P19402, ICt0X+, 2Ct0X\ 2D3 (syn=2Dt0X+), 3C, (syn=3Ctox+), 4C, (syn=4Ct0X+), 56, III-I, NN-Clostridium (61), NBItOX+, al, CAI, HMT, HM2, PF15 P-23, P-46, Q-05, Q-oe, Q-16, Q-21, Q-26, Q-40, Q-46, S111, SA02, WA01, WA03, Wm, W523, 80, C, CA2, CA3, CPTI, CPT4, cl, c4, c5, HM7, H11/A1, H18/Ax, FWS23, Hi58ZA1, K2ZA1, K21ZS23, ML, NA2t0X; Pf2, Pf3, Pf4, S9ZS3, S41ZA1, S44ZS23, a2, 41, 112ZS23, 214/S23, 233/Ai, 234/S23, 235/S23, 11-1, 11-2, 11-3, NN-Clostridium (12), CAI, FI, K, S2, 1, 5 and NN-Clostridium (8).

Bacteria of the genus Corynebacterium can be infected by the following phages: CGKI (defective), A, A2, A3, A101, A128, A133, A137, A139, A155, A182, B, BF, B17, B18, B51, B271, B275, B276, B277, B279, B282, C, capi, CCI, CGI, CG2, CG33, CL31, Cog, (syn=CG5), D, E, F, H, H-I, hqi, hq2, 11ZH33, li/31, J, K, K, (syn=Ktox″), L, L, (syn=Ltox+), M, MC-I, MC-2, MC-3, MC-4, MLMa, N, O, ovi, ov2, ov3, P, P, R, RP6, RS29, S, T, U, U61, ub2, UH1, UH3, uh3, uh5, uh6, β, (syn=βtox+), βhv64, βvir, γ, (syn=ytox−), γI9, δ, (syn=δ′ox+), p, (syn=ptox−), Φ9, φ984, ω, IA, 1/1180, 2, 2/1180, 5/1180, 5ad/9717, 7/4465, 8/4465, 8ad/10269, 10/9253, 13Z9253, 15/3148, 21/9253, 28, 29, 55, 2747, 2893, 4498 and 5848.

Bacteria of the genus Enterococcus are infected by the following phage: DF78, FI, F2, 1, 2, 4, 14, 41, 867, DI, SB24, 26V, 182, 225, C2, C2F, E3, E62, DS96, H24, M35, P3, P9, SBIOI, S2, 2611, 5, 182a, 705, 873, 881, 940, 1051, 1057, 21096C, NN-Enterococcus (1), PEI, FI, F3, F4, VD13, 1, 200, 235 and 341.

Bacteria of the genus Erysipelothrix can be infected by the following phage: NN-Eiysipelothrix (1).

Bacteria of the genus Escherichia can be infected by the following phages: BW73, B278, D6, D108, E, EI, E24, E41, FI-2, FI-4, FI-5, H18A, Ff8B, i, MM, Mu, (syn=mu), (syn=MuI), (syn=Mu-I), (syn=MU-I), (syn=MuI), (syn=p), 025, PhI-5, Pk, PSP3, PI, PID, P2, P4 (defective), SI, Wφ, φK13, φR73 (defective), φI, φ2, φ7, φ92, ψ (defective), 7 A, 8φ, 9φ, 15 (defective), 18, 28-1, 186, 299, HH-Escherichia (2), AB48, CM, C4, C16, DD-VI, (syn=Dd-Vi), (syn=DDVI), (syn=DDVi), E4, E7, E28, FII, F13, H, HI, H3, H8, K3, M, N, ND-2, ND-3, ND4, ND-5, ND6, ND-7, Ox-1 (syn=OXI), (syn=HF), Ox-2 (syn=Ox2), (syn=OX2), Ox-3, Ox-4, Ox-5, (syn=OX5), Ox-6, (syn=66F), (syn=φ66t), (syn=φ66t-)5 0111, Ph1-1, R642, R643, R649, R669, S, SaI-I, SaI-2, SaI-3, SaI-4, SaI-5, SaI-6, φC23, φC45, TulI*-6, (syn=TulI*), TulP-24, TulI*46, TulP-60, T2, (syn=ganuTia), (syn=y), (syn=PC), (syn=P.C.), (syn=T-2), (syn=T2), (syn=P4), T4, (syn=T-4), (syn=T4), T6, T35, al, 1, IA, 3, (syn=Ac3), 3A, 3T+, (syn=3), (syn=MI), 5φ, (syn=φ5), 9266Q, CFO103, HK620, J, K, KIF, m59, no. A, no. E, no. 3, no. 9, N4, sd, (syn=Sd), (syn=SD), (syn=Sa)3 (syn=sd), (syn=SD), (syn=CD), T3, (syn=T-3), (syn=T3), T7, (syn=T-7), (syn=T7), WPK, W31, ΔH, φC3888, φK3, φK7, φK12, φV-1, Φ04-CF, Φ05, Φ06, Φ07, φI, φI.2, φ20, φ95, φ263, φIO92, φI, φII, (syn=φW), Ω8, 1, 3, 7, 8, 26, 27, 28-2, 29, 30, 31, 32, 38, 39, 42, 933W, NN-Escherichia (1), Esc-7-11, AC30, CVX-5, CI, DDUP, ECI, EC2, E21, E29, FI, F26S, F27S, Hi, HK022, HK97, (syn=(PHK97), HK139, HK253, HK256, K7, ND-I, no. D, PA-2, q, S2, TI, (syn=α), (syn=P28), (syn=T-1), (syn=Tx), T3C, T5, (syn=T-5), (syn=T5), UC-I, w, β4, γ2, A (syn=lambda), (syn=Φλ), ΦD326, φγ, Φ06, Φ7, Φ10, φ80, χ, (syn=χi), (syn=χX), (syn=φχi), 2, 4, 4A, 6, 8A, 102, 150, 168, 174, 3000, AC6, AC7, AC28, AC43, AC50, AC57, AC81, AC95, HK243, KIO, ZG/3A, 5, 5A, 21EL, H19-J, 933H, O157 typing phages 1 to 16, JES-2013, 121Q, 172-1, 1720a-02, ADB-2, AKFV33, av-05, bV_EcoS_AHP42, bV_EcoS_AHP24, bC_EcoS_AHS24, bV_EcoS_AKS96, CBA120.

Bacteria of the genus Fusobacterium are infected by the following phage: NN-Fusobacterium (2), fv83-554/3, fv88-531/2, 227, fv2377, fv2527 and fv8501.

Bacteria of the genus Haemophilus are infected by the following phage: HPI, S2 and N3.

Bacteria of the genus Helicobacter are infected by the following phage: HPI and AA-Helicobacter (1).

Bacteria of the genus Klebsiella are infected by the following phage: A10-2, K14B, K16B, K19, (syn=K19), K114, K115, K121, K128, K129, K132, K133, K135, K1106B, K1171 B, KI181B, K1832B, A10-1, AO-1, AO-2, AO-3, FC3-10, K, KI1, (syn=KII), K12, (syn=K12), K13, (syn=K13), (syn=KI 70/11), K14, (syn=K14), K15, (syn=K15), K16, (syn=K16), K17, (syn=K17), K18, (syn=K18), K119, (syn=K19), K127, (syn=K127), K131, (syn=K131), K135, KI171B, 11, VI, IX, C1-1, K14B, K18, K111, K112, K113, K116, K117, K118, K120, K122, K123, K124, K126, K130, K134, K1106B, KIi65B, K1328B, KLXI, K328, P5046, 11, 380, III, IV, VII, VIII, FC3-11, K12B, (syn=K12B), K125, (syn=K125), K142B, (syn=K142), (syn=K142B), K1181 B, (syn=KII 81), (syn=K1181B), K1765/1, (syn=K1765/1), K1842B, (syn=K1832B), K1937B, (syn=K1937B), LI, φ28, 7, 231, 483, 490, 632 and 864/100.

Bacteria of the genus Lepitospira are infected by the following phage: LEI, LE3, LE4 and -NN-Leptospira (1).

Bacteria of the genus Listeria are infected by the following phage: A511, 01761, 4211, 4286, (syn=B054), A005, A006, A020, A500, A502, A511, AI 18, A620, A640, B012, B021, B024, B025, B035, B051, B053, B054, B055, B056, BIOI, BI IO, B545, B604, B653, C707, D441, HSO47, HIOG, H8/73, H19, H21, H43, H46, H107, H108, HI 10, H163/84, H312, H340, H387, H391/73, H684/74, H924A, PSA, U153, φMLUP5, (syn=P35), 00241, 00611, 02971A, 02971C, 5/476, 5/911, 5/939, 5/11302, 5/11605, 5/11704, 184, 575, 633, 699/694, 744, 900, 1090, 1317, 1444, 1652, 1806, 1807, 1921/959, 1921/11367, 1921/11500, 1921/11566, 1921/12460, 1921/12582, 1967, 2389, 2425, 2671, 2685, 3274, 3550, 3551, 3552, 4276, 4277, 4292, 4477, 5337, 5348/11363, 5348/11646, 5348/12430, 5348/12434, 10072, 11355C, 11711A, 12029, 12981, 13441, 90666, 90816, 93253, 907515, 910716 and NN-Lisferia (15).

Bacteria of the genus Morganella are infected by the following phage: 47.

Bacteria of the genus Mycobacterium are infected by the following phage: 13, AGI, ALi, ATCC 11759, A2, B.C3, BG2, BKI, BK5, butyricum, B-I, B5, B7, B30, B35, Clark, CI, C2, DNAIII, DSP1, D4, D29, GS4E, (syn=GS4E), GS7, (syn=GS-7), (syn=GS7), IPa, lacticola, Legendre, Leo, L5, (syn=ΦL-5), MC-I, MC-3, MC-4, minetti, MTPHI I, Mx4, MyF3P/59a, phlei, (syn=phlei 1), phlei 4, Polonus II, rabinovitschi, smegmatis, TM4, TM9, TMIO, TM20, Y7, YIO, φ630, IB, IF, IH, 1/1, 67, 106, 1430, BI, (syn=BoI), B24, D, D29, F-K, F-S, HP, Polonus I, Roy, RI, (syn=RI-Myb), (syn=Ri), 11, 31, 40, 50, 103a, 103b, 128, 3111-D, 3215-D and NN-Mycobacterium (1).

Bacteria of the genus Neisseria are infected by the following phage: Group I, group II and NPI.

Bacteria of the genus Nocardia are infected by the following phage: MNP8, NJ-L, NS-8, N5 and TtiN-Nocardia.

Bacteria of the genus Proteus are infected by the following phage: Pm5, 13vir, 2/44, 4/545, 6/1004, 13/807, 20/826, 57, 67b, 78, 107/69, 121, 9/0, 22/608, 30/680, PmI, Pm3, Pm4, Pm6, Pm7, Pm9, PmIO, PmI I, Pv2, πI, φm, 7/549, 9B/2, 10A/31, 12/55, 14, 15, 16/789, 17/971, 19A/653, 23/532, 25/909, 26/219, 27/953, 32A/909, 33/971, 34/13, 65, 5006M, 7480b, VI, 13/3a, Clichy 12, π2600, φχ7, 1/1004, 5/742, 9, 12, 14, 22, 24/860, 2600/D52, Pm8 and 24/2514.

Bacteria of the genus Providencia are infected by the following phage: PL25, PL26, PL37, 9211/9295, 9213/921 Ib, 9248, 7/R49, 7476/322, 7478/325, 7479, 7480, 9000/9402 and 9213/921 Ia.

Bacteria of the genus Pseudomonas are infected by the following phage: Pf I, (syn=Pf-I), Pf2, Pf3, PP7, PRRI, 7s, im-Pseudomonas (1), A1-1, A1-2, B 17, B89, CB3, Col 2, Col 11, Col 18, Col 21, C154, C163, C167, C2121, E79, F8, ga, gb, H22, K1, M4, N2, Nu, PB-I, (syn=PBI), pfI6, PMN17, PPI, PP8, Psal, PsPI, PsP2, PsP3, PsP4, PsP5, PS3, PS17, PTB80, PX4, PX7, PYOI, PYO2, PYOS, PYO6, PYO9, PYOIO, PYO13, PYO14, PYO16, PYO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, PIK, SLPI, SL2, S2, UNL-I, wy, Yai, Ya4, Yan, φBE, φCTX, φC17, φKZ, (syn=ϵKZ), φ-LT, φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, HN-Pseudomonas (23), A856, B26, CI-I, CI-2, C5, D, gh-1, FI 16, HF, H90, K5, K6, KI 04, K109, K166, K267, N4, N5, O6N-25P, PE69, Pf, PPN25, PPN35, PPN89, PPN91, PP2, PP3, PP4, PP6, PP7, PP8, PP56, PP87, PPI 14, PP206, PP207, PP306, PP651, Psp231a, Pssy401, Pssy9220, psi, PTB2, PTB20, PTB42, PXI, PX3, PXIO, PX12, PX14, PYO70, PYO71, R, SH6, SH133, tf, YaS, Ya7, φBS, ΦKf77, φ-MC, φmnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119×, 145, 147, 170, 267, 284, 308, 525, NN-Pseudomonas (5), af, A7, B3, B33, B39, BI-I, C22, D3, D37, D40, D62, D3112, F7, F10, g, gd, ge, gξ HwI2, Jb 19, KFI, L°, OXN-32P, 06N-52P, PCH-I, PC13-1, PC35-1, PH2, PH51, PH93, PH132, PMW, PM13, PM57, PM61, PM62, PM63, PM69, PM105, PMI 13, PM681, PM682, PO4, PPI, PP4, PPS, PP64, PP65, PP66, PP71, PP86, PP88, PP92, PP401, PP711, PP891, Pssy41, Pssy42, Pssy403, Pssy404, Pssy420, Pssy923, PS4, PS-IO, Pz, SDI, SLI, SL3, SLS, SM, φC5, φCII, φCII-1, φC13, φC15, φMO, φX, φO4, φII, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, HN-Pseudomonas (6), G101, M6, M6a, LI, PB2, PssyI5, Pssy4210, Pssy4220, PYO12, PYO34, PYO49, PYO50, PYO51, PYO52, PYO53, PYO57, PYO59, PYO200, PX2, PX5, SL4, φO3, φO6 and 1214.

Bacteria of the genus Rickettsia are infected by the following phage: NN-Rickettsia.

Bacteria of the genus Salmonella are infected by the following phage: b, Beccles, CT, d, Dundee, f, Fels 2, GI, GUI, GVI, GVIII, k, K, i, j, L, 01, (syn=0-1), (syn=O1), (syn=O-I), (syn=7), 02, 03, P3, P9a, PIO, Sab3, Sab5, SanIS, SanI7, SI, Taunton, ViI, (syn=ViI), 9, imSalmonella (1), N-I, N-5, N-IO, N-17, N-22, 11, 12, 16-19, 20.2, 36, 449C/C178, 966A/C259, a, B.A.O.R., e, G4, GUI, L, LP7, M, MG40, N-18, PSA68, P4, P9c, P22, (syn=P22), (syn=PLT22), (syn=PLT22), P22al, P22-4, P22-7, P22-11, SNT-I, SNT-2, SP6, ViIIi, ViIV, ViV, ViVI, ViVII, Worksop, Sj5, c34, 1.37, 1(40), (syn=φ[40]), 1.422, 2, 2.5, 3b, 4, 5, 6.14(18), 8, 14(6.7), 10, 27, 28B, 30, 31, 32, 33, 34, 36, 37, 39, 1412, SNT-3, 7-11, 40.3, c, C236, C557, C625, C966N, g, GV, G5, GI 73, h, IRA, Jersey, MB78, P22-1, P22-3, P22-12, SabI, Sab2, Sab2, Sab4, SanI, San2, San3, San4, San6, San7, San8, San9, SanI3, SanI4, SanI6, SanI8, SanI9, San20, San21, San22, San23, San24, San25, San26, SasLI, SasL2, SasL3, SasL4, SasL5, SIBL, SII, ViII, φI, 1, 2, 3a, 3aI, 1010, Ym-Salmonella (1), N-4, SasL6 and 27.

Bacteria of the genus Serratia are infected by the following phage: A2P, PS20, SMB3, SMP, SMPS, SM2, V40, V56, ic, ΦCP-3, ΦCP-6, 3M, 10/1a, 20A, 34CC, 34H, 38T, 345G, 345P, 501B, SMB2, SMP2, BC, BT, CW2, CW3, CW4, CW5, Lt232, L2232, L34, L.228, SLP, SMPA, V.43, σ, φCWI, ΦCP6-1, ΦCP6-2, ΦCP6-5, 3T, 5, 8, 9F, 10/1, 20E, 32/6, 34B, 34CT, 34P, 37, 41, 56, 56D, 56P, 60P, 61/6, 74/6, 76/4, 101/8900, 226, 227, 228, 229F, 286, 289, 290F, 512, 764a, 2847/10, 2847/10a, L.359 and SMBI.

Bacteria of the genus Shigella are infected by the following phage: Fsa, (syn=a), FSD2d, (syn=D2d), (syn=W2d), FSD2E, (syn=W2e), fv, F6, f7.8, H-Sh, PES, P90, SfII, Sh, SHm, SHrv, (syn=HIV), SHvi, (syn=HVI), SHVvm, (syn=HVIII), SKy66, (syn=gamma 66), (syn=γββ), (syn=γ66b), SKm, (syn=SIIIb)5 (syn=UI), SKw, (syn=Siva), (syn=IV), SIC™, (syn=SIVA.), (syn=IVA), SKvi, (syn=KVI), (syn=Svi), (syn=VI), SKvm, (syn=Svm), (syn=VIII), SKVTIIA, (syn=SvmA), (syn=VIIIA), STvi, STK, STx1, STxn, S66, W2, (syn=D2c), (syn=D20), ΦI, ΦIVb 3-SO-R, 8368-SO-R, F7, (syn=FS7), (syn=K29), F10, (syn=FSIO), (syn=K31), 11, (syn=alfa), (syn=FSa), (syn=KI 8), (syn=a), 12, (syn=a), (syn=K19), SG33, (syn=G35), (syn=SO-35/G), SG35, (syn=SO-55/G), SG3201, (syn=SO-3201/G), SHn, (syn=HII), SHv, (syn=SHV), SHx, SHX, SKn, (syn=K2), (syn=KII), (syn=Sn), (syn=SsI1), (syn=11), SKrv, (syn=Sm), (syn=SsIV), (syn=IV), SK1Va, (syn=Swab), (syn=SsIVa), (syn=IVa), SKV, (syn=K4), (syn=KV), (syn=SV), (syn=SsV), (syn=V), SKx, (syn=K9), (syn=KX), (syn=SX), (syn=SsX), (syn=X), STV, (syn=T35), (syn=35-50-R), STvm, (syn=T8345), (syn=8345-SO-S-R), W1, (syn=D8), (syn=FSD8), W2a, (syn=D2A), (syn=FS2a), DD-2, Sf6, FSi, (syn=FI), SF6, (syn=F6), SG42, (syn=SO-42/G), SG3203, (syn=SO-3203/G), SKF12, (syn=SsF12), (syn=F12), (syn=F12), STn, (syn=1881-SO-R), y66, (syn=gamma 66a), (syn=Ssy66), φ2, BII, DDVII, (syn=DD7), FSD2b, (syn=W2B), FS2, (syn=F2), (syn=F2), FS4, (syn=F4), (syn=F4), FS5, (syn=F5), (syn=F5), FS9, (syn=F9), (syn=F9), F11, P2-SO-S, SG36, (syn=SO-36/G), (syn=G36), SG3204, (syn=SO-3204/G), SG3244, (syn=SO-3244/G), SHi, (syn=HI), SHvrr, (syn=HVII), SHK, (syn=HIX), SHx1, SHxπ, (syn=HXn), SKI, KI, (syn=S1), (syn=SsI), SKVII, (syn=KVII), (syn=Svπ), (syn=SsVII), SKIX, (syn=KIX), (syn=Six), (syn=SsIX), SKXII, (syn=KXII), (syn=Sxn), (syn=SsXII), STi, STffI, STrv, STVi, STvπ, S70, S206, U2-S0-S, 3210-SO-S, 3859-SO-S, 4020-SO-S, φ3, φ5, φ7, φ8, φ9, φIO, φII, φI3, φI4, φI8, SHm, (syn=Hπi), SHχi, (syn=HXt) and SKxI, (syn=KXI), (syn=Sχi), (syn=SsXI), (syn=XI).

Bacteria of the genus Staphylococcus are infected by the following phage: A, EW, K, Ph5, Ph9, PhIO, PhI3, PI, P2, P3, P4, P8, P9, PIO, RG, SB-i, (syn=Sb-I), S3K, Twort, ΦSK311, Φ812, 06, 40, 58, 119, 130, 131, 200, 1623, STCI, (syn=stcI), STC2, (syn=stc2), 44AHJD, 68, ACI, AC2, A6″C″, A9″C″, b581, CA-1, CA-2, CA-3, CA-4, CA-5, DI I, L39x35, L54a, M42, NI, N2, N3, N4, N5, N7, N8, NIO, NiI, N12, N13, N14, N16, Ph6, PhI2, PhI4, UC-18, U4, U15, SI, S2, S3, S4, S5, X2, Z1, φB5-2, φD, ω, 11, (syn=φII), (syn=P11-M15), 15, 28, 28A, 29, 31, 31 B, 37, 42D, (syn=P42D), 44A, 48, 51, 52, 52A, (syn=P52A), 52B, 53, 55, 69, 71, (syn=P71), 71A, 72, 75, 76, 77, 79, 80, 80a, 82, 82A, 83 A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, 12100, AC3, A8, AIO, A13, b594n, D, HK2, N9, N15, P52, P87, SI, S6, Z4, φRE, 3A, 3B, 3C, 6, 7, 16, 21, 42B, 42C, 42E, 44, 47, 47A5 47C, 51, 54, 54×1, 70, 73, 75, 78, 81, 82, 88, 93, 94, 101, 105, 110, 115, 129/16, 174, 594n, 1363/14, 2460 and mS-Staphylococcus (1).

Bacteria of the genus Streptococcus are infected by the following phage: EJ-I, NN-Streptococais (1), a, C1, FL0Ths, H39, Cp-I, Cp-5, Cp-7, Cp-9, Cp-I0, AT298, A5, aIO/J1, aIO/J2, aIO/J5, aIO/J9, A25, BTI 1, b6, CAI, c20-1, c20-2, DP-1, Dp-4, DTI, ET42, eIO, FA101, FEThs, Fκ, FKKIOI, FKLIO, FKP74, FKH, FLOThs, FyIOI, fI, F10, F20140/76, g, GT-234, HB3, (syn=HB-3), HB-623, HB-746, M102, O1205, φO1205, PST, PO, PI, P2, P3, P5, P6, P8, P9, P9, P12, P13, P14, P49, P50, P51, P52, P53, P54, P55, P56, P57, P58, P59, P64, P67, P69, P71, P73, P75, P76, P77, P82, P83, P88, sc, sch, sf, SfII 1, (syn=SFiII), (syn=(syn=ΦSfiII), (syn=φSfiI I), sfiI9, (syn=SFiI9), (syn=φSFiI9), (syn=φSfiI9), Sfi21, (syn=SFi21), (syn=φSFi21), (syn=φSfi21), ST0, STX, st2, ST2, ST4, S3, (syn=φS3), s265, Φ17, φ42, Φ57, φ80, φ81, φ82, φ83, φ84, φ85, φ86, φ87, φ88, φ89, φ90, φ91, φ92, φ93, φ94, φ95, φ96, φ97, φ98, φ99, φIOO, φIOI, φIO2, φ227, Φ7201, ω1, ω2, ω3, ω4, ω5, ω6, ω8, ωIO, 1, 6, 9, 1OF, 12/12, 14, 17SR, 19S, 24, 50/33, 50/34, 55/14, 55/15, 70/35, 70/36, 71/ST15, 71/45, 71/46, 74F, 79/37, 79/38, 80/J4, 80/J9, 80/ST16, 80/15, 80/47, 80/48, 101, 103/39, 103/40, 121/41, 121/42, 123/43, 123/44, 124/44, 337/ST17 and m Streptococcus (34).

Bacteria of the genus Treponema are infected by the following phage: NN-Treponema (1).

Bacteria of the genus Vibrio are infected by the following phage: CTXΦ, fs, (syn=si), fs2, Ivpf5, VfI2, Vf33, VPIΦ, VSK, v6, 493, CP-TI, ET25, kappa, K139, Labol, )XN-69P, OXN-86, O6N-21P, PB-I, P147, rp-1, SE3, VA-I, (syn=VcA-I), VcA-2, VPI, VP2, VP4, VP7, VP8, VP9, VPIO, VP17, VP18, VP19, X29, (syn=29 d'Herelle), t, ΦHAW1-1, ΦHAW1-2, ΦHAW1-3, ΦHAWI-4, ΦHAW1-5, ΦHAW1-6, ΦHAW1-7, XHAWI-8, ΦHAW1-9, ΦHAW1-10, ΦHC1-1, ΦHC1-2, ΦHC1-3, ΦHC1-4, ΦHC2-1, >H02-2, ΦHC2-3, ΦHC2-4, ΦHC3-1, ΦHC3-2, ΦHC3-3, ΦHD1S-1, ΦHD1S-2, ΦHD2S-1, ΦHD2S-2, ΦHD2S-3, ΦHD2S-4, ΦHD2S-5, ΦHDO-1, ΦHDO-2, ΦHDO-3, ΦHDO-4, ΦHDO-5, ΦHDO-6, ΦKL-33, ΦKL-34, ΦKL-35, ΦKL-36, ΦKWH-2, ΦKWH-3, ΦKWH-4, ΦMARQ-1, ΦMARQ-2, ΦMARQ-3, ΦMOAT-1, 1)0139, ΦPEL1A-1, ΦPEL1A-2, ΦPEL8A-1, ΦPEL8A-2, ΦPEL8A-3, ΦPEL8C-1, ΦPEL8C-2, ΦPEL13A-1, ΦPEL13B-1, ΦPEL13B-2, ΦPEL13B-3, ΦPEL13B-4, ΦPEL13B-5, ΦPEL13B-6, ΦPEL13B-7, ΦPEL13B-8, ΦPEL13B-9, ΦPEL13B-10, φVP143, φVP253, Φ16, ΦI38, 1-II, 5, 13, 14, 16, 24, 32, 493, 6214, 7050, 7227, II, (syn=group II), (syn==φ2), V, VIII, ˜m-Vibrio (13), KVP20, KVP40, nt-1, 06N-22P, P68, e1, e2, e3, e4, e5, FK, G, I, K, nt-6, NI, N2, N3, N4, N5, 06N-34P, OXN-72P, OXN-85P, OXN-100P, P, Ph-I, PL163/10, Q, S, T, φ92, 1-9, 37, 51, 57, 70A-8, 72A-4, 72A-10, 110A-4, 333, 4996, I (syn=group I), III (syn=group III), VI, (syn=A-Saratov), VII, IX, X, HN-Vibrio (6), pAI, 7, 7-8, 70A-2, 71A-6, 72A-5, 72A-8, 108A-10, 109A-6, 109A-8, I IOA-1, 110A-5, 110A-7, hv-1, OXN-52P, P13, P38, P53, P65, P108, PiII, TPI3 VP3, VP6, VP12, VP13, 70A-3, 70A-4, 70A-10, 72A-1, 108A-3, 109-B1, 110A-2, 149, (syn=T149), IV, (syn=group IV), NN-Vibrio (22), VPS, VPII, VP15, VP16, a1, a2, a3a, a3b, 353B and HN-Vibrio (7).

Bacteria of the genus Yersinia are infected by the following phage: H, H-1, H-2, H-3, H-4, Lucas 110, Lucas 303, Lucas 404, YerA3, YerA7, YerA20, YerA41, 3/M64-76, 5/G394-76, 6/C753-76, 8/C239-76, 9/F18167, 1701, 1710, PST, 1/F2852-76, D'Herelle, EV, H, Kotljarova, PTB, R, Y, YerA41, φYerO3-12, 3, 4/C1324-76, 7/F783-76, 903, 1/M6176 and Yer2AT.

More preferably, the bacteriophage is selected in the group consisting of Salmonella virus SKML39, Shigella virus AG3, Dickeya virus Limestone, Dickeya virus RC2014, Escherichia virus CBA120, Escherichia virus PhaxI, Salmonella virus 38, Salmonella virus Det7, Salmonella virus GG32, Salmonella virus PM10, Salmonella virus SFP10, Salmonella virus SH19, Salmonella virus SJ3, Escherichia virus ECML4, Salmonella virus Marshall, Salmonella virus Maynard, Salmonella virus SJ2, Salmonella virus STML131, Salmonella virus ViI, Erwinia virus Ea2809, Klebsiella virus 0507KN21, Serratia virus IME250, Serratia virus MAM1, Campylobacter virus CP21, Campylobacter virus CP220, Campylobacter virus CPt10, Campylobacter virus IB635, Campylobacter virus CP81, Campylobacter virus CP30A, Campylobacter virus CPX, Campylobacter virus NCTC12673, Erwinia virus Ea214, Erwinia virus M7, Escherichia virus AYO145A, Escherichia virus EC6, Escherichia virus HY02, Escherichia virus JH2, Escherichia virus TP1, Escherichia virus VpaE1, Escherichia virus wV8, Salmonella virus FelixO1, Salmonella virus HB2014, Salmonella virus Mushroom, Salmonella virus UAB87, Citrobacter virus Moogle, Citrobacter virus Mordin, Escherichia virus SUSP1, Escherichia virus SUSP2, Aeromonas virus phiO18P, Haemophilus virus HP1, Haemophilus virus HP2, Pasteurella virus F108, Vibrio virus K139, Vibrio virus Kappa, Burkholderia virus phi52237, Burkholderia virus phiE122, Burkholderia virus phiE202, Escherichia virus 186, Escherichia virus P4, Escherichia virus P2, Escherichia virus Wphi, Mannheimia virus PHL101, Pseudomonas virus phiCTX, Ralstonia virus RSA1, Salmonella virus Fels2, Salmonella virus PsP3, Salmonella virus SopEphi, Yersinia virus L413C, Staphylococcus virus G1, Staphylococcus virus G15, Staphylococcus virus JD7, Staphylococcus virus K, Staphylococcus virus MCE2014, Staphylococcus virus P108, Staphylococcus virus Rodi, Staphylococcus virus S253, Staphylococcus virus S25-4, Staphylococcus virus SA12, Listeria virus A511, Listeria virus P100, Staphylococcus virus Remus, Staphylococcus virus SA11, Staphylococcus virus Stau2, Bacillus virus Camphawk, Bacillus virus SPO1, Bacillus virus BCP78, Bacillus virus TsarBomba, Staphylococcus virus Twort, Enterococcus virus phiEC24C, Lactobacillus virus Lb338-1, Lactobacillus virus LP65, Enterobacter virus PG7, Escherichia virus CC31, Klebsiella virus JD18, Klebsiella virus PKO111, Escherichia virus Bp7, Escherichia virus IME08, Escherichia virus JS10, Escherichia virus JS98, Escherichia virus QL01, Escherichia virus VR5, Enterobacter virus Eap3, Klebsiella virus KP15, Klebsiella virus KP27, Klebsiella virus Matisse, Klebsiella virus Miro, Citrobacter virus Merlin, Citrobacter virus Moon, Escherichia virus JSE, Escherichia virus phi1, Escherichia virus RB49, Escherichia virus HX01, Escherichia virus J509, Escherichia virus RB69, Shigella virus UTAM, Salmonella virus S16, Salmonella virus STML198, Vibrio virus KVP40, Vibrio virus nt1, Vibrio virus VaIKK3, Escherichia virus VR7, Escherichia virus VR20, Escherichia virus VR25, Escherichia virus VR26, Shigella virus SP18, Escherichia virus AR1, Escherichia virus C40, Escherichia virus E112, Escherichia virus ECML134, Escherichia virus HY01, Escherichia virus Ime09, Escherichia virus RB3, Escherichia virus RB14, Escherichia virus T4, Shigella virus Pss1, Shigella virus ShfI2, Yersinia virus D1, Yersinia virus PST, Acinetobacter virus 133, Aeromonas virus 65, Aeromonas virus Aeh1, Escherichia virus RB16, Escherichia virus RB32, Escherichia virus RB43, Pseudomonas virus 42, Cronobacter virus CR3, Cronobacter virus CR8, Cronobacter virus CR9, Cronobacter virus PBES02, Pectobacterium virus phiTE, Cronobacter virus GAP31, Escherichia virus 4MG, Salmonella virus SE1, Salmonella virus SSE121, Escherichia virus FFH2, Escherichia virus FV3, Escherichia virus JES2013, Escherichia virus V5, Brevibacillus virus Abouo, Brevibacillus virus Davies, Bacillus virus Agate, Bacillus virus Bobb, Bacillus virus Bp8pC, Erwinia virus Deimos, Erwinia virus Ea35-70, Erwinia virus RAY, Erwinia virus Simmy50, Erwinia virus SpecialG, Acinetobacter virus AB1, Acinetobacter virus AB2, Acinetobacter virus AbC62, Acinetobacter virus AP22, Arthrobacter virus ArV1, Arthrobacter virus Trina, Bacillus virus AvesoBmore, Bacillus virus B4, Bacillus virus Bigbertha, Bacillus virus Riley, Bacillus virus Spock, Bacillus virus Troll, Bacillus virus Bastille, Bacillus virus CAM003, Bacillus virus Bc431, Bacillus virus Bcp1, Bacillus virus BCP82, Bacillus virus BM15, Bacillus virus Deepblue, Bacillus virus JBP901, Burkholderia virus Bcep1, Burkholderia virus Bcep43, Burkholderia virus Bcep781, Burkholderia virus BcepNY3, Xanthomonas virus OP2, Burkholderia virus BcepMu, Burkholderia virus phiE255, Aeromonas virus 44RR2, Mycobacterium virus Alice, Mycobacterium virus Bxz1, Mycobacterium virus Dandelion, Mycobacterium virus HyRo, Mycobacterium virus 13, Mycobacterium virus Nappy, Mycobacterium virus Sebata, Clostridium virus phiC2, Clostridium virus phiCD27, Clostridium virus phiCD119, Bacillus virus CP51, Bacillus virus JL, Bacillus virus Shanette, Escherichia virus CVM10, Escherichia virus ep3, Erwinia virus Asesino, Erwinia virus EaH2, Pseudomonas virus EL, Halomonas virus HAP1, Vibrio virus VP882, Brevibacillus virus Jimmer, Brevibacillus virus Osiris, Pseudomonas virus Ab03, Pseudomonas virus KPP10, Pseudomonas virus PAKP3, Sinorhizobium virus M7, Sinorhizobium virus M12, Sinorhizobium virus N3, Erwinia virus Machina, Arthrobacter virus Brent, Arthrobacter virus Jawnski, Arthrobacter virus Martha, Arthrobacter virus Sonny, Edwardsiella virus MSW3, Edwardsiella virus PEi21, Escherichia virus Mu, Shigella virus SfMu, Halobacterium virus phiH, Bacillus virus Grass, Bacillus virus NIT1, Bacillus virus SPG24, Aeromonas virus 43, Escherichia virus P1, Pseudomonas virus CAb1, Pseudomonas virus CAb02, Pseudomonas virus JG004, Pseudomonas virus PAKP1, Pseudomonas virus PAKP4, Pseudomonas virus PaP1, Burkholderia virus BcepF1, Pseudomonas virus 141, Pseudomonas virus Ab28, Pseudomonas virus DL60, Pseudomonas virus DL68, Pseudomonas virus F8, Pseudomonas virus JG024, Pseudomonas virus KPP12, Pseudomonas virus LBL3, Pseudomonas virus LMA2, Pseudomonas virus PB1, Pseudomonas virus SN, Pseudomonas virus PA7, Pseudomonas virus phiKZ, Rhizobium virus RHEph4, Ralstonia virus RSF1, Ralstonia virus RSL2, Ralstonia virus RSL1, Aeromonas virus 25, Aeromonas virus 31, Aeromonas virus Aes12, Aeromonas virus Aes508, Aeromonas virus AS4, Stenotrophomonas virus IME13, Staphylococcus virus IPLAC1C, Staphylococcus virus SEP1, Salmonella virus SPN3US, Bacillus virus 1, Geobacillus virus GBSV1, Yersinia virus R1RT, Yersinia virus TG1, Bacillus virus G, Bacillus virus PBS1, Microcystis virus Ma-LMM01, Vibrio virus MAR, Vibrio virus VHML, Vibrio virus VP585, Bacillus virus BPS13, Bacillus virus Hakuna, Bacillus virus Megatron, Bacillus virus WPh, Acinetobacter virus AB3, Acinetobacter virus Abp1, Acinetobacter virus Fri1, Acinetobacter virus IME200, Acinetobacter virus PD6A3, Acinetobacter virus PDAB9, Acinetobacter virus phiAB1, Escherichia virus K30, Klebsiella virus K5, Klebsiella virus K11, Klebsiella virus Kp1, Klebsiella virus KP32, Klebsiella virus KpV289, Klebsiella virus F19, Klebsiella virus K244, Klebsiella virus Kp2, Klebsiella virus KP34, Klebsiella virus KpV41, Klebsiella virus KpV71, Klebsiella virus KpV475, Klebsiella virus SU503, Klebsiella virus SU552A, Pantoea virus Limelight, Pantoea virus Limezero, Pseudomonas virus LKA1, Pseudomonas virus phiKMV, Xanthomonas virus f20, Xanthomonas virus f30, Xylella virus Prado, Erwinia virus Era103, Escherichia virus K5, Escherichia virus K1-5, Escherichia virus K1 E, Salmonella virus SP6, Escherichia virus T7, Kluyvera virus Kvp1, Pseudomonas virus gh1, Prochlorococcus virus PSSP7, Synechococcus virus P60, Synechococcus virus Syn5, Streptococcus virus Cpl, Streptococcus virus Cpl, Staphylococcus virus 44AHJD, Streptococcus virus C1, Bacillus virus B103, Bacillus virus GA1, Bacillus virus phi29, Kurthia virus 6, Actinomyces virus Av1, Mycoplasma virus P1, Escherichia virus 24B, Escherichia virus 933W, Escherichia virus Min27, Escherichia virus PA28, Escherichia virus Stx2 II, Shigella virus 7502Stx, Shigella virus POCJ13, Escherichia virus 191, Escherichia virus PA2, Escherichia virus TL2011, Shigella virus VASD, Burkholderia virus Bcep22, Burkholderia virus Bcepi102, Burkholderia virus Bcepmigl, Burkholderia virus DC1, Bordetella virus BPP1, Burkholderia virus BcepC6B, Cellulophaga virus Cba41, Cellulophaga virus Cba172, Dinoroseobacter virus DFL12, Erwinia virus Ea9-2, Erwinia virus Frozen, Escherichia virus phiV10, Salmonella virus Epsilon15, Salmonella virus SPN1S, Pseudomonas virus F116, Pseudomonas virus H66, Escherichia virus APECS, Escherichia virus APEC7, Escherichia virus Bp4, Escherichia virus EC1 UPM, Escherichia virus ECBP1, Escherichia virus G7C, Escherichia virus IME11, Shigella virus Sb1, Achromobacter virus Axp3, Achromobacter virus JWAlpha, Edwardsiella virus KF1, Pseudomonas virus KPP25, Pseudomonas virus R18, Pseudomonas virus Ab09, Pseudomonas virus LIT1, Pseudomonas virus PA26, Pseudomonas virus Ab22, Pseudomonas virus CHU, Pseudomonas virus LUZ24, Pseudomonas virus PAA2, Pseudomonas virus PaP3, Pseudomonas virus PaP4, Pseudomonas virus TL, Pseudomonas virus KPP21, Pseudomonas virus LUZ7, Escherichia virus N4, Salmonella virus 9NA, Salmonella virus SP069, Salmonella virus BTP1, Salmonella virus HK620, Salmonella virus P22, Salmonella virus ST64T, Shigella virus Sf6, Bacillus virus Page, Bacillus virus Palmer, Bacillus virus Pascal, Bacillus virus Pony, Bacillus virus Pookie, Escherichia virus 172-1, Escherichia virus ECB2, Escherichia virus NJ01, Escherichia virus phiEco32, Escherichia virus Septimal 1, Escherichia virus SU10, Brucella virus Pr, Brucella virus Tb, Escherichia virus Pollock, Salmonella virus FSL SP-058, Salmonella virus FSL SP-076, Helicobacter virus 1961 P, Helicobacter virus KHP30, Helicobacter virus KHP40, Hamiltonella virus APSE1, Lactococcus virus KSY1, Phormidium virus WMP3, Phormidium virus WMP4, Pseudomonas virus 119X, Roseobacter virus SIO1, Vibrio virus VpV262, Vibrio virus VC8, Vibrio virus VP2, Vibrio virus VPS, Streptomyces virus Amela, Streptomyces virus phiCAM, Streptomyces virus Aaronocolus, Streptomyces virus Caliburn, Streptomyces virus Danzina, Streptomyces virus Hydra, Streptomyces virus Izzy, Streptomyces virus Lannister, Streptomyces virus Lika, Streptomyces virus Sujidade, Streptomyces virus Zemlya, Streptomyces virus ELB20, Streptomyces virus R4, Streptomyces virus phiHau3, Mycobacterium virus Acadian, Mycobacterium virus Baee, Mycobacterium virus Reprobate, Mycobacterium virus Adawi, Mycobacterium virus Bane1, Mycobacterium virus BrownCNA, Mycobacterium virus Chrisnmich, Mycobacterium virus Cooper, Mycobacterium virus JAMaL, Mycobacterium virus Nigel, Mycobacterium virus Stinger, Mycobacterium virus Vincenzo, Mycobacterium virus Zemanar, Mycobacterium virus Apizium, Mycobacterium virus Manad, Mycobacterium virus Oline, Mycobacterium virus Osmaximus, Mycobacterium virus Pg1, Mycobacterium virus Soto, Mycobacterium virus Suffolk, Mycobacterium virus Athena, Mycobacterium virus Bernardo, Mycobacterium virus Gadjet, Mycobacterium virus Pipefish, Mycobacterium virus Godines, Mycobacterium virus Rosebush, Mycobacterium virus Babsiella, Mycobacterium virus Brujita, Mycobacterium virus Che9c, Mycobacterium virus Sbash, Mycobacterium virus Hawkeye, Mycobacterium virus Plot, Salmonella virus AG11, Salmonella virus Ent1, Salmonella virus f185E, Salmonella virus Jersey, Salmonella virus L13, Salmonella virus LSPA1, Salmonella virus SE2, Salmonella virus SETP3, Salmonella virus SETP7, Salmonella virus SETP13, Salmonella virus SP101, Salmonella virus SS3e, Salmonella virus wksI3, Escherichia virus K1G, Escherichia virus K1H, Escherichia virus K1ind1, Escherichia virus K1ind2, Salmonella virus SP31, Leuconostoc virus Lmd1, Leuconostoc virus LN03, Leuconostoc virus LN04, Leuconostoc virus LN12, Leuconostoc virus LN6B, Leuconostoc virus P793, Leuconostoc virus 1A4, Leuconostoc virus Ln8, Leuconostoc virus Ln9, Leuconostoc virus LN25, Leuconostoc virus LN34, Leuconostoc virus LNTR3, Mycobacterium virus Bongo, Mycobacterium virus Rey, Mycobacterium virus Butters, Mycobacterium virus Michelle, Mycobacterium virus Charlie, Mycobacterium virus Pipsqueaks, Mycobacterium virus Xeno, Mycobacterium virus Panchino, Mycobacterium virus Phrann, Mycobacterium virus Redi, Mycobacterium virus Skinnyp, Gordonia virus BaxterFox, Gordonia virus Yeezy, Gordonia virus Kita, Gordonia virus Zirinka, Gorrdonia virus Nymphadora, Mycobacterium virus Bignuz, Mycobacterium virus Brusacoram, Mycobacterium virus Donovan, Mycobacterium virus Fishburne, Mycobacterium virus Jebeks, Mycobacterium virus Malithi, Mycobacterium virus Phayonce, Enterobacter virus F20, Klebsiella virus 1513, Klebsiella virus KLPN1, Klebsiella virus KP36, Klebsiella virus PKP126, Klebsiella virus Sushi, Escherichia virus AHP42, Escherichia virus AHS24, Escherichia virus AKS96, Escherichia virus C119, Escherichia virus E41c, Escherichia virus Eb49, Escherichia virus Jk06, Escherichia virus KP26, Escherichia virus Rogue1, Escherichia virus ACGM12, Escherichia virus Rtp, Escherichia virus ADB2, Escherichia virus JMPW1, Escherichia virus JMPW2, Escherichia virus T1, Shigella virus PSf2, Shigella virus Shf11, Citrobacter virus Stevie, Escherichia virus TLS, Salmonella virus SP126, Cronobacter virus Esp2949-1, Pseudomonas virus Ab18, Pseudomonas virus Ab19, Pseudomonas virus PaMx11, Arthrobacter virus Amigo, Propionibacterium virus Anatole, Propionibacterium virus B3, Bacillus virus Andromeda, Bacillus virus Blastoid, Bacillus virus Curly, Bacillus virus Eoghan, Bacillus virus Finn, Bacillus virus Glittering, Bacillus virus Riggi, Bacillus virus Taylor, Gordonia virus Attis, Mycobacterium virus Barnyard, Mycobacterium virus Konstantine, Mycobacterium virus Predator, Mycobacterium virus Bernal13, Staphylococcus virus 13, Staphylococcus virus 77, Staphylococcus virus 108PVL, Mycobacterium virus Bron, Mycobacterium virus Faith1, Mycobacterium virus Joedirt, Mycobacterium virus Rumpelstiltskin, Lactococcus virus bIL67, Lactococcus virus c2, Lactobacillus virus c5, Lactobacillus virus Ld3, Lactobacillus virus Ld17, Lactobacillus virus Ld25A, Lactobacillus virus LLKu, Lactobacillus virus phiLdb, Cellulophaga virus Cba121, Cellulophaga virus Cba171, Cellulophaga virus Cba181, Cellulophaga virus ST, Bacillus virus 250, Bacillus virus IEBH, Mycobacterium virus Ardmore, Mycobacterium virus Avani, Mycobacterium virus Boomer, Mycobacterium virus Che8, Mycobacterium virus Che9d, Mycobacterium virus Deadp, Mycobacterium virus Dlane, Mycobacterium virus Dorothy, Mycobacterium virus Dotproduct, Mycobacterium virus Drago, Mycobacterium virus Fruitloop, Mycobacterium virus Gumbie, Mycobacterium virus lbhubesi, Mycobacterium virus Llij, Mycobacterium virus Mozy, Mycobacterium virus Mutaforma13, Mycobacterium virus Pacc40, Mycobacterium virus PMC, Mycobacterium virus Ramsey, Mycobacterium virus Rockyhorror, Mycobacterium virus SG4, Mycobacterium virus Shaunal, Mycobacterium virus Shilan, Mycobacterium virus Spartacus, Mycobacterium virus Taj, Mycobacterium virus Tweety, Mycobacterium virus Wee, Mycobacterium virus Yoshi, Salmonella virus Chi, Salmonella virus FSLSP030, Salmonella virus FSLSP088, Salmonella virus iEPS5, Salmonella virus SPN19, Mycobacterium virus 244, Mycobacterium virus Bask21, Mycobacterium virus CJW1, Mycobacterium virus Eureka, Mycobacterium virus Kostya, Mycobacterium virus Porky, Mycobacterium virus Pumpkin, Mycobacterium virus Sirduracell, Mycobacterium virus Toto, Mycobacterium virus Corndog, Mycobacterium virus Firecracker, Rhodobacter virus RcCronus, Pseudomonas virus D3112, Pseudomonas virus DMS3, Pseudomonas virus FHA0480, Pseudomonas virus LPB1, Pseudomonas virus MP22, Pseudomonas virus MP29, Pseudomonas virus MP38, Pseudomonas virus PA1 KOR, Pseudomonas virus D3, Pseudomonas virus PMG1, Arthrobacter virus Decurro, Gordonia virus Demosthenes, Gordonia virus Katyusha, Gordonia virus Kvothe, Propionibacterium virus B22, Propionibacterium virus Doucette, Propionibacterium virus E6, Propionibacterium virus G4, Burkholderia virus phi6442, Burkholderia virus phi1026b, Burkholderia virus phiE125, Edwardsiella virus eiAU, Mycobacterium virus Ff47, Mycobacterium virus Muddy, Mycobacterium virus Gaia, Mycobacterium virus Giles, Arthrobacter virus Captnmurica, Arthrobacter virus Gordon, Gordonia virus GordTnk2, Paenibacillus virus Harrison, Escherichia virus EK99P1, Escherichia virus HK578, Escherichia virus JL1, Escherichia virus SSL2009a, Escherichia virus YD2008s, Shigella virus EP23, Sodalis virus S01, Escherichia virus HK022, Escherichia virus HK75, Escherichia virus HK97, Escherichia virus HK106, Escherichia virus HK446, Escherichia virus HK542, Escherichia virus HK544, Escherichia virus HK633, Escherichia virus mEp234, Escherichia virus mEp235, Escherichia virus mEpX1, Escherichia virus mEpX2, Escherichia virus mEp043, Escherichia virus mEp213, Escherichia virus mEp237, Escherichia virus mEp390, Escherichia virus mEp460, Escherichia virus mEp505, Escherichia virus mEp506, Brevibacillus virus Jenst, Achromobacter virus 83-24, Achromobacter virus JWX, Arthrobacter virus Kellezzio, Arthrobacter virus Kitkat, Arthrobacter virus Bennie, Arthrobacter virus DrRobert, Arthrobacter virus Glenn, Arthrobacter virus HunterDalle, Arthrobacter virus Joann, Arthrobacter virus Korra, Arthrobacter virus Preamble, Arthrobacter virus Pumancara, Arthrobacter virus Wayne, Mycobacterium virus Alma, Mycobacterium virus Arturo, Mycobacterium virus Astro, Mycobacterium virus Backyardigan, Mycobacterium virus BBPiebs31, Mycobacterium virus Benedict, Mycobacterium virus Bethlehem, Mycobacterium virus Billknuckles, Mycobacterium virus Bruns, Mycobacterium virus Bxb1, Mycobacterium virus Bxz2, Mycobacterium virus Che12, Mycobacterium virus Cuco, Mycobacterium virus D29, Mycobacterium virus Doom, Mycobacterium virus Ericb, Mycobacterium virus Euphoria, Mycobacterium virus George, Mycobacterium virus Gladiator, Mycobacterium virus Goose, Mycobacterium virus Hammer, Mycobacterium virus Heldan, Mycobacterium virus Jasper, Mycobacterium virus JC27, Mycobacterium virus Jeffabunny, Mycobacterium virus JHC117, Mycobacterium virus KBG, Mycobacterium virus Kssjeb, Mycobacterium virus Kugel, Mycobacterium virus L5, Mycobacterium virus Lesedi, Mycobacterium virus LHTSCC, Mycobacterium virus lockley, Mycobacterium virus Marcell, Mycobacterium virus Microwolf, Mycobacterium virus Mrgordo, Mycobacterium virus Museum, Mycobacterium virus Nepal, Mycobacterium virus Packman, Mycobacterium virus Peaches, Mycobacterium virus Perseus, Mycobacterium virus Pukovnik, Mycobacterium virus Rebeuca, Mycobacterium virus Redrock, Mycobacterium virus Ridgecb, Mycobacterium virus Rockstar, Mycobacterium virus Saintus, Mycobacterium virus Skipole, Mycobacterium virus Solon, Mycobacterium virus Switzer, Mycobacterium virus SWU1, Mycobacterium virus Ta17a, Mycobacterium virus Tiger, Mycobacterium virus Timshel, Mycobacterium virus Trixie, Mycobacterium virus Turbido, Mycobacterium virus Twister, Mycobacterium virus U2, Mycobacterium virus Violet, Mycobacterium virus Wonder, Escherichia virus DE3, Escherichia virus HK629, Escherichia virus HK630, Escherichia virus Lambda, Arthrobacter virus Laroye, Mycobacterium virus Halo, Mycobacterium virus Liefie, Mycobacterium virus Marvin, Mycobacterium virus Mosmoris, Arthrobacter virus Circum, Arthrobacter virus Mudcat, Escherichia virus N15, Escherichia virus 9 g, Escherichia virus JenK1, Escherichia virus JenP1, Escherichia virus JenP2, Pseudomonas virus NP1, Pseudomonas virus PaMx25, Mycobacterium virus Baka, Mycobacterium virus Courthouse, Mycobacterium virus Littlee, Mycobacterium virus Omega, Mycobacterium virus Optimus, Mycobacterium virus Thibault, Polaribacter virus P12002L, Polaribacter virus P12002S, Nonlabens virus P12024L, Nonlabens virus P12024S, Thermus virus P23-45, Thermus virus P74-26, Listeria virus LP26, Listeria virus LP37, Listeria virus LP110, Listeria virus LP114, Listeria virus P70, Propionibacterium virus ATCC29399BC, Propionibacterium virus ATCC29399BT, Propionibacterium virus Attacne, Propionibacterium virus Keiki, Propionibacterium virus Kubed, Propionibacterium virus Lauchelly, Propionibacterium virus MrAK, Propionibacterium virus Ouroboros, Propionibacterium virus P91, Propionibacterium virus P105, Propionibacterium virus P144, Propionibacterium virus P1001, Propionibacterium virus P1.1, Propionibacterium virus P100A, Propionibacterium virus P100D, Propionibacterium virus P101A, Propionibacterium virus P104A, Propionibacterium virus PA6, Propionibacterium virus Pacnes201215, Propionibacterium virus PAD20, Propionibacterium virus PAS50, Propionibacterium virus PHLOO9M11, Propionibacterium virus PHL025M00, Propionibacterium virus PHL037M02, Propionibacterium virus PHL041M10, Propionibacterium virus PHL060L00, Propionibacterium virus PHL067M01, Propionibacterium virus PHL070N00, Propionibacterium virus PHL071 N05, Propionibacterium virus PHL082M03, Propionibacterium virus PHL092M00, Propionibacterium virus PHL095N00, Propionibacterium virus PHL111M01, Propionibacterium virus PHL112N00, Propionibacterium virus PHL113M01, Propionibacterium virus PHL114L00, Propionibacterium virus PHL116M00, Propionibacterium virus PHL117M00, Propionibacterium virus PHL117M01, Propionibacterium virus PHL132N00, Propionibacterium virus PHL141N00, Propionibacterium virus PHL151 M00, Propionibacterium virus PHL151N00, Propionibacterium virus PHL152M00, Propionibacterium virus PHL163M00, Propionibacterium virus PHL171M01, Propionibacterium virus PHL179M00, Propionibacterium virus PHL194M00, Propionibacterium virus PHL199M00, Propionibacterium virus PHL301M00, Propionibacterium virus PHL308M00, Propionibacterium virus Pirate, Propionibacterium virus Procrassl, Propionibacterium virus SKKY, Propionibacterium virus Solid, Propionibacterium virus Stormborn, Propionibacterium virus Wizzo, Pseudomonas virus PaMx28, Pseudomonas virus PaMx74, Mycobacterium virus Patience, Mycobacterium virus PBI1, Rhodococcus virus Pepy6, Rhodococcus virus Poco6, Propionibacterium virus PFR1, Streptomyces virus phiBT1, Streptomyces virus phiC31, Streptomyces virus TG1, Caulobacter virus Karma, Caulobacter virus Magneto, Caulobacter virus phiCbK, Caulobacter virus Rogue, Caulobacter virus Swift, Staphylococcus virus 11, Staphylococcus virus 29, Staphylococcus virus 37, Staphylococcus virus 53, Staphylococcus virus 55, Staphylococcus virus 69, Staphylococcus virus 71, Staphylococcus virus 80, Staphylococcus virus 85, Staphylococcus virus 88, Staphylococcus virus 92, Staphylococcus virus 96, Staphylococcus virus 187, Staphylococcus virus 52a, Staphylococcus virus 80alpha, Staphylococcus virus CNPH82, Staphylococcus virus EW, Staphylococcus virus IPLA5, Staphylococcus virus IPLA7, Staphylococcus virus IPLA88, Staphylococcus virus PH15, Staphylococcus virus phiETA, Staphylococcus virus phiETA2, Staphylococcus virus phiETA3, Staphylococcus virus phiMR11, Staphylococcus virus phiMR25, Staphylococcus virus phiNM1, Staphylococcus virus phiNM2, Staphylococcus virus phiNM4, Staphylococcus virus SAP26, Staphylococcus virus X2, Enterococcus virus FL1, Enterococcus virus FL2, Enterococcus virus FL3, Lactobacillus virus ATCC8014, Lactobacillus virus phiJL1, Pediococcus virus cIP1, Aeromonas virus pIS4A, Listeria virus LP302, Listeria virus PSA, Methanobacterium virus psiM1, Roseobacter virus RDJL1, Roseobacter virus RDJL2, Rhodococcus virus RER2, Enterococcus virus BC611, Enterococcus virus IMEEF1, Enterococcus virus SAP6, Enterococcus virus VD13, Streptococcus virus SPQS1, Mycobacterium virus Papyrus, Mycobacterium virus Send513, Burkholderia virus KL1, Pseudomonas virus 73, Pseudomonas virus Ab26, Pseudomonas virus Kakheti25, Escherichia virus Cajan, Escherichia virus Seurat, Staphylococcus virus SEP9, Staphylococcus virus Sextaec, Streptococcus virus 858, Streptococcus virus 2972, Streptococcus virus ALQ132, Streptococcus virus 01205, Streptococcus virus Sfil 1, Streptococcus virus 7201, Streptococcus virus DT1, Streptococcus virus phiAbc2, Streptococcus virus Sfi19, Streptococcus virus Sfi21, Paenibacillus virus Diva, Paenibacillus virus Hb10c2, Paenibacillus virus Rani, Paenibacillus virus Shelly, Paenibacillus virus Sitara, Paenibacillus virus Willow, Lactococcus virus 712, Lactococcus virus ASCC191, Lactococcus virus ASCC273, Lactococcus virus ASCC281, Lactococcus virus ASCC465, Lactococcus virus ASCC532, Lactococcus virus Bibb29, Lactococcus virus bIL170, Lactococcus virus CB13, Lactococcus virus CB14, Lactococcus virus CB19, Lactococcus virus CB20, Lactococcus virus jj50, Lactococcus virus P2, Lactococcus virus P008, Lactococcus virus ski, Lactococcus virus S14, Bacillus virus Slash, Bacillus virus Stahl, Bacillus virus Staley, Bacillus virus Stills, Gordonia virus Bachita, Gordonia virus ClubL, Gordonia virus OneUp, Gordonia virus Smoothie, Gordonia virus Soups, Bacillus virus SPbeta, Vibrio virus MAR10, Vibrio virus SSP002, Escherichia virus AKFV33, Escherichia virus BF23, Escherichia virus DT57C, Escherichia virus EPS7, Escherichia virus FFH1, Escherichia virus H8, Escherichia virus slur09, Escherichia virus T5, Salmonella virus 1189705aI2, Salmonella virus Shivani, Salmonella virus SPC35, Salmonella virus Stitch, Arthrobacter virus Tank, Tsukamurella virus TIN2, Tsukamurella virus TIN3, Tsukamurella virus TIN4, Rhodobacter virus RcSpartan, Rhodobacter virus RcTitan, Mycobacterium virus Anaya, Mycobacterium virus Angelica, Mycobacterium virus Crimd, Mycobacterium virus Fionnbarth, Mycobacterium virus Jaws, Mycobacterium virus Larva, Mycobacterium virus Macncheese, Mycobacterium virus Pixie, Mycobacterium virus TM4, Bacillus virus BMBtp2, Bacillus virus TP21, Geobacillus virus Tp84, Staphylococcus virus 47, Staphylococcus virus 3a, Staphylococcus virus 42e, Staphylococcus virus IPLA35, Staphylococcus virus phi12, Staphylococcus virus phiSLT, Mycobacterium virus 32HC, Rhodococcus virus RGL3, Paenibacillus virus Vegas, Gordonia virus Vendetta, Bacillus virus Wbeta, Mycobacterium virus Wildcat, Gordonia virus Twister6, Gordonia virus Wizard, Gordonia virus Hotorobo, Gordonia virus Monty, Gordonia virus Woes, Xanthomonas virus CP1, Xanthomonas virus OP1, Xanthomonas virus phi17, Xanthomonas virus Xop411, Xanthomonas virus Xp10, Streptomyces virus TP1604, Streptomyces virus YDN12, Alphaproteobacteria virus phiJI001, Pseudomonas virus LKO4, Pseudomonas virus M6, Pseudomonas virus MP1412, Pseudomonas virus PAE1, Pseudomonas virus Yua, Pseudoalteromonas virus PM2, Pseudomonas virus phi6, Pseudomonas virus phi8, Pseudomonas virus phi12, Pseudomonas virus phi13, Pseudomonas virus phi2954, Pseudomonas virus phiNN, Pseudomonas virus phiYY, Vibrio virus fs1, Vibrio virus VGJ, Ralstonia virus RS603, Ralstonia virus RSM1, Ralstonia virus RSM3, Escherichia virus M13, Escherichia virus 122, Salmonella virus IKe, Acholeplasma virus L51, Vibrio virus fs2, Vibrio virus VFJ, Escherichia virus Ifi, Propionibacterium virus B5, Pseudomonas virus Pf1, Pseudomonas virus Pf3, Ralstonia virus PE226, Ralstonia virus RSS1, Spiroplasma virus SVTS2, Stenotrophomonas virus PSH1, Stenotrophomonas virus SMA6, Stenotrophomonas virus SMA7, Stenotrophomonas virus SMA9, Vibrio virus CTXphi, Vibrio virus KSF1, Vibrio virus VCY, Vibrio virus Vf33, Vibrio virus VfO3K6, Xanthomonas virus Cf1c, Spiroplasma virus C74, Spiroplasma virus R8A2B, Spiroplasma virus SkV1CR23x, Escherichia virus FI, Escherichia virus Qbeta, Escherichia virus BZ13, Escherichia virus MS2, Escherichia virus alpha3, Escherichia virus 1D21, Escherichia virus 1D32, Escherichia virus 1D62, Escherichia virus NC28, Escherichia virus NC29, Escherichia virus NC35, Escherichia virus phiK, Escherichia virus St1, Escherichia virus WA45, Escherichia virus G4, Escherichia virus 1D52, Escherichia virus Talmos, Escherichia virus phiX174, Bdellovibrio virus MAC1, Bdellovibrio virus MH2K, Chlamydia virus Chp1, Chlamydia virus Chp2, Chlamydia virus CPAR39, Chlamydia virus CPG1, Spiroplasma virus SpV4, Acholeplasma virus L2, Pseudomonas virus PR4, Pseudomonas virus PRD1, Bacillus virus AP50, Bacillus virus Bam35, Bacillus virus GIL16, Bacillus virus Wip1, Escherichia virus phi80, Escherichia virus RB42, Escherichia virus T2, Escherichia virus T3, Escherichia virus T6, Escherichia virus VT2-Sa, Escherichia virus VT1-Sakai, Escherichia virus VT2-Sakai, Escherichia virus CP-933V, Escherichia virus P27, Escherichia virus Stx2phi-1, Escherichia virus Stx1phi, Escherichia virus Stx2phi-11, Escherichia virus CP-1639 based on the Escherichia virus BP-4795, Escherichia virus 86, Escherichia virus Min27, Escherichia virus 2851, Escherichia virus 1717, Escherichia virus YYZ-2008, Escherichia virus ECO26 PO6, Escherichia virus ECO103_P15, Escherichia virus ECO103_P12, Escherichia virus ECO111_P16, Escherichia virus ECO111_P11, Escherichia virus VT2phi 272, Escherichia virus TL-2011c, Escherichia virus P13374, Escherichia virus Sp5.

In one embodiment, the bacterial virus particles target E. coli and includes the capsid of a bacteriophage selected in the group consisting of BW73, B278, D6, D108, E, EI, E24, E41, F1-2, F1-4, F1-5, H18A, FfI8B, i, MM, Mu, 025, PhI-5, Pk, PSP3, PI, PID, P2, P4, SI, Wφ, φK13, φI, φ2, φ7, φ92, 7 A, 8℠, 9φ, 18, 28-1, 186, 299, HH-Escherichia (2), AB48, CM, C4, C16, DD-VI, E4, E7, E28, FII, F13, H, HI, H3, H8, K3, M, N, ND-2, ND-3, ND4, ND-5, ND6, ND-7, Ox-I, Ox-2, Ox-3, Ox-4, Ox-5, Ox-6, Ph1-1, RB42, RB43, RB49, RB69, S, SaI-1, SaI-2, SaI-3, SaI-4, SaI-5, SaI-6, φC23, φC45, TulI*-6, TulP-24, TulI*46, TulP-60, T2, T4, T6, T35, al, 1, IA, 3, 3A, 3T+, 5φ, 9266Q, CFO103, HK620, J, K, KIF, m59, no. A, no. E, no. 3, no. 9, N4, sd, T3, T7, WPK, W31, ΔH, φC3888, φK3, φK7, φK12, φV-1, Φ04-CF, Φ05, Φ06, Φ07, φI, φI.2, φ20, φ95, φ263, φIO92, φI, φII, Ω8, 1, 3, 7, 8, 26, 27, 28-2, 29, 30, 31, 32, 38, 39, 42, 933W, NN-Escherichia (1), Esc-7-11, AC30, CVX-5, CI, DDUP, ECI, EC2, E21, E29, F1, F26S, F27S, Hi, HK022, HK97, HK139, HK253, HK256, K7, ND-1, PA-2, q, S2, TI, ), T3C, T5, UC-I, w, β4, γ2, λ, ΦD326, φγ, Φ06, Φ7, Φ10, φ80, χ, 2, 4, 4A, 6, 8A, 102, 150, 168, 174, 3000, AC6, AC7, AC28, AC43, AC50, AC57, AC81, AC95, HK243, KIO, ZG/3A, 5, 5A, 21EL, H19-J and 933H.

In some embodiment the vectors disclosed herein may be used in combination with prebiotics. Prebiotics include, but are not limited to, amino acids, biotin, fructo-oligosaccharide, galacto-oligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carrageenan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

In other embodiment, the vectors disclosed herein may be used in combination with probiotics. Probiotics include, but are not limited to lactobacilli, bifidobacteria, streptococci, enterococci, propionibacteria, saccharomycetes, lactobacilli, bifidobacteria, or proteobacteria.

The probiotic strain can take over the ecological niche left by elimination of the AMR strains from the patient's intestine, further preventing AMR strains from bouncing back.

### Pharmaceutical and Veterinary Compositions and In Situ Administration Methods

The invention encompasses pharmaceutical and veterinary compositions comprising at least one, at least two, at least three, at least four, at least five, at least six, at least seven, at least eight, at least nine, at least ten or more vectors of the invention, in particular different vectors of the invention, and, optionally antibiotics.

The invention encompasses pharmaceutical and veterinary compositions comprising at one, at least two, at least three, at least four, at least five, at least six, at least seven, at least eight, at least nine, at least ten or more engineered phages of the invention, in particular different engineered phages of the invention, and optionally antibiotics.

The invention encompasses a pharmaceutical agent which reduces the amount of AMR bacteria in a subject.

In some embodiments, the selective decolonization occurs before the subject or patient undergoes invasive procedures (surgery, intubation, catheterization, etc.) or harsh conditioning procedures (immunosuppression, irradiation, etc.) that typically compromise epithelial barriers and promote dissemination of bacteria and infections in patients. In one embodiment, the selective decolonization occurs before the subject or patient undergoes surgery. In a preferred embodiment, the surgery is a solid organ transplant surgery of the kidney or liver. In some embodiments, the subject or patient is in the ICU and/or is to undergo surgery, dialysis, or a transplant.

Preferably, the subject or patient presents a skin, nasal, or intestinal carriage of AMR bacteria. In some embodiments, the subject or patient presents Methicillin resistant Staphylococcus aureus (MRSA), an Extended Spectrum beta lactamase (ESBL) and/or Carbapenemase-producing (CP) Enterobacteria.

In some embodiments, the subject or patient carries Klebsiella pneumoniae of the K-type K2, K1, K62, K64, K23, K28, K54, K16, K5, K27 or combinations thereof. In some embodiments, the subject or patient carries Klebsiella pneumoniae of the K-type K2, K1, K62, K64 or combinations thereof. In some embodiments, the subject or patient carries Klebsiella pneumoniae of the K-type K23, K28, K54, K16, or combinations thereof. In some embodiments, the subject or patient carries Klebsiella pneumoniae of the K-type K5, K27, or combinations thereof. In some embodiments, the subject or patients carries Klebsiella pneumoniae of the K-type K24, K2, K23, K71, K62, K60, K41, K28, K22, K37, K17, K16, K64, K1, K15, K50, K51, K58, or combinations thereof. In some embodiments, the subject or patient carries Klebsiella pneumoniae of the K-type K24, K2, K23, K71, or combinations thereof. In some embodiments, the subject or patients carries Klebsiella pneumoniae of the K-type K62, K60, K41, K28, K22, K37, K17, K16, K64, K1, K15, K50, K51, K58, or combinations thereof. In some embodiments, the subject or patient carries Klebsiella pneumoniae of the O-type O1, O2, O3, O5, O4, O12, OL101, OL104 or combinations thereof. In some embodiments, the subject or patient carries Klebsiella pneumoniae of the O-type O1, O2, O3, O5, or combinations thereof. In some embodiments, the subject or patient carries Klebsiella pneumoniae of the O-type O4, O12, OL101, OL104 or combinations thereof. In some embodiments, the subject or patient carries Klebsiella pneumoniae strain MLST258.

In some embodiments, the subject or patient carries Escherichia coli of the K type K1, K5 or combinations thereof. In some embodiments, the subject or patient carries Escherichia coli of the O-type O1, O2, O6, O25, O18, or combinations thereof. In some embodiments, the subject or patient carries Escherichia coli of the O-type O1, O2, O3, O4, O6, O7, O8, O16, O25, O75, or combinations thereof. In some embodiments, the subject or patient carries bacteria selected from the group consisting of Escherichia coli strains MLST131, ST73, ST95, ST12, ST10, ST14, ST2278, ST69, and combinations thereof.

The subject treated in the context of the invention is a healthy subject or a patient.

By “healthy subject” is meant herein a subject who does not suffer from a bacterial infection, in particular from an infection caused by antibiotic-resistant bacteria, at the time of administration of the vector of the invention.

By “patient” is meant herein a subject who does not suffer from a bacterial infection, in particular from an infection caused by antibiotic-resistant bacteria, at the time of administration of the vector of the invention, but suffers from another disease such as a disease necessitating invasive or non-invasive medical procedures, for example transplant, in particular solid organ transplant or hematopoietic stem cells transplant, dialysis, immunosuppression or irradiation. In a particular embodiment, said patient suffers from cirrhosis.

In a particular embodiment, said patient suffers from cancer, in particular from a hematologic malignancy. In a particular embodiment, said patient suffers chemotherapy-induced neutropenia and/or chemotherapy-induced gastrointestinal mucositis.

In a particular embodiment, said patient suffers from neutropenia and//or mucositis.

In a particular embodiment, said patient is immunosuppressed. In an alternative embodiment, said patient is not immunosuppressed.

In a particular embodiment, said patient undergoes prolonged hospitalization.

As will be understood by the skilled person, despite the healthy subject or patient treated in the context of the invention is not suffering from a bacterial infection, in particular from an infection caused by antibiotic-resistant bacteria, at the time of administration of the vector of the invention, the healthy subject or patient can carry AMR bacteria.

The invention encompasses in situ administration of the pharmaceutical or veterinary composition to the bacteria in a subject. Any method known to the skilled artisan can be used to contact the composition with the bacterial target in situ.

In a particular embodiment, the pharmaceutical or veterinary composition of the invention is administered before and/or after the subject or patient undergoes invasive procedures (surgery, intubation, catheterization, etc.) or harsh conditioning procedures (immunosuppression, irradiation, etc.). In one embodiment, the pharmaceutical or veterinary composition of the invention is administered before and/or after the subject or patient undergoes surgery. In a preferred embodiment, the surgery is a solid organ transplant surgery of the kidney or liver. In some embodiments, the subject or patient is in the ICU and/or is to undergo surgery, dialysis, or a transplant. In one embodiment, the pharmaceutical or veterinary composition of the invention is administered before and/or after the subject or patient ungers hematopoietic stem cell transplantation.

In the context of the invention, the pharmaceutical or veterinary composition of the invention is administered before the subject or patient presents any sign of bacterial infection, in particular any sign of AR bacterial infection.

In one embodiment, the composition comprises an effective amount of at least one, 2, 3, 4, 5, 6, 7, 8, 9, 10 or more, antibiotic, phage, recombinant phage, packaged phagemid, plasmid, plasmid inside a carrier bacterium, or combination thereof.

In one embodiment, the antibiotic is selected from methicillin, streptomycin, vancomycin, clindamycin, metronidazole, sulphadoxine, trimethoprim, or any combination of 1, 2, 3, 4, 5 or 6 of these antibiotics.

In various embodiments, the phage, recombinant phage, packaged phagemid or plasmid encodes a nuclease selected from CRISPR-Cas, TALENs and variants, zinc finger nuclease (ZFN) and ZFN variants, natural, evolved or engineered meganuclease or recombinase variants.

In various embodiments, the phage, recombinant phage, packaged phagemid or plasmid encodes a base editor or a prime editor.

The pharmaceutical or veterinary composition according to the invention may further comprise a pharmaceutically acceptable vehicle. A solid pharmaceutically acceptable vehicle may include one or more substances which may also act as flavoring agents, lubricants, solubilizers, suspending agents, dyes, fillers, glidants, compression aids, inert binders, sweeteners, preservatives, dyes, coatings, or tablet-disintegrating agents. Suitable solid vehicles include, for example calcium phosphate, magnesium stearate, talc, sugars, lactose, dextrin, starch, gelatin, cellulose, polyvinylpyrrolidone, low melting waxes and ion exchange resins.

The pharmaceutical or veterinary composition may be prepared as a sterile solid composition that may be suspended at the time of administration using sterile water, saline, or other appropriate sterile injectable medium. The pharmaceutical or veterinary compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The particles according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for enteral administration include sterile solutions, emulsions, and suspensions.

The bacterial delivery vehicles according to the invention, in particular the bacterial virus particles according to the invention, may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and enteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for enteral administration. The liquid vehicle for pressurized compositions can be a halogenated hydrocarbon or other pharmaceutically acceptable propellant.

In some embodiments, the invention encompasses pharmaceutical or veterinary composition formulated for delayed or gradual enteric release. In preferred embodiments, formulations or pharmaceutical preparations of the invention are formulated for delivery of the vector into the distal small bowel and/or the colon. The formulation can allow the vector to pass through stomach acid and pancreatic enzymes and bile, and reach undamaged to be viable in the distal small bowel and colon.

In some embodiments, the pharmaceutical or veterinary composition is micro-encapsulated, formed into tablets and/or placed into capsules, preferably enteric-coated capsules.

In some embodiments, the pharmaceutical or veterinary compositions are formulated for delayed or gradual enteric release, using cellulose acetate (CA) and polyethylene glycol (PEG). In some embodiments, the pharmaceutical or veterinary compositions are formulated for delayed or gradual enteric release using a hydroxypropylmethylcellulose (HPMC), a microcrystalline cellulose (MCC) and magnesium stearate. the pharmaceutical or veterinary compositions are formulated for delayed or gradual enteric release using e.g., a poly(meth)acrylate, e.g. a methacrylic acid copolymer B, a methyl methacrylate and/or a methacrylic acid ester, or a polyvinylpyrrolidone (PVP).

In some embodiments, the pharmaceutical or veterinary compositions are formulated for delayed or gradual enteric release using a release-retarding matrix material such as: an acrylic polymer, a cellulose, a wax, a fatty acid, shellac, zein, hydrogenated vegetable oil, hydrogenated castor oil, polyvinylpyrrolidone, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinyl acetates, polyvinylacetate copolymers or any combination thereof.

In some embodiments, the pharmaceutical or veterinary compositions are formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candellia wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

In some embodiments, the pharmaceutical or veterinary compositions are a powder that can be included into a tablet or a suppository. In alternative embodiments, a formulation or pharmaceutical preparation of the invention can be a “powder for reconstitution” as a liquid to be drunk or otherwise administered.

In some embodiments, the pharmaceutical or veterinary compositions can be administered in a cream, gel, lotion, liquid, feed, or aerosol spray. In some embodiments, a bacteriophage is immobilized to a solid surface using any substance known in the art and any technology known in the art, for example, but not limited to immobilization of bacteriophages onto polymeric beads using technology as outlined in U.S. Pat. No. 7,482,115, which is incorporated herein by reference. Phages may be immobilized onto appropriately sized polymeric beads so that the coated beads may be added to aerosols, creams, gels or liquids. The size of the polymeric beads may be from about 0.1 μm to 500 μm, for example 50 μm to 100 μm. The coated polymeric beads may be incorporated into animal feed, including pelleted feed and feed in any other format, incorporated into any other edible device used to present phage to the animals, added to water offered to animals in a bowl, presented to animals through water feeding systems. In some embodiments, the compositions are used for treatment of surface wounds and other surface infections using creams, gels, aerosol sprays and the like.

In some embodiments, the pharmaceutical or veterinary compositions can be administered by inhalation, in the form of a suppository or pessary, topically (e.g., in the form of a lotion, solution, cream, ointment or dusting powder), epi- or transdermally (e.g., by use of a skin patch), orally (e.g., as a tablet, which may contain excipients such as starch or lactose), as a capsule, ovule, elixirs, solutions, or suspensions (each optionally containing flavoring, coloring agents and/or excipients), or they can be injected parenterally (e.g., intravenously, intramuscularly or subcutaneously). For parenteral administration, the compositions may be used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner. In a preferred embodiment, a bacteriophage and/or polypeptide of the present invention is administered topically, either as a single agent, or in combination with other antibiotic treatments, as described herein or known in the art.

In some embodiments, the pharmaceutical or veterinary compositions can also be dermally or transdermally administered. For topical application to the skin, the pharmaceutical or veterinary composition can be combined with one or a combination of carriers, which can include but are not limited to, an aqueous liquid, an alcohol base liquid, a water soluble gel, a lotion, an ointment, a nonaqueous liquid base, a mineral oil base, a blend of mineral oil and petrolatum, lanolin, liposomes, proteins carriers such as serum albumin or gelatin, powdered cellulose carmel, and combinations thereof. A topical mode of delivery may include a smear, a spray, a bandage, a time-release patch, a liquid-absorbed wipe, and combinations thereof. The pharmaceutical or veterinary composition can be applied to a patch, wipe, bandage, etc., either directly or in a carrier(s). The patches, wipes, bandages, etc., may be damp or dry, wherein the phage and/or polypeptide (e.g., a lysin) is in a lyophilized form on the patch. The carriers of topical compositions may comprise semi-solid and gel-like vehicles that include a polymer thickener, water, preservatives, active surfactants, or emulsifiers, antioxidants, sun screens, and a solvent or mixed solvent system. U.S. Pat. No. 5,863,560 discloses a number of different carrier combinations that can aid in the exposure of skin to a medicament, and its contents are incorporated herein.

For intranasal or administration by inhalation, the pharmaceutical or veterinary composition is conveniently delivered in the form of a dry powder inhaler or an aerosol spray presentation from a pressurized container, pump, spray, or nebuliser with the use of a suitable propellant, e.g., dichlorodifluoromethane, trichlorofluoromethane, dichlorotetrafluoroethane, a hydrofluoroalkane such as 1,1,1,2-tetrafluoroethane or 1,1,1,2,3,3,3-heptafluoropropane, carbon dioxide, or other suitable gas. In the case of a pressurized aerosol, the dosage unit may be determined by providing a valve to deliver a metered amount. The pressurized container, pump, spray, or nebuliser may contain a solution or suspension of the active compound, e.g., using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of the bacteriophage and/or polypeptide of the invention and a suitable powder base such as lactose or starch.

For administration in the form of a suppository or pessary, the pharmaceutical or veterinary composition can be applied topically in the form of a gel, hydrogel, lotion, solution, cream, ointment, or dusting powder. Compositions of the invention may also be administered by the ocular route. For ophthalmic use, the compositions of the invention can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

Dosages and desired drug concentrations of the pharmaceutical and veterinary composition compositions of the present invention may vary depending on the particular use. The determination of the appropriate dosage or route of administration is within the skill of an ordinary physician. Animal experiments can provide reliable guidance for the determination of effective doses in human therapy.

For transdermal administration, the pharmaceutical or veterinary composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compounds can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

### Subject, Regimen and Administration

The subject or patient according to the invention is an animal, preferably a mammal, even more preferably a human. However, the term “subject” or “patient” can also refer to non-human animals, in particular mammals such as dogs, cats, horses, cows, pigs, sheep, donkeys, rabbits, ferrets, gerbils, hamsters, chinchillas, rats, mice, guinea pigs and non-human primates, among others, or non-mammals such as poultry, that are in need of treatment.

The human subject or patient according to the invention may be a human at the prenatal stage, a new-born, a child, an infant, an adolescent or an adult at any age.

In a preferred embodiment, the subject or patient is being prepared for an invasive procedure, such as a surgery, intubation, catheterization, etc., or a harsh conditioning procedure, such as immunosuppression, irradiation, etc.

The subject treated in the context of the invention is a healthy subject or a patient.

By “healthy subject” is meant herein a subject who does not suffer from a bacterial infection, in particular from an infection caused by antibiotic-resistant bacteria, at the time of administration of the vector of the invention.

By “patient” is meant herein a subject who does not suffer from a bacterial infection, in particular from an infection caused by antibiotic-resistant bacteria, at the time of administration of the vector of the invention, but suffers from another disease such as a disease necessitating invasive or non-invasive medical procedures, for example transplant, in particular solid organ transplant or hematopoietic stem cells transplant, dialysis, immunosuppression or irradiation.

As will be understood by the skilled person, despite the healthy subject or patient treated in the context of the invention is not suffering from a bacterial infection, in particular from an infection caused by antibiotic-resistant bacteria, at the time of administration of the vector of the invention, the healthy subject or patient can carry AMR bacteria.

In some embodiments, the treatment is administered several times, preferably 2, 3, 4, 5, or 6 times.

The form of the pharmaceutical or veterinary compositions, the route of administration and the dose of administration of delivery vehicles according to the invention, preferably of a payload, vector or engineered phage according to the invention, particularly of a payload packaged into a delivery vehicle according to the invention, preferably of a packaged plasmid or phagemid into a bacterial virus particle according to the invention, or of a pharmaceutical or veterinary composition according to the invention can be adjusted by the man skilled in the art according to the type and level of the colonization (e.g. depending on the bacteria species involved and its localization in the patient's or subject's body), and to the patient or subject, in particular its age, weight, sex, and general physical condition.

Particularly, the amount of delivery vehicles according to the invention, preferably a payload, vector or an engineered phage according to the invention, particularly a payload packaged into a delivery vehicle according to the invention, preferably a packaged plasmid or phagemid into a bacterial virus particle according to the invention, or of a pharmaceutical or veterinary composition according to the invention, to be administered has to be determined by standard procedure well known by those of ordinary skills in the art. Physiological data of the patient or subject (e.g. age, size, and weight) and the routes of administration have to be taken into account to determine the appropriate dosage, so as a therapeutically effective amount will be administered to the patient or subject.

For example, the total amount of delivery vehicles, particularly a payload packaged into a delivery vehicle or an engineered phage according to the invention, preferably a plasmid or phagemid packaged into a bacterial virus particle according to the invention, for each administration is comprised between 10⁴ and 10¹⁵ delivery vehicles.

### Definitions

«Vector»

As used herein, the term “vector” refers to any construct of sequences that are capable of expression of a polypeptide in a given host cell. If a vector is used then the choice of vector is dependent upon the method that will be used to transform host bacteria as is well known to those skilled in the art. Vectors can include, without limitation, plasmid vectors and recombinant phage vectors, or any other vector known in that art suitable for delivering a nucleic acid sequence of the invention to target bacteria. The skilled artisan is well aware of the genetic elements that must be present on the vector in order to successfully transform, select and optionally propagate host cells comprising any of the isolated nucleotides or nucleic acid sequences of the invention.

«Delivery Vehicle»

As used herein, the term «delivery vehicle» refers to any vehicle that allows the transfer of a payload into a bacterium.

There are several types of delivery vehicle encompassed by the present invention including, without limitation, bacteriophage scaffold, virus scaffold, bacterial virus particle, chemical based delivery vehicle (e.g., cyclodextrin, calcium phosphate, cationic polymers, cationic liposomes), protein-based or peptide-based delivery vehicle, lipid-based delivery vehicle, nanoparticle-based delivery vehicles, non-chemical-based delivery vehicles (e.g., transformation, electroporation, sonoporation, optical transfection), particle-based delivery vehicles (e.g., gene gun, magnetofection, impalefection, particle bombardment, cell-penetrating peptides) or donor bacteria (conjugation).

Any combination of delivery vehicles is also encompassed by the present invention.

The delivery vehicle can refer to a bacteriophage derived scaffold and can be obtained from a natural, evolved or engineered capsid.

In some embodiments, the delivery vehicle is the payload as bacteria are naturally competent to take up a payload from the environment on their own.

«Payload»

As used herein, the term «payload» refers to any nucleic acid sequence or amino acid sequence, or a combination of both (such as, without limitation, peptide nucleic acid or peptide-oligonucleotide conjugate) transferred into a bacterium preferably with a delivery vehicle.

The term «payload» may also refer to a plasmid, a vector or a cargo.

The payload can be a phagemid or phasmid obtained from natural, evolved or engineered bacteriophage genome. The payload can also be composed only in part of phagemid or phasmid obtained from natural, evolved or engineered bacteriophage genome.

In some embodiments, the payload is the delivery vehicle as bacteria are naturally competent to take up a payload from the environment on their own.

«Nucleic Acid»

As used herein, the term “nucleic acid” refers to a sequence of at least two nucleotides covalently linked together which can be single-stranded or double-stranded or contains portion of both single-stranded and double-stranded sequence. Nucleic acids of the present invention can be naturally occurring, recombinant or synthetic. The nucleic acid can be in the form of a circular sequence or a linear sequence or a combination of both forms. The nucleic acid can be DNA, both genomic or cDNA, or RNA or a combination of both. The nucleic acid may contain any combination of deoxyribonucleotides and ribonucleotides, and any combination of bases, including uracil, adenine, thymine, cytosine, guanine, inosine, xanthine, hypoxanthine, isocytosine, 5-hydroxymethylcytosine and isoguanine. Other examples of modified bases that can be used in the present invention are detailed in Chemical Reviews 2016, 116 (20) 12655-12687. The term “nucleic acid” also encompasses any nucleic acid analogs which may contain other backbones comprising, without limitation, phosphoramide, phosphorothioate, phosphorodithioate, O-methylphophoroamidite linkage and/or deoxyribonucleotides and ribonucleotides nucleic acids. Any combination of the above features of a nucleic acid is also encompassed by the present invention.

“Phagemid” and “Packaged Phagemid”

As used herein the term “phagemid” or “phasmid” are equivalent and refer to a recombinant DNA vector comprising at least one sequence of a bacteriophage genome and which is preferably not able of producing progeny, more particularly a vector that derives from both a plasmid and a bacteriophage genome. A phagemid of the disclosure comprises a phage packaging site and optionally an origin of replication (ori), in particular a bacterial and/or phage origin of replication. In one embodiment, the phagemid does not comprise an origin of replication and thus cannot replicate by itself once injected into a bacterium. Alternatively, the phagemid comprises a plasmid origin of replication, in particular a bacterial and/or phage origin of replication.

As used herein, the term “packaged phagemid” refers to a phagemid which is encapsidated in a bacteriophage scaffold, bacterial virus particle or capsid. Particularly, it refers to a bacteriophage scaffold, bacterial virus particle or capsid devoid of a bacteriophage genome. The packaged phagemid may be produced with a helper phage strategy, well known from the man skilled in the art. The helper phage comprises all the genes coding for the structural and functional proteins that are indispensable for the phagemid according to the invention to be encapsidated. The packaged phagemid may be produced with a satellite virus strategy, also known from the man skilled in the art. Satellite virus are subviral agent and are composed of nucleic acid that depends on the co-infection of a host cell with a helper virus for all the morphogenetic functions, whereas for all its episomal functions (integration and immunity, multicopy plasmid replication) the satellite is completely autonomous from the helper. In one embodiment, the satellite genes can encode proteins that promote capsid size reduction of the helper phage, as described for the P4 Sid protein that controls the P2 capsid size to fit its smaller genome.

«Peptide»

As used herein, the term “peptide” refers both to a short chain of at least 2 amino acids linked between each other and to a part of, a subset of, or a fragment of a protein which part, subset or fragment being not expressed independently from the rest of the protein. In some instances, a peptide is a protein. In some other instances, a peptide is not a protein and peptide only refers to a part, a subset or a fragment of a protein. Preferably, the peptide is from 4, 5, 6, 7, 8, 9, 10, 11, 12, 13, 14, 15 amino acids to 4, 5, 6, 7, 8, 9, 10, 11, 12, 13, 14, 15, 20, 25, 30, 40, 50, 100, 200 amino acids in size.

The invention further concerns the following clauses:

1. A method of selectively removing antibiotic resistance from an antibiotic resistant bacterial strain(s) in a healthy subject or patient carrying antibiotic resistant bacterial strain(s), wherein the method comprises:
administering to the subject or patient a vector, said vector encoding a DNA modifying enzyme inactivating the antibiotic resistance gene (s) responsible for antibiotic-resistance of bacterial strain(s),
thereby selectively removing antibiotic resistance from an antibiotic resistant bacterial strain(s) in the microbiota and preventing antibiotic resistance related infection in the subject.
   2. The method of clause 1, wherein the DNA modifying enzyme is a nuclease.
   3. The method of clause 1, wherein the DNA modifying enzyme is a base editor.
   4. The method of clause 1, wherein the vector is inside a bacterial delivery vehicle.
   5. The method of clause 1, wherein the vector is not inside a bacterial delivery vehicle.
   6. The method of clause 1, wherein the vector further comprises a conditional origin of replication which is inactive in the targeted antibiotic resistant bacterial strain(s) but is active in a donor bacterial cell.
   7. The method of clause 1, wherein antibiotic resistance bacteria are removed from the gut of the healthy subject or patient.
   8. The method of clause 1, wherein the selective decolonization occurs before the subject or patient undergoes surgery.
   9. The method of clause 8, wherein the surgery is a solid organ transplant surgery of the kidney or liver.
   10. The method of clause 1, wherein said microbiota is a gut microbiota.
   11. The method of clause 1, wherein said antibiotic is methicillin, vancomycin, or a carbapenem class or beta-lactam class antibiotic.
   12. The method of clause 1, wherein the nuclease is a CRISPR associated nuclease.
   13. The method of clause 1, further comprising obtaining a microbiota sample from the subject or patient and confirming the presence of said antibiotic resistant bacterial strain(s) in the sample.
   14. Composition or combination comprising at least one, in particular at least 2, 3 or 4, recombinant phage(s) for use in a method of preventing antibiotic resistant Escherichia coli related infection in a patient carrying antibiotic resistant Escherichia coli strain(s),
wherein said recombinant phage comprises a nucleic acid encoding a DNA modifying enzyme inactivating the antibiotic resistance gene(s) responsible for antibiotic-resistance of E. coli strain(s),
thereby selectively removing antibiotic resistance from antibiotic resistant E. coli strain(s) in the microbiota of said patient and preventing antibiotic resistant E. coli related infection in the patient.
   15. Composition or combination for use according to clause 14, wherein said patient suffers from a hematological malignancy such as hematological cancer.

### EXAMPLES

### Example 1 Packaged Phagemid Construction

A packaged phagemid has been designed consisting of a bacterial delivery vehicle derived from phage lambda and engineered to include host range determinants (stf and gpj) that allow to target clinical isolates of ESBL E. coli involved in hospital-acquired infections in patients. The bacterial delivery vehicle contains a DNA payload encoding for the Fn Cpf1 nuclease and either one or two (multiplexing) crRNAs (also named ctx guide) that target conserved regions of the ESBL gene CTX-M.

To produce the packaged phagemids, the phagemids were transformed in an E. coli strain carrying the lambda prophage lacking a packaging site but otherwise possessing all the machinery for the induction of the lambda phage lytic cycle as well as the DNA packaging system. The cl repressor of the lambda prophage carries mutations making it thermosensitive and enabling the induction of the lytic cycle through temperature change. The cells containing the lambda phagemid were grown at 30° C. in liquid LB media. At an OD600 of 0.6, the culture was shifted to 42° C. for 25 minutes to induce the entry into lytic cycle. After that, cells were shifted back to 37° C. for 3 hours to allow for virion assembly containing the lambda phagemid. Cells were then centrifuged and washed in lambda buffer (10 mM Tris pH 7.5, 100 mM NaCl, 10 mM MgSO4). Chloroform was added and the sample was spun down at 17,000 g for 5 minutes. Finally, the aqueous phase was collected and filtered through a 0.2 pm pore-size filter. The titer of the packaged phagemids was measured by performing a transduction assay in vitro using strain MG-GFP as a recipient. MG-GFP is a derivative of strain MG1655 with a gfp fluorescent reporter gene and an ampicillin resistance gene inserted in the chromosome.

Sequences corresponding to the various features of the packaged phagemid are listed below:

Tip: 1A2 gpJ

(SEQ ID NO: 22)
ATGGGTAAAGGAAGCAGTAAGGGGCATACCCCGCGCGAAGCGAAG
GACAACCTGAAGTCCACGCAGTTGCTGAGTGTGATCGATGCCATC
AGCGAAGGGCCGATTGAAGGTCCGGTGGATGGCTTAAAAAGCGTG
CTGCTGAACAGTACGCCGGTGCTGGACACTGAGGGGAATACCAAC
ATATCCGGTGTCACGGTGGTGTTCCGGGCTGGTGAGCAGGAGCAG
ACTCCGCCGGAGGGATTTGAATCCTCCGGCTCCGAGACGGTGCTG
GGTACGGAAGTGAAATATGACACGCCGATCACCCGCACCATTACG
TCTGCAAACATCGACCGTCTGCGCTTTACCTTCGGTGTACAGGCA
CTGGTGGAAACCACCTCAAAGGGTGACAGGAATCCGTCGGAAGTC
CGCCTGCTGGTTCAGATACAACGTAACGGTGGCTGGGTGACGGAA
AAAGACATCACCATTAAGGGCAAAACCACCTCGCAGTATCTGGCC
TCGGTGGTGATGGGTAACCTGCCGCCGCGCCCGTTTAATATCCGG
ATGCGCAGGATGACGCCGGACAGCACCACAGACCAGCTGCAGAAC
AAAACGCTCTGGTCGTCATACACTGAAATCATCGATGTGAAACAG
TGCTACCCGAACACGGCACTGGTCGGCGTGCAGGTGGACTCGGAG
CAGTTCGGCAGCCAGCAGGTGAGCCGTAATTATCATCTGCGCGGG
CGTATTCTGCAGGTGCCGTCGAACTATAACCCGCAGACGCGGCAA
TACAGCGGTATCTGGGACGGAACGTTTAAACCGGCATACAGCAAC
AACATGGCCTGGTGTCTGTGGGATATGCTGACCCATCCGCGCTAC
GGCATGGGGAAACGTCTTGGTGCGGCGGATGTGGATAAATGGGCG
CTGTATGTCATCGGCCAGTACTGCGACCAGTCAGTGCCGGACGGC
TTTGGCGGCACGGAGCCGCGCATCACCTGTAATGCGTACCTGACC
ACACAGCGTAAGGCGTGGGATGTGCTCAGCGATTTCTGCTCGGCG
ATGCGCTGTATGCCGGTATGGAACGGGCAGACGCTGACGTTCGTG
CAGGACCGACCGTCGGATAAGACGTGGACCTATAACCGCAGTAAT
GTGGTGATGCCGGATGATGGCGCGCCGTTCCGCTACAGCTTCAGC
GCCCTGAAGGACCGCCATAATGCCGTTGAGGTGAACTGGATTGAC
CCGAACAACGGCTGGGAGACGGCGACAGAGCTTGTTGAAGATACG
CAGGCCATTGCCCGTTACGGTCGTAATGTTACGAAGATGGATGCC
TTTGGCTGTACCAGCCGGGGGCAGGCACACCGCGCCGGGCTGTGG
CTGATTAAAACAGAACTGCTGGAAACGCAGACCGTGGATTTCAGC
GTCGGCGCAGAAGGGCTTCGCCATGTACCGGGCGATGTTATTGAA
ATCTGCGATGATGACTATGCCGGTATCAGCACCGGTGGTCGTGTG
CTGGCGGTGAACAGCCAGACCCGGACGCTGACGCTCGACCGTGAA
ATCACGCTGCCATCCTCCGGTACCGCGCTGATAAGCCTGGTTGAC
GGAAGTGGCAATCCGGTCAGCGTGGAGGTTCAGTCCGTCACCGAC
GGCGTGAAGGTAAAAGTGAGCCGTGTTCCTGACGGTGTTGCTGAA
TACAGCGTATGGGAGCTGAAGCTGCCGACGCTGCGCCAGCGACTG
TTCCGCTGCGTGAGTATCCGTGAGAACGACGACGGCACGTATGCC
ATCACCGCCGTGCAGCATGTGCCGGAAAAAGAGGCCATCGTGGAT
AACGGGGCGCACTTTGACGGCGAACAGAGTGGCACGGTGAATGGT
GTCACGCCGCCAGCGGTGCAGCACCTGACCGCAGAAGTCACTGCA
GACAGCGGGGAATATCAGGTGCTGGCGCGATGGGACACACCGAAG
GTGGTGAAGGGCGTGAGTTTCCTGCTCCGTCTGACCGTAACAGCG
GACGACGGCAGTGAGCGGCTGGTCAGCACGGCCCGGACGACGGAA
ACCACATACCGCTTCACGCAACTGGCGCTGGGGAACTACAGGCTG
ACAGTCCGGGCGGTAAATGCGTGGGGGCAGCAGGGCGATCCGGCG
TCGGTATCGTTCCGGATTGCCGCACCGGCAGCACCGTCGAGGATT
GAGCTGACGCCGGGCTATTTTGAGATAAGGGCCACGCCGCATCTT
GCCGTTTATGACCCGACGGTACAGTTTGAGTTCTGGTTCTCGGAA
AAGCAGATTGCGGATATCAGACAGGTTGAAACCAGCACGCGTTAT
CTTGGTACGGCGCTGTAGTGGATAGGGGGGAGTATGAATATGAAA
GGGGGGGATGATTATTAGTTTTATATGGGGAGTGTGAACACCGTT
GGCAAATCGGCATTCGTGGAGGCCGTCGGTCGGGCGAGCGATGAT
GCGGAAGGTTAGGTGGATTTTTTGAAAGGGAAGATAAGGGAATGG
GATGTGGGGAAGGAGGTGCTGGAAAAAGTCGAGCTGACGGAGGAT
AACGCCAGCAGACTGGAGGAGTTTTCGAAAGAGTGGAAGGATGCC
AGTGATAAGTGGAATGCCATGTGGGCTGTCAAAATTGAGCAGACC
AAAGACGGCAAACATTATGTCGCGGGTATTGGCCTCAGCATGGAG
GACACGGAGGAAGGCAAACTGAGCCAGTTTCTGGTTGCCGCCAAT
CGTATCGCATTTATTGACCCGGCAAACGGGAATGAAACGCCGATG
TTTGTGGCGCAGGGCAACCAGATATTCATGAACGACGTGTTCCTG
AAGCGCCTGACGGCCCCCACCATTACCAGCGGCGGCAATCCTCCG
GCCTTTTCCCTGACACCGGACGGAAAGCTGACCGCTAAAAATGCG
GATATCAGCGGTAACGTGAATGCGAACTCCGGGACGCTCAACAAC
GTCACGATTAACGAGAACTGTCGGGTTCTGGGAAAATTGTCCGCG
AACCAGATTGAAGGCGATCTCGTTAAAACAGTGGGCAAAGCTTTC
CCCCGGGACTCCCGTGCACCGGAGCGGTGGCCATCAGGAACCATT
ACCGTCAGGGTTTATGACGATCAGCCGTTTGACCGGCAGATTGTT
ATTCCGGCGGTGGCATTCAGCGGCGCTAAACATGAGAAAGAGCAT
ACTGATATTTACTCCTCATGCCGTCTGATAGTGCGGAAAAACGGT
GCTGAAATTTATAACCGTACCGCGCTGGATAATACGCTGATTTAC
AGTGGCGTTATTGATATGCCTGCCGGTCACGGTCACATGACACTG
GAGTTTTCGGTGTCAGCATGGCTGGTAAATAACTGGTATCCCACA
GCAAGTATCAGCGATTTGCTGGTTGTGGTGATGAAGAAAGCCACT
GCAGGCATCACGATTAGC

Amino acid sequence

(SEQ ID NO: 23)
MGKGSSKGHTPREAKDNLKSTQLLSVIDAISEGPIEGPVDGLKSV
LLNSTPVLDTEGNTNISGVTVVFRAGEQEQTPPEGFESSGSETVL
GTEVKYDTPITRTITSANIDRLRFTFGVQALVETTSKGDRNPSEV
RLLVQIQRNGGWVTEKDITIKGKTTSQYLASVVMGNLPPRPFNIR
MRRMTPDSTTDQLQNKTLWSSYTEIIDVKQCYPNTALVGVQVDSE
QFGSQQVSRNYHLRGRILQVPSNYNPQTRQYSGIWDGTFKPAYSN
NMAWCLWDMLTHPRYGMGKRLGAADVDKWALYVIGQYCDQSVPDG
FGGTEPRITCNAYLTTQRKAWDVLSDFCSAMRCMPVWNGQTLTFV
QDRPSDKTWTYNRSNVVMPDDGAPFRYSFSALKDRHNAVEVNWID
PNNGWETATELVEDTQAIARYGRNVTKMDAFGCTSRGQAHRAGLW
LIKTELLETQTVDFSVGAEGLRHVPGDVIEICDDDYAGISTGGRV
LAVNSQTRTLTLDREITLPSSGTALISLVDGSGNPVSVEVQSVTD
GVKVKVSRVPDGVAEYSVWELKLPTLRQRLFRCVSIRENDDGTYA
ITAVQHVPEKEAIVDNGAHFDGEQSGTVNGVTPPAVQHLTAEVTA
DSGEYQVLARWDTPKVVKGVSFLLRLTVTADDGSERLVSTARTTE
TTYRFTQLALGNYRLTVRAVNAWGQQGDPASVSFRIAAPAAPSRI
ELTPGYFQITATPHLAVYDPTVQFEFWFSEKQIADIRQVETSTRY
LGTALYWIAASINIKPGHDYYFYIRSVNTVGKSAFVEAVGRASDD
AEGYLDFFKGKITESHLGKELLEKVELTEDNASRLEEFSKEWKDA
SDKWNAMWAVKIEQTKDGKHYVAGIGLSMEDTEEGKLSQFLVAAN
RIAFIDPANGNETPMFVAQGNQIFMNDVFLKRLTAPTITSGGNPP
AFSLTPDGKLTAKNADISGNVNANSGTLNNVTINENCRVLGKLSA
NQIEGDLVKTVGKAFPRDSRAPERWPSGTITVRVYDDQPFDRQIV
IPAVAFSGAKHEKEHTDIYSSCRLIVRKNGAEIYNRTALDNTLIY
SGVIDMPAGHGHMTLEFSVSAWLVNNWYPTASISDLLVVVMKKAT
AGITIS

Tail Fiber protein: stf-27

STF27—amino acid sequence

(SEQ ID NO: 24)
MAVKISGVLKDGTGKPVQNCTIQLKARRNSTTVVVNTVGSENPDE
AGRYSMDVEYGQYSVILQVDGFPPSHAGTITVYEDSQPGTLNDFL
CAMTEDDARPEVLRRLELMVEEVARNASVVAQSTADAKKSETAAA
SSKNAAKTSETNAANSAQAAAASQTASANSATAAKKSETSAKNSE
TATKASEKNAKSSQTAAKTSETNAKDSEANAKVSETAAANSAKAS
AASQTAAKASEDAAREYANQTAEPYRYVLQPLPDVWIPFNDSLDM
ITGYSPGYKKVKIGDNVVQVASDKQVNFSRASTATYINKSGELKT
AEINEPRFECDGLLIEGQRTNFFQNSTDPSKWNKSTSLDVTETGT
DSFGFNYGRFVVQDSIVGTSKAHTIIGLYSSTGGVDTSGDEKHVT
ISCRVKSEVDNIAVRILFEHYDGEVRTSIGAANLNLTTRIISKTG
QTSRVTARSVKDDATGWIFFEATLKADTTENTVGGFVQYSPDTGQ
MVTSGDYLDVTTPQIEAGTGASSFIVTGTAPATRASDMVTVPIKN
NLYNLPFTVLCEVHKNWYKTPNVAPRVFDTGGHQTGAGIVMGFGS
SGGYDGFPYCDIGGSDRRINENAGLEKMLIGMRVKSERSTCVVSN
GKLSSETKTKWEYIRSTATIRIGGQTTAGLRHLFGHVRNFRLWHK
ELTDAQLGEVVE

>STF27 accessory protein—amino acid sequence

(SEQ ID NO: 25)
VRDFTLRFSDKADFRAFLRKLNWEEDEELQNAVLVDEIGFTFRET
DVSDDGEPEYTRNEGYFVNIRLLDDGFEDSVFREWVVTPERPLRE
WF

STF27—DNA sequence

(SEQ ID NO: 26)
ATGGCAGTAAAGATTTCAGGAGTCCTGAAAGACGGCACAGGAAAAC
CGGTACAGAACTGCACCATTCAGCTGAAAGCCAGACGTAACAGCA
CCACGGTGGTGGTGAACACGGTGGGCTCAGAGAATCCGGATGAAG
CCGGGCGTTACAGCATGGATGTGGAGTACGGTCAGTACAGTGTCA
TCCTGCAGGTTGACGGTTTTCCACCATCGCACGCCGGGACCATCA
CCGTGTATGAAGATTCACAACCGGGGACGCTGAATGATTTTCTCT
GTGCCATGACGGAGGATGATGCCCGGCCGGAGGTGCTGCGTCGTC
TTGAACTGATGGTGGAAGAGGTGGCGCGTAACGCGTCCGTGGTGG
CACAGAGTACGGCAGACGCGAAGAAATCAGAAACGGCAGCAGCCT
CATCGAAGAATGCGGCGAAAACCTCAGAAACGAATGCAGCTAACA
GCGCACAGGCGGCAGCGGCCTCGCAGACTGCATCGGCAAACTCCG
CGACAGCAGCCAAAAAATCAGAAACCAGCGCGAAAAATAGCGAGA
CAGCCACAAAGGCCAGCGAAAAAAACGCAAAATCCAGCCAGACGG
CAGCGAAAACCAGTGAGACGAATGCCAAAGACAGTGAAGCCAACG
CAAAGGTGAGCGAAACAGCGGCGGCGAACTCGGCGAAAGCATCGG
CAGCAAGCCAGACGGCAGCAAAAGCAAGTGAAGATGCTGCCAGAG
AATACGCAAACCAGACAGCAGAGCCGTACAGATATGTTTTACAGC
CGCTGCCGGATGTGTGGATACCCTTTAATGATTCGCTGGATATGA
TTACGGGCTATTCTCCGGGTTATAAAAAAGTGAAGATTGGTGATA
ATGTGGTTCAGGTTGCCAGTGATAAACAGGTTAATTTCAGTCGCG
CATCAACGGCAACATATATCAACAAATCTGGCGAACTGAAAACGG
CGGAAATTAATGAGCCGCGATTTGAGTGTGATGGCCTGCTTATTG
AGGGACAAAGAACGAACTTCTTCCAGAACAGTACAGACCCTTCGA
AGTGGAATAAGTCAACTTCACTGGACGTTACAGAAACAGGCACAG
ATAGTTTCGGGTTTAATTATGGTCGGTTTGTCGTACAGGATTCGA
TTGTTGGTACAAGTAAAGCGCATACCATTATCGGACTGTATTCGA
GTACCGGAGGGGTTGATACTTCAGGGGACGAAAAGCATGTAACTA
TATCCTGTCGGGTAAAAAGTGAAGTTGATAATATCGCCGTTCGTA
TTTTATTTGAACATTATGATGGGGAGGTAAGGACATCAATAGGAG
CAGCAAACCTGAACCTTACCACCCGCATAATTAGCAAGACAGGTC
AGACAAGCCGTGTTACAGCAAGGTCTGTTAAGGATGATGCAACTG
GCTGGATATTTTTTGAGGCTACATTAAAAGCAGATACAACAGAAA
ATACGGTTGGTGGTTTTGTCCAGTATTCTCCGGATACAGGGCAGA
TGGTTACATCAGGGGATTATCTCGATGTAACCACTCCACAGATTG
AGGCTGGTACAGGCGCATCATCTTTTATTGTTACGGGGACGGCAC
CGGCAACGCGGGCAAGCGATATGGTGACAGTCCCAATCAAGAATA
ACCTTTATAATCTTCCTTTTACGGTTCTTTGTGAGGTACATAAGA
ACTGGTATAAAACGCCAAATGTAGCGCCGCGTGTTTTTGATACCG
GCGGTCATCAAACCGGAGCGGGGATCGTAATGGGGTTTGGTTCAT
CAGGTGGGTACGACGGTTTTCCGTATTGCGATATAGGTGGTTCAG
ACCGACGAATAAATGAAAATGCCGGGCTGGAAAAAATGCTTATTG
GTATGCGGGTAAAGTCCGAACGGTCCACATGTGTAGTCAGTAACG
GTAAGTTAAGCAGCGAAACTAAAACCAAATGGGAATATATCCGGA
GTACAGCAACCATTCGCATTGGTGGACAAACTACAGCAGGATTAC
GCCATTTATTTGGGCATGTGAGGAATTTTCGTCTCTGGCATAAAG
AGCTAACAGATGCGCAGCTTGGGGAGGTTGTGGAG

STF27 accessory protein—DNA sequence

(SEQ ID NO: 27)
GTGAGAGATTTCACGTTGCGTTTCAGTGATAAAGCAGATTTCAGG
GCATTTCTCAGGAAACTTAACTGGGAAGAGGACGAAGAGCTGCAG
AATGCCGTTCTGGTTGATGAGATTGGTTTTACGTTCAGGGAGACA
GATGTTTCTGATGACGGAGAACCAGAATACACGCGAAACGAAGGG
TACTTTGTTAATATCCGTCTTCTTGACGATGGATTTGAGGATTCC
GTGTTCCGTGAGTGGGTGGTTACACCAGAGCGCCCGCTCAGGGAG
TGGTTT

payload

p775: RBS1-AK272-CTX1

Payload encoding a Fn Cpf1 and an associated crRNA targeting a conserved region of CTX-M1 gene variants (“CTX1”: GCCAGATCACCGCGATATCGTTGG, SEQ ID NO: 28).

(SEQ ID NO: 29)
ACTGTCGGAATGACAAATGGTTCCAATTATTGAACACCCTTCGGG
GTGTTTTTTTGTTTCTGGTTTCCCGAGGCCGGCCTGCGCTAGCGG
AGTGTATACTGGCTTACTATGTTGGCACTGATGAGGGTGTAAGTG
AAGTGCTTCATGTGGCAGGAGAAAAAAGGCTGCATCGGTGCGTCA
GCAGAATATGTGATACAGGATATATTCCGCTTCCTCGCTCACTGA
CTCGCTACGCTCGGTCGTTCGACTGTGGCGAGCGGAAATGGCTTA
CGAACGGGGCGGAGATTTCCTGGAAGATGCCAGGAAGATACTTAA
CAGGGAAGTGAGAGGGTCGCGGCAAAGCCGTTTTTCCATAGGCTC
CGCCCCCCTGACAAGCATCACGAAATCTGACGCTCAAATCAGTGG
TGGCGAAACCTGACAGGACTATAAAGATACCAGGCGTTTCCCCCT
GGCGGCTCCCTCGTGCGCTCTCCTGTTCCTGCCTTTCGGTTTGCC
GGTGTCATTCCTCTGTTACGGCCGAGTTTGTCTCATTCCACGCCT
GACACTCAGTTCCGGGTAGGCAGTTCGCTCCAAGCTGGACTGTAT
GCACGAACCCCCCGTTCAGTCCGACCGCTGCGCCTTATCCGGTAA
CTATCGTCTTGAGTCCAACCCGGAAAGACATGCAAAAGCACCACT
GGCAGCAGCCACTGGTAATTGATTTAGAGGAGTTAGTCTTGAAGT
CATGCGCCGGATAAGGCTAAACTGAAAGGACAAGTTTTGGCGACT
GCGCTCCTCCAAGCCAGTTACCTCGGTTCAAAGAGTTGGTAGCTC
AGAGAACCTTCGAAAAACCGCCCTGCAAGGCGGTTTTTTCGTTTT
CAGAGCAAGAGATTACGCGCAGACCAAAACGATCTCAAGAAGATC
ATCTTATTAATCAGATAAAATATTTCTAGATTTCAGTGCAATTTA
TCTCTTCAAATGTAGCACCGGCGCGCCGTGACCAATTATTGAAGG
CCGCTAACGCGGCCTTTTTTTGTTTCTGGTATCCCGAATGGAGCG
ACTTCTCCCCAAAAAGCCTCGCTTTCAGCACCTGTCGTTTCCTTT
CTTTTCAGAGGGTATTTTAAATAAAAACATTAAGTTATGACGAAG
AAGAACGGAAACGCCTTAAACCGGAAAATTTTCATAAATAGCGAA
AACCCGCGAGGTCGCCGCCCCGTAACCTGTCGGATCACCGGAAAG
GACCCGTAAAGTGATAATGATTATCATCTACATATCACAACGTGC
GTAAAGGGTAAGTATGAAGGTCGTGTACTCCATCGCTACCAAATT
CCAGAAAACAGACGCTTTCGAGCGTCTTTTTTCGTTTTGGTCACG
ACGTACGGTGGAAGATTCGTTACCAATTGACAGCTAGCTCAGTCC
TAGGTATATACATACATGCTTGTTTGTTTGTAAACTACTGTTTTC
ATTAAAGAGGAGAAAGGAAGCCATGTCCATCTATCAGGAGTTTGT
TAACAAGTATTCCCTGTCTAAAACCCTGCGTTTTGAACTGATCCC
GCAGGGCAAAACTTTGGAAAACATTAAAGCGCGTGGCCTGATTCT
GGATGACGAAAAACGTGCAAAGGATTACAAGAAAGCTAAACAGAT
CATCGACAAATATCACCAGTTCTTTATCGAAGAAATTCTGTCGTC
GGTGTGCATCAGTGAGGATCTGTTACAGAATTATTCTGATGTATA
CTTTAAACTTAAAAAGTCCGATGACGATAATCTGCAAAAAGATTT
CAAGTCAGCCAAAGATACCATCAAGAAACAGATCTCAGAATATAT
TAAAGATAGCGAAAAGTTCAAAAACCTGTTTAACCAAAACCTCAT
TGATGCTAAGAAAGGCCAAGAATCTGACCTGATCTTATGGCTGAA
ACAGAGCAAAGATAACGGCATTGAACTGTTCAAAGCTAATAGCGA
CATCACCGATATTGATGAAGCGCTCGAAATCATCAAGTCTTTCAA
AGGCTGGACGACGTATTTCAAAGGTTTTCATGAAAACCGTAAGAA
TGTATATTCGAGCAACGATATTCCGACCTCTATTATTTATCGTAT
CGTGGACGACAACCTGCCGAAGTTTCTGGAAAACAAAGCGAAATA
TGAATCTCTGAAAGACAAAGCACCGGAAGCTATTAACTATGAACA
GATCAAGAAAGATCTGGCGGAAGAACTGACCTTCGACATCGACTA
TAAAACCTCCGAAGTTAACCAGCGTGTTTTCTCACTGGACGAGGT
TTTCGAAATCGCTAATTTCAACAATTACCTGAATCAATCTGGCAT
CACCAAATTCAACACCATTATTGGTGGCAAATTTGTTAACGGCGA
AAACACCAAGCGTAAGGGCATCAACGAATACATTAACCTGTATAG
CCAACAAATCAACGACAAAACCCTGAAAAAGTATAAAATGTCCGT
TCTGTTTAAACAGATTTTATCGGACACCGAATCTAAATCCTTCGT
AATTGATAAACTGGAAGATGATAGCGACGTTGTCACCACGATGCA
GAGCTTTTATGAGCAGATTGCGGCGTTCAAAACCGTGGAAGAGAA
ATCTATTAAAGAAACTCTGTCCCTGCTCTTTGACGACCTCAAAGC
GCAGAAACTAGATCTGTCTAAGATTTACTTTAAAAACGACAAATC
TCTGACCGATCTCAGTCAACAAGTTTTCGATGACTATAGCGTGAT
CGGCACGGCAGTTTTGGAATACATCACCCAACAAATCGCGCCGAA
AAATCTGGACAACCCGTCCAAGAAGGAACAGGAACTGATTGCAAA
GAAAACAGAAAAAGCTAAATACCTGAGCTTAGAAACTATCAAACT
GGCACTTGAGGAATTTAATAAACATCGTGATATTGATAAACAGTG
TCGTTTTGAGGAAATTCTGGCGAACTTTGCGGCAATCCCGATGAT
CTTCGACGAAATTGCTCAAAACAAAGACAATCTGGCGCAGATCTC
TATCAAGTACCAGAATCAGGGTAAGAAAGATCTGCTTCAAGCATC
TGCGGAGGACGATGTGAAAGCAATTAAAGACTTATTAGATCAGAC
GAATAACTTATTACACAAGCTCAAAATCTTCCACATCAGCCAGAG
CGAGGACAAGGCGAACATTCTGGATAAAGATGAACACTTCTATCT
GGTGTTCGAAGAATGTTACTTCGAACTGGCAAACATCGTCCCTCT
CTACAATAAAATCCGCAACTACATCACGCAGAAGCCTTACTCTGA
CGAGAAATTCAAACTGAACTTCGAAAACAGCACGCTGGCGAACGG
CTGGGATAAGAACAAAGAGCCGGACAACACCGCAATCCTGTTCAT
CAAAGACGACAAATACTATCTGGGCGTAATGAACAAGAAGAACAA
CAAGATCTTCGACGATAAAGCGATCAAAGAAAACAAGGGTGAAGG
CTATAAGAAAATCGTGTACAAGCTCCTGCCGGGTGCGAATAAAAT
GTTACCGAAAGTGTTCTTTTCCGCGAAAAGCATCAAATTCTACAA
CCCGTCTGAGGATATTCTGCGCATCCGCAATCATAGCACGCACAC
TAAAAACGGTAGCCCGCAGAAAGGGTATGAAAAATTCGAATTTAA
TATAGAGGACTGCCGTAAGTTCATCGACTTCTATAAACAGAGCAT
TTCCAAACATCCGGAATGGAAAGACTTCGGCTTCCGTTTCTCTGA
CACTCAGCGCTATAATAGCATCGACGAGTTCTACCGCGAAGTGGA
GAATCAGGGCTATAAACTGACCTTCGAGAACATTAGTGAGTCGTA
CATCGACTCCGTTGTGAATCAGGGTAAACTGTACCTGTTTCAGAT
CTATAATAAAGACTTTAGCGCGTACAGCAAAGGCCGTCCGAATCT
GCACACCCTTTACTGGAAAGCATTATTTGACGAACGTAACCTGCA
AGATGTGGTGTATAAACTGAACGGTGAGGCGGAACTTTTCTACCG
TAAACAGAGTATCCCGAAGAAAATCACGCATCCGGCAAAAGAAGC
TATTGCCAACAAAAACAAAGACAACCCGAAGAAAGAATCAGTATT
CGAATATGACCTGATCAAAGATAAACGTTTCACCGAAGATAAGTT
CTTTTTCCACTGTCCGATTACCATCAACTTCAAATCTAGCGGTGC
GAACAAGTTCAACGATGAAATTAACTTATTACTGAAAGAGAAAGC
TAATGACGTACACATCTTATCTATTGATCGCGGTGAACGTCATTT
AGCATACTATACACTGGTAGATGGTAAAGGTAATATTATTAAACA
GGATACTTTCAATATTATCGGTAATGACCGTATGAAAACCAACTA
TCACGATAAGCTGGCGGCGATCGAAAAAGATCGTGATTCTGCGCG
TAAAGATTGGAAGAAAATTAACAATATCAAAGAAATGAAAGAAGG
CTATCTGAGCCAAGTGGTGCACGAGATCGCAAAACTGGTGATTGA
ATATAACGCTATCGTGGTTTTCGAAGATCTGAACTTTGGTTTTAA
ACGTGGTCGCTTCAAAGTAGAAAAACAGGTGTACCAAAAACTGGA
AAAAATGCTGATTGAAAAACTGAACTATCTGGTTTTTAAAGACAA
CGAATTTGACAAAACGGGTGGCGTACTCCGTGCCTATCAGCTGAC
CGCTCCGTTCGAAACGTTCAAGAAAATGGGTAAACAAACGGGGAT
TATCTATTATGTGCCAGCTGGTTTCACCTCCAAGATTTGTCCAGT
TACGGGCTTCGTTAACCAGCTGTACCCGAAATACGAGAGCGTTAG
CAAATCTCAAGAATTTTTCAGCAAATTCGACAAGATCTGCTATAA
TCTGGATAAAGGCTATTTCGAGTTCAGCTTCGATTACAAAAACTT
CGGCGATAAAGCGGCTAAAGGTAAGTGGACTATTGCTAGCTTTGG
TAGCCGTCTGATTAACTTTCGCAACTCCGACAAAAACCATAATTG
GGACACGCGTGAAGTGTATCCGACCAAAGAACTGGAAAAATTACT
GAAAGACTATTCCATCGAATATGGTCATGGGGAGTGCATTAAAGC
GGCGATTTGCGGTGAATCCGATAAGAAATTTTTCGCCAAACTGAC
CAGCGTGCTTAACACCATTCTGCAAATGCGTAATTCTAAAACGGG
TACGGAGCTGGACTACCTGATTTCTCCGGTAGCCGACGTTAACGG
CAACTTCTTCGATTCTCGTCAAGCACCGAAAAATATGCCACAAGA
CGCGGATGCCAACGGTGCATACCATATCGGCTTAAAAGGCTTAAT
GTTATTAGGCCGTATCAAGAATAATCAGGAGGGCAAGAAATTAAA
TCTGGTTATCAAAAACGAAGAATACTTCGAGTTCGTTCAGAATCG
TAACAATTAATGTATGCTTAAGCAGCTCGGTACCAAAGACGAACA
ATAAGACGCTGAAAAGCGTCTTTTTTCGTTTTGGTCCTGTTGCGG
CGCGATAGTGTGAACATGCTATAGACTTCTGGTGCTACCCGACTG
ACAATTAATCATCCGGCTCGTATAATGCTAGCAATTTCTACTGTT
GTAGATGCCAGATCACCGCGATATCGTTGGTCGAGACGAACAATA
AGGCCTCCCTAACGGGGGGCCTTTTTTATTGATAACAAAAGTAAC
TTCGAGCTTGTCTACCTCCTAGCACCATTATTGCAATTAATAAAC
AACTAACGGACAATTCTACCTAACAGTTTTCATATATGACGAGCA
GTTAAGTGATGAGTAAAGGTGAGGAATTATTTACTGGTGTTGTTC
CGATCTTAGTTGAACTGGACGGCGATGTTAACGGTCATAAATTCA
GTGTTCGTGGTGAAGGTGAAGGTGATGCAACCAACGGTAAGCTGA
CCCTGAAATTCATCTGCACTACTGGAAAATTACCAGTACCGTGGC
CTACTCTGGTGACTACCCTGACCTATGGTGTTCAGTGTTTTTCTC
GTTACCCTGACCACATGAAGCAACATGATTTCTTCAAATCTGCAA
TGCCGGAAGGTTATGTACAGGAGCGCACCATTTCTTTCAAAGACG
ATGGCACGTATAAAACCCGTGCAGAGGTTAAATTTGAAGGTGACA
CTCTGGTGAATCGTATTGAACTGAAAGGCATTGATTTCAAAGAGG
ACGGCAATATTTTAGGCCACAAACTGGAATATAACTTCAACTCCC
ATAACGTTTACATCACCGCAGACAAACAGAAGAACGGTATCAAAG
CTAACTTCAAAATTCGCCATAACGTTGAAGATGGTAGCGTACAGC
TGGCGGATCATTACCAACAGAACACTCCGATTGGAGATGCTCCTG
TTTTACTGCCGGATAACCACTACCTGTCCACCCAGTCTAAACTGT
CGAAGGATCCGAACGAAAAGCGCGACCACATGGTGTTATTAGAGT
TCGTTACCGCTAGTGGTATCACGCACGGTATGGATGAACTCTACA
AATAAGACGAACAATAAGGGGAGCGGGAAACCGCTCCCCTTTTTT
ATTGATAACAAAAGTAAATTGCACGCTGATAGTCTCCCAATTGCG
AAGGACCAAAACGAAAAAACACCCTTTCGGGTGTCTTTTCTGGAA
TTTGGTACCGAGTACTAGGTATCGTGTAAGTAGCGAAGGCCCGTA
CGCGAGATAAACTGCTAGGCAACCGCGACTCTACGACTGGTGCTC
GATTTAATTTCGCTGACGTAAAGAAATTATCGGCAGTGCGTCAAC
TGCCGTATCTTTATCTTAATTAGGTAGTTGGACAAGCCCTTGAAA
GAAATAGCAAGAGCCTGCCTCTCTATTGAAGTCACGGCGAAAGTC
GGGTAGAAATCAAAGAAAGCAGAAATTAAATCGGAGTAACACTAA
GGTGGGATAACTCCGTAACTGACTACGCCTTTCTCTAGACTTTAC
TTGACCAGATACACTGTCTTTGACACGTTGAAGGATTAGAGCAAT
CAAATCCAAGACTGGCTAAGCACGAAGCAACTCTTGAGTGTTAAA
AAGTTATCTCCTGTATTCGGGAAGCGGGTACTAGAAGATTGCAGG
GACTCCGACGTTAAGTAAATTACAAAGTAATAAGTATCGTTCAGG
ATCACGTTACCGCAATAAGAAGCGAGAATAATATAATTTCCGAAG
TGCTTACCCCAGTAGTGACTATTCCTATAACCCTTCTGAGTGTCC
GGAGGCGGAAATTTGCCACGAAAGAGAAAGTATTTCCCCGACAAT
AATAAAGGGGCGCTCCTCAGCTTTTCCACTTGGTTGGGTAAGCTA
GGCAACTCTGAAAGGAGTTTCGGCGAATTGAAGCCGACAGCTTTG
AATTGTTTTAGGGGCGTTATTCGAGGGCAATCGGAGCTAACTTCA
AGACTACTTCTTTGTTGAATACTAAATAGTGCAAAGGTCGTGTTT
CCTCAAGGATACTCCGCTAACAATATAGGATTCCAATCAGATTCA
GCACTGGCGGTACGGGTGTTGCGGTGAGGCGTTCGGGTTTACGGC
TCGAAGCTAGCACGGTAGGAAGCCTGACAATCACCAAGCAAAAGG
GCCGTCGAAGGCCCACAAGATACGAAAGCTCTCGAAGCCTTATCC
TTGACCGATCCACCTATTTAGGCAGTTACGCACAAAAGCTACCCA
ATAATCCGTGACAGGCACAATATCACGGAACAAAACCGAAAACTC
TCGTACACGGTTAGGTTTTCGCTAGGAAGAATAAACCTCTATCTT
GATTATAAGAAGGCTCCCCAAGCACCCCCAAAACCGAAATAGCGG
TTTGCAATAAGGGACAAGTTACGAGTGTAGACACGCAGAATTATC
CAGCCTTTAGTCTTTAGGAAGGCAAAGCTATTGTACGCGGTAGCC
GTCGTAGCAATTTACCAACTGTAGAATTATTGGACACACGTAGGA
AGGGCTTACAGTTGAAGTTTAATAAGGTCACACGCAAAACCGCTA
AGGAATAATCGCACCGTTAGCGAAAGAATATTTCAGAGCGGTTAG
TAAAGGTTGAGTAAAGTGAGATTCCAAAGTGAGCCTTTATAAAAA
GTAAAGAGCTATAATAAAACCGTCGAGCAGAAAACAATCGCCTGA
AATCTCAAGCACGTTGCCCTTTCTAACGTCGCTAAGGTTTCGTAA
ACCCGTTTGATTAGGAAGAAGAATAAGTAACCCGATTAGGTTTGA
GATCGCGGGTTATCGGTTTGGATTAAAAGTGGATACCAGCGGAGT
CAACGCCGACGCAAACGTACAGTGATCCAATCCTGTTGCACGGTC
AAGCACAATCAGCTCGCAAGATCTTGGAATAGTGTGCCCAACAGT
TTAGTTGAGGGCCACGTTCCGACTACAAGTTGCTTCAAGAGGGGA
ATTTGGATTTGGCAATAGCCCCCCGTTTCTACCTCAAGAGGCGAC
GAGTATTAACCGCGCCAGCTGTCGGCACAAGGGCCAAAGAAGATT
CCAATTTCTTATTCCCGAATAACCTCCGAATCCCTGCGGGAAAAT
CACCGACCGAATAGCCTAGAAGCAAGGGGGAACAGATAGGTATAA
TTAGCTTAAGAGAGTACCAGCCGTGACAACAGCGTAGTAACCACA
AACTTACGCTGGGGCTTCTTTGGCGGATTTTTACAGATACTAACA
AGGTGATTTGAAGTACCTTAGTTGAGGATTTAAACGCGCTATCCG
GTAATCTCCAAATTGGGAAATACCGTTCAAAGAGGGCTAGAATTA
CTTAAAAGCCTTCACACCGCCTGCGCTATACGCGCCCACTCTCCC
GTTTATCCGTCCAAGCGGAAGCAGGGCGATCCTCCGCTAAGATAT
TCTTACGTGTAACGTAGCTAAGTATCCCAAATAGCTGGCGTACGC
GTTGAACACCGCCTAGAGGATCGTGACTCGCCGGACGAGCGTGTT
ATTGGGGACTTACGCCAGCGTAGACTACAACGCGCCCAGATTAAC
CCTGCACGTATTGCCTTGAATAACGTACTAATCTCTCCGGCTCTC
GACAATCTATCGAGCGACTCGATTATCAACGGGTGTCTTGCAGTT
CTAATCTCTTGCCCCCGCCCGTAATAGCCTCCAAGAGATTGAAGA
TAGTAAAGGGCAAGAGCTGATTCGGCGTTGAAGGATAGCGGACTT
TCGGTCAACCACAATTCCCCACTCGACAAAACCAGCCGTGCGAAT
AACTCTGAAAGTACAAGCAACCCAAGAGGGCTGAGCCTAAACTCA
GCTAATTCCTAAGTGAGCTAAAGACTCGAAGTGACAGCTCTTAAT
AAATAGAGCGGGAACGTCGAACGGTCGTGAAAGTAATAGTACAAC
GGGTATTAACTTACTGAGGATATTGCTTGAAGCTGTACCGTTTTA
TTGGGTGAACGAATAAGATCCAGCAATTCAGCCAAAGAAGCTACC
AATTTTTAGTTTAAGAGTGTCACGTCTGACCTCGCGGGTAGATTG
CCGAACGTAGAGCTTACGAGCCAGCGGAAACAGTAGCCGCAGGAT
AAGTAAGGGGAGTAAGTGATCGAACGAATCAGAAGTGACAATATA
CTTAGGCTGGATCTCGTCCCGTGAATCCCAACCCTCACCAACTAC
GAGATAAGAGGTAAGCCAAAAATCGACTTGGTGGCGACCAACGAC
TGTTCCCCCCCTGTAACTAATCGTTCCGTCAAAACCTGACTTACT
TCAAGGCCAATTCCAAGCGCAAACAATACCGTCCTAGTTCTTCGG
TTAAGTTTCCGAAGTAGGAGTGAGCCTACCTCCGTTTGCGTCTTG
TTACCACTGACCCAGCTATTTACTTTGTATTGCCTGCAATCGAAT
TTCTGAACTCTCAGATAGTGGGGATAACGGGAAAGTTCCTATATT
TGCGAACTAACTTAGCCGTCCACCTCGAAGCTACCTACTCACACC
CACCCCGCGCGGGGTAAATAAGGCACTAATCCCAGCTGAGAGCTG
GCGTAGCACTTAGCCACAAGTTAATTAACAGTTGTCTGGTAGTTT
GGCGGTATTAGGAAGATCCTAGAAGCAAGGCAGAGTTAGTTCTAA
CCTAAAGCCACAAATAAGACAGGTTGCCAAAGCCCGCCGGAAATT
AAATCTTGCTCAGTTCGGTAACGGAGTTTCCCTCCCGCGTACTTA
ATTCCCAATAAGAAACGCGCCCAAGTCCTATCAGGCAAAATTCAG
CCCCTTCCCGTGTTAGAACGAGGGTAAAAATACAAGCCGATTGAA
CAAGGGTTGGGGGCTTCAAATCGTCGTTTACCCCACTTTACAACG
GAGATTAAGTAGTTCACCCTATAGTACGAAGCAGAACTATTTCGA
GGGGCGTGCAATAATCGAATCTTCTGCGGTTGACTTAACACGCTA
GGGACGTGCCCTCGATTCAATCGAAGGTACTCCTACTCAGACTGC
CTCACACCCAGCTAGTCACTGAGCGATAAAATTGACCCGCCCTCT
AGGGAAGCGAGTACGTCCCAAAGGGCTCCGGACAGGGCTATATAG
GAGAGTTTGATCTCGCCCCGACAACTGCAACCCTCAACTCCCTTA
GATAATATTGTTAGCCGAAGTTGCACGACCCGCCGTCCACGGACT
GCTCTTAGGGTGTGGCTCCTTAATCTGACAACGTGCAACCCCTAT
CGAAGTCGATTGTTTCTGCGAAAGGTGTTGTCCTAATAGTCCCGA
AATTTGGCCCTTGTAGGTGTGAAACCACTTAGCTTCGCGCCGTAG
TCCTAAAGGCCCACCTATTGACTTTGTTTCGGGTAGCACTAGGAA
TCTTAACAATTTGAATTTGGACGTGGAACGCGTACACCTTAATCT
CCGAATAATTCTAGGGATTTGGAAGTCCTCTACGTTGACACACCT
ACACTGCTCGAAGTAAATATACGAATAACGCGGGCCTCGCGGAGC
CGTTCCGAATCGTCACGTGTTCGTTTACTGTTAATTGGTGGCAAA
TAAGCAATATCGTAGTCCGTCAGGCCCAGCCCTGTTATCCACGGC
GTTATTTGTCAAATTGCGTAGAACTGGATTGACTGCCTGACAATA
CCTAATTATCGGTACGAAGTCCCCGAATCTGTCGGGCTATTTCAC
TAATACTTTCCAAACGCCCCGTATCCAAGAAGAACGAATTTATCC
ACGCTCCCGTCTTTGGGACGAATACCGCTACAAGTGGACAGAGGA
TCGGTACGGGCCTCTAATAAATCCAACACTCTACGCCCTCTTCAA
GAGCTAGAAGAACAGGGTGCAGTTGGAAAGGGAATTATTTCGTAA
GGCGAGCCAATACCGTAATTAATTCGGAAGAGTTAACACGATTGG
AAGTAGGAATAGTTTCTAACCACGGTTACTAATCCTAATAACGGA
ACGCTGTCTGATAGATTAGTGTCAGCGCTCGGTACCAAAGAAAAA
TAAAAAGACGCTGAAAAGCGTCTTTTTATTTTTCGGTCCAGTGTA
ACTCAGGCAAAAGCACGTAATATTCGTACTTTCTTCCTCCGTAAG
CGTCACCCACATTCCTTAAAGAGTGCATGTGCATATTTTGTTATC
AATAAAAAAGGCCGCGATTTGCGGCCTTATTGTTCGTCTTGCCGG
ATTACGCCCCGCCCTGCCACTCATCGCAGTATTGTTGTAATTCAT
TAAGCATTCTGCCGACATGGAAGCCATCACAAACGGCATGATGAA
CTTGGATCGCCAGTGGCATTAACACCTTGTCGCCTTGCGTATAAT
ATTTTCCCATAGTGAAAACGGGGGCGAAGAAGTTGTCCATATTTG
CTACGTTTAAATCAAAACTGGTGAAACTCACCCACGGATTGGCAC
TGACGAAAAACATATTTTCGATAAACCCTTTAGGGAAATATGCTA
AGTTTTCACCGTAACACGCCACATCTTGACTATATATGTGTAGAA
ACTGCCGGAAATCGTCGTGGTATTCTGACCAGAGCGATGAAAACG
TTTCAGTTTGCTCATGGAAAACGGTGTAACAAGGGTGAACACTAT
CCCATATCACCAGCTCACCGTCTTTCATTGCCATACGAAACTCCG
GATGTGCATTCATCAGGCGGGCAAGAATGTGAATAAAGGCCGGAT
AAAACTTGTGCTTATTTTTCTTTACGGTTTTTAAAAAGGCCGTAA
TATCCAGCTGAACGGTTTGGTTATAGGTGCACTGAGCAACTGACT
GGAATGCCTCAAAATGTTCTTTACGATGCCATTGACTTATATCAA
CTGTAGTATATCCAGTGATTTTTTTCTCCATTTTAGCTTCCTTAG
CTTGCGAAATCTCGATAACTCAAAAAATAGTAGTGATCTTATTTC
ATTATGGTGAAAGTTGTCTTACGTGCAACATTTTCGCAAAAAGTT
GGCGCTTTATCAAC

p776: RBS1-AK272-CTX3

Payload encoding a Fn Cpf1 and an associated crRNA targeting a conserved region of CTX-M9 gene variants (“CTX3”: TCGCGGTGATGAACGCTTTCCAAT, SEQ ID NO:

30).

(SEQ ID NO: 31)
ACTGTCGGAATGACAAATGGTTCCAATTATTGAACACCCTTCGGG
GTGTTTTTTTGTTTCTGGTTTCCCGAGGCCGGCCTGCGCTAGCGG
AGTGTATACTGGCTTACTATGTTGGCACTGATGAGGGTGTAAGTG
AAGTGCTTCATGTGGCAGGAGAAAAAAGGCTGCATCGGTGCGTCA
GCAGAATATGTGATACAGGATATATTCCGCTTCCTCGCTCACTGA
CTCGCTACGCTCGGTCGTTCGACTGTGGCGAGCGGAAATGGCTTA
CGAACGGGGCGGAGATTTCCTGGAAGATGCCAGGAAGATACTTAA
CAGGGAAGTGAGAGGGTCGCGGCAAAGCCGTTTTTCCATAGGCTC
CGCCCCCCTGACAAGCATCACGAAATCTGACGCTCAAATCAGTGG
TGGCGAAACCTGACAGGACTATAAAGATACCAGGCGTTTCCCCCT
GGCGGCTCCCTCGTGCGCTCTCCTGTTCCTGCCTTTCGGTTTGCC
GGTGTCATTCCTCTGTTACGGCCGAGTTTGTCTCATTCCACGCCT
GACACTCAGTTCCGGGTAGGCAGTTCGCTCCAAGCTGGACTGTAT
GCACGAACCCCCCGTTCAGTCCGACCGCTGCGCCTTATCCGGTAA
CTATCGTCTTGAGTCCAACCCGGAAAGACATGCAAAAGCACCACT
GGCAGCAGCCACTGGTAATTGATTTAGAGGAGTTAGTCTTGAAGT
CATGCGCCGGATAAGGCTAAACTGAAAGGACAAGTTTTGGCGACT
GCGCTCCTCCAAGCCAGTTACCTCGGTTCAAAGAGTTGGTAGCTC
AGAGAACCTTCGAAAAACCGCCCTGCAAGGCGGTTTTTTCGTTTT
CAGAGCAAGAGATTACGCGCAGACCAAAACGATCTCAAGAAGATC
ATCTTATTAATCAGATAAAATATTTCTAGATTTCAGTGCAATTTA
TCTCTTCAAATGTAGCACCGGCGCGCCGTGACCAATTATTGAAGG
CCGCTAACGCGGCCTTTTTTTGTTTCTGGTATCCCGAATGGAGCG
ACTTCTCCCCAAAAAGCCTCGCTTTCAGCACCTGTCGTTTCCTTT
CTTTTCAGAGGGTATTTTAAATAAAAACATTAAGTTATGACGAAG
AAGAACGGAAACGCCTTAAACCGGAAAATTTTCATAAATAGCGAA
AACCCGCGAGGTCGCCGCCCCGTAACCTGTCGGATCACCGGAAAG
GACCCGTAAAGTGATAATGATTATCATCTACATATCACAACGTGC
GTAAAGGGTAAGTATGAAGGTCGTGTACTCCATCGCTACCAAATT
CCAGAAAACAGACGCTTTCGAGCGTCTTTTTTCGTTTTGGTCACG
ACGTACGGTGGAAGATTCGTTACCAATTGACAGCTAGCTCAGTCC
TAGGTATATACATACATGCTTGTTTGTTTGTAAACTACTGTTTTC
ATTAAAGAGGAGAAAGGAAGCCATGTCCATCTATCAGGAGTTTGT
TAACAAGTATTCCCTGTCTAAAACCCTGCGTTTTGAACTGATCCC
GCAGGGCAAAACTTTGGAAAACATTAAAGCGCGTGGCCTGATTCT
GGATGACGAAAAACGTGCAAAGGATTACAAGAAAGCTAAACAGAT
CATCGACAAATATCACCAGTTCTTTATCGAAGAAATTCTGTCGTC
GGTGTGCATCAGTGAGGATCTGTTACAGAATTATTCTGATGTATA
CTTTAAACTTAAAAAGTCCGATGACGATAATCTGCAAAAAGATTT
CAAGTCAGCCAAAGATACCATCAAGAAACAGATCTCAGAATATAT
TAAAGATAGCGAAAAGTTCAAAAACCTGTTTAACCAAAACCTCAT
TGATGCTAAGAAAGGCCAAGAATCTGACCTGATCTTATGGCTGAA
ACAGAGCAAAGATAACGGCATTGAACTGTTCAAAGCTAATAGCGA
CATCACCGATATTGATGAAGCGCTCGAAATCATCAAGTCTTTCAA
AGGCTGGACGACGTATTTCAAAGGTTTTCATGAAAACCGTAAGAA
TGTATATTCGAGCAACGATATTCCGACCTCTATTATTTATCGTAT
CGTGGACGACAACCTGCCGAAGTTTCTGGAAAACAAAGCGAAATA
TGAATCTCTGAAAGACAAAGCACCGGAAGCTATTAACTATGAACA
GATCAAGAAAGATCTGGCGGAAGAACTGACCTTCGACATCGACTA
TAAAACCTCCGAAGTTAACCAGCGTGTTTTCTCACTGGACGAGGT
TTTCGAAATCGCTAATTTCAACAATTACCTGAATCAATCTGGCAT
CACCAAATTCAACACCATTATTGGTGGCAAATTTGTTAACGGCGA
AAACACCAAGCGTAAGGGCATCAACGAATACATTAACCTGTATAG
CCAACAAATCAACGACAAAACCCTGAAAAAGTATAAAATGTCCGT
TCTGTTTAAACAGATTTTATCGGACACCGAATCTAAATCCTTCGT
AATTGATAAACTGGAAGATGATAGCGACGTTGTCACCACGATGCA
GAGCTTTTATGAGCAGATTGCGGCGTTCAAAACCGTGGAAGAGAA
ATCTATTAAAGAAACTCTGTCCCTGCTCTTTGACGACCTCAAAGC
GCAGAAACTAGATCTGTCTAAGATTTACTTTAAAAACGACAAATC
TCTGACCGATCTCAGTCAACAAGTTTTCGATGACTATAGCGTGAT
CGGCACGGCAGTTTTGGAATACATCACCCAACAAATCGCGCCGAA
AAATCTGGACAACCCGTCCAAGAAGGAACAGGAACTGATTGCAAA
GAAAACAGAAAAAGCTAAATACCTGAGCTTAGAAACTATCAAACT
GGCACTTGAGGAATTTAATAAACATCGTGATATTGATAAACAGTG
TCGTTTTGAGGAAATTCTGGCGAACTTTGCGGCAATCCCGATGAT
CTTCGACGAAATTGCTCAAAACAAAGACAATCTGGCGCAGATCTC
TATCAAGTACCAGAATCAGGGTAAGAAAGATCTGCTTCAAGCATC
TGCGGAGGACGATGTGAAAGCAATTAAAGACTTATTAGATCAGAC
GAATAACTTATTACACAAGCTCAAAATCTTCCACATCAGCCAGAG
CGAGGACAAGGCGAACATTCTGGATAAAGATGAACACTTCTATCT
GGTGTTCGAAGAATGTTACTTCGAACTGGCAAACATCGTCCCTCT
CTACAATAAAATCCGCAACTACATCACGCAGAAGCCTTACTCTGA
CGAGAAATTCAAACTGAACTTCGAAAACAGCACGCTGGCGAACGG
CTGGGATAAGAACAAAGAGCCGGACAACACCGCAATCCTGTTCAT
CAAAGACGACAAATACTATCTGGGCGTAATGAACAAGAAGAACAA
CAAGATCTTCGACGATAAAGCGATCAAAGAAAACAAGGGTGAAGG
CTATAAGAAAATCGTGTACAAGCTCCTGCCGGGTGCGAATAAAAT
GTTACCGAAAGTGTTCTTTTCCGCGAAAAGCATCAAATTCTACAA
CCCGTCTGAGGATATTCTGCGCATCCGCAATCATAGCACGCACAC
TAAAAACGGTAGCCCGCAGAAAGGGTATGAAAAATTCGAATTTAA
TATAGAGGACTGCCGTAAGTTCATCGACTTCTATAAACAGAGCAT
TTCCAAACATCCGGAATGGAAAGACTTCGGCTTCCGTTTCTCTGA
CACTCAGCGCTATAATAGCATCGACGAGTTCTACCGCGAAGTGGA
GAATCAGGGCTATAAACTGACCTTCGAGAACATTAGTGAGTCGTA
CATCGACTCCGTTGTGAATCAGGGTAAACTGTACCTGTTTCAGAT
CTATAATAAAGACTTTAGCGCGTACAGCAAAGGCCGTCCGAATCT
GCACACCCTTTACTGGAAAGCATTATTTGACGAACGTAACCTGCA
AGATGTGGTGTATAAACTGAACGGTGAGGCGGAACTTTTCTACCG
TAAACAGAGTATCCCGAAGAAAATCACGCATCCGGCAAAAGAAGC
TATTGCCAACAAAAACAAAGACAACCCGAAGAAAGAATCAGTATT
CGAATATGACCTGATCAAAGATAAACGTTTCACCGAAGATAAGTT
CTTTTTCCACTGTCCGATTACCATCAACTTCAAATCTAGCGGTGC
GAACAAGTTCAACGATGAAATTAACTTATTACTGAAAGAGAAAGC
TAATGACGTACACATCTTATCTATTGATCGCGGTGAACGTCATTT
AGCATACTATACACTGGTAGATGGTAAAGGTAATATTATTAAACA
GGATACTTTCAATATTATCGGTAATGACCGTATGAAAACCAACTA
TCACGATAAGCTGGCGGCGATCGAAAAAGATCGTGATTCTGCGCG
TAAAGATTGGAAGAAAATTAACAATATCAAAGAAATGAAAGAAGG
CTATCTGAGCCAAGTGGTGCACGAGATCGCAAAACTGGTGATTGA
ATATAACGCTATCGTGGTTTTCGAAGATCTGAACTTTGGTTTTAA
ACGTGGTCGCTTCAAAGTAGAAAAACAGGTGTACCAAAAACTGGA
AAAAATGCTGATTGAAAAACTGAACTATCTGGTTTTTAAAGACAA
CGAATTTGACAAAACGGGTGGCGTACTCCGTGCCTATCAGCTGAC
CGCTCCGTTCGAAACGTTCAAGAAAATGGGTAAACAAACGGGGAT
TATCTATTATGTGCCAGCTGGTTTCACCTCCAAGATTTGTCCAGT
TACGGGCTTCGTTAACCAGCTGTACCCGAAATACGAGAGCGTTAG
CAAATCTCAAGAATTTTTCAGCAAATTCGACAAGATCTGCTATAA
TCTGGATAAAGGCTATTTCGAGTTCAGCTTCGATTACAAAAACTT
CGGCGATAAAGCGGCTAAAGGTAAGTGGACTATTGCTAGCTTTGG
TAGCCGTCTGATTAACTTTCGCAACTCCGACAAAAACCATAATTG
GGACACGCGTGAAGTGTATCCGACCAAAGAACTGGAAAAATTACT
GAAAGACTATTCCATCGAATATGGTCATGGGGAGTGCATTAAAGC
GGCGATTTGCGGTGAATCCGATAAGAAATTTTTCGCCAAACTGAC
CAGCGTGCTTAACACCATTCTGCAAATGCGTAATTCTAAAACGGG
TACGGAGCTGGACTACCTGATTTCTCCGGTAGCCGACGTTAACGG
CAACTTCTTCGATTCTCGTCAAGCACCGAAAAATATGCCACAAGA
CGCGGATGCCAACGGTGCATACCATATCGGCTTAAAAGGCTTAAT
GTTATTAGGCCGTATCAAGAATAATCAGGAGGGCAAGAAATTAAA
TCTGGTTATCAAAAACGAAGAATACTTCGAGTTCGTTCAGAATCG
TAACAATTAATGTATGCTTAAGCAGCTCGGTACCAAAGACGAACA
ATAAGACGCTGAAAAGCGTCTTTTTTCGTTTTGGTCCTGTTGCGG
CGCGATAGTGTGAACATGCTATAGACTTCTGGTGCTACCCGACTG
ACAATTAATCATCCGGCTCGTATAATGCTAGCAATTTCTACTGTT
GTAGATTCGCGGTGATGAACGCTTTCCAATTCGAGACGAACAATA
AGGCCTCCCTAACGGGGGGCCTTTTTTATTGATAACAAAAGTAAC
TTCGAGCTTGTCTACCTCCTAGCACCATTATTGCAATTAATAAAC
AACTAACGGACAATTCTACCTAACAGTTTTCATATATGACGAGCA
GTTAAGTGATGAGTAAAGGTGAGGAATTATTTACTGGTGTTGTTC
CGATCTTAGTTGAACTGGACGGCGATGTTAACGGTCATAAATTCA
GTGTTCGTGGTGAAGGTGAAGGTGATGCAACCAACGGTAAGCTGA
CCCTGAAATTCATCTGCACTACTGGAAAATTACCAGTACCGTGGC
CTACTCTGGTGACTACCCTGACCTATGGTGTTCAGTGTTTTTCTC
GTTACCCTGACCACATGAAGCAACATGATTTCTTCAAATCTGCAA
TGCCGGAAGGTTATGTACAGGAGCGCACCATTTCTTTCAAAGACG
ATGGCACGTATAAAACCCGTGCAGAGGTTAAATTTGAAGGTGACA
CTCTGGTGAATCGTATTGAACTGAAAGGCATTGATTTCAAAGAGG
ACGGCAATATTTTAGGCCACAAACTGGAATATAACTTCAACTCCC
ATAACGTTTACATCACCGCAGACAAACAGAAGAACGGTATCAAAG
CTAACTTCAAAATTCGCCATAACGTTGAAGATGGTAGCGTACAGC
TGGCGGATCATTACCAACAGAACACTCCGATTGGAGATGCTCCTG
TTTTACTGCCGGATAACCACTACCTGTCCACCCAGTCTAAACTGT
CGAAGGATCCGAACGAAAAGCGCGACCACATGGTGTTATTAGAGT
TCGTTACCGCTAGTGGTATCACGCACGGTATGGATGAACTCTACA
AATAAGACGAACAATAAGGGGAGCGGGAAACCGCTCCCCTTTTTT
ATTGATAACAAAAGTAAATTGCACGCTGATAGTCTCCCAATTGCG
AAGGACCAAAACGAAAAAACACCCTTTCGGGTGTCTTTTCTGGAA
TTTGGTACCGAGTACTAGGTATCGTGTAAGTAGCGAAGGCCCGTA
CGCGAGATAAACTGCTAGGCAACCGCGACTCTACGACTGGTGCTC
GATTTAATTTCGCTGACGTAAAGAAATTATCGGCAGTGCGTCAAC
TGCCGTATCTTTATCTTAATTAGGTAGTTGGACAAGCCCTTGAAA
GAAATAGCAAGAGCCTGCCTCTCTATTGAAGTCACGGCGAAAGTC
GGGTAGAAATCAAAGAAAGCAGAAATTAAATCGGAGTAACACTAA
GGTGGGATAACTCCGTAACTGACTACGCCTTTCTCTAGACTTTAC
TTGACCAGATACACTGTCTTTGACACGTTGAAGGATTAGAGCAAT
CAAATCCAAGACTGGCTAAGCACGAAGCAACTCTTGAGTGTTAAA
AAGTTATCTCCTGTATTCGGGAAGCGGGTACTAGAAGATTGCAGG
GACTCCGACGTTAAGTAAATTACAAAGTAATAAGTATCGTTCAGG
ATCACGTTACCGCAATAAGAAGCGAGAATAATATAATTTCCGAAG
TGCTTACCCCAGTAGTGACTATTCCTATAACCCTTCTGAGTGTCC
GGAGGCGGAAATTTGCCACGAAAGAGAAAGTATTTCCCCGACAAT
AATAAAGGGGCGCTCCTCAGCTTTTCCACTTGGTTGGGTAAGCTA
GGCAACTCTGAAAGGAGTTTCGGCGAATTGAAGCCGACAGCTTTG
AATTGTTTTAGGGGCGTTATTCGAGGGCAATCGGAGCTAACTTCA
AGACTACTTCTTTGTTGAATACTAAATAGTGCAAAGGTCGTGTTT
CCTCAAGGATACTCCGCTAACAATATAGGATTCCAATCAGATTCA
GCACTGGCGGTACGGGTGTTGCGGTGAGGCGTTCGGGTTTACGGC
TCGAAGCTAGCACGGTAGGAAGCCTGACAATCACCAAGCAAAAGG
GCCGTCGAAGGCCCACAAGATACGAAAGCTCTCGAAGCCTTATCC
TTGACCGATCCACCTATTTAGGCAGTTACGCACAAAAGCTACCCA
ATAATCCGTGACAGGCACAATATCACGGAACAAAACCGAAAACTC
TCGTACACGGTTAGGTTTTCGCTAGGAAGAATAAACCTCTATCTT
GATTATAAGAAGGCTCCCCAAGCACCCCCAAAACCGAAATAGCGG
TTTGCAATAAGGGACAAGTTACGAGTGTAGACACGCAGAATTATC
CAGCCTTTAGTCTTTAGGAAGGCAAAGCTATTGTACGCGGTAGCC
GTCGTAGCAATTTACCAACTGTAGAATTATTGGACACACGTAGGA
AGGGCTTACAGTTGAAGTTTAATAAGGTCACACGCAAAACCGCTA
AGGAATAATCGCACCGTTAGCGAAAGAATATTTCAGAGCGGTTAG
TAAAGGTTGAGTAAAGTGAGATTCCAAAGTGAGCCTTTATAAAAA
GTAAAGAGCTATAATAAAACCGTCGAGCAGAAAACAATCGCCTGA
AATCTCAAGCACGTTGCCCTTTCTAACGTCGCTAAGGTTTCGTAA
ACCCGTTTGATTAGGAAGAAGAATAAGTAACCCGATTAGGTTTGA
GATCGCGGGTTATCGGTTTGGATTAAAAGTGGATACCAGCGGAGT
CAACGCCGACGCAAACGTACAGTGATCCAATCCTGTTGCACGGTC
AAGCACAATCAGCTCGCAAGATCTTGGAATAGTGTGCCCAACAGT
TTAGTTGAGGGCCACGTTCCGACTACAAGTTGCTTCAAGAGGGGA
ATTTGGATTTGGCAATAGCCCCCCGTTTCTACCTCAAGAGGCGAC
GAGTATTAACCGCGCCAGCTGTCGGCACAAGGGCCAAAGAAGATT
CCAATTTCTTATTCCCGAATAACCTCCGAATCCCTGCGGGAAAAT
CACCGACCGAATAGCCTAGAAGCAAGGGGGAACAGATAGGTATAA
TTAGCTTAAGAGAGTACCAGCCGTGACAACAGCGTAGTAACCACA
AACTTACGCTGGGGCTTCTTTGGCGGATTTTTACAGATACTAACA
AGGTGATTTGAAGTACCTTAGTTGAGGATTTAAACGCGCTATCCG
GTAATCTCCAAATTGGGAAATACCGTTCAAAGAGGGCTAGAATTA
CTTAAAAGCCTTCACACCGCCTGCGCTATACGCGCCCACTCTCCC
GTTTATCCGTCCAAGCGGAAGCAGGGCGATCCTCCGCTAAGATAT
TCTTACGTGTAACGTAGCTAAGTATCCCAAATAGCTGGCGTACGC
GTTGAACACCGCCTAGAGGATCGTGACTCGCCGGACGAGCGTGTT
ATTGGGGACTTACGCCAGCGTAGACTACAACGCGCCCAGATTAAC
CCTGCACGTATTGCCTTGAATAACGTACTAATCTCTCCGGCTCTC
GACAATCTATCGAGCGACTCGATTATCAACGGGTGTCTTGCAGTT
CTAATCTCTTGCCCCCGCCCGTAATAGCCTCCAAGAGATTGAAGA
TAGTAAAGGGCAAGAGCTGATTCGGCGTTGAAGGATAGCGGACTT
TCGGTCAACCACAATTCCCCACTCGACAAAACCAGCCGTGCGAAT
AACTCTGAAAGTACAAGCAACCCAAGAGGGCTGAGCCTAAACTCA
GCTAATTCCTAAGTGAGCTAAAGACTCGAAGTGACAGCTCTTAAT
AAATAGAGCGGGAACGTCGAACGGTCGTGAAAGTAATAGTACAAC
GGGTATTAACTTACTGAGGATATTGCTTGAAGCTGTACCGTTTTA
TTGGGTGAACGAATAAGATCCAGCAATTCAGCCAAAGAAGCTACC
AATTTTTAGTTTAAGAGTGTCACGTCTGACCTCGCGGGTAGATTG
CCGAACGTAGAGCTTACGAGCCAGCGGAAACAGTAGCCGCAGGAT
AAGTAAGGGGAGTAAGTGATCGAACGAATCAGAAGTGACAATATA
CTTAGGCTGGATCTCGTCCCGTGAATCCCAACCCTCACCAACTAC
GAGATAAGAGGTAAGCCAAAAATCGACTTGGTGGCGACCAACGAC
TGTTCCCCCCCTGTAACTAATCGTTCCGTCAAAACCTGACTTACT
TCAAGGCCAATTCCAAGCGCAAACAATACCGTCCTAGTTCTTCGG
TTAAGTTTCCGAAGTAGGAGTGAGCCTACCTCCGTTTGCGTCTTG
TTACCACTGACCCAGCTATTTACTTTGTATTGCCTGCAATCGAAT
TTCTGAACTCTCAGATAGTGGGGATAACGGGAAAGTTCCTATATT
TGCGAACTAACTTAGCCGTCCACCTCGAAGCTACCTACTCACACC
CACCCCGCGCGGGGTAAATAAGGCACTAATCCCAGCTGAGAGCTG
GCGTAGCACTTAGCCACAAGTTAATTAACAGTTGTCTGGTAGTTT
GGCGGTATTAGGAAGATCCTAGAAGCAAGGCAGAGTTAGTTCTAA
CCTAAAGCCACAAATAAGACAGGTTGCCAAAGCCCGCCGGAAATT
AAATCTTGCTCAGTTCGGTAACGGAGTTTCCCTCCCGCGTACTTA
ATTCCCAATAAGAAACGCGCCCAAGTCCTATCAGGCAAAATTCAG
CCCCTTCCCGTGTTAGAACGAGGGTAAAAATACAAGCCGATTGAA
CAAGGGTTGGGGGCTTCAAATCGTCGTTTACCCCACTTTACAACG
GAGATTAAGTAGTTCACCCTATAGTACGAAGCAGAACTATTTCGA
GGGGCGTGCAATAATCGAATCTTCTGCGGTTGACTTAACACGCTA
GGGACGTGCCCTCGATTCAATCGAAGGTACTCCTACTCAGACTGC
CTCACACCCAGCTAGTCACTGAGCGATAAAATTGACCCGCCCTCT
AGGGAAGCGAGTACGTCCCAAAGGGCTCCGGACAGGGCTATATAG
GAGAGTTTGATCTCGCCCCGACAACTGCAACCCTCAACTCCCTTA
GATAATATTGTTAGCCGAAGTTGCACGACCCGCCGTCCACGGACT
GCTCTTAGGGTGTGGCTCCTTAATCTGACAACGTGCAACCCCTAT
CGAAGTCGATTGTTTCTGCGAAAGGTGTTGTCCTAATAGTCCCGA
AATTTGGCCCTTGTAGGTGTGAAACCACTTAGCTTCGCGCCGTAG
TCCTAAAGGCCCACCTATTGACTTTGTTTCGGGTAGCACTAGGAA
TCTTAACAATTTGAATTTGGACGTGGAACGCGTACACCTTAATCT
CCGAATAATTCTAGGGATTTGGAAGTCCTCTACGTTGACACACCT
ACACTGCTCGAAGTAAATATACGAATAACGCGGGCCTCGCGGAGC
CGTTCCGAATCGTCACGTGTTCGTTTACTGTTAATTGGTGGCAAA
TAAGCAATATCGTAGTCCGTCAGGCCCAGCCCTGTTATCCACGGC
GTTATTTGTCAAATTGCGTAGAACTGGATTGACTGCCTGACAATA
CCTAATTATCGGTACGAAGTCCCCGAATCTGTCGGGCTATTTCAC
TAATACTTTCCAAACGCCCCGTATCCAAGAAGAACGAATTTATCC
ACGCTCCCGTCTTTGGGACGAATACCGCTACAAGTGGACAGAGGA
TCGGTACGGGCCTCTAATAAATCCAACACTCTACGCCCTCTTCAA
GAGCTAGAAGAACAGGGTGCAGTTGGAAAGGGAATTATTTCGTAA
GGCGAGCCAATACCGTAATTAATTCGGAAGAGTTAACACGATTGG
AAGTAGGAATAGTTTCTAACCACGGTTACTAATCCTAATAACGGA
ACGCTGTCTGATAGATTAGTGTCAGCGCTCGGTACCAAAGAAAAA
TAAAAAGACGCTGAAAAGCGTCTTTTTATTTTTCGGTCCAGTGTA
ACTCAGGCAAAAGCACGTAATATTCGTACTTTCTTCCTCCGTAAG
CGTCACCCACATTCCTTAAAGAGTGCATGTGCATATTTTGTTATC
AATAAAAAAGGCCGCGATTTGCGGCCTTATTGTTCGTCTTGCCGG
ATTACGCCCCGCCCTGCCACTCATCGCAGTATTGTTGTAATTCAT
TAAGCATTCTGCCGACATGGAAGCCATCACAAACGGCATGATGAA
CTTGGATCGCCAGTGGCATTAACACCTTGTCGCCTTGCGTATAAT
ATTTTCCCATAGTGAAAACGGGGGCGAAGAAGTTGTCCATATTTG
CTACGTTTAAATCAAAACTGGTGAAACTCACCCACGGATTGGCAC
TGACGAAAAACATATTTTCGATAAACCCTTTAGGGAAATATGCTA
AGTTTTCACCGTAACACGCCACATCTTGACTATATATGTGTAGAA
ACTGCCGGAAATCGTCGTGGTATTCTGACCAGAGCGATGAAAACG
TTTCAGTTTGCTCATGGAAAACGGTGTAACAAGGGTGAACACTAT
CCCATATCACCAGCTCACCGTCTTTCATTGCCATACGAAACTCCG
GATGTGCATTCATCAGGCGGGCAAGAATGTGAATAAAGGCCGGAT
AAAACTTGTGCTTATTTTTCTTTACGGTTTTTAAAAAGGCCGTAA
TATCCAGCTGAACGGTTTGGTTATAGGTGCACTGAGCAACTGACT
GGAATGCCTCAAAATGTTCTTTACGATGCCATTGACTTATATCAA
CTGTAGTATATCCAGTGATTTTTTTCTCCATTTTAGCTTCCTTAG
CTTGCGAAATCTCGATAACTCAAAAAATAGTAGTGATCTTATTTC
ATTATGGTGAAAGTTGTCTTACGTGCAACATTTTCGCAAAAAGTT
GGCGCTTTATCAAC

p844: RBS1-AK272-19-CTX1/3-4nt

Payload encoding a Fn Cpf1 and an associated duplex of crRNA targeting different conserved regions of CTX-M gene variants (“CTX1”: GCCAGATCACCGCGATATCGTTGG, SEQ ID NO: 28; “CTX3”: TCGCGGTGATGAACGCTTTCCAAT, SEQ ID NO: 30).

(SEQ ID NO: 32)
ACTGTCGGAATGACAAATGGTTCCAATTATTGAACACCCTTCGGG
GTGTTTTTTTGTTTCTGGTTTCCCGAGGCCGGCCTGCGCTAGCGG
AGTGTATACTGGCTTACTATGTTGGCACTGATGAGGGTGTAAGTG
AAGTGCTTCATGTGGCAGGAGAAAAAAGGCTGCATCGGTGCGTCA
GCAGAATATGTGATACAGGATATATTCCGCTTCCTCGCTCACTGA
CTCGCTACGCTCGGTCGTTCGACTGTGGCGAGCGGAAATGGCTTA
CGAACGGGGCGGAGATTTCCTGGAAGATGCCAGGAAGATACTTAA
CAGGGAAGTGAGAGGGTCGCGGCAAAGCCGTTTTTCCATAGGCTC
CGCCCCCCTGACAAGCATCACGAAATCTGACGCTCAAATCAGTGG
TGGCGAAACCTGACAGGACTATAAAGATACCAGGCGTTTCCCCCT
GGCGGCTCCCTCGTGCGCTCTCCTGTTCCTGCCTTTCGGTTTGCC
GGTGTCATTCCTCTGTTACGGCCGAGTTTGTCTCATTCCACGCCT
GACACTCAGTTCCGGGTAGGCAGTTCGCTCCAAGCTGGACTGTAT
GCACGAACCCCCCGTTCAGTCCGACCGCTGCGCCTTATCCGGTAA
CTATCGTCTTGAGTCCAACCCGGAAAGACATGCAAAAGCACCACT
GGCAGCAGCCACTGGTAATTGATTTAGAGGAGTTAGTCTTGAAGT
CATGCGCCGGATAAGGCTAAACTGAAAGGACAAGTTTTGGCGACT
GCGCTCCTCCAAGCCAGTTACCTCGGTTCAAAGAGTTGGTAGCTC
AGAGAACCTTCGAAAAACCGCCCTGCAAGATCTTATTAATCAGAT
AAAATATTTCTAGATTTCAGTGCAATTTATCTCTTCAAATGTAGC
ACCGGCGCGCCGTGACCAATTATTGAAGGCCGCTAACGCGGCCTT
TTTTTGTTTCTGGTATCCCGAATGGAGCGACTTCTCCCCAAAAAG
CCTCGCTTTCAGCACCTGTCGTTTCCTTTCTTTTCAGAGGGTATT
TTAAATAAAAACATTAAGTTATGACGAAGAAGAACGGAAACGCCT
TAAACCGGAAAATTTTCATAAATAGCGAAAACCCGCGAGGTCGCC
GCCCCGTAACCTGTCGGATCACCGGAAAGGACCCGTAAAGTGATA
ATGATTATCATCTACATATCACAACGTGCGTAAAGGGTAAGTATG
AAGGTCGTGTACTCCATCGCTACCAAATTCCAGAAAACAGACGCT
TTCGAGCGTCTTTTTTCGTTTTGGTCACGACGTACGGTGGAAGAT
TCGTTACCAATTGACAGCTAGCTCAGTCCTAGGTATATACATACA
TGCTTGTTTGTTTGTAAACTACTGTTTTCATTAAAGAGGAGAAAG
GAAGCCATGTCCATCTATCAGGAGTTTGTTAACAAGTATTCCCTG
TCTAAAACCCTGCGTTTTGAACTGATCCCGCAGGGCAAAACTTTG
GAAAACATTAAAGCGCGTGGCCTGATTCTGGATGACGAAAAACGT
GCAAAGGATTACAAGAAAGCTAAACAGATCATCGACAAATATCAC
CAGTTCTTTATCGAAGAAATTCTGTCGTCGGTGTGCATCAGTGAG
GATCTGTTACAGAATTATTCTGATGTATACTTTAAACTTAAAAAG
TCCGATGACGATAATCTGCAAAAAGATTTCAAGTCAGCCAAAGAT
ACCATCAAGAAACAGATCTCAGAATATATTAAAGATAGCGAAAAG
TTCAAAAACCTGTTTAACCAAAACCTCATTGATGCTAAGAAAGGC
CAAGAATCTGACCTGATCTTATGGCTGAAACAGAGCAAAGATAAC
GGCATTGAACTGTTCAAAGCTAATAGCGACATCACCGATATTGAT
GAAGCGCTCGAAATCATCAAGTCTTTCAAAGGCTGGACGACGTAT
TTCAAAGGTTTTCATGAAAACCGTAAGAATGTATATTCGAGCAAC
GATATTCCGACCTCTATTATTTATCGTATCGTGGACGACAACCTG
CCGAAGTTTCTGGAAAACAAAGCGAAATATGAATCTCTGAAAGAC
AAAGCACCGGAAGCTATTAACTATGAACAGATCAAGAAAGATCTG
GCGGAAGAACTGACCTTCGACATCGACTATAAAACCTCCGAAGTT
AACCAGCGTGTTTTCTCACTGGACGAGGTTTTCGAAATCGCTAAT
TTCAACAATTACCTGAATCAATCTGGCATCACCAAATTCAACACC
ATTATTGGTGGCAAATTTGTTAACGGCGAAAACACCAAGCGTAAG
GGCATCAACGAATACATTAACCTGTATAGCCAACAAATCAACGAC
AAAACCCTGAAAAAGTATAAAATGTCCGTTCTGTTTAAACAGATT
TTATCGGACACCGAATCTAAATCCTTCGTAATTGATAAACTGGAA
GATGATAGCGACGTTGTCACCACGATGCAGAGCTTTTATGAGCAG
ATTGCGGCGTTCAAAACCGTGGAAGAGAAATCTATTAAAGAAACT
CTGTCCCTGCTCTTTGACGACCTCAAAGCGCAGAAACTAGATCTG
TCTAAGATTTACTTTAAAAACGACAAATCTCTGACCGATCTCAGT
CAACAAGTTTTCGATGACTATAGCGTGATCGGCACGGCAGTTTTG
GAATACATCACCCAACAAATCGCGCCGAAAAATCTGGACAACCCG
TCCAAGAAGGAACAGGAACTGATTGCAAAGAAAACAGAAAAAGCT
AAATACCTGAGCTTAGAAACTATCAAACTGGCACTTGAGGAATTT
AATAAACATCGTGATATTGATAAACAGTGTCGTTTTGAGGAAATT
CTGGCGAACTTTGCGGCAATCCCGATGATCTTCGACGAAATTGCT
CAAAACAAAGACAATCTGGCGCAGATCTCTATCAAGTACCAGAAT
CAGGGTAAGAAAGATCTGCTTCAAGCATCTGCGGAGGACGATGTG
AAAGCAATTAAAGACTTATTAGATCAGACGAATAACTTATTACAC
AAGCTCAAAATCTTCCACATCAGCCAGAGCGAGGACAAGGCGAAC
ATTCTGGATAAAGATGAACACTTCTATCTGGTGTTCGAAGAATGT
TACTTCGAACTGGCAAACATCGTCCCTCTCTACAATAAAATCCGC
AACTACATCACGCAGAAGCCTTACTCTGACGAGAAATTCAAACTG
AACTTCGAAAACAGCACGCTGGCGAACGGCTGGGATAAGAACAAA
GAGCCGGACAACACCGCAATCCTGTTCATCAAAGACGACAAATAC
TATCTGGGCGTAATGAACAAGAAGAACAACAAGATCTTCGACGAT
AAAGCGATCAAAGAAAACAAGGGTGAAGGCTATAAGAAAATCGTG
TACAAGCTCCTGCCGGGTGCGAATAAAATGTTACCGAAAGTGTTC
TTTTCCGCGAAAAGCATCAAATTCTACAACCCGTCTGAGGATATT
CTGCGCATCCGCAATCATAGCACGCACACTAAAAACGGTAGCCCG
CAGAAAGGGTATGAAAAATTCGAATTTAATATAGAGGACTGCCGT
AAGTTCATCGACTTCTATAAACAGAGCATTTCCAAACATCCGGAA
TGGAAAGACTTCGGCTTCCGTTTCTCTGACACTCAGCGCTATAAT
AGCATCGACGAGTTCTACCGCGAAGTGGAGAATCAGGGCTATAAA
CTGACCTTCGAGAACATTAGTGAGTCGTACATCGACTCCGTTGTG
AATCAGGGTAAACTGTACCTGTTTCAGATCTATAATAAAGACTTT
AGCGCGTACAGCAAAGGCCGTCCGAATCTGCACACCCTTTACTGG
AAAGCATTATTTGACGAACGTAACCTGCAAGATGTGGTGTATAAA
CTGAACGGTGAGGCGGAACTTTTCTACCGTAAACAGAGTATCCCG
AAGAAAATCACGCATCCGGCAAAAGAAGCTATTGCCAACAAAAAC
AAAGACAACCCGAAGAAAGAATCAGTATTCGAATATGACCTGATC
AAAGATAAACGTTTCACCGAAGATAAGTTCTTTTTCCACTGTCCG
ATTACCATCAACTTCAAATCTAGCGGTGCGAACAAGTTCAACGAT
GAAATTAACTTATTACTGAAAGAGAAAGCTAATGACGTACACATC
TTATCTATTGATCGCGGTGAACGTCATTTAGCATACTATACACTG
GTAGATGGTAAAGGTAATATTATTAAACAGGATACTTTCAATATT
ATCGGTAATGACCGTATGAAAACCAACTATCACGATAAGCTGGCG
GCGATCGAAAAAGATCGTGATTCTGCGCGTAAAGATTGGAAGAAA
ATTAACAATATCAAAGAAATGAAAGAAGGCTATCTGAGCCAAGTG
GTGCACGAGATCGCAAAACTGGTGATTGAATATAACGCTATCGTG
GTTTTCGAAGATCTGAACTTTGGTTTTAAACGTGGTCGCTTCAAA
GTAGAAAAACAGGTGTACCAAAAACTGGAAAAAATGCTGATTGAA
AAACTGAACTATCTGGTTTTTAAAGACAACGAATTTGACAAAACG
GGTGGCGTACTCCGTGCCTATCAGCTGACCGCTCCGTTCGAAACG
TTCAAGAAAATGGGTAAACAAACGGGGATTATCTATTATGTGCCA
GCTGGTTTCACCTCCAAGATTTGTCCAGTTACGGGCTTCGTTAAC
CAGCTGTACCCGAAATACGAGAGCGTTAGCAAATCTCAAGAATTT
TTCAGCAAATTCGACAAGATCTGCTATAATCTGGATAAAGGCTAT
TTCGAGTTCAGCTTCGATTACAAAAACTTCGGCGATAAAGCGGCT
AAAGGTAAGTGGACTATTGCTAGCTTTGGTAGCCGTCTGATTAAC
TTTCGCAACTCCGACAAAAACCATAATTGGGACACGCGTGAAGTG
TATCCGACCAAAGAACTGGAAAAATTACTGAAAGACTATTCCATC
GAATATGGTCATGGGGAGTGCATTAAAGCGGCGATTTGCGGTGAA
TCCGATAAGAAATTTTTCGCCAAACTGACCAGCGTGCTTAACACC
ATTCTGCAAATGCGTAATTCTAAAACGGGTACGGAGCTGGACTAC
CTGATTTCTCCGGTAGCCGACGTTAACGGCAACTTCTTCGATTCT
CGTCAAGCACCGAAAAATATGCCACAAGACGCGGATGCCAACGGT
GCATACCATATCGGCTTAAAAGGCTTAATGTTATTAGGCCGTATC
AAGAATAATCAGGAGGGCAAGAAATTAAATCTGGTTATCAAAAAC
GAAGAATACTTCGAGTTCGTTCAGAATCGTAACAATTAATGTATG
CTTAAGCAGCTCGGTACCAAAGACGAACAATAAGACGCTGAAAAG
CGTCTTTTTTCGTTTTGGTCCTGTTGCGGCGCGATAGTGTGAACA
TGCTATAGACTTCTGGTGCTACCCGACTGACAATTAATCATCCGG
CTCGTATAATGCTAGCAATTTCTACTGTTGTAGATGCCAGATCAC
CGCGATATCGTTGGAATTTCTACTGTTGTAGATTCGCGGTGATGA
ACGCTTTCCAATTCGAGACGAACAATAAGGCCTCCCTAACGGGGG
GCCTTTTTTATTGATAACAAAAGTAACTTCGAGCTTGTCTACCTC
CTAGCACCATTATTGCAATTAATAAACAACTAACGGACAATTCTA
CCTAACAGTTTTCATATATGACGAGCAGTTAAGTGATGAGTAAAG
GTGAGGAATTATTTACTGGTGTTGTTCCGATCTTAGTTGAACTGG
ACGGCGATGTTAACGGTCATAAATTCAGTGTTCGTGGTGAAGGTG
AAGGTGATGCAACCAACGGTAAGCTGACCCTGAAATTCATCTGCA
CTACTGGAAAATTACCAGTACCGTGGCCTACTCTGGTGACTACCC
TGACCTATGGTGTTCAGTGTTTTTCTCGTTACCCTGACCACATGA
AGCAACATGATTTCTTCAAATCTGCAATGCCGGAAGGTTATGTAC
AGGAGCGCACCATTTCTTTCAAAGACGATGGCACGTATAAAACCC
GTGCAGAGGTTAAATTTGAAGGTGACACTCTGGTGAATCGTATTG
AACTGAAAGGCATTGATTTCAAAGAGGACGGCAATATTTTAGGCC
ACAAACTGGAATATAACTTCAACTCCCATAACGTTTACATCACCG
CAGACAAACAGAAGAACGGTATCAAAGCTAACTTCAAAATTCGCC
ATAACGTTGAAGATGGTAGCGTACAGCTGGCGGATCATTACCAAC
AGAACACTCCGATTGGAGATGCTCCTGTTTTACTGCCGGATAACC
ACTACCTGTCCACCCAGTCTAAACTGTCGAAGGATCCGAACGAAA
AGCGCGACCACATGGTGTTATTAGAGTTCGTTACCGCTAGTGGTA
TCACGCACGGTATGGATGAACTCTACAAATAAGACGAACAATAAG
GGGAGCGGGAAACCGCTCCCCTTTTTTATTGATAACAAAAGTAAA
TTGCACGCTGATAGTCTCCCAATTGCGAAGGACCAAAACGAAAAA
ACACCCTTTCGGGTGTCTTTTCTGGAATTTGGTACCGAGTACTAG
GTATCGTGTAAGTAGCGAAGGCCCGTACGCGAGATAAACTGCTAG
GCAACCGCGACTCTACGACTGGTGCTCGATTTAATTTCGCTGACG
TAAAGAAATTATCGGCAGTGCGTCAACTGCCGTATCTTTATCTTA
ATTAGGTAGTTGGACAAGCCCTTGAAAGAAATAGCAAGAGCCTGC
CTCTCTATTGAAGTCACGGCGAAAGTCGGGTAGAAATCAAAGAAA
GCAGAAATTAAATCGGAGTAACACTAAGGTGGGATAACTCCGTAA
CTGACTACGCCTTTCTCTAGACTTTACTTGACCAGATACACTGTC
TTTGACACGTTGAAGGATTAGAGCAATCAAATCCAAGACTGGCTA
AGCACGAAGCAACTCTTGAGTGTTAAAAAGTTATCTCCTGTATTC
GGGAAGCGGGTACTAGAAGATTGCAGGGACTCCGACGTTAAGTAA
ATTACAAAGTAATAAGTATCGTTCAGGATCACGTTACCGCAATAA
GAAGCGAGAATAATATAATTTCCGAAGTGCTTACCCCAGTAGTGA
CTATTCCTATAACCCTTCTGAGTGTCCGGAGGCGGAAATTTGCCA
CGAAAGAGAAAGTATTTCCCCGACAATAATAAAGGGGCGCTCCTC
AGCTTTTCCACTTGGTTGGGTAAGCTAGGCAACTCTGAAAGGAGT
TTCGGCGAATTGAAGCCGACAGCTTTGAATTGTTTTAGGGGCGTT
ATTCGAGGGCAATCGGAGCTAACTTCAAGACTACTTCTTTGTTGA
ATACTAAATAGTGCAAAGGTCGTGTTTCCTCAAGGATACTCCGCT
AACAATATAGGATTCCAATCAGATTCAGCACTGGCGGTACGGGTG
TTGCGGTGAGGCGTTCGGGTTTACGGCTCGAAGCTAGCACGGTAG
GAAGCCTGACAATCACCAAGCAAAAGGGCCGTCGAAGGCCCACAA
GATACGAAAGCTCTCGAAGCCTTATCCTTGACCGATCCACCTATT
TAGGCAGTTACGCACAAAAGCTACCCAATAATCCGTGACAGGCAC
AATATCACGGAACAAAACCGAAAACTCTCGTACACGGTTAGGTTT
TCGCTAGGAAGAATAAACCTCTATCTTGATTATAAGAAGGCTCCC
CAAGCACCCCCAAAACCGAAATAGCGGTTTGCAATAAGGGACAAG
TTACGAGTGTAGACACGCAGAATTATCCAGCCTTTAGTCTTTAGG
AAGGCAAAGCTATTGTACGCGGTAGCCGTCGTAGCAATTTACCAA
CTGTAGAATTATTGGACACACGTAGGAAGGGCTTACAGTTGAAGT
TTAATAAGGTCACACGCAAAACCGCTAAGGAATAATCGCACCGTT
AGCGAAAGAATATTTCAGAGCGGTTAGTAAAGGTTGAGTAAAGTG
AGATTCCAAAGTGAGCCTTTATAAAAAGTAAAGAGCTATAATAAA
ACCGTCGAGCAGAAAACAATCGCCTGAAATCTCAAGCACGTTGCC
CTTTCTAACGTCGCTAAGGTTTCGTAAACCCGTTTGATTAGGAAG
AAGAATAAGTAACCCGATTAGGTTTGAGATCGCGGGTTATCGGTT
TGGATTAAAAGTGGATACCAGCGGAGTCAACGCCGACGCAAACGT
ACAGTGATCCAATCCTGTTGCACGGTCAAGCACAATCAGCTCGCA
AGATCTTGGAATAGTGTGCCCAACAGTTTAGTTGAGGGCCACGTT
CCGACTACAAGTTGCTTCAAGAGGGGAATTTGGATTTGGCAATAG
CCCCCCGTTTCTACCTCAAGAGGCGACGAGTATTAACCGCGCCAG
CTGTCGGCACAAGGGCCAAAGAAGATTCCAATTTCTTATTCCCGA
ATAACCTCCGAATCCCTGCGGGAAAATCACCGACCGAATAGCCTA
GAAGCAAGGGGGAACAGATAGGTATAATTAGCTTAAGAGAGTACC
AGCCGTGACAACAGCGTAGTAACCACAAACTTACGCTGGGGCTTC
TTTGGCGGATTTTTACAGATACTAACAAGGTGATTTGAAGTACCT
TAGTTGAGGATTTAAACGCGCTATCCGGTAATCTCCAAATTGGGA
AATACCGTTCAAAGAGGGCTAGAATTACTTAAAAGCCTTCACACC
GCCTGCGCTATACGCGCCCACTCTCCCGTTTATCCGTCCAAGCGG
AAGCAGGGCGATCCTCCGCTAAGATATTCTTACGTGTAACGTAGC
TAAGTATCCCAAATAGCTGGCGTACGCGTTGAACACCGCCTAGAG
GATCGTGACTCGCCGGACGAGCGTGTTATTGGGGACTTACGCCAG
CGTAGACTACAACGCGCCCAGATTAACCCTGCACGTATTGCCTTG
AATAACGTACTAATCTCTCCGGCTCTCGACAATCTATCGAGCGAC
TCGATTATCAACGGGTGTCTTGCAGTTCTAATCTCTTGCCCCCGC
CCGTAATAGCCTCCAAGAGATTGAAGATAGTAAAGGGCAAGAGCT
GATTCGGCGTTGAAGGATAGCGGACTTTCGGTCAACCACAATTCC
CCACTCGACAAAACCAGCCGTGCGAATAACTCTGAAAGTACAAGC
AACCCAAGAGGGCTGAGCCTAAACTCAGCTAATTCCTAAGTGAGC
TAAAGACTCGAAGTGACAGCTCTTAATAAATAGAGCGGGAACGTC
GAACGGTCGTGAAAGTAATAGTACAACGGGTATTAACTTACTGAG
GATATTGCTTGAAGCTGTACCGTTTTATTGGGTGAACGAATAAGA
TCCAGCAATTCAGCCAAAGAAGCTACCAATTTTTAGTTTAAGAGT
GTCACGTCTGACCTCGCGGGTAGATTGCCGAACGTAGAGCTTACG
AGCCAGCGGAAACAGTAGCCGCAGGATAAGTAAGGGGAGTAAGTG
ATCGAACGAATCAGAAGTGACAATATACTTAGGCTGGATCTCGTC
CCGTGAATCCCAACCCTCACCAACTACGAGATAAGAGGTAAGCCA
AAAATCGACTTGGTGGCGACCAACGACTGTTCCCCCCCTGTAACT
AATCGTTCCGTCAAAACCTGACTTACTTCAAGGCCAATTCCAAGC
GCAAACAATACCGTCCTAGTTCTTCGGTTAAGTTTCCGAAGTAGG
AGTGAGCCTACCTCCGTTTGCGTCTTGTTACCACTGACCCAGCTA
TTTACTTTGTATTGCCTGCAATCGAATTTCTGAACTCTCAGATAG
TGGGGATAACGGGAAAGTTCCTATATTTGCGAACTAACTTAGCCG
TCCACCTCGAAGCTACCTACTCACACCCACCCCGCGCGGGGTAAA
TAAGGCACTAATCCCAGCTGAGAGCTGGCGTAGCACTTAGCCACA
AGTTAATTAACAGTTGTCTGGTAGTTTGGCGGTATTAGGAAGATC
CTAGAAGCAAGGCAGAGTTAGTTCTAACCTAAAGCCACAAATAAG
ACAGGTTGCCAAAGCCCGCCGGAAATTAAATCTTGCTCAGTTCGG
TAACGGAGTTTCCCTCCCGCGTACTTAATTCCCAATAAGAAACGC
GCCCAAGTCCTATCAGGCAAAATTCAGCCCCTTCCCGTGTTAGAA
CGAGGGTAAAAATACAAGCCGATTGAACAAGGGTTGGGGGCTTCA
AATCGTCGTTTACCCCACTTTACAACGGAGATTAAGTAGTTCACC
CTATAGTACGAAGCAGAACTATTTCGAGGGGCGTGCAATAATCGA
ATCTTCTGCGGTTGACTTAACACGCTAGGGACGTGCCCTCGATTC
AATCGAAGGTACTCCTACTCAGACTGCCTCACACCCAGCTAGTCA
CTGAGCGATAAAATTGACCCGCCCTCTAGGGAAGCGAGTACGTCC
CAAAGGGCTCCGGACAGGGCTATATAGGAGAGTTTGATCTCGCCC
CGACAACTGCAACCCTCAACTCCCTTAGATAATATTGTTAGCCGA
AGTTGCACGACCCGCCGTCCACGGACTGCTCTTAGGGTGTGGCTC
CTTAATCTGACAACGTGCAACCCCTATCGAAGTCGATTGTTTCTG
CGAAAGGTGTTGTCCTAATAGTCCCGAAATTTGGCCCTTGTAGGT
GTGAAACCACTTAGCTTCGCGCCGTAGTCCTAAAGGCCCACCTAT
TGACTTTGTTTCGGGTAGCACTAGGAATCTTAACAATTTGAATTT
GGACGTGGAACGCGTACACCTTAATCTCCGAATAATTCTAGGGAT
TTGGAAGTCCTCTACGTTGACACACCTACACTGCTCGAAGTAAAT
ATACGAATAACGCGGGCCTCGCGGAGCCGTTCCGAATCGTCACGT
GTTCGTTTACTGTTAATTGGTGGCAAATAAGCAATATCGTAGTCC
GTCAGGCCCAGCCCTGTTATCCACGGCGTTATTTGTCAAATTGCG
TAGAACTGGATTGACTGCCTGACAATACCTAATTATCGGTACGAA
GTCCCCGAATCTGTCGGGCTATTTCACTAATACTTTCCAAACGCC
CCGTATCCAAGAAGAACGAATTTATCCACGCTCCCGTCTTTGGGA
CGAATACCGCTACAAGTGGACAGAGGATCGGTACGGGCCTCTAAT
AAATCCAACACTCTACGCCCTCTTCAAGAGCTAGAAGAACAGGGT
GCAGTTGGAAAGGGAATTATTTCGTAAGGCGAGCCAATACCGTAA
TTAATTCGGAAGAGTTAACACGATTGGAAGTAGGAATAGTTTCTA
ACCACGGTTACTAATCCTAATAACGGAACGCTGTCTGATAGATTA
GTGTCAGCGCTCGGTACCAAAGAAAAATAAAAAGACGCTGAAAAG
CGTCTTTTTATTTTTCGGTCCAGTGTAACTCAGGCAAAAGCACGT
AATATTCGTACTTTCTTCCTCCGTAAGCGTCACCCACATTCCTTA
AAGAGTGCATGTGCATATTTTGTTATCAATAAAAAAGGCCGCGAT
TTGCGGCCTTATTGTTCGTCTTGCCGGATTACGCCCCGCCCTGCC
ACTCATCGCAGTATTGTTGTAATTCATTAAGCATTCTGCCGACAT
GGAAGCCATCACAAACGGCATGATGAACTTGGATCGCCAGTGGCA
TTAACACCTTGTCGCCTTGCGTATAATATTTTCCCATAGTGAAAA
CGGGGGCGAAGAAGTTGTCCATATTTGCTACGTTTAAATCAAAAC
TGGTGAAACTCACCCACGGATTGGCACTGACGAAAAACATATTTT
CGATAAACCCTTTAGGGAAATATGCTAAGTTTTCACCGTAACACG
CCACATCTTGACTATATATGTGTAGAAACTGCCGGAAATCGTCGT
GGTATTCTGACCAGAGCGATGAAAACGTTTCAGTTTGCTCATGGA
AAACGGTGTAACAAGGGTGAACACTATCCCATATCACCAGCTCAC
CGTCTTTCATTGCCATACGAAACTCCGGATGTGCATTCATCAGGC
GGGCAAGAATGTGAATAAAGGCCGGATAAAACTTGTGCTTATTTT
TCTTTACGGTTTTTAAAAAGGCCGTAATATCCAGCTGAACGGTTT
GGTTATAGGTGCACTGAGCAACTGACTGGAATGCCTCAAAATGTT
CTTTACGATGCCATTGACTTATATCAACTGTAGTATATCCAGTGA
TTTTTTTCTCCATTTTAGCTTCCTTAGCTTGCGAAATCTCGATAA
CTCAAAAAATAGTAGTGATCTTATTTCATTATGGTGAAAGTTGTC
TTACGTGCAACATTTTCGCAAAAAGTTGGCGCTTTATCAAC.

### Example 2: In Vitro ESBL Elimination Assay

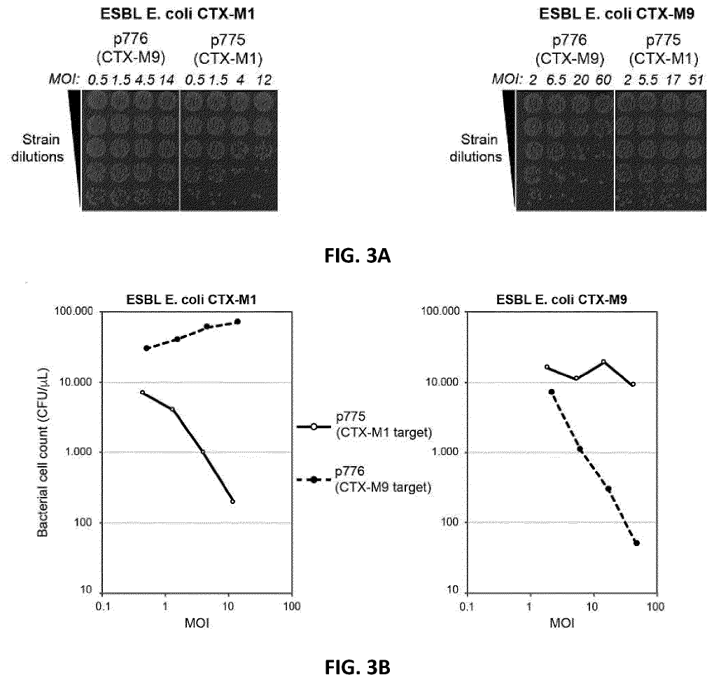

Sequence specific elimination of clinical strains of ESBL E. coli using packaged phagemids. Two clinical E. coli strains carrying different ESBL gene variants (CTX-M1 and CTX-M9) were transduced with packaged phagemids targeting either CTX-M1 or CTX-M and comprising a chloramphenicol resistance gene. Strains were then plated on LB agar supplemented with chloramphenicol at different Multiplicity Of Infection (MOI). Results of a spot assay and associated cell counts are shown in FIG. 3.

### Example 3: In Vivo ESBL Elimination Assay

This example shows the efficient elimination of ESBL E. coli strains using packaged phagemids of the invention.

Materials and Methods

Groups of streptomycin-treated mice were orally administered with one of four ESBL E. coli strains (further made resistant to streptomycin for mice colonization purpose), namely strains s17367 (herein referred to Target strain #1), s20545 (herein referred to Target strain #2), s20546 (herein referred to Target strain #3) and s20547 (herein referred to Target strain #4), all carrying the CTX-M1 gene, to establish a durable intestinal colonization with these bacterial strains for several days.

Packaged phagemids comprising a payload encoding a Fn Cpf1 nuclease and a ctx1 guide (as disclosed in Example 1) were produced as disclosed in Example 1, within a bacterial delivery vehicle comprising an 1A2 gpJ protein, as disclosed in Example 1, and a STF29 protein of sequence SEQ ID NO: 4 (encoded by nucleic acid of sequence SEQ ID NO: 5, and produced with its chaperone protein of sequence SEQ ID NO: 6, encoded by a nucleic acid of sequence SEQ ID NO: 7).

Colonized mice were then given orally 100 p1 of packaged phagemids (approximately 2×10¹² particles) along with 100 p1 of a buffer (sucrose and bicarbonate in water) in order to temporarily neutralize the gastric pH.

The bacterial colonization levels were measured non-invasively by plating dilutions of stool recovered from each animal individually onto agar plates. These levels were compared before treatment was initiated (termed ‘T0’) and 8 hours after the treatment (termed ‘T8h’) for each animal, and the median for each group of mice was graphed.

Results

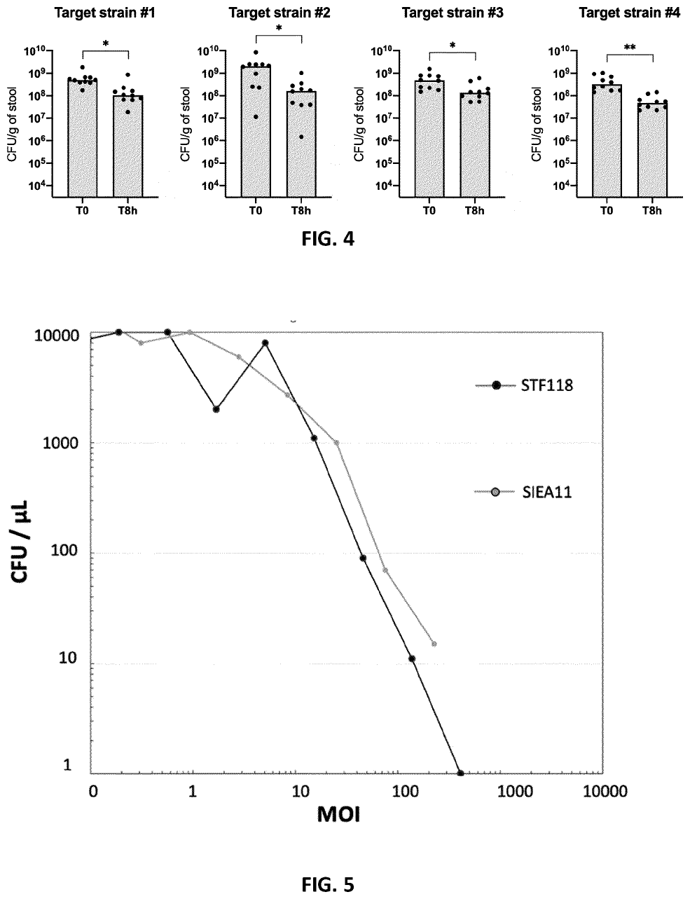

As shown on FIG. 4, the packaged phagemids caused a statistically significant reduction in bacterial burden recovered from the stool 8 hours after oral administration for all four tested ESBL E. coli strains. The average reduction in bacterial colonization was between 0.5 and 1.0 log₁₀.

### Example 4: In Vitro Sequence-Specific Killing of Klebsiella pneumoniae Using Lambda-Derived Particles

The Klebsiella pneumoniae strain 166A1, belonging to MLST type 45, was selected as a candidate strain for in vitro sequence-specific nuclease-mediated killing assays. The strain 166A1 contains a bla-oxa-48 gene and the nuclease-containing payload was programmed to target a region within this gene (SEQ ID NO: 9). Two Lambda-derived particles engineered to contain a modified gpJ (A8, SEQ ID NO: 10) and either chimeric STF118 (SEQ ID NO: 11, SEQ ID NO: 12, SEQ ID NO: 13 and SEQ ID NO: 14) or chimeric STF SIEA11 (SEQ ID NO: 15, SEQ ID NO: 16, SEQ ID NO: 17 and SEQ ID NO: 18) were packaged with a payload containing a nuclease programmed to target the bla-oxa-48 gene (SEQ ID NO: 19) following the production method described in Example 1.

The K. pneumoniae 166A1 strain was grown overnight in LB supplemented with 5 mM CaCl₂) at 37° C. with shaking. Next day, the culture was diluted 1:100 in fresh LB supplemented with 5 mM CaCl₂) and grown for 2 hours at 37° C. with shaking. The culture OD600 was then measured and adjusted to OD 0.025 using fresh LB supplemented with 5 mM CaCl₂).

The phagemids were serially diluted 3-fold and 10 μL of each dilution added to a 96-well plate horizontally, to which 90 μL of the cell culture adjusted to OD600 0.025 were then added. The plate was then incubated for 30 min at 37° C. After that, each transduction well was serially diluted 10 fold vertically, 10 μL of each dilution spotted on an LB-agar plate and incubated at 37° C. for 18 h. Next day, the cell count of each dilution was determined by counting the CFUs and plotted against the MOI (FIG. 5).

As can be seen from FIG. 5, Lambda particles, originally derived from an E. coli-specific phage, can be engineered to efficiently target Klebsiella pneumoniae and mediate 2-logs of sequence-specific killing at MOIs <50.

### REFERENCES

Bode, L., Kluytmans, J., Wertheim, H., Bogaers, D., Vandenbroucke-Grauls, C., Roosendaal, R., Troelstra, A., Box, A., Voss, A., Tweel, I., Belkum, A., Verbrugh, H., Vos, M. (2010). Preventing Surgical-Site Infections in Nasal Carriers of Staphylococcus aureus New England Journal of Medicine 362(1), 9-17.
Jonge, P., Nobrega, F., Brouns, S., Dutilh, B. (2018). Molecular and Evolutionary Determinants of Bacteriophage Host Range Trends in Microbiology.
Gargiullo, L., Chierico, F., D'Argenio, P., Putignani, L. (2019). Gut Microbiota Modulation for Multidrug-Resistant Organism Decolonization: Present and Future Perspectives Frontiers in Microbiology 10( ), 1704.
Kokai-Kun, J., Walsh, S., Chanturiya, T., Mond, J. (2003). Lysostaphin Cream Eradicates Staphylococcus aureus Nasal Colonization in a Cotton Rat Model Antimicrobial Agents and Chemotherapy 47(5), 1589-1597.
Lambelet P, Tascini C, Fortunato S, Stefanelli A, Simonetti F, Vettori C, Leonildi A, Menichetti F. (2017) Oral gentamicin therapy for Carbapenem-resistant Klebsiella pneumoniae gut colonization in hematologic patients: a single center experience. New Microbiol. 40(3):161-164.
Manley, K., Fraenkel, M., Mayall, B., Power, D. (2007). Probiotic treatment of vancomycin-resistant enterococci: a randomised controlled trial Medical Journal of Australia 186(9), 454-457.
Millan, A. (2018). Evolution of Plasmid-Mediated Antibiotic Resistance in the Clinical Context Trends in Microbiology.
Pastagia, M., Euler, C., Chahales, P., Fuentes-Duculan, J., Krueger, J., Fischetti, V. (2011). A Novel Chimeric Lysin Shows Superiority to Mupirocin for Skin Decolonization of Methicillin-Resistant and -Sensitive Staphylococcus aureus Strains Antimicrobial Agents and Chemotherapy 55(2), 738-744.
Perl, T., Cullen, J., Wenzel, R., Zimmerman, M., Pfaller, M., Sheppard, D., Twombley, J., French, P., Herwaldt, L., Team, M. (2002). Intranasal Mupirocin to Prevent Postoperative Staphylococcus aureus Infections New England Journal of Medicine 346(24), 1871-1877.
Rieg, S., Kupper, F., With, K., Serr, A., Bohnert, J., Kern, W. (2015). Intestinal decolonization of Enterobacteriaceae producing extended-spectrum β-lactamases (ESBL): a retrospective observational study in patients at risk for infection and a brief review of the literature BMC Infectious Diseases 15(1), 475.
Sakr, A., Bregeon, F., Rolain, J., Blin, 0. (2019). Staphylococcus aureus nasal decolonization strategies: a review Expert Review of Anti-infective Therapy
Saha, S., Tariq, R., Tosh, P., Pardi, D., Khanna, S. (2019). Fecal Microbiota Transplantation for Eradicating Carriage of Multidrug-Resistant Organisms: A Systematic Review Clinical Microbiology and Infection 25(8), 958-963.
Salomão, M., Heluany-Filho, M., Menegueti, M., Kraker, M., Martinez, R., Bellissimo-Rodrigues, F. (2016). A randomized clinical trial on the effectiveness of a symbiotic product to decolonize patients harboring multidrug-resistant Gram-negative bacilli. Revista da Sociedade Brasileira de Medicina Tropical 49(5), 559-566.
Satlin M J, Chavda K D, Baker T M, Chen L, Shashkina E, Soave R, Small C B, Jacobs S E, Shore T B, van Besien K, Westblade L F, Schuetz A N, Fowler V G Jr, Jenkins S G, Walsh T J, Kreiswirth B N. (2018) Colonization With Levofloxacin-resistant Extended-spectrum β-Lactamase-producing Enterobacteriaceae and Risk of Bacteremia in Hematopoietic Stem Cell Transplant Recipients. Clin Infect Dis. 67(11):1720-1728.
Szachta, P., Ignyś, I., Cichy, W. (2011). An evaluation of the ability of the probiotic strain Lactobacillus rhamnosus GG to eliminate the gastrointestinal carrier state of vancomycin-resistant enterococci in colonized children. Journal of clinical gastroenterology 45(10), 872-7.
Tannock, G., Tiong, I., Priest, P., Munro, K., Taylor, C., Richardson, A., Schultz, M. (2010). Testing probiotic strain Escherichia coli Nissle 1917 (Mutaflor) for its ability to reduce carriage of multidrug-resistant E. coli by elderly residents in long-term care facilities. Journal of medical microbiology 60(Pt 3), 366-70.
Walker, E., Vasquez, J., Dula, R., & Bullock, H. (2003). Mupirocin-Resistant, Methicillin-Resistant Staphylococcus aureus: Does Mupirocin Remain Effective? Infection Control & Hospital Epidemiology, Volume 24, Issue 5, May 2003, pp. 342-346.

1-17. (canceled)

18. A method of selectively removing antibiotic resistance from an antibiotic resistant bacterial strain(s) in a healthy subject carrying antibiotic resistant bacterial strain(s) or in a patient carrying antibiotic resistant bacterial strain(s), wherein the method comprises administering to the subject or patient at least one vector or recombinant phage,
said vector encoding a DNA modifying enzyme inactivating the antibiotic resistance gene(s) responsible for antibiotic-resistance of bacterial strain(s) or said recombinant phage comprising a nucleic acid encoding a DNA modifying enzyme inactivating the antibiotic resistance gene(s) responsible for antibiotic-resistance of bacterial strain(s),
thereby selectively removing antibiotic resistance from an antibiotic resistant bacterial strain(s) in the microbiota of said subject or patient.

19. A method of preventing antibiotic resistance related infection in a healthy subject carrying antibiotic resistant bacterial strain(s) or in a patient carrying antibiotic resistant bacterial strain(s), wherein the method comprises administering to the subject or patient at least one vector or recombinant phage,
said vector encoding a DNA modifying enzyme inactivating the antibiotic resistance gene(s) responsible for antibiotic-resistance of bacterial strain(s) or said recombinant phage comprising a nucleic acid encoding a DNA modifying enzyme inactivating the antibiotic resistance gene(s) responsible for antibiotic-resistance of bacterial strain(s),
thereby selectively removing antibiotic resistance from an antibiotic resistant bacterial strain(s) in the microbiota of said subject or patient and preventing antibiotic resistance related infection in the subject or patient.

20. The method according to claim 18, wherein the DNA modifying enzyme is a nuclease.

21. The method according to claim 18, wherein the DNA modifying enzyme is a base editor.

22. The method according to claim 18, wherein the vector or recombinant phage further comprises a conditional origin of replication which is inactive in the targeted antibiotic resistant bacterial strain(s) but is active in a donor bacterial cell.

23. The method according to claim 18, wherein antibiotic resistance bacteria are removed from the gut of the healthy subject or patient.

24. The method according to claim 18, wherein the selective decolonization occurs before the subject or patient undergoes surgery and/or undergoes cancer treatment and/or immunosuppressive treatment.

25. The method according to claim 24, wherein the surgery is a solid organ transplant surgery of the kidney or liver.

26. The method according to claim 18, wherein said microbiota is a gut microbiota.

27. The method according to claim 18, wherein said antibiotic is methicillin, vancomycin, or a carbapenem class or beta-lactam class antibiotic.

28. The method according to claim 18, wherein the nuclease is a CRISPR associated nuclease.

29. The method according to claim 18, wherein the method further comprises obtaining a microbiota sample from the subject or patient and confirming the presence of said antibiotic resistant bacterial strain(s) in the sample.

30. The method according to claim 18, wherein at least two distinct vectors or recombinant phages are administered, preferably four distinct vectors or recombinant phages, each vector or recombinant phage targeting a different bacteria and/or encoding a DNA modifying enzyme targeting a different nucleic acid sequence inside the targeted bacteria.

31. The method according to claim 30, wherein said patient suffers from a hematological malignancy such as hematological cancer, and/or wherein said patient undergoes cancer treatment and/or immunosuppressive treatment.

32. The method according to claim 19, wherein antibiotic resistance bacteria are removed from the gut of the healthy subject or patient.

33. The method according to claim 19, wherein the selective decolonization occurs before the subject or patient undergoes surgery and/or undergoes cancer treatment and/or immunosuppressive treatment.

34. The method according to claim 19, wherein the nuclease is a CRISPR associated nuclease.

35. The method according to claim 19, wherein the method further comprises obtaining a microbiota sample from the subject or patient and confirming the presence of said antibiotic resistant bacterial strain(s) in the sample.

36. The method according to claim 19, wherein at least two distinct vectors or recombinant phages are administered, preferably four distinct vectors or recombinant phages, each vector or recombinant phage targeting a different bacteria and/or encoding a DNA modifying enzyme targeting a different nucleic acid sequence inside the targeted bacteria.

37. A method of preventing antibiotic resistant Escherichia coli related infection in a patient carrying antibiotic resistant Escherichia coli strain(s), wherein said method comprises administering a composition or combination comprising at least 4 recombinant phages,
wherein said recombinant phage comprises a nucleic acid encoding a DNA modifying enzyme inactivating the antibiotic resistance gene(s) responsible for antibiotic-resistance of E. coli strain(s),
thereby selectively removing antibiotic resistance from antibiotic resistant E. coli strain(s) in the microbiota of said patient and preventing antibiotic resistant E. coli related infection in the patient,
wherein said patient suffers from a hematological malignancy such as hematological cancer, and optionally undergoes cancer treatment and/or immunosuppressive treatment.


SEQUENCE LISTING (32 sequences)

SEQ ID NO: 1 - DNA, 282 bp, Artificial Sequence
  Primase ori deltaGAAABCC
tttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagcagcgccattactggcatggtggccagtaaggtagataaggtagacaaggggaggcacaactcaaaactttttaaacgagggggtaaaa

SEQ ID NO: 2 - DNA, 282 bp, Artificial Sequence
  Primase ori devoid of restriction sites
tttgttgcaatggctgtctaccctgtctacctgagtaaagaaaaatacatttaattcagtatattaacttgggtagacagcctttttttactgtctaccttctgtctaccctctctacctgattttacctgaatcagacagggaggtagacacggggtagacagtggataaaagcactctaccccactgaaagcagtgccattactggcatggttgccagtaaggttgataaggtagacaaggggagggacaactcaaaactttttaaacgagggggtaaaa

SEQ ID NO: 3 - DNA, 282 bp, Artificial Sequence
  primase ori from the PICI of the Escherichia coli strain CFT073
tttgttgcaatggctgtctaccctgtctacctgagtaaagaaaaatacatttaattcagtacattaacttgggtagacagcctttttttactgtctacctactatctaccctctctacctgattttacctgaatcagacagggaggtagatacggggtagatagtggataaaagcactctaccccactgaaagccgcgccattactggcatggtggccagtaaggtagataaggtagacaaggggaggcacaactcaaaactttttaaacgagggggtaaaa

SEQ ID NO: 4 - protein, 687 aa, Artificial Sequence
  STF29
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSETAAASSKNAAKTSETNAANSAQAAAASQTASANSATAAKKSETSAKNSETATKASEKNAKSSQTAAKTSETNAKDSEANAKVSETAAANSAKASAASQTAAKASEDAAREYANQTAEPYRYVLQPLPDVWIPFNDSLDMITGYSPGYKKVKIGDNVVQVASDKQVNFSRASTATYINKSGELKTAEINEPRFECDGLLIEGQRTNFFQNSTDPSKWNKSTSLDVTETGTDSFGFNYGRFVVQDSIVGTSKAHTIIGLYSSTGGVDTSGDEKHVTISCRVKSEVDNIAVRILFEHYDGEVRTSIGAANLNLTTRIISKTGQTSRVTARSVKDDATGWIFFEATLKADTTENTVGGFVQYSPDTGQMVTSGDYLDVTTPQIEAGTGASSFIVTGTAPATRASDMVTVPIKNNLYNLPFTVLCEVHKNWYKTPNVAPRVFDTGGHQTGAGIVMGFGSSGGYDGFPYCDIGGSDRRINENAGLEKMLIGMRVKSERSTCVVSNGKLSSETKTKWEYIRSTATIRIGGQTTAGLRHLFGHVRNFRLWHKELTDAQLGEVVE

SEQ ID NO: 5 - DNA, 2061 bp, Artificial Sequence
  STF29
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagaaacggcagcagcctcatcgaagaatgcggcgaaaacctcagaaacgaatgcagctaacagcgcacaggcggcagcggcctcgcagactgcatcggcaaactccgcgacagcagccaaaaaatcagaaaccagcgcgaaaaatagcgagacagccacaaaggccagcgaaaaaaacgcaaaatccagccagacggcagcgaaaaccagtgagacgaatgccaaagacagtgaagccaacgcaaaggtgagcgaaacagcggcggcgaactcggcgaaagcatcggcagcaagccagacggcagcaaaagcaagtgaagatgctgccagagaatacgcaaaccagacagcagagccgtacagatatgttttacagccgctgccggatgtgtggataccctttaatgattcgctggatatgattacgggctattctccgggttataaaaaagtgaagattggtgataatgtggttcaggttgccagtgataaacaggttaatttcagtcgcgcatcaacggcaacatatatcaacaaatctggcgaactgaaaacggcggaaattaatgagccgcgatttgagtgtgatggcctgcttattgagggacaaagaacgaacttcttccagaacagtacagacccttcgaagtggaataagtcaacttcactggacgttacagaaacaggcacagatagtttcgggtttaattatggtcggtttgtcgtacaggattcgattgttggtacaagtaaagcgcataccattatcggactgtattcgagtaccggaggggttgatacttcaggggacgaaaagcatgtaactatatcctgtcgggtaaaaagtgaagttgataatatcgccgttcgtattttatttgaacattatgatggggaggtaaggacatcaataggagcagcaaacctgaaccttaccacccgcataattagcaagacaggtcagacaagccgtgttacagcaaggtctgttaaggatgatgcaactggctggatattttttgaggctacattaaaagcagatacaacagaaaatacggttggtggttttgtccagtattctccggatacagggcagatggttacatcaggggattatctcgatgtaaccactccacagattgaggctggtacaggcgcatcatcttttattgttacggggacggcaccggcaacgcgggcaagcgatatggtgacagtcccaatcaagaataacctttataatcttccttttacggttctttgtgaggtacataagaactggtataaaacgccaaatgtagcgccgcgtgtttttgataccggcggtcatcaaaccggagcggggatcgtaatggggtttggttcatcaggtgggtacgacggttttccgtattgcgatataggtggttcagaccgacgaataaatgaaaatgccgggctggaaaaaatgcttattggtatgcgggtaaagtccgaacggtccacatgtgtagtcagtaacggtaagttaagcagcgaaactaaaaccaaatgggaatatatccggagtacagcaaccattcgcattggtggacaaactacagcaggattacgccatttatttgggcatgtgaggaattttcgtctctggcataaagagctaacagatgcgcagcttggggaggttgtggag

SEQ ID NO: 6 - protein, 92 aa, Artificial Sequence
  STF29 accessory protein
MRDFTLRFSDKADFRAFLRKLNWEEDEELQNAVLVDEIGFTFRETDVSDDGEPEYTRNEGYFVNIRLLDDGFEDSVFREWVVTPERPLREWF

SEQ ID NO: 7 - DNA, 276 bp, Artificial Sequence
  STF29 accessory protein-encoding nucleic acid
gtgagagatttcacgttgcgtttcagtgataaagcagatttcagggcatttctcaggaaacttaactgggaagaggacgaagagctgcagaatgccgttctggttgatgagattggttttacgttcagggagacagatgtttctgatgacggagaaccagaatacacgcgaaacgaagggtactttgttaatatccgtcttcttgacgatggatttgaggattccgtgttccgtgagtgggtggttacaccagagcgcccgctcagggagtggttt

SEQ ID NO: 8 - DNA, 13 bp, Artificial Sequence
  restriction site
  misc_feature (5)..(10): n is a, c, g, or t
twcannnnnntgg

SEQ ID NO: 9 - DNA, 12299 bp, Artificial Sequence
  nuclease-containing payload targeting bla-oxa- 48 gene
actgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatggcgtggttaaggatgaacaccaatcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaac

SEQ ID NO: 10 - protein, 1132 aa, Artificial Sequence
  A8
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGKITESHLGKELLEKVELTEDNASRLEEFSKEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGSVNANSGTLNNVTINENCQIKGKLSANQIEGDIVKTVSKSFPRTNSYASGTITVRISDDQKFDRQVMIPPVLFRGGKHENFNSNNQQSYWYSTCRLRVTRNGQEIFNQSTTDAQGVFSSVIDMPAGQGTLTLTFTVSSSGANNWTPTTSISDLLVVVMKKSTAGISIS

SEQ ID NO: 11 - DNA, 2475 bp, Artificial Sequence
  nucleic acid sequence encoding chimeric STF118
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggtggtaatggatgagactaatcgtaaagcgccattaaacagccctgcactgaccggaacgccaacgacgccaactgcgcgacagggaacgaataatactcagatcgcaaacacggctttcgttatggccgcgattgccgcccttgtagactcgtcgcctgacgcactgaatacgctgaacgagctggcagcggcgctgggcaacgacccgaattttgctaccactatgactaatgcgcttgcgggtaagcaaccgaaagatgctaccctggcggcgctggcggggcttgctactgcggcagacaggtttccgtattttacggggaatgatgttgccagtctggcaactctgacaaaagtcgggcgggatattcttgcgaaatcgaccgtttccgccgttatcgaatatctcggtttacaggaaacggtaaaccgagccgggaacgccgtgcaaaaaaatggcgataccttgtccggtggacttacttttgaaaacgactcaatccttgcctggattcgaaatactgactgggcgaagattggatttaaaaatgatgccgatggtgacactgattcatatatgtggtttgaaacaggtgacaacggcaatgaatacttcaaatggagaagtcgccagagcaccacaacaaaagacctgatgaatcttaaatgggatgctctgtatgttcttgttaaagcccttttcagcagtgaagtaaaaatatctacagtcaatgcactgaggatatttaattcatcttttggtgctatttttcgccgttctgaagaaaacctgtatatcatccctacacgagaaaatgagggtgaaaatggagatattgggccattaaggccattcggcatcaacttaagaacaggagttgtgtctgttggtaatggtgccaggattgatggcgggctggcacttggcacgaataacgcgttgggtgggaactctattgttcttggtgataacgacaccggatttaaacaaaatggcgatggtaatctggatgtttatgctaataacgtccatgttatgcgctttgtttccggaagcattcaaagtaataagaccataaatattacggggcgtgttaatccctcggattacggtaactttgattcccgctatgtgagagatatcagacttggcacacgtgttgtccagaccatgcagaaaggggtgatgtatgagaaagcagggcacgtaattaccgggcttggtattgtcggtgaagtcgatggtgatgaccccgcagtattcaggccaatacaaaaatacatcaatggcacatggtataacgtcgcacaggtgtaa

SEQ ID NO: 12 - protein, 824 aa, Artificial Sequence
  chimeric STF118
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKVVMDETNRKAPLNSPALTGTPTTPTARQGTNNTQIANTAFVMAAIAALVDSSPDALNTLNELAAALGNDPNFATTMTNALAGKQPKDATLAALAGLATAADRFPYFTGNDVASLATLTKVGRDILAKSTVSAVIEYLGLQETVNRAGNAVQKNGDTLSGGLTFENDSILAWIRNTDWAKIGFKNDADGDTDSYMWFETGDNGNEYFKWRSRQSTTTKDLMNLKWDALYVLVKALFSSEVKISTVNALRIFNSSFGAIFRRSEENLYIIPTRENEGENGDIGPLRPFGINLRTGVVSVGNGARIDGGLALGTNNALGGNSIVLGDNDTGFKQNGDGNLDVYANNVHVMRFVSGSIQSNKTINITGRVNPSDYGNFDSRYVRDIRLGTRVVQTMQKGVMYEKAGHVITGLGIVGEVDGDDPAVFRPIQKYINGTWYNVAQV

SEQ ID NO: 13 - DNA, 528 bp, Artificial Sequence
  nucleic acid sequence encoding accessory protein for STF118
atgcagcatttaaaaaatattactgcgggtaatccaaaaactgttgcccaatatcaactgacaaaaaattttgatgttatctggttatggtccgaagagggaaaaaactggtatgaggaagtaagtaattttcaggaagacacgataaagattgtttacgacgagaataatataattgtcggcatcaccagagatgcttcaacgcttaaccctgaaggtttcagcgttgtcgaggttcctgatattacctccaaccgacgtgctgatgactcaggtaaatggatgtttaaggatggtgccgtgattaagcggatttatacggcagatgaacaggagcaacaggcagaatcacaaaaggcagctttactttccgaagctgaatccgtgattttgccgctggaacgcgctgtcaggctgaatatggcgacggatgaggaacgcagccgactggaagcatgggaacgctacagcgttctggtcagtcgtgtggatcctgcaaatcccgaatggccggaaatgccgcaataa

SEQ ID NO: 14 - protein, 175 aa, Artificial Sequence
  accessory protein for STF118
MQHLKNITAGNPKTVAQYQLTKNFDVIWLWSEEGKNWYEEVSNFQEDTIKIVYDENNIIVGITRDASTLNPEGFSVVEVPDITSNRRADDSGKWMFKDGAVIKRIYTADEQEQQAESQKAALLSEAESVILPLERAVRLNMATDEERSRLEAWERYSVLVSRVDPANPEWPEMPQ

SEQ ID NO: 15 - DNA, 3291 bp, Artificial Sequence
  nucleic acid encoding chimeric STF SIEA11
atggcagtaaagatttcaggagtcctgaaagacggcacaggaaaaccggtacagaactgcaccattcagctgaaagccagacgtaacagcaccacggtggtggtgaacacggtgggctcagagaatccggatgaagccgggcgttacagcatggatgtggagtacggtcagtacagtgtcatcctgcaggttgacggttttccaccatcgcacgccgggaccatcaccgtgtatgaagattcacaaccggggacgctgaatgattttctctgtgccatgacggaggatgatgcccggccggaggtgctgcgtcgtcttgaactgatggtggaagaggtggcgcgtaacgcgtccgtggtggcacagagtacggcagacgcgaagaaatcagccggcgatgccagtgcatcagctgctcaggtcgcggcccttgtgactgatgcaactgactcagcacgcgccgccagcacgtccgccggacaggctgcatcgtcagctcaggaagcgtcctccggcgcagaagcggcatcagcaaaggccactgaagcggaaaaaagtgccgcagccgcagagtcctcaaaaaacgcggcggccaccagtgccggtgcggcgaaaacgtcagaaacgaatgctgcagcgtcacaacaatcagccgccacgtctgcctccaccgcggccacgaaagcgtcagaggccgccacttcagcacgagatgcggtggcctcaaaagaggcagcaaaatcatcagaaacgaacgcatcatcaagtgccggtcgtgcagcttcctcggcaacggcggcagaaaattctgccagggcggcaaaaacgtccgagacgaatgccaggtcatctgaaacagcagcggaacggagcgcctctgccgcggcagacgcaaaaacagcggcggcggggagtgcgtcaacggcatccacgaaggcgacagaggctgcgggaagtgcggtatcagcatcgcagagcaaaagtgcggcagaagcggcggcaatacgtgcaaaaaattcggcaaaacgtgcagaagatatagcttcagctgtcgcgcttgaggatgcggacacaacgagaaaggggatagtgcagctcagcagtgcaaccaacagcacgtctgaaacgcttgctgcaacgccaaaggcggttaaggcagcaaatgacaacgcaaattcacgtctggcgaaaaatcagaatggtgcagatatccaggataaatcagcttttctggacaatgttggcgttaccagcctgacgtttatgaaaaacaatggcgaaatgccggttgatgctgatctgaatacgtttggttctgttaaggcttattcaggtatctggtctaaagcaacgtccaccaacgcaacactggagaaaaacttccctgaagataatgctgtcggtgtgcttgaggtttttactggcggcaattttgcaggcacgcaacgctataccacacgtgacggaaatttgtatatccgcaaactcattggaacatggaatggtaatgatggaccatggggagcatggcgccatgttcaggctgtaacgcgagctctaagtacgaccattgaccttaactctctcggtggcgcagaacatttaggtctatggagaaacagcagttcagcaatagcttcttttgaacgacattaccccgagcagggaggagacgcgcagggcattctggaaattttcgaaggtgggctatatggacgcacacagcgttatacaacccgtaacgggactatgtatattcgcggcctgacagccaaatgggatgcagaaaatccacagtgggaagactggaaccaaattggttatcagaccagtagtaccttctatgaggatgacctggatgatttgatgtctccaggtatttacagtgtgacaggcaaagcgacccacaccccaatccaggggcagtctggttttctggaagtcatcaggcgcaaggatggtgtctatgttttgcaacgttacacgaccacaggaaccagcgcagctacaaaagaccgtttatatgagcgagtgtttcttggtggctcatttaacgcgtggggggagtggcgacagatttataactcaaactctttgccgttagagttgggtatcggtggcgcagtggcaaaactcaccagcctggactggcagacatacgattttgtgccgggcagtctgataaccgttcggctggataacatgaccaatattcccgacggtatggactggggcgtcattgatggcaacctgataaacatctcagtcggtccgagtgatgattctggttcgggacgctcaatgcatgtatggcgcagcactgtaagtaaagccaactaccgcttttttatggtgcgtatttcaggaaatccgggaagccgcacgatcacgacaagacgtgtgccaattatcgacgaagcccagacatggggcgcgaaacagacattcagtgctggcctttctggtgaactgtccggcaatgcggcgacagcaacaaagctgaaaacagcccgtaaaattaataacgtttcgtttgatggaacatcagatattaacctgacgccgaaaaatattggtgcatttgcttcaggaaaaacaggagacaccgttgcgaatgataaagccgttggatggaactggagtagcggagcctataacgcaactattggtggggcatcaacgttaattcttcattttaatatcggggaaggaagttgtcccgccgcccagtttcgcgttaattataagaacggtggtattttttatcgttctgctcgtgacggttacggattcgaggctgactggtctgagttttataccacaacgcgaaaacctacagcgggagatgtcggtgcactgccgttatctggtggtcaattgaatggtgctctgggtataggaacatccagtgctcttggcggtaattcgattgttttgggtgataatgacacgggctttaaacaaaatggtgatggtaatctggatgtttatgctaatagcgtccatgttatgcgctttgtctccggaagcgttcaaagtaataaaaccataaatattacggggcgtgttaatccctcggattacggtaactttgattcccgctatgtgagagatgtcagacttggcacacgtgttgtccagaccatgcagaaaggggtgatgtatgagaaagcagggcacgtaattaccgggcttggtattgtcggtgaagtcgatggtgatgaccccgcagtattcagaccaatacaaaaatacatcaatggcacatggtataacgtcgcacaggtgtaa

SEQ ID NO: 16 - protein, 1096 aa, Artificial Sequence
  chimeric STF SIEA11
MAVKISGVLKDGTGKPVQNCTIQLKARRNSTTVVVNTVGSENPDEAGRYSMDVEYGQYSVILQVDGFPPSHAGTITVYEDSQPGTLNDFLCAMTEDDARPEVLRRLELMVEEVARNASVVAQSTADAKKSAGDASASAAQVAALVTDATDSARAASTSAGQAASSAQEASSGAEAASAKATEAEKSAAAAESSKNAAATSAGAAKTSETNAAASQQSAATSASTAATKASEAATSARDAVASKEAAKSSETNASSSAGRAASSATAAENSARAAKTSETNARSSETAAERSASAAADAKTAAAGSASTASTKATEAAGSAVSASQSKSAAEAAAIRAKNSAKRAEDIASAVALEDADTTRKGIVQLSSATNSTSETLAATPKAVKAANDNANSRLAKNQNGADIQDKSAFLDNVGVTSLTFMKNNGEMPVDADLNTFGSVKAYSGIWSKATSTNATLEKNFPEDNAVGVLEVFTGGNFAGTQRYTTRDGNLYIRKLIGTWNGNDGPWGAWRHVQAVTRALSTTIDLNSLGGAEHLGLWRNSSSAIASFERHYPEQGGDAQGILEIFEGGLYGRTQRYTTRNGTMYIRGLTAKWDAENPQWEDWNQIGYQTSSTFYEDDLDDLMSPGIYSVTGKATHTPIQGQSGFLEVIRRKDGVYVLQRYTTTGTSAATKDRLYERVFLGGSFNAWGEWRQIYNSNSLPLELGIGGAVAKLTSLDWQTYDFVPGSLITVRLDNMTNIPDGMDWGVIDGNLINISVGPSDDSGSGRSMHVWRSTVSKANYRFFMVRISGNPGSRTITTRRVPIIDEAQTWGAKQTFSAGLSGELSGNAATATKLKTARKINNVSFDGTSDINLTPKNIGAFASGKTGDTVANDKAVGWNWSSGAYNATIGGASTLILHFNIGEGSCPAAQFRVNYKNGGIFYRSARDGYGFEADWSEFYTTTRKPTAGDVGALPLSGGQLNGALGIGTSSALGGNSIVLGDNDTGFKQNGDGNLDVYANSVHVMRFVSGSVQSNKTINITGRVNPSDYGNFDSRYVRDVRLGTRVVQTMQKGVMYEKAGHVITGLGIVGEVDGDDPAVFRPIQKYINGTWYNVAQV

SEQ ID NO: 17 - DNA, 528 bp, Artificial Sequence
  nucleic acid encoding accessory protein for STF SIEA11
atgcagcatttaaaaaatattactgcgggtaatccaaaaactgttgcccaatatcaactgacaaaaaattttgatgttatctggttatggtccgaagagggaaaaaactggtatgaggaagtaagtaattttcaggaagacacgataaagattgtttacgatgagaataatataattgtcggcatcaccagagatgcttcaacgctcaaccctgaaggttttagcgttgtcgaggttcctgatattaccgccaaccgacgtgctgatgactcaggtaaatggatgtttaaggatggtgccgtgattaagcggatttatacggcagacgaacagctgcaactggcggaattacagaagtcagctttgctttccgaagctgaaactatcattcagccactggaacgctctgtcagactgaatatggcaacagatgaggagcgtagccgactggaagcatgggaacgctacagtgttctggtcagccgtgtggatcctgcaaatcctgaatggccggaaatgccgcaataa

SEQ ID NO: 18 - protein, 175 aa, Artificial Sequence
  accessory protein for STF SIEA11
MQHLKNITAGNPKTVAQYQLTKNFDVIWLWSEEGKNWYEEVSNFQEDTIKIVYDENNIIVGITRDASTLNPEGFSVVEVPDITANRRADDSGKWMFKDGAVIKRIYTADEQLQLAELQKSALLSEAETIIQPLERSVRLNMATDEERSRLEAWERYSVLVSRVDPANPEWPEMPQ

SEQ ID NO: 19 - DNA, 747 bp, Artificial Sequence
  bla-oxa-48  gene
atgcctgcggtagcaaaggaatggcaagaaaacaaaagttggaatgctcactttactgaacataaatcacagggcgtagttgtgctctggaatgagaataagcagcaaggatttaccaataatcttaaacgggcgaaccaagcatttttacccgcatctacctttaaaattcccaatagcttgatcgccctcgatttgggcgtggttaaggatgaacaccaagtctttaagtgggatggacagacgcgcgatatcgccacttggaatcgcgatcataatctaatcaccgcgatgaaatattcagttgtgcctgtttatcaagaatttgcccgccaaattggcgaggcacgtatgagcaagatgctacatgctttcgattatggtaatgaggacatttcgggcaatgtagacagtttctggctcgacggtggtattcgaatttcggccacggagcaaatcagctttttaagaaagctgtatcacaataagttacacgtatcggagcgcagccagcgtattgtcaaacaagccatgctgaccgaagccaatggtgactatattattcgggctaaaactggatactcgactagaatcgaacctaagattggctggtgggtcggttgggttgaacttgatgataatgtgtggttttttgcgatgaatatggatatgcccacatcggatggtttagggctgcgccaagccatcacaaaagaagtgctcaaacaggaaaaaattattccctag

SEQ ID NO: 20 - protein, 584 aa, Artificial Sequence
  PICI primase-helicase
MKLAPNVKQQSRGIKHKETEVIIFAGSDAWSHAKQWQEHDARMAGDNEPPVWLGEQQLSELDKLQIVPEGRKSVRIFRAGYLAPVMIKAIGQKLAAAGVQDANFYPDGMHGQKVENWREYLARERQNLSDGLVIELPVKQKAQLSQMADSERAQLLADRFDGVCVHPESEIVHVWCGGVWCPVSTMELSREMVAIYSEHRATFSKRVINNAVEALKVIAEPMGEPSGDLLPFANGALDLKTGEFSPHTPENWITTHNGIEYTPPAPGENIRDNAPNFHKWLEHAAGKDPRKMMRICAALYMIMANRYDWQMFIEATGDGGSGKSTFTHIASLLAGKQNTVSAEMTSLDDAGGRAQVVGSRLIVLADQPKYTGEGTGIKKITGGDPVEINPKYEKRFTAVIRAVVLATNNNPMIFTERAGGVARRRVIFRFDNIVSEAEKDRELPEKIAAEIPVIIRRLLANFADPEKARALLIEQRDGDEALAIKQQTDPVIEFCQFLNFLEEARGLMMGGGGDSVKYTTRNSLYRVYLAFMAYAGRSKPLNVNDFGKAMKPAAKVYGHEYITRKVKGVTQTNAITTDDCDAFL

SEQ ID NO: 21 - DNA, 1752 bp, Artificial Sequence
  PICI primase-helicase
atgaaactggcaccgaacgtaaaacagcagtcacgcggcataaaacacaaagaaacagaagtcattatttttgcgggtagtgatgcctggtcacacgcaaaacaatggcaggaacatgacgcgcgtatggccggagataatgagcctcctgtgtggcttggggagcagcagttatccgaactggataagctgcaaattgtgccggaaggcagaaaatccgtgcgcatattcagggccggatatcttgcgccagtaatgataaaggcgattggtcagaagctggcggcggcaggcgtacaggatgcaaatttttaccctgatggtatgcacggtcagaaggtggagaactggcgcgaatatctggcccgtgagcgccagaatctttctgatggtctggtcattgagcttccggtaaagcaaaaggcgcaactttcgcagatggcggacagtgagcgcgcgcagctgcttgccgatcgctttgatggcgtttgcgtacatcctgaaagtgaaatcgttcacgtatggtgcggcggggtatggtgtccggtcagcacaatggagctgagccgcgaaatggtggcgatctattcagagcacagggccactttcagcaagcgcgtaatcaataacgccgtggaagcgttaaaagttattgccgaaccaatgggcgagccgtccggcgatttgctgccgttcgccaatggtgcgcttgacctgaaaacgggggaattttccccgcacacgccggagaactggatcaccacgcacaacggcattgagtacacgccaccagcacccggggagaacatccgcgataacgcgccaaactttcataaatggcttgagcacgcagccggaaaagacccgcgcaagatgatgcgtatatgtgccgcgctgtacatgattatggcgaaccggtacgactggcagatgtttattgaggccaccggagacggcgggagcggtaaaagtacattcacacacatagccagccttctggcagggaaacaaaacacggtaagcgctgaaatgacatcgcttgatgatgctggtgggcgtgcgcaggttgtcgggagtcgtcttatcgtcctggcagaccagccgaaatatacaggcgaaggaacgggcatcaagaaaatcacgggcggcgaccccgtggaaattaacccgaaatatgaaaagcgttttacggcggtaatcagggcggtggtgctggcaaccaataacaatccgatgatattcaccgaacgggccggaggtgtggcacgtcgtcgggtgatattccggttcgataacatcgtaagcgaggcagaaaaagacagggagctaccggaaaagatcgcggctgaaatccctgtcattatccgccgcttgctggcgaactttgccgaccctgaaaaggcacgggctttactcattgaacagcgtgacggtgatgaagcactggcaataaagcaacagacggatccggttattgagttttgccagttcctgaattttctggaggaagcacgcggcctgatgatgggcggcggtggcgattcagtgaagtacacgaccagaaacagcctttaccgcgtctatctggcgtttatggcgtacgcaggcaggagcaaaccgctaaacgtaaatgactttggcaaggctatgaagccagccgcgaaagtttacggacatgaatatattacgcggaaagttaaaggagtaacgcagactaacgcaataacaacagacgattgcgacgcgttttta

SEQ ID NO: 22 - DNA, 3393 bp, Artificial Sequence
  nucleic acid encoding 1A2 gpJ
atgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacccgaacaacggctgggagacggcgacagagcttgttgaagatacgcaggccattgcccgttacggtcgtaatgttacgaagatggatgcctttggctgtaccagccgggggcaggcacaccgcgccgggctgtggctgattaaaacagaactgctggaaacgcagaccgtggatttcagcgtcggcgcagaagggcttcgccatgtaccgggcgatgttattgaaatctgcgatgatgactatgccggtatcagcaccggtggtcgtgtgctggcggtgaacagccagacccggacgctgacgctcgaccgtgaaatcacgctgccatcctccggtaccgcgctgataagcctggttgacggaagtggcaatccggtcagcgtggaggttcagtccgtcaccgacggcgtgaaggtaaaagtgagccgtgttcctgacggtgttgctgaatacagcgtatgggagctgaagctgccgacgctgcgccagcgactgttccgctgcgtgagtatccgtgagaacgacgacggcacgtatgccatcaccgccgtgcagcatgtgccggaaaaagaggccatcgtggataacggggcgcactttgacggcgaacagagtggcacggtgaatggtgtcacgccgccagcggtgcagcacctgaccgcagaagtcactgcagacagcggggaatatcaggtgctggcgcgatgggacacaccgaaggtggtgaagggcgtgagtttcctgctccgtctgaccgtaacagcggacgacggcagtgagcggctggtcagcacggcccggacgacggaaaccacataccgcttcacgcaactggcgctggggaactacaggctgacagtccgggcggtaaatgcgtgggggcagcagggcgatccggcgtcggtatcgttccggattgccgcaccggcagcaccgtcgaggattgagctgacgccgggctattttcagataaccgccacgccgcatcttgccgtttatgacccgacggtacagtttgagttctggttctcggaaaagcagattgcggatatcagacaggttgaaaccagcacgcgttatcttggtacggcgctgtactggatagccgccagtatcaatatcaaaccgggccatgattattacttttatatccgcagtgtgaacaccgttggcaaatcggcattcgtggaggccgtcggtcgggcgagcgatgatgcggaaggttacctggattttttcaaaggcaagataaccgaatcccatctcggcaaggagctgctggaaaaagtcgagctgacggaggataacgccagcagactggaggagttttcgaaagagtggaaggatgccagtgataagtggaatgccatgtgggctgtcaaaattgagcagaccaaagacggcaaacattatgtcgcgggtattggcctcagcatggaggacacggaggaaggcaaactgagccagtttctggttgccgccaatcgtatcgcatttattgacccggcaaacgggaatgaaacgccgatgtttgtggcgcagggcaaccagatattcatgaacgacgtgttcctgaagcgcctgacggcccccaccattaccagcggcggcaatcctccggccttttccctgacaccggacggaaagctgaccgctaaaaatgcggatatcagcggtaacgtgaatgcgaactccgggacgctcaacaacgtcacgattaacgagaactgtcgggttctgggaaaattgtccgcgaaccagattgaaggcgatctcgttaaaacagtgggcaaagctttcccccgggactcccgtgcaccggagcggtggccatcaggaaccattaccgtcagggtttatgacgatcagccgtttgaccggcagattgttattccggcggtggcattcagcggcgctaaacatgagaaagagcatactgatatttactcctcatgccgtctgatagtgcggaaaaacggtgctgaaatttataaccgtaccgcgctggataatacgctgatttacagtggcgttattgatatgcctgccggtcacggtcacatgacactggagttttcggtgtcagcatggctggtaaataactggtatcccacagcaagtatcagcgatttgctggttgtggtgatgaagaaagccactgcaggcatcacgattagc

SEQ ID NO: 23 - protein, 1131 aa, Artificial Sequence
  1A2 gpJ
MGKGSSKGHTPREAKDNLKSTQLLSVIDAISEGPIEGPVDGLKSVLLNSTPVLDTEGNTNISGVTVVFRAGEQEQTPPEGFESSGSETVLGTEVKYDTPITRTITSANIDRLRFTFGVQALVETTSKGDRNPSEVRLLVQIQRNGGWVTEKDITIKGKTTSQYLASVVMGNLPPRPFNIRMRRMTPDSTTDQLQNKTLWSSYTEIIDVKQCYPNTALVGVQVDSEQFGSQQVSRNYHLRGRILQVPSNYNPQTRQYSGIWDGTFKPAYSNNMAWCLWDMLTHPRYGMGKRLGAADVDKWALYVIGQYCDQSVPDGFGGTEPRITCNAYLTTQRKAWDVLSDFCSAMRCMPVWNGQTLTFVQDRPSDKTWTYNRSNVVMPDDGAPFRYSFSALKDRHNAVEVNWIDPNNGWETATELVEDTQAIARYGRNVTKMDAFGCTSRGQAHRAGLWLIKTELLETQTVDFSVGAEGLRHVPGDVIEICDDDYAGISTGGRVLAVNSQTRTLTLDREITLPSSGTALISLVDGSGNPVSVEVQSVTDGVKVKVSRVPDGVAEYSVWELKLPTLRQRLFRCVSIRENDDGTYAITAVQHVPEKEAIVDNGAHFDGEQSGTVNGVTPPAVQHLTAEVTADSGEYQVLARWDTPKVVKGVSFLLRLTVTADDGSERLVSTARTTETTYRFTQLALGNYRLTVRAVNAWGQQGDPASVSFRIAAPAAPSRIELTPGYFQITATPHLAVYDPTVQFEFWFSEKQIADIRQVETSTRYLGTALYWIAASINIKPGHDYYFYIRSVNTVGKSAFVEAVGRASDDAEGYLDFFKGKITESHLGKELLEKVELTEDNASRLEEFSKEWKDASDKWNAMWAVKIEQTKDGKHYVAGIGLSMEDTEEGKLSQFLVAANRIAFIDPANGNETPMFVAQGNQIFMNDVFLKRLTAPTITSGGNPPAFSLTPDGKLTAKNADISGNVNANSGTLNNVTINENCRVLGKLSANQIEGDLVKTVGKAFPRDSRAPERWPSGTITVRVYDDQPFDRQIVIPAVAFSGAKHEKEHTDIYSSCRLIVRKNGAEIYNRTALDNTLIYSGVIDMPAGHGHMTLEFSVSAWLVNNWYPTASISDLLVVVMKKATAGITIS

SEQ ID NO: 24 - protein, 687 aa, Artificial Sequence; identical to SEQ ID NO: 4
  STF27

SEQ ID NO: 25 - protein, 92 aa, Artificial Sequence
  STF27 accessory protein
VRDFTLRFSDKADFRAFLRKLNWEEDEELQNAVLVDEIGFTFRETDVSDDGEPEYTRNEGYFVNIRLLDDGFEDSVFREWVVTPERPLREWF

SEQ ID NO: 26 - DNA, 2061 bp, Artificial Sequence; identical to SEQ ID NO: 5
  nucleic acid encoding STF27

SEQ ID NO: 27 - DNA, 276 bp, Artificial Sequence; identical to SEQ ID NO: 7
  nucleic acid encoding STF27 accessory protein

SEQ ID NO: 28 - DNA, 24 bp, Artificial Sequence
  CTX1 crRNA
gccagatcaccgcgatatcgttgg

SEQ ID NO: 29 - DNA, 12299 bp, Artificial Sequence
  p775 payload
actgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatgccagatcaccgcgatatcgttggtcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaac

SEQ ID NO: 30 - DNA, 24 bp, Artificial Sequence
  CTX3 crRNA
tcgcggtgatgaacgctttccaat

SEQ ID NO: 31 - DNA, 12299 bp, Artificial Sequence
  p776 payload
actgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagattcgcggtgatgaacgctttccaattcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaac

SEQ ID NO: 32 - DNA, 12342 bp, Artificial Sequence
  p844 payload
actgtcggaatgacaaatggttccaattattgaacacccttcggggtgtttttttgtttctggtttcccgaggccggcctgcgctagcggagtgtatactggcttactatgttggcactgatgagggtgtaagtgaagtgcttcatgtggcaggagaaaaaaggctgcatcggtgcgtcagcagaatatgtgatacaggatatattccgcttcctcgctcactgactcgctacgctcggtcgttcgactgtggcgagcggaaatggcttacgaacggggcggagatttcctggaagatgccaggaagatacttaacagggaagtgagagggtcgcggcaaagccgtttttccataggctccgcccccctgacaagcatcacgaaatctgacgctcaaatcagtggtggcgaaacctgacaggactataaagataccaggcgtttccccctggcggctccctcgtgcgctctcctgttcctgcctttcggtttgccggtgtcattcctctgttacggccgagtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggataaggctaaactgaaaggacaagttttggcgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcaccggcgcgccgtgaccaattattgaaggccgctaacgcggcctttttttgtttctggtatcccgaatggagcgacttctccccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtaaagggtaagtatgaaggtcgtgtactccatcgctaccaaattccagaaaacagacgctttcgagcgtcttttttcgttttggtcacgacgtacggtggaagattcgttaccaattgacagctagctcagtcctaggtatatacatacatgcttgtttgtttgtaaactactgttttcattaaagaggagaaaggaagccatgtccatctatcaggagtttgttaacaagtattccctgtctaaaaccctgcgttttgaactgatcccgcagggcaaaactttggaaaacattaaagcgcgtggcctgattctggatgacgaaaaacgtgcaaaggattacaagaaagctaaacagatcatcgacaaatatcaccagttctttatcgaagaaattctgtcgtcggtgtgcatcagtgaggatctgttacagaattattctgatgtatactttaaacttaaaaagtccgatgacgataatctgcaaaaagatttcaagtcagccaaagataccatcaagaaacagatctcagaatatattaaagatagcgaaaagttcaaaaacctgtttaaccaaaacctcattgatgctaagaaaggccaagaatctgacctgatcttatggctgaaacagagcaaagataacggcattgaactgttcaaagctaatagcgacatcaccgatattgatgaagcgctcgaaatcatcaagtctttcaaaggctggacgacgtatttcaaaggttttcatgaaaaccgtaagaatgtatattcgagcaacgatattccgacctctattatttatcgtatcgtggacgacaacctgccgaagtttctggaaaacaaagcgaaatatgaatctctgaaagacaaagcaccggaagctattaactatgaacagatcaagaaagatctggcggaagaactgaccttcgacatcgactataaaacctccgaagttaaccagcgtgttttctcactggacgaggttttcgaaatcgctaatttcaacaattacctgaatcaatctggcatcaccaaattcaacaccattattggtggcaaatttgttaacggcgaaaacaccaagcgtaagggcatcaacgaatacattaacctgtatagccaacaaatcaacgacaaaaccctgaaaaagtataaaatgtccgttctgtttaaacagattttatcggacaccgaatctaaatccttcgtaattgataaactggaagatgatagcgacgttgtcaccacgatgcagagcttttatgagcagattgcggcgttcaaaaccgtggaagagaaatctattaaagaaactctgtccctgctctttgacgacctcaaagcgcagaaactagatctgtctaagatttactttaaaaacgacaaatctctgaccgatctcagtcaacaagttttcgatgactatagcgtgatcggcacggcagttttggaatacatcacccaacaaatcgcgccgaaaaatctggacaacccgtccaagaaggaacaggaactgattgcaaagaaaacagaaaaagctaaatacctgagcttagaaactatcaaactggcacttgaggaatttaataaacatcgtgatattgataaacagtgtcgttttgaggaaattctggcgaactttgcggcaatcccgatgatcttcgacgaaattgctcaaaacaaagacaatctggcgcagatctctatcaagtaccagaatcagggtaagaaagatctgcttcaagcatctgcggaggacgatgtgaaagcaattaaagacttattagatcagacgaataacttattacacaagctcaaaatcttccacatcagccagagcgaggacaaggcgaacattctggataaagatgaacacttctatctggtgttcgaagaatgttacttcgaactggcaaacatcgtccctctctacaataaaatccgcaactacatcacgcagaagccttactctgacgagaaattcaaactgaacttcgaaaacagcacgctggcgaacggctgggataagaacaaagagccggacaacaccgcaatcctgttcatcaaagacgacaaatactatctgggcgtaatgaacaagaagaacaacaagatcttcgacgataaagcgatcaaagaaaacaagggtgaaggctataagaaaatcgtgtacaagctcctgccgggtgcgaataaaatgttaccgaaagtgttcttttccgcgaaaagcatcaaattctacaacccgtctgaggatattctgcgcatccgcaatcatagcacgcacactaaaaacggtagcccgcagaaagggtatgaaaaattcgaatttaatatagaggactgccgtaagttcatcgacttctataaacagagcatttccaaacatccggaatggaaagacttcggcttccgtttctctgacactcagcgctataatagcatcgacgagttctaccgcgaagtggagaatcagggctataaactgaccttcgagaacattagtgagtcgtacatcgactccgttgtgaatcagggtaaactgtacctgtttcagatctataataaagactttagcgcgtacagcaaaggccgtccgaatctgcacaccctttactggaaagcattatttgacgaacgtaacctgcaagatgtggtgtataaactgaacggtgaggcggaacttttctaccgtaaacagagtatcccgaagaaaatcacgcatccggcaaaagaagctattgccaacaaaaacaaagacaacccgaagaaagaatcagtattcgaatatgacctgatcaaagataaacgtttcaccgaagataagttctttttccactgtccgattaccatcaacttcaaatctagcggtgcgaacaagttcaacgatgaaattaacttattactgaaagagaaagctaatgacgtacacatcttatctattgatcgcggtgaacgtcatttagcatactatacactggtagatggtaaaggtaatattattaaacaggatactttcaatattatcggtaatgaccgtatgaaaaccaactatcacgataagctggcggcgatcgaaaaagatcgtgattctgcgcgtaaagattggaagaaaattaacaatatcaaagaaatgaaagaaggctatctgagccaagtggtgcacgagatcgcaaaactggtgattgaatataacgctatcgtggttttcgaagatctgaactttggttttaaacgtggtcgcttcaaagtagaaaaacaggtgtaccaaaaactggaaaaaatgctgattgaaaaactgaactatctggtttttaaagacaacgaatttgacaaaacgggtggcgtactccgtgcctatcagctgaccgctccgttcgaaacgttcaagaaaatgggtaaacaaacggggattatctattatgtgccagctggtttcacctccaagatttgtccagttacgggcttcgttaaccagctgtacccgaaatacgagagcgttagcaaatctcaagaatttttcagcaaattcgacaagatctgctataatctggataaaggctatttcgagttcagcttcgattacaaaaacttcggcgataaagcggctaaaggtaagtggactattgctagctttggtagccgtctgattaactttcgcaactccgacaaaaaccataattgggacacgcgtgaagtgtatccgaccaaagaactggaaaaattactgaaagactattccatcgaatatggtcatggggagtgcattaaagcggcgatttgcggtgaatccgataagaaatttttcgccaaactgaccagcgtgcttaacaccattctgcaaatgcgtaattctaaaacgggtacggagctggactacctgatttctccggtagccgacgttaacggcaacttcttcgattctcgtcaagcaccgaaaaatatgccacaagacgcggatgccaacggtgcataccatatcggcttaaaaggcttaatgttattaggccgtatcaagaataatcaggagggcaagaaattaaatctggttatcaaaaacgaagaatacttcgagttcgttcagaatcgtaacaattaatgtatgcttaagcagctcggtaccaaagacgaacaataagacgctgaaaagcgtcttttttcgttttggtcctgttgcggcgcgatagtgtgaacatgctatagacttctggtgctacccgactgacaattaatcatccggctcgtataatgctagcaatttctactgttgtagatgccagatcaccgcgatatcgttggaatttctactgttgtagattcgcggtgatgaacgctttccaattcgagacgaacaataaggcctccctaacggggggccttttttattgataacaaaagtaacttcgagcttgtctacctcctagcaccattattgcaattaataaacaactaacggacaattctacctaacagttttcatatatgacgagcagttaagtgatgagtaaaggtgaggaattatttactggtgttgttccgatcttagttgaactggacggcgatgttaacggtcataaattcagtgttcgtggtgaaggtgaaggtgatgcaaccaacggtaagctgaccctgaaattcatctgcactactggaaaattaccagtaccgtggcctactctggtgactaccctgacctatggtgttcagtgtttttctcgttaccctgaccacatgaagcaacatgatttcttcaaatctgcaatgccggaaggttatgtacaggagcgcaccatttctttcaaagacgatggcacgtataaaacccgtgcagaggttaaatttgaaggtgacactctggtgaatcgtattgaactgaaaggcattgatttcaaagaggacggcaatattttaggccacaaactggaatataacttcaactcccataacgtttacatcaccgcagacaaacagaagaacggtatcaaagctaacttcaaaattcgccataacgttgaagatggtagcgtacagctggcggatcattaccaacagaacactccgattggagatgctcctgttttactgccggataaccactacctgtccacccagtctaaactgtcgaaggatccgaacgaaaagcgcgaccacatggtgttattagagttcgttaccgctagtggtatcacgcacggtatggatgaactctacaaataagacgaacaataaggggagcgggaaaccgctccccttttttattgataacaaaagtaaattgcacgctgatagtctcccaattgcgaaggaccaaaacgaaaaaacaccctttcgggtgtcttttctggaatttggtaccgagtactaggtatcgtgtaagtagcgaaggcccgtacgcgagataaactgctaggcaaccgcgactctacgactggtgctcgatttaatttcgctgacgtaaagaaattatcggcagtgcgtcaactgccgtatctttatcttaattaggtagttggacaagcccttgaaagaaatagcaagagcctgcctctctattgaagtcacggcgaaagtcgggtagaaatcaaagaaagcagaaattaaatcggagtaacactaaggtgggataactccgtaactgactacgcctttctctagactttacttgaccagatacactgtctttgacacgttgaaggattagagcaatcaaatccaagactggctaagcacgaagcaactcttgagtgttaaaaagttatctcctgtattcgggaagcgggtactagaagattgcagggactccgacgttaagtaaattacaaagtaataagtatcgttcaggatcacgttaccgcaataagaagcgagaataatataatttccgaagtgcttaccccagtagtgactattcctataacccttctgagtgtccggaggcggaaatttgccacgaaagagaaagtatttccccgacaataataaaggggcgctcctcagcttttccacttggttgggtaagctaggcaactctgaaaggagtttcggcgaattgaagccgacagctttgaattgttttaggggcgttattcgagggcaatcggagctaacttcaagactacttctttgttgaatactaaatagtgcaaaggtcgtgtttcctcaaggatactccgctaacaatataggattccaatcagattcagcactggcggtacgggtgttgcggtgaggcgttcgggtttacggctcgaagctagcacggtaggaagcctgacaatcaccaagcaaaagggccgtcgaaggcccacaagatacgaaagctctcgaagccttatccttgaccgatccacctatttaggcagttacgcacaaaagctacccaataatccgtgacaggcacaatatcacggaacaaaaccgaaaactctcgtacacggttaggttttcgctaggaagaataaacctctatcttgattataagaaggctccccaagcacccccaaaaccgaaatagcggtttgcaataagggacaagttacgagtgtagacacgcagaattatccagcctttagtctttaggaaggcaaagctattgtacgcggtagccgtcgtagcaatttaccaactgtagaattattggacacacgtaggaagggcttacagttgaagtttaataaggtcacacgcaaaaccgctaaggaataatcgcaccgttagcgaaagaatatttcagagcggttagtaaaggttgagtaaagtgagattccaaagtgagcctttataaaaagtaaagagctataataaaaccgtcgagcagaaaacaatcgcctgaaatctcaagcacgttgccctttctaacgtcgctaaggtttcgtaaacccgtttgattaggaagaagaataagtaacccgattaggtttgagatcgcgggttatcggtttggattaaaagtggataccagcggagtcaacgccgacgcaaacgtacagtgatccaatcctgttgcacggtcaagcacaatcagctcgcaagatcttggaatagtgtgcccaacagtttagttgagggccacgttccgactacaagttgcttcaagaggggaatttggatttggcaatagccccccgtttctacctcaagaggcgacgagtattaaccgcgccagctgtcggcacaagggccaaagaagattccaatttcttattcccgaataacctccgaatccctgcgggaaaatcaccgaccgaatagcctagaagcaagggggaacagataggtataattagcttaagagagtaccagccgtgacaacagcgtagtaaccacaaacttacgctggggcttctttggcggatttttacagatactaacaaggtgatttgaagtaccttagttgaggatttaaacgcgctatccggtaatctccaaattgggaaataccgttcaaagagggctagaattacttaaaagccttcacaccgcctgcgctatacgcgcccactctcccgtttatccgtccaagcggaagcagggcgatcctccgctaagatattcttacgtgtaacgtagctaagtatcccaaatagctggcgtacgcgttgaacaccgcctagaggatcgtgactcgccggacgagcgtgttattggggacttacgccagcgtagactacaacgcgcccagattaaccctgcacgtattgccttgaataacgtactaatctctccggctctcgacaatctatcgagcgactcgattatcaacgggtgtcttgcagttctaatctcttgcccccgcccgtaatagcctccaagagattgaagatagtaaagggcaagagctgattcggcgttgaaggatagcggactttcggtcaaccacaattccccactcgacaaaaccagccgtgcgaataactctgaaagtacaagcaacccaagagggctgagcctaaactcagctaattcctaagtgagctaaagactcgaagtgacagctcttaataaatagagcgggaacgtcgaacggtcgtgaaagtaatagtacaacgggtattaacttactgaggatattgcttgaagctgtaccgttttattgggtgaacgaataagatccagcaattcagccaaagaagctaccaatttttagtttaagagtgtcacgtctgacctcgcgggtagattgccgaacgtagagcttacgagccagcggaaacagtagccgcaggataagtaaggggagtaagtgatcgaacgaatcagaagtgacaatatacttaggctggatctcgtcccgtgaatcccaaccctcaccaactacgagataagaggtaagccaaaaatcgacttggtggcgaccaacgactgttccccccctgtaactaatcgttccgtcaaaacctgacttacttcaaggccaattccaagcgcaaacaataccgtcctagttcttcggttaagtttccgaagtaggagtgagcctacctccgtttgcgtcttgttaccactgacccagctatttactttgtattgcctgcaatcgaatttctgaactctcagatagtggggataacgggaaagttcctatatttgcgaactaacttagccgtccacctcgaagctacctactcacacccaccccgcgcggggtaaataaggcactaatcccagctgagagctggcgtagcacttagccacaagttaattaacagttgtctggtagtttggcggtattaggaagatcctagaagcaaggcagagttagttctaacctaaagccacaaataagacaggttgccaaagcccgccggaaattaaatcttgctcagttcggtaacggagtttccctcccgcgtacttaattcccaataagaaacgcgcccaagtcctatcaggcaaaattcagccccttcccgtgttagaacgagggtaaaaatacaagccgattgaacaagggttgggggcttcaaatcgtcgtttaccccactttacaacggagattaagtagttcaccctatagtacgaagcagaactatttcgaggggcgtgcaataatcgaatcttctgcggttgacttaacacgctagggacgtgccctcgattcaatcgaaggtactcctactcagactgcctcacacccagctagtcactgagcgataaaattgacccgccctctagggaagcgagtacgtcccaaagggctccggacagggctatataggagagtttgatctcgccccgacaactgcaaccctcaactcccttagataatattgttagccgaagttgcacgacccgccgtccacggactgctcttagggtgtggctccttaatctgacaacgtgcaacccctatcgaagtcgattgtttctgcgaaaggtgttgtcctaatagtcccgaaatttggcccttgtaggtgtgaaaccacttagcttcgcgccgtagtcctaaaggcccacctattgactttgtttcgggtagcactaggaatcttaacaatttgaatttggacgtggaacgcgtacaccttaatctccgaataattctagggatttggaagtcctctacgttgacacacctacactgctcgaagtaaatatacgaataacgcgggcctcgcggagccgttccgaatcgtcacgtgttcgtttactgttaattggtggcaaataagcaatatcgtagtccgtcaggcccagccctgttatccacggcgttatttgtcaaattgcgtagaactggattgactgcctgacaatacctaattatcggtacgaagtccccgaatctgtcgggctatttcactaatactttccaaacgccccgtatccaagaagaacgaatttatccacgctcccgtctttgggacgaataccgctacaagtggacagaggatcggtacgggcctctaataaatccaacactctacgccctcttcaagagctagaagaacagggtgcagttggaaagggaattatttcgtaaggcgagccaataccgtaattaattcggaagagttaacacgattggaagtaggaatagtttctaaccacggttactaatcctaataacggaacgctgtctgatagattagtgtcagcgctcggtaccaaagaaaaataaaaagacgctgaaaagcgtctttttatttttcggtccagtgtaactcaggcaaaagcacgtaatattcgtactttcttcctccgtaagcgtcacccacattccttaaagagtgcatgtgcatattttgttatcaataaaaaaggccgcgatttgcggccttattgttcgtcttgccggattacgccccgccctgccactcatcgcagtattgttgtaattcattaagcattctgccgacatggaagccatcacaaacggcatgatgaacttggatcgccagtggcattaacaccttgtcgccttgcgtataatattttcccatagtgaaaacgggggcgaagaagttgtccatatttgctacgtttaaatcaaaactggtgaaactcacccacggattggcactgacgaaaaacatattttcgataaaccctttagggaaatatgctaagttttcaccgtaacacgccacatcttgactatatatgtgtagaaactgccggaaatcgtcgtggtattctgaccagagcgatgaaaacgtttcagtttgctcatggaaaacggtgtaacaagggtgaacactatcccatatcaccagctcaccgtctttcattgccatacgaaactccggatgtgcattcatcaggcgggcaagaatgtgaataaaggccggataaaacttgtgcttatttttctttacggtttttaaaaaggccgtaatatccagctgaacggtttggttataggtgcactgagcaactgactggaatgcctcaaaatgttctttacgatgccattgacttatatcaactgtagtatatccagtgatttttttctccattttagcttccttagcttgcgaaatctcgataactcaaaaaatagtagtgatcttatttcattatggtgaaagttgtcttacgtgcaacattttcgcaaaaagttggcgctttatcaac